(19) United States
(12) Patent Application Publication
(10) Pub. No.: US 2023/0200224 A1
(43) Pub. Date: Jun. 22, 2023

(54) NITROGEN-CONTAINING COMPOUND, AND ELECTRONIC ELEMENT AND ELECTRONIC DEVICE HAVING SAME

(71) Applicant: SHAANXI LIGHTE OPTOELECTRONICS MATERIAL CO., LTD., Xi'an (CN)

(72) Inventors: Linnan MA, Xi'an (CN); Peng NAN, Xi'an (CN); Youngkook KIM, Xi'an (CN); Yingwen LI, Xi'an (CN)

(21) Appl. No.: 18/003,917

(22) PCT Filed: Oct. 26, 2021
(86) PCT No.: PCT/CN2021/126432

§ 371 Date: Dec. 30, 2022

(30) Foreign Application Priority Data
Oct. 30, 2020 (CN) 202011195453.6
Dec. 2, 2020 (CN) 202011404791.6

(51) Int. Cl. H10K 85/60; C07D 307/77; C07D 307/91; C07D 409/12; C07D 333/50; H10K 50/15

(52) U.S. Cl. CPC H10K 85/636; C07D 307/77; C07D 307/91; H10K 85/633; C07D 409/12; C07D 333/50; H10K 50/15; H10K 85/6574; H10K 85/615; H10K 85/624; H10K 85/6576

(57) ABSTRACT

The present application belongs to the technical field of organic materials, and provides a nitrogen-containing compound, an electronic element, and an electronic device. The nitrogen-containing compound has a structure shown in formula 1. The nitrogen-containing compound can improve the performance of the electronic element,

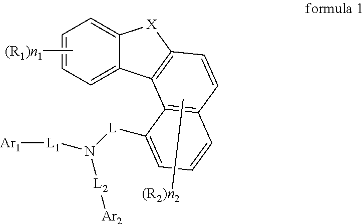

### CROSS REFERENCE TO RELATED APPLICATION

The present application claims priority to Chinese Patent Application 202011195453.6 filed on Oct. 30, 2020 and Chinese Patent Application 202011404791.6 filed on Dec. 2, 2020, and the full contents of the Chinese patent applications are cited herein as a part of the present application.

### TECHNICAL FIELD

The present application relates to the technical field of organic materials, and in particular to a nitrogen-containing compound, and an electronic element and electronic device having the same.

### BACKGROUND

With the development of electronic technology and the progress of material science, electronic devices for realizing electroluminescence or photoelectric conversion are more and more extensively used. Such an electronic device usually includes: a cathode and an anode that are arranged oppositely, and a functional layer arranged between the cathode and the anode. The functional layer includes a plurality of organic or inorganic film layers, and generally includes an energy conversion layer, a hole transport layer (HTL) arranged between the energy conversion layer and the anode, and an electron transport layer (ETL) arranged between the energy conversion layer and the cathode.

When the electronic device is an organic light-emitting device (OLED), the electronic device generally includes an anode, an HTL, an electroluminescent layer as an energy conversion layer, an ETL, and a cathode that are successively stacked. When a voltage is applied to the cathode and the anode, an electric field is generated at each of the two electrodes; and under the action of the electric field, both electrons at a cathode side and holes at an anode side move towards the electroluminescent layer and are combined in the electroluminescent layer to form excitons, and the excitons in an excited state release energy outwards, thereby causing the electroluminescent layer to emit light.

A large number of organic electroluminescent materials with excellent performance have been successively developed, for example, WO2019147030A1, WO2019216574A1, and WO2020032574A1 each disclose a material that can be used to prepare an HTL in an OLED, but it is still necessary to further develop new materials for further improving the performance of electronic devices.

The information disclosed in the background art is merely intended to facilitate the comprehension to the background of the present application, and thus may include information that does not constitute the prior art known to those of ordinary skill in the art.

### SUMMARY

The present application is intended to provide a nitrogen-containing compound, and an electronic element and electronic device having the same. The nitrogen-containing compound can improve the performance of the electronic element and electronic device.

To achieve the objective of the present application, the present application adopts the following technical solutions:

In a first aspect of the present application, a nitrogen-containing compound with a structure shown in formula 1 is provided:

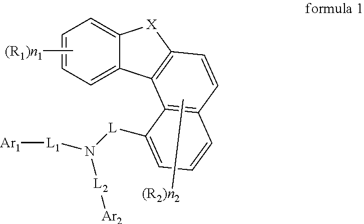

wherein X is selected from O and S;

R₁ and R₂ are the same or different, and are each independently selected from the group consisting of deuterium, cyano, halogen, alkyl with 1 to 5 carbon atoms, trialkylsilyl with 3 to 9 carbon atoms, substituted or unsubstituted aryl with 6 to 12 carbon atoms, and substituted or unsubstituted heteroaryl with 3 to 10 carbon atoms:

n₁ represents the number of R₁, and n₁ is selected from 0, 1, 2, 3, and 4; and when n₁ is greater than 1, any two R₁ are the same or different;
n₂ represents the number of R₂, and n₂ is selected from 0, 1, 2, 3, 4, and 5; and when n₂ is greater than 1, any two R₂ are the same or different;
L is selected from the group consisting of substituted or unsubstituted arylene with 6 to 30 carbon atoms and substituted or unsubstituted heteroarylene with 3 to 30 carbon atoms;
L₁ and L₂ are the same or different, and are each independently selected from the group consisting of a single bond, substituted or unsubstituted arylene with 6 to 30 carbon atoms, and substituted or unsubstituted heteroarylene with 3 to 30 carbon atoms;
Ar₁ and Ar₂ are the same or different, and are each independently selected from the group consisting of substituted or unsubstituted aryl with 6 to 30 carbon atoms and substituted or unsubstituted heteroaryl with 3 to 30 carbon atoms; and
substituents on R₁, R₂, L, L₁, L₂, Ar₁, and Ar₂ are the same or different, and are each independently selected from the group consisting of deuterium, halogen, cyano, alkyl with 1 to 10 carbon atoms, haloalkyl with 1 to 12 carbon atoms, trialkylsilyl with 3 to 18 carbon atoms, aryl with 6 to 20 carbon atoms, heteroaryl with 6 to 20 carbon atoms, cycloalkyl with 3 to 10 carbon atoms, heterocycloalkyl with 2 to 12 carbon atoms, alkoxy with 1 to 12 carbon atoms, and alkylthio with 1 to 12 carbon atoms.

The present application provides a nitrogen-containing compound, wherein naphtho[2,1-b]benzofuran and naphtho[2,1-b]benzothiophene are adopted as a parent nucleus, which can effectively inhibit the intermolecular interaction and has excellent thermal stability. In addition, an arylamine structure with excellent hole transport performance is introduced at position 1 of the parent nucleus by aromatic hydrocarbyl (L), which increases the rigidity of the compound and significantly improves the thermal stability, such that the structural stability can be maintained for a long time at a high temperature. When the nitrogen-containing compound is used as a hole transport material for an OLED, the light-emitting efficiency and service life of the OLED can be both improved.

In a second aspect of the present application, an electronic element is provided, including: an anode and a cathode that are arranged oppositely, and a functional layer arranged between the anode and the cathode, wherein the functional layer includes the nitrogen-containing compound described in the first aspect.

In a third aspect of the present application, an electronic device is provided, including the electronic element described in the second aspect.

Other features and advantages of the present application will be described in detail in the following DETAILED DESCRIPTION section.

### BRIEF DESCRIPTION OF THE DRAWINGS

The above and other features and advantages of the present application will become more apparent by describing exemplary embodiments in detail with reference to the accompanying drawings.

FIG. 1 is a schematic structural diagram of an OLED according to an embodiment of the present application; and

FIG. 2 is a schematic structural diagram of an electronic device according to an embodiment of the present application.

### REFERENCE NUMERALS

100 anode; 200 cathode; 300 functional layer; 310 hole injection layer (HIL); 321 HTL; 322 electron blocking layer (EBL); 330 organic electroluminescent layer; 340 hole blocking layer (HBL); 350 ETL; 360 electron injection layer (EIL); and 400 first electronic device.

### DETAILED DESCRIPTION

Exemplary embodiments will be described below comprehensively with reference to the accompanying drawings. However, the exemplary embodiments can be implemented in various forms and should not be construed as being limited to examples described herein. On the contrary, these embodiments are provided such that the present application is comprehensive and complete, and fully conveys the concept of the exemplary embodiments to those skilled in the art. The described features, structures, or characteristics may be incorporated into one or more embodiments in any suitable manner. In the following description, many specific details are provided to give a full understanding of the embodiments of the present application.

In the figures, a thickness of each of regions and layers may be exaggerated for clarity. The same reference numerals in the figures indicate the same or similar structures, and thus their detailed descriptions will be omitted.

The present application provides a nitrogen-containing compound with a structure shown in formula 1:

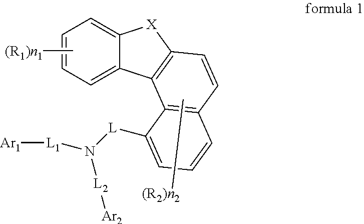

wherein X is selected from O and S;

R₁ and R₂ are the same or different, and are each independently selected from the group consisting of deuterium, cyano, halogen, alkyl with 1 to 5 carbon atoms, trialkylsilyl with 3 to 9 carbon atoms, substituted or unsubstituted aryl with 6 to 12 carbon atoms, and substituted or unsubstituted heteroaryl with 3 to 10 carbon atoms;

n₁ represents the number of R₁, and n₁ is selected from 0, 1, 2, 3, and 4; and when n₁ is greater than 1, any two R₁ are the same or different;

n₂ represents the number of R₂, and n₂ is selected from 0, 1, 2, 3, 4, and 5; and when n₂ is greater than 1, any two R₂ are the same or different;

L is selected from the group consisting of substituted or unsubstituted arylene with 6 to 30 carbon atoms and substituted or unsubstituted heteroarylene with 3 to 30 carbon atoms;

L₁ and L₂ are the same or different, and are each independently selected from the group consisting of a single bond, substituted or unsubstituted arylene with 6 to 30 carbon atoms, and substituted or unsubstituted heteroarylene with 3 to 30 carbon atoms;

Ar₁ and Ar₂ are the same or different, and are each independently selected from the group consisting of substituted or unsubstituted aryl with 6 to 30 carbon atoms and substituted or unsubstituted heteroaryl with 3 to 30 carbon atoms; and

substituents on R₁, R₂, L, L₁, L₂, Ar₁, and Ar₂ are the same or different, and are each independently selected from the group consisting of deuterium, halogen, cyano, alkyl with 1 to 10 carbon atoms, haloalkyl with 1 to 12 carbon atoms, trialkylsilyl with 3 to 18 carbon atoms, aryl with 6 to 20 carbon atoms, heteroaryl with 6 to 20 carbon atoms, cycloalkyl with 3 to 10 carbon atoms, heterocycloalkyl with 2 to 12 carbon atoms, alkoxy with 1 to 12 carbon atoms, and alkylthio with 1 to 12 carbon atoms.

The nitrogen-containing compound provided in the present application has excellent hole transport performance and can be used between an anode and an energy conversion layer in each of an OLED and a photoelectric conversion device to improve the hole transport efficiency between the anode and the energy conversion layer, thereby improving the light-emitting efficiency and service life of the OLED.

In the present application, the number of carbon atoms in a group refers to the number of all carbon atoms. For example, in substituted arylene with 10 carbon atoms, the number of all carbon atoms in the arylene and substituents thereon is 10. Exemplarily, 9,9-dimethylfluorenyl is substituted aryl with 15 carbon atoms.

In the present application, unless otherwise specifically defined, the term “hetero” means that a functional group includes at least one heteroatom such as B, N, O, S, Se, Si, or P, and the rest atoms in the functional group are carbon and hydrogen.

The description manners used in the present application such as “ . . . is(are) each independently” and “each of . . . is independently selected from” and “ . . . each is(are) independently selected from the group consisting of” can be used interchangeably, and should be understood in a broad sense, which can mean that, in different groups, specific options expressed by the same symbol do not affect each other; or in the same group, specific options expressed by the same symbol do not affect each other. For example,

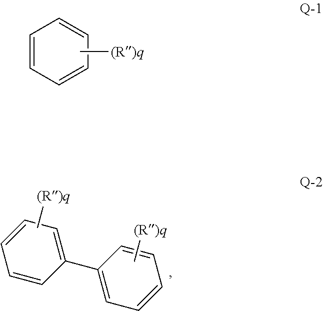

wherein q is each independently selected from 0, 1, 2, or 3 and substituents R″ each are independently selected from the group consisting of hydrogen, fluorine, and chlorine″ means that, in formula Q-1, there are q substituents R″ on the benzene ring, the substituents R″ can be the same or different, and options for each substituent R″ do not affect each other; and in formula Q-2, there are q substituents R″ on each benzene ring of the biphenyl, the numbers q of substituents R″ on the two benzene rings can be the same or different, the substituents R″ can be the same or different, and options for each substituent R″ do not affect each other.

In the present application, the term “optional” or “optionally” means that the event or environment subsequently described may occur or may not occur, which includes situations where the event or environment occurs or does not occur. For example, “optionally, any two adjacent substituents xx form a ring” means that the two substituents may form a ring, but do not necessarily form a ring, which includes situations where the two adjacent substituents form a ring and the two adjacent substituents do not form a ring.

In the present application, the term “substituted or unsubstituted” means that there is no substituent or there is one or more substituents. The substituents may include, but are not limited to, deuterium, halogen, cyano, alkyl, haloalkyl, trialkylsilyl, aryl, heteroaryl, cycloalkyl, heterocycloalkyl, alkoxy, and alkylthio.

In the present application, alkyl with 1 to 10 carbon atoms may include linear alkyl with 1 to 10 carbon atoms and branched alkyl with 3 to 10 carbon atoms. For example, there can be 1, 2, 3, 4, 5, 6, 7, 8, 9, or 10 carbon atoms in the alkyl. Specific examples of the alkyl may include, but are not limited to, methyl, ethyl, n-propyl, isopropyl, n-butyl, isobutyl, tert-butyl, n-pentyl, isopentyl, neopentyl, n-hexyl, n-heptyl, n-octyl, 2-ethylhexyl, nonyl, decyl, and 3,7-dimethyloctyl.

In the present application, the aryl refers to any functional group or substituent derived from an aromatic hydrocarbon ring. The aryl may refer to a monocyclic aryl group (such as phenyl) or a polycyclic aryl group. In other words, the aryl may refer a monocyclic aryl group, a fused-ring aryl group, two or more monocyclic aryl groups that are conjugated through carbon-carbon bonds, a monocyclic aryl group and a fused-ring aryl group that are conjugated through carbon-carbon bonds, and two or more fused-ring aryl groups that are conjugated through carbon-carbon bonds. That is, two or more aromatic groups conjugated through carbon-carbon bonds can also be regarded as the aryl of the present application. For example, the fused-ring aryl group may include a bicyclic fused aryl group (such as naphthyl) and a tricyclic fused aryl group (such as phenanthryl, fluorenyl, and anthracenyl). The aryl does not include heteroatoms such as B, N, O, S, Se, Si, and P. For example, in the present application, biphenyl, terphenyl, and the like are aryl. Examples of the aryl may include, but are not limited to, phenyl, naphthyl, fluorenyl, anthracenyl, phenanthryl, biphenyl, terphenyl, tetraphenyl, pentaphenyl, hexaphenyl, benzo[9,10]phenanthryl, pyrenyl, pyrylo, benzofluoranthenyl, and chrysenyl. The arylene involved in the present application refers to a divalent group obtained after one hydrogen atom is further removed from aryl.

In the present application, substituted aryl refers to aryl in which one or more hydrogen atoms are substituted by another group. For example, at least one hydrogen atom is substituted by deuterium. F, Cl, I, CN, hydroxyl, amino, branched alkyl, linear alkyl, cycloalkyl, alkoxy, alkylamino, alkylthio, aryl, heteroaryl, or another group. Specific examples of heteroaryl-substituted aryl may include, but are not limited to, dibenzofuranyl-substituted phenyl, dibenzothienyl-substituted phenyl, and pyridyl-substituted phenyl. It should be appreciated that the number of carbon atoms in substituted aryl refers to the total number of carbon atoms in the aryl and substituents thereon.

In the present application, the heteroaryl refers to a monovalent aromatic ring with at least one heteroatom or a derivative thereof. The heteroatom is at least one selected from the group consisting of B, O, N, P, Si, Se, and S. The heteroaryl can be monocyclic heteroaryl or polycyclic heteroaryl. In other words, the heteroaryl may refer to a single aromatic ring system or multiple aromatic ring systems conjugated through carbon-carbon bonds, wherein each aromatic ring system is an aromatic monocyclic ring or an aromatic fused ring. For example, the heteroaryl may include, but is not limited to, thienyl, furyl, pyrrolyl, imidazolyl, thiazolyl, oxazolyl, oxadiazolyl, triazolyl, pyridyl, bipyridyl, pyrimidinyl, triazinyl, acridinyl, pyridazinyl, pyrazinyl, quinolinyl, quinazolinyl, quinoxalinyl, phenoxazinyl, phthalazinyl, pyridopyrimidinyl, pyridopyrazinyl, pyrazinopyrazinyl, isoquinolinyl, indolyl, carbazolyl, benzoxazolyl, benzimidazolyl, benzothiazolyl, benzocarbazolyl, benzothienyl, dibenzothienyl, thienothienyl, benzofuranyl, phenanthrolinyl, isoxazolyl, thiadiazolyl, benzothiazolyl, phenothiazinyl, silylfluorenyl, dibenzofuranyl, N-phenylcarbazolyl, N-pyridylcarbazolyl, and N-methylcarbazolyl. The thienyl, furyl, phenanthrolinyl, and the like are heteroaryl with a single aromatic ring system; and the N-arylcarbazolyl, N-heteroarylcarbazolyl, and the like are heteroaryl with multiple ring systems conjugated through carbon-carbon bonds. The heteroarylene involved in the present application refers to a divalent group obtained after one hydrogen atom is further removed from heteroaryl.

In the present application, the substituted heteroaryl may refer to heteroaryl in which one or more hydrogen atoms are substituted by groups such as D, halogen, —CN, aryl, heteroaryl, trialkylsilyl, alkyl, cycloalkyl, and haloalkyl. Specific examples of aryl-substituted heteroaryl may include, but are not limited to, phenyl-substituted dibenzofuranyl, phenyl-substituted dibenzothienyl, and phenyl-substituted pyridyl. It should be understood that the number of carbon atoms in the substituted heteroaryl refers to the total number of carbon atoms in the heteroaryl and substituents thereon.

In the present application, there can be 6 to 20 (such as 6, 7, 8, 9, 10, 11, 12, 13, 14, 15, 16, 17, 18, 19, or 20) carbon atoms in aryl as a substituent; and specific examples of the aryl as a substituent may include, but are not limited to, phenyl, biphenyl, naphthyl, anthracenyl, phenanthryl, and chrysenyl.

In the present application, there can be 6 to 20 (such as 6, 7, 8, 9, 10, 11, 12, 13, 14, 15, 16, 17, 18, 19, or 20) carbon atoms in heteroaryl as a substituent; and specific examples of the heteroaryl as a substituent may include, but are not limited to, carbazolyl, dibenzofuranyl, dibenzothienyl, quinolinyl, quinazolinyl, quinoxalinyl, and isoquinolinyl.

In the present application, the explanation of aril can also be applied to arylene, and the explanation of heteroaryl can also be applied to heteroarylene.

In the present application, a ring system formed by n atoms is an n-membered ring. For example, phenyl is 6-membered aryl. A 6-10 membered aromatic ring refers to a benzene ring, an indene ring, a naphthalene ring, or the like.

The “ring” in the present application may include a saturated ring and an unsaturated ring, wherein for example, the saturated ring refers to cycloalkyl and heterocycloalkyl and the unsaturated ring refers to cycloalkenyl, heterocycloalkenyl, aryl, and heteroaryl.

In the present application,

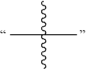

refers to a position attached to other substituents or binding sites.

In the present application, a non-positional bond refers to a single bond and

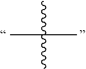

extending from a ring system, which means that one end of the bond can be attached to any position in the ring system through which the bond penetrates, and the other end is attached to the remaining part in the compound molecule. For example, as shown in the following formula (f), the naphthyl represented by the formula (f) is attached to the remaining part in the molecule through two non-positional bonds that penetrate through the bicyclic ring, which indicates any possible attachment modes shown in formula (f-1) to formula (f-10).

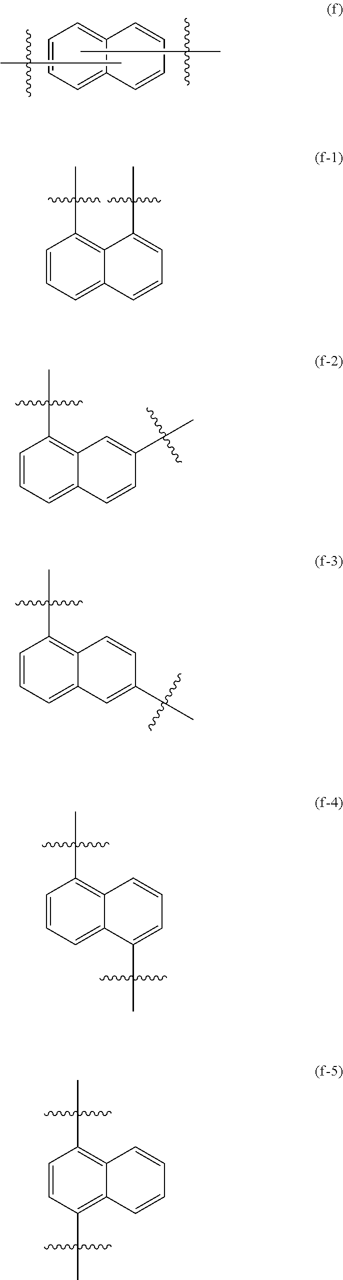

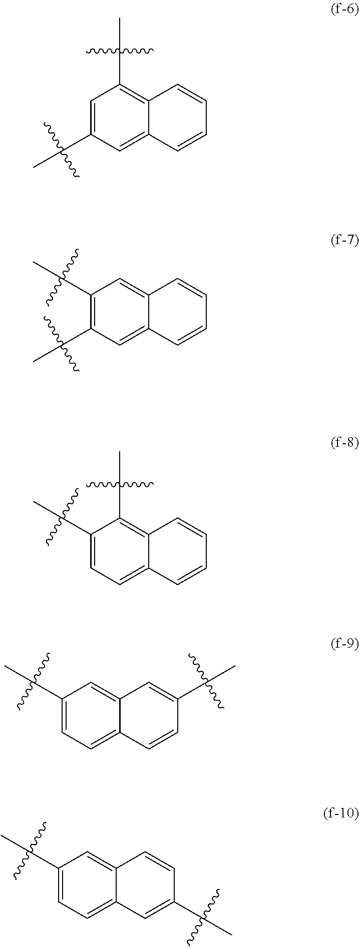 [SMILES: *c1cc(*)c2ccccc2c1 ; *c1cc2ccccc2cc1* ; *c1ccc2ccccc2c1* ; *c1ccc2ccc(*)cc2c1 ; *c1ccc2cc(*)ccc2c1]

For example, as shown in the following formula (X′), the phenanthryl represented by the formula (X′) is attached to the remaining part in the molecule through a non-positional bond extending from the middle of a benzene ring at a side, which indicates any possible attachment modes shown in formula X′-1 to formula (X′-4).

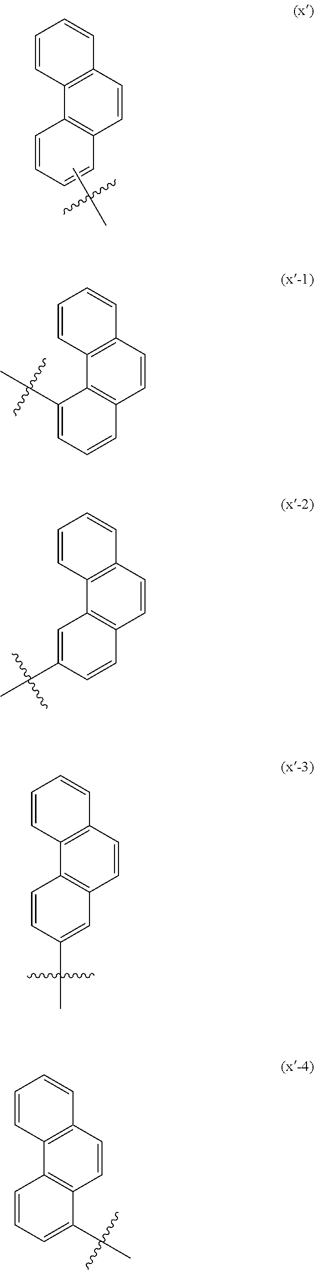

In the present application, a non-positional substituent refers to a substituent linked through a single bond extending from the center of a ring system, which means that the substituent can be attached to any possible position in the ring system. For example, as shown in the following formula (Y), the substituent R′ represented by the formula (Y) is attached to a quinoline ring through a non-positional bond, which indicates any possible attachment modes shown in formula (Y-1) to formula (Y-7).

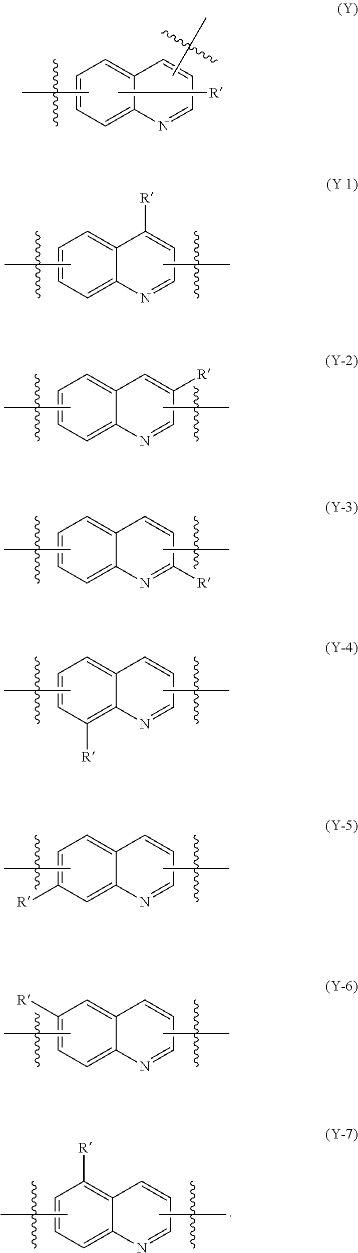

In the present application, the halogen can be, for example, fluorine, chlorine, bromine, or iodine.

In the present application, specific examples of trialkylsilyl may include, but are not limited to, trimethylsilyl and triethylsilyl.

In the present application, specific examples of triarylsilyl may include, but are not limited to, triphenylsilyl.

In the present application, specific examples of haloalkyl may include, but are not limited to, trifluoromethyl.

In the present application, specific examples of cycloalkyl may include, but are not limited to, cyclopropyl, cyclopentyl, cyclohexyl, and adamantyl.

Optionally, substituents on R₁, R₂, L, L₁, and L₂ are each independently selected from the group consisting of deuterium, fluorine, cyano, alkyl with 1 to 5 carbon atoms, trimethylsilyl, phenyl, naphthyl, and cycloalkyl with 3 to 6 carbon atoms.

In an embodiment, R₁ and R₂ are each independently selected from the group consisting of deuterium, cyano, fluorine, alkyl with 1 to 5 carbon atoms, trimethylsilyl, phenyl, naphthyl, biphenyl, and pyridyl.

Optionally, R₁ and R₂ are each independently selected from the group consisting of isopropyl, dibenzofuranyl, and dibenzothienyl.

In an embodiment, L is selected from the group consisting of substituted or unsubstituted arylene with 6 to 12 carbon atoms and substituted or unsubstituted heteroarylene with 3 to 12 carbon atoms. For example, L is selected from the group consisting of substituted or unsubstituted arylene with 6, 7, 8, 9, 10, 11, or 12 carbon atoms and substituted or unsubstituted heteroarylene with 3, 4, 5, 6, 7, 8, 9, 10, 11, or 12 carbon atoms.

In an embodiment, L₁ and L₂ are each independently selected from the group consisting of a single bond, substituted or unsubstituted arylene with 6 to 12 carbon atoms, and substituted or unsubstituted heteroarylene with 3 to 12 carbon atoms. For example, L₁ and L₂ are each independently selected from the group consisting of a single bond, substituted or unsubstituted arylene with 6, 7, 8, 9, 10, 11, or 12 carbon atoms, and substituted or unsubstituted heteroarylene with 3, 4, 5, 6, 7, 8, 9, 10, 11, or 12 carbon atoms.

Optionally, L is selected from the group consisting of substituted or unsubstituted phenylene, substituted or unsubstituted naphthylene, and substituted or unsubstituted biphenylene.

Optionally, L₁ and L₂ are each independently selected from the group consisting of a single bond, substituted or unsubstituted phenylene, substituted or unsubstituted naphthylene, and substituted or unsubstituted biphenylene.

Optionally, L is selected from the group consisting of the following groups:

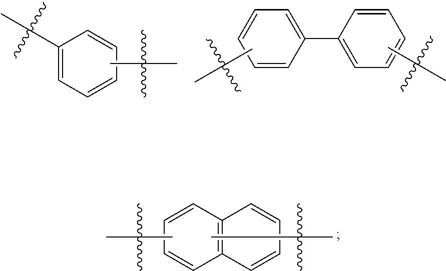

and

L₁ and L₂ are each independently selected from the group consisting of a single bond and the following groups:

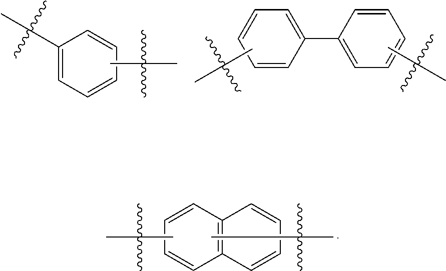

Optionally, L is the following group:

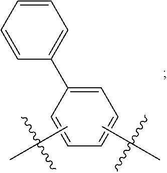

and

L₁ and L₂ are each independently selected from the group consisting of a single bond and the following group:

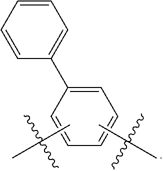

Optionally, L is selected from the group consisting of the following groups:

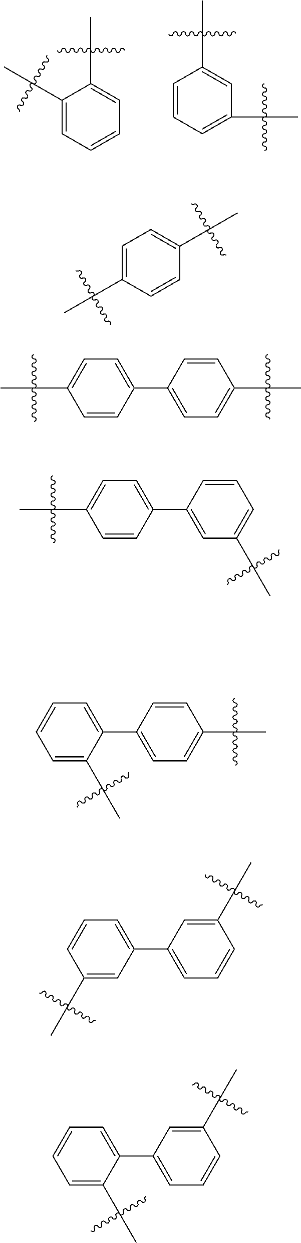

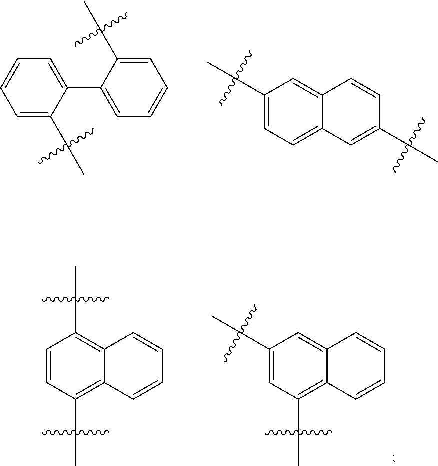 [SMILES: *c1ccccc1-c1ccccc1* ; *c1ccc2cc(*)ccc2c1 ; *c1ccc(*)c2ccccc12 ; *c1cc(*)c2ccccc2c1]

L₁ and L₂ are each independently selected from the group consisting of a single bond and the following groups:

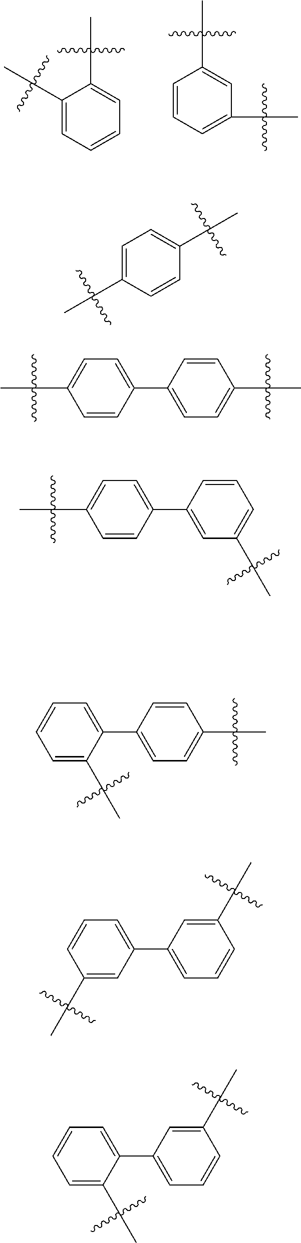

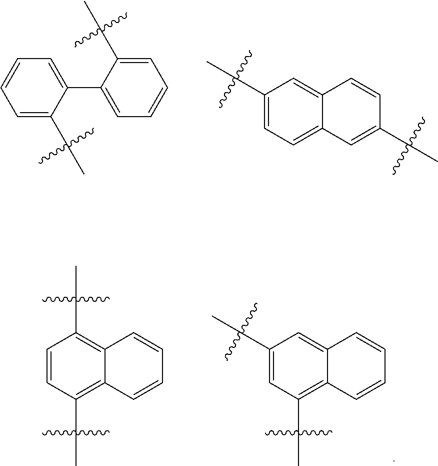 [SMILES: *c1ccccc1-c1ccccc1* ; *c1ccc2cc(*)ccc2c1 ; *c1ccc(*)c2ccccc12 ; *c1cc(*)c2ccccc2c1]

Optionally, L is selected from the group consisting of the following groups:

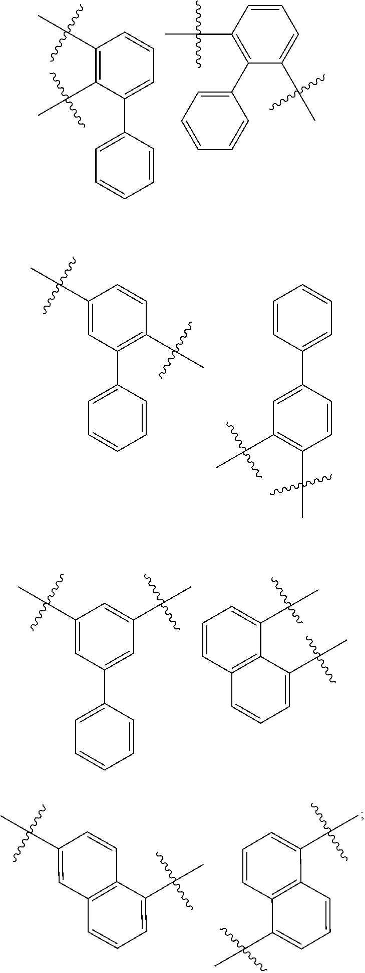

and

L₁ and L₂ are each independently selected from the group consisting of a single bond and the following groups:

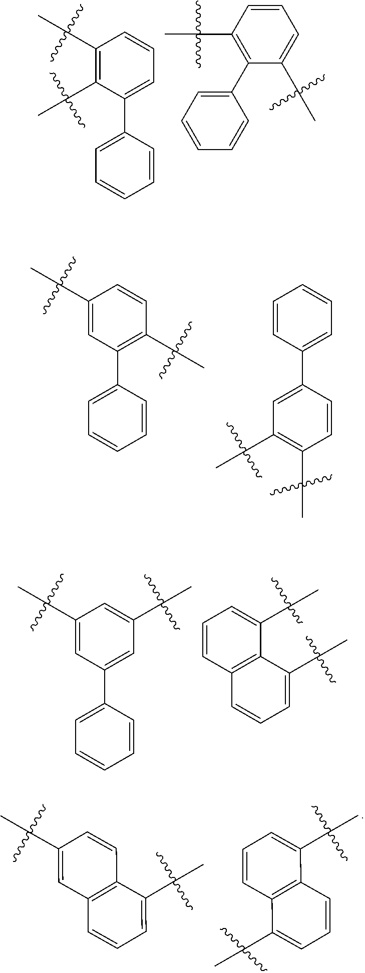

Optionally, Ar₁ and Ar₂ are each independently selected from the group consisting of the following groups shown in formula i-1 to formula i-7:

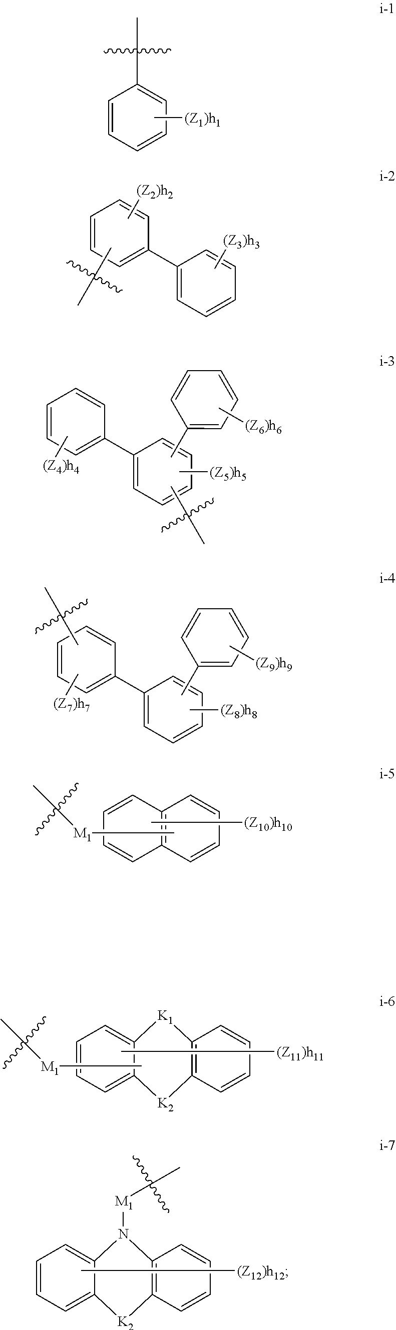

wherein M₁ is selected from the group consisting of a single bond,

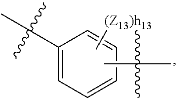

and

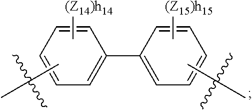

Z₁ is selected from the group consisting of deuterium, halogen, cyano, alkyl with 1 to 5 carbon atoms, cycloalkyl with 3 to 10 carbon atoms, and trialkylsilyl with 3 to 18 carbon atoms;

Z₂ to Z₉ and Z₁₃ to Z₁₅ are each independently selected from the group consisting of deuterium, halogen, cyano, alkyl with 1 to 5 carbon atoms, cycloalkyl with 3 to 10 carbon atoms, heteroaryl with 6 to 18 carbon atoms, and trialkylsilyl with 3 to 18 carbon atoms;

Z₁₀ to Z₁₂ are each independently selected from the group consisting of deuterium, halogen, cyano, alkyl with 1 to 5 carbon atoms, cycloalkyl with 3 to 10 carbon atoms, aryl with 6 to 20 carbon atoms, heteroaryl with 6 to 18 carbon atoms, and trialkylsilyl with 3 to 18 carbon atoms;

h₁ to h₁₅ are collectively represented by hₖ and Z₁ to Z₁₅ are collectively represented by Zₖ, wherein k is a variable and is any integer from 1 to 15, and hₖ indicates the number of substituents Zₖ; when k is 5, hₖ is selected from 0, 1, 2, and 3; when k is selected from 2, 7, 8, 13, 14, and 15, hₖ is selected from 0, 1, 2, 3, and 4; when k is selected from 1, 3, 4, 6, and 9, hₖ is selected from 0, 1, 2, 3, 4, and 5; when k is selected from 10 and 11, hₖ is selected from 0, 1, 2, 3, 4, 5, 6, and 7; when k is 12, hₖ is selected from 0, 1, 2, 3, 4, 5, 6, 7, and 8; and when hₖ is greater than 1, any two substituents Zₖ are the same or different;

K₁ is selected from the group consisting of O, S, N(Z₁₆), C(Z₁₇Z₁₈), and Si(Z₁₉Z₂₀), wherein Z₁₆, Z₁₇, Z₁₈, Z₁₉, and Z₂₀ are each independently selected from the group consisting of alkyl with 1 to 5 carbon atoms, aryl with 6 to 12 carbon atoms, and heteroaryl with 6 to 12 carbon atoms, or Z₁₇ and Z₁₈ are linked to form a saturated or unsaturated ring with 3 to 15 carbon atoms together with atoms attached to the two, or Z₁₉ and Z₂₀ are linked to form a saturated or unsaturated ring with 3 to 15 atoms together with atoms attached to the two (For example, in formula j-6

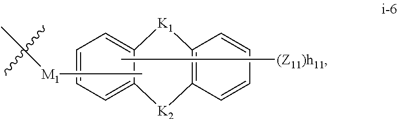

when M₁ is a single bond, Z₁₁ is hydrogen, K₂ is a single bond, and K₁ is C(Z₁₇Z₁₈), Z₁₇ and Z₁₈ can be linked to form a 5-13 membered saturated or unsaturated ring together with atoms attached to the two, or Z₁₇ and Z₁₈ can exist independently of each other; when Z₁₇ and Z₁₈ form a ring, the ring can be a 5-membered ring (such as

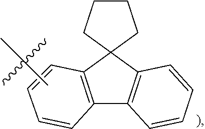

a 6-membered ring (such as

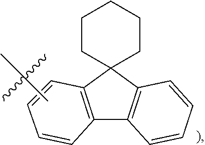

or a 13-membered ring (such as

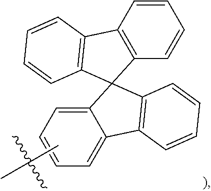

Z₁₇ and Z₁₈ can also form a ring with another number of ring carbon atoms, which will not be listed here. The present application has no specific limitation on the number of ring carbon atoms in the ring); and

K₂ is selected from the group consisting of a single bond, O, S, N(Z₂₁), C(Z₂₂Z₂₃), and Si(Z₂₄Z₂₅), wherein Z₂₁, Z₂₂, Z₂₃, Z₂₄, and Z₂₅ are each independently selected from the group consisting of alkyl with 1 to 5 carbon atoms, aryl with 6 to 12 carbon atoms, and heteroaryl with 6 to 12 carbon atoms, or Z₂₂ and Z₂₃ are linked to form a saturated or unsaturated ring with 3 to 15 carbon atoms together with atoms attached to the two, or Z₂₄ and Z₂₅ are linked to form a saturated or unsaturated ring with 3 to 15 carbon atoms together with atoms attached to the two. The present application has no specific limitation on the number of ring carbon atoms in the ring formed by Z₂₂ and Z₂₃ and the number of ring carbon atoms in the ring formed by Z₂₄ and Z₂₅, and the number of ring carbon atoms in a ring formed by Z₂₂ and Z₂₃ or Z₂₄ and Z₂₅ is defined as the same as that in the ring formed by Z₁₇ and Z₁₈, which will not be repeated here.

In an embodiment, Ar₁ and Ar₂ are each independently selected from the group consisting of substituted or unsubstituted aryl with 6 to 25 carbon atoms and substituted or unsubstituted heteroaryl with 12 to 18 carbon atoms. For example, Ar₁ and Ar₂ are each independently selected from the group consisting of substituted or unsubstituted aryl with 6, 7, 8, 9, 10, 11, 12, 13, 14, 15, 16, 17, 18, 19, 20, 21, 22, 23, 24, or 25 carbon atoms and substituted or unsubstituted heteroaryl with 12, 13, 14, 15, 16, 17, or 18 carbon atoms.

Optionally, Ar₁ and Ar₂ are each independently a substituted or unsubstituted group W; an unsubstituted group W is selected from the group consisting of the following groups:

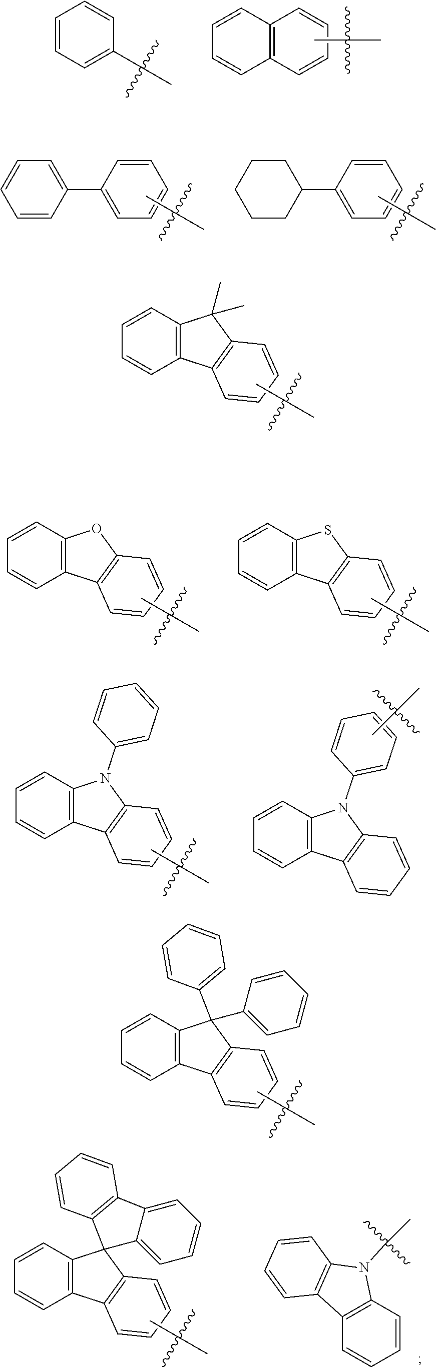

when the group W is substituted, a substituent on the group W is selected from the group consisting of deuterium, fluorine, cyano, trimethylsilyl, methyl, ethyl, isopropyl, tert-butyl, cyclopropyl, cyclohexyl, phenyl, naphthyl, biphenyl, dibenzofuranyl, dibenzothienyl, and carbazolyl; and when there are two or more substituents on the group W, the two or more substituents are the same or different.

Optionally, Ar₁ and Ar₂ are each independently a substituted or unsubstituted group W′; an unsubstituted group W′ is selected from the group consisting of the following groups:

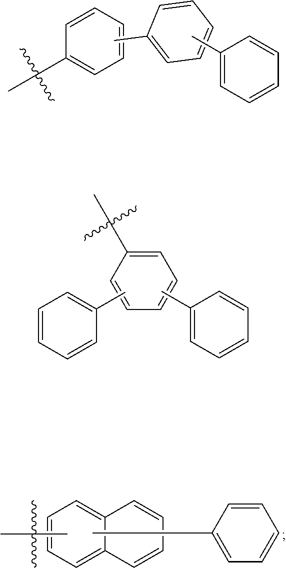

when the group W′ is substituted, a substituent on the group W′ is selected from the group consisting of deuterium, fluorine, cyano, trimethylsilyl, methyl, ethyl, isopropyl, tert-butyl, cyclopropyl, cyclohexyl, phenyl, naphthyl, biphenyl, dibenzofuranyl, dibenzothienyl, and carbazolyl; and when there are two or more substituents on the group W′, the two or more substituents are the same or different.

Optionally, substituents on Ar₁ and Ar₂ are each independently selected from the group consisting of deuterium, fluorine, cyano, alkyl with 1 to 5 carbon atoms, trimethylsilyl, aryl with 6 to 12 carbon atoms, heteroaryl with 12 to 18 carbon atoms, and cycloalkyl with 3 to 6 carbon atoms.

Optionally, substituents on Ar₁ and Ar₂ are each independently selected from the group consisting of deuterium, fluorine, cyano, trimethylsilyl, methyl, ethyl, isopropyl, tert-butyl, cyclopropyl, cyclohexyl, phenyl, naphthyl, biphenyl, dibenzofuranyl, dibenzothienyl, and carbazolyl.

Optionally, Ar₁ and Ar₂ are each independently selected from the group consisting of substituted or unsubstituted phenyl, substituted or unsubstituted naphthyl, substituted or unsubstituted biphenyl, substituted or unsubstituted terphenyl, substituted or unsubstituted fluorenyl, substituted or unsubstituted dibenzofuranyl, substituted or unsubstituted dibenzothienyl, substituted or unsubstituted carbazolyl, and substituted or unsubstituted spirobifluorenyl; and

substituents on Ar₁ and Ar₂ are each independently selected from the group consisting of deuterium, fluorine, cyano, trimethylsilyl, methyl, ethyl, isopropyl, tert-butyl, cyclopropyl, cyclohexyl, phenyl, naphthyl, biphenyl, dibenzofuranyl, dibenzothienyl, and carbazolyl.

Optionally, Ar₁ and Ar₂ are each independently selected from the group consisting of the following groups:

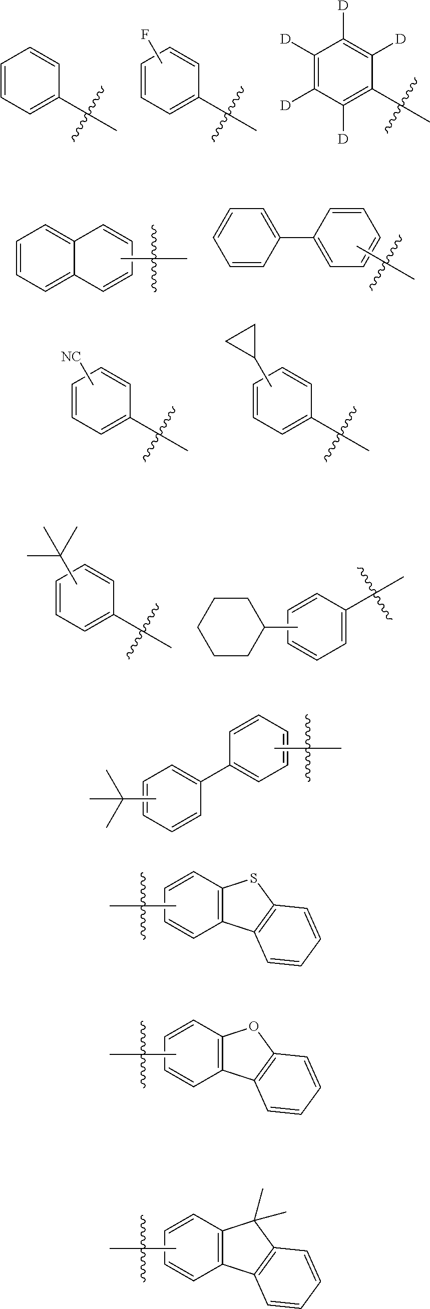

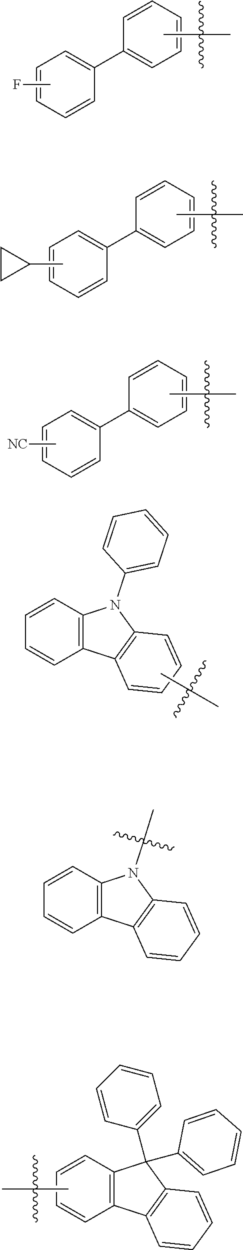

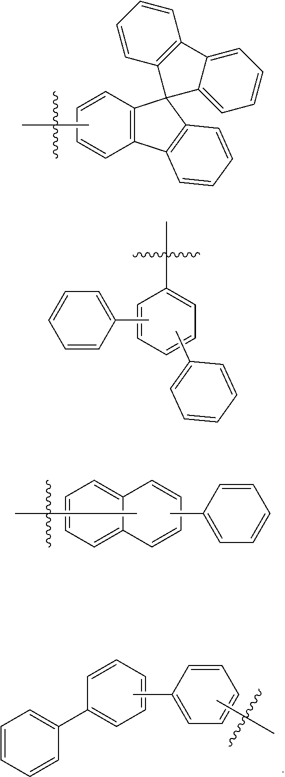

Optionally, Ar₁ and Ar₂ are each independently selected from the group consisting of the following groups:

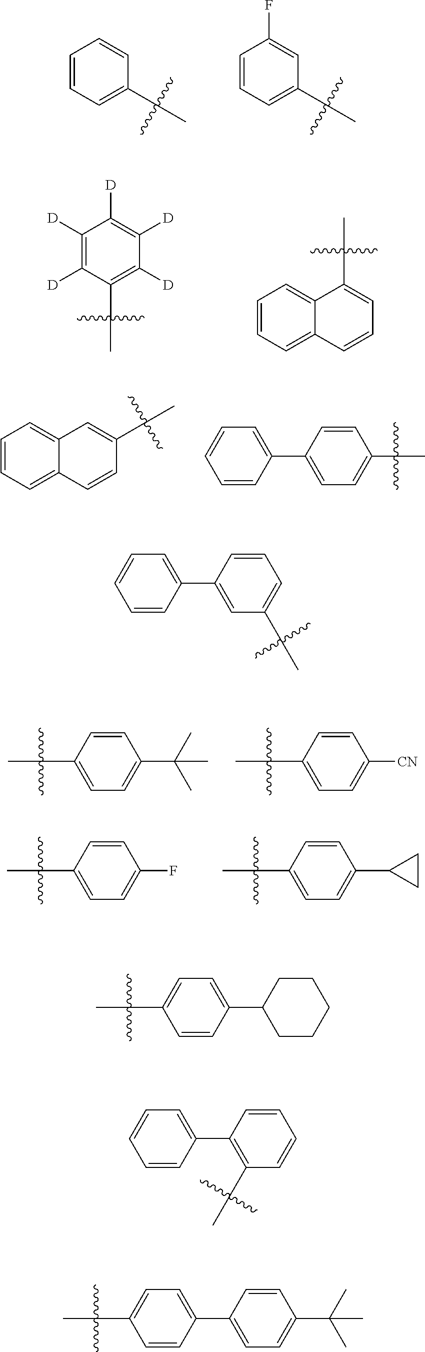

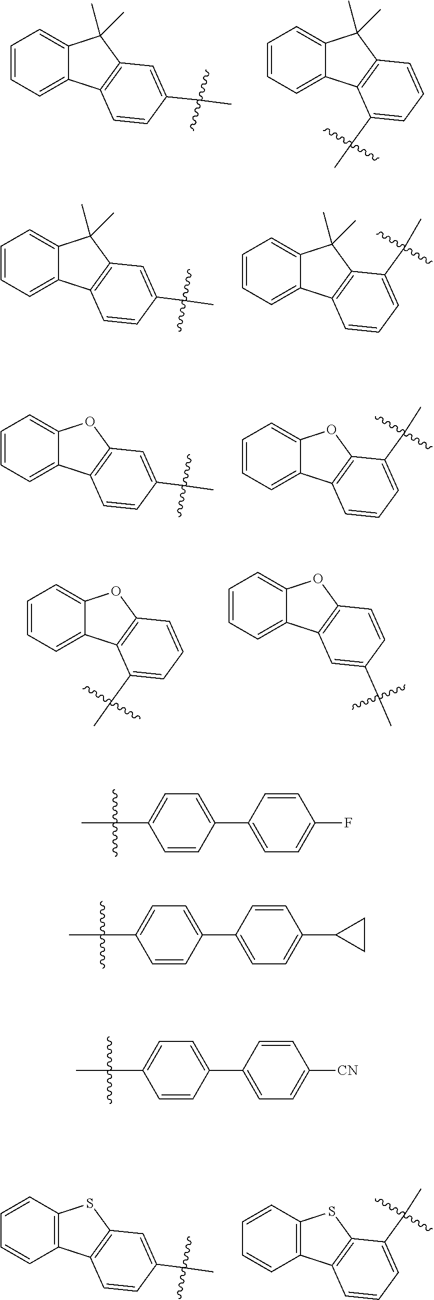

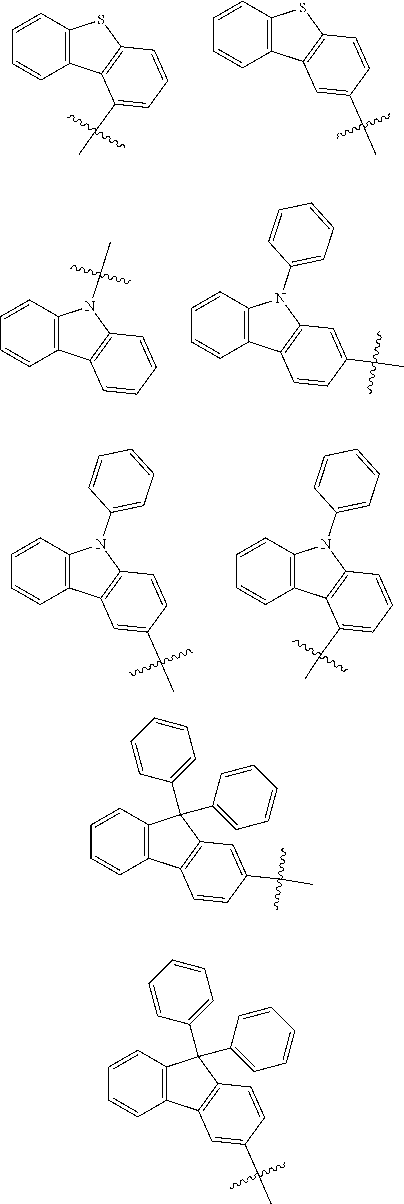

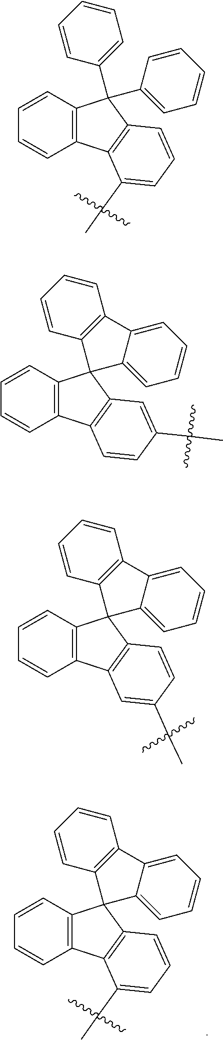 [SMILES: *c1cccc2c1-c1ccccc1C2(c1ccccc1)c1ccccc1 ; *c1ccc2c(c1)C1(c3ccccc3-c3ccccc31)c1ccccc1-2 ; *c1ccc2c(c1)-c1ccccc1C21c2ccccc2-c2ccccc21 ; *c1cccc2c1-c1ccccc1C21c2ccccc2-c2ccccc21]

Optionally, Ar₁ and Ar₂ are each independently selected from the group consisting of the following groups:

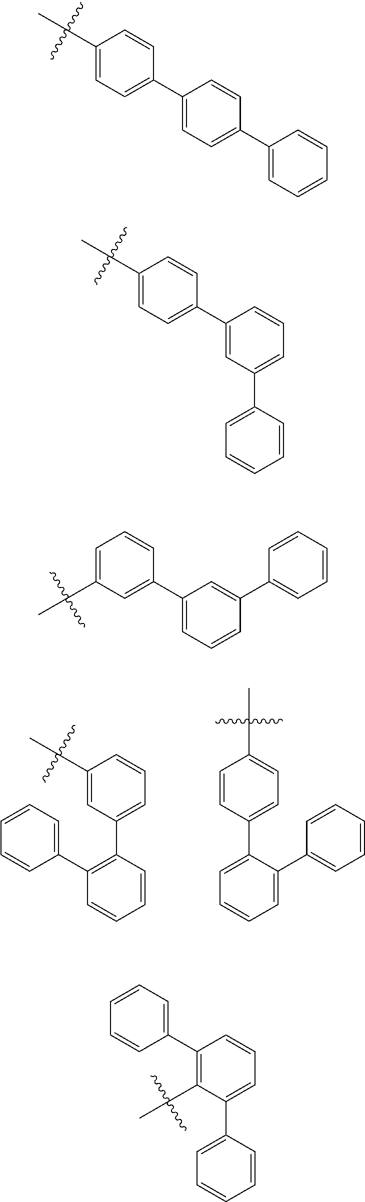 [SMILES: *c1ccc(-c2ccc(-c3ccccc3)cc2)cc1 ; *c1ccc(-c2cccc(-c3ccccc3)c2)cc1 ; *c1cccc(-c2cccc(-c3ccccc3)c2)c1 ; *c1cccc(-c2ccccc2-c2ccccc2)c1 ; *c1ccc(-c2ccccc2-c2ccccc2)cc1 ; *c1c(-c2ccccc2)cccc1-c1ccccc1]

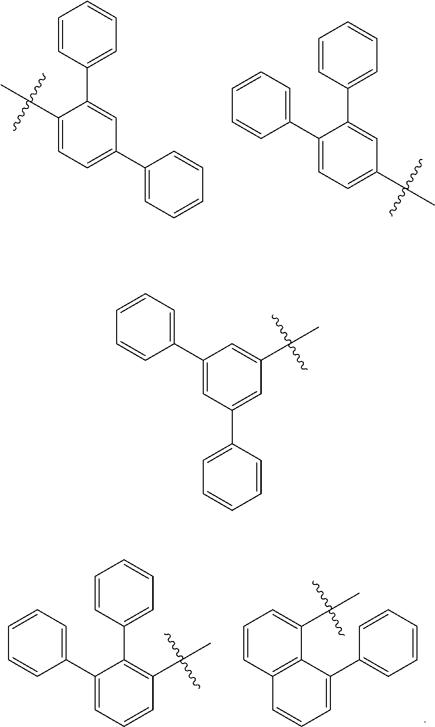 [SMILES: *c1ccc(-c2ccccc2)cc1-c1ccccc1 ; *c1ccc(-c2ccccc2)c(-c2ccccc2)c1 ; *c1cc(-c2ccccc2)cc(-c2ccccc2)c1 ; *c1cccc(-c2ccccc2)c1-c1ccccc1 ; *c1cccc2cccc(-c3ccccc3)c12]

Optionally, the nitrogen-containing compound is selected from the group consisting of the following compounds:

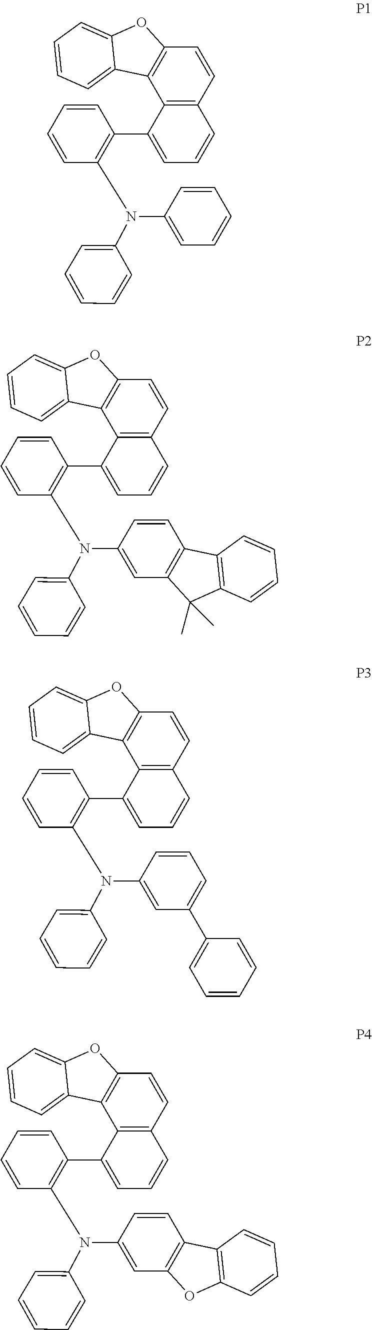 [SMILES: c1ccc(N(c2ccccc2)c2ccccc2-c2cccc3ccc4oc5ccccc5c4c23)cc1 ; CC1(C)c2ccccc2-c2ccc(N(c3ccccc3)c3ccccc3-c3cccc4ccc5oc6ccccc6c5c34)cc21 ; c1ccc(-c2cccc(N(c3ccccc3)c3ccccc3-c3cccc4ccc5oc6ccccc6c5c34)c2)cc1 ; c1ccc(N(c2ccc3c(c2)oc2ccccc23)c2ccccc2-c2cccc3ccc4oc5ccccc5c4c23)cc1]

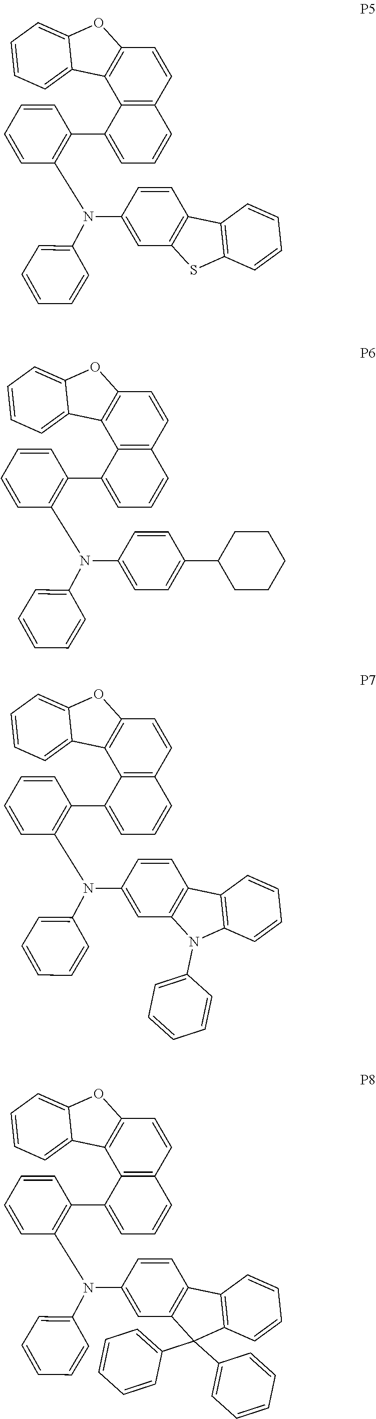 [SMILES: c1ccc(N(c2ccc3c(c2)sc2ccccc23)c2ccccc2-c2cccc3ccc4oc5ccccc5c4c23)cc1 ; c1ccc(N(c2ccc(C3CCCCC3)cc2)c2ccccc2-c2cccc3ccc4oc5ccccc5c4c23)cc1 ; c1ccc(N(c2ccc3c4ccccc4n(-c4ccccc4)c3c2)c2ccccc2-c2cccc3ccc4oc5ccccc5c4c23)cc1 ; c1ccc(N(c2ccc3c(c2)C(c2ccccc2)(c2ccccc2)c2ccccc2-3)c2ccccc2-c2cccc3ccc4oc5ccccc5c4c23)cc1]

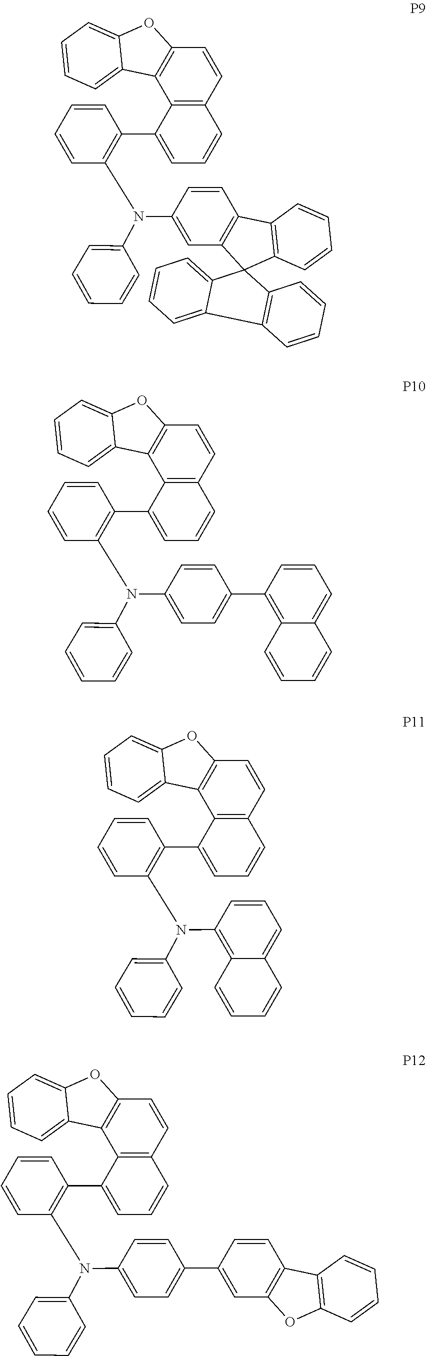 [SMILES: c1ccc(N(c2ccc3c(c2)C2(c4ccccc4-c4ccccc42)c2ccccc2-3)c2ccccc2-c2cccc3ccc4oc5ccccc5c4c23)cc1 ; c1ccc(N(c2ccc(-c3cccc4ccccc34)cc2)c2ccccc2-c2cccc3ccc4oc5ccccc5c4c23)cc1 ; c1ccc(N(c2ccccc2-c2cccc3ccc4oc5ccccc5c4c23)c2cccc3ccccc23)cc1 ; c1ccc(N(c2ccc(-c3ccc4c(c3)oc3ccccc34)cc2)c2ccccc2-c2cccc3ccc4oc5ccccc5c4c23)cc1]

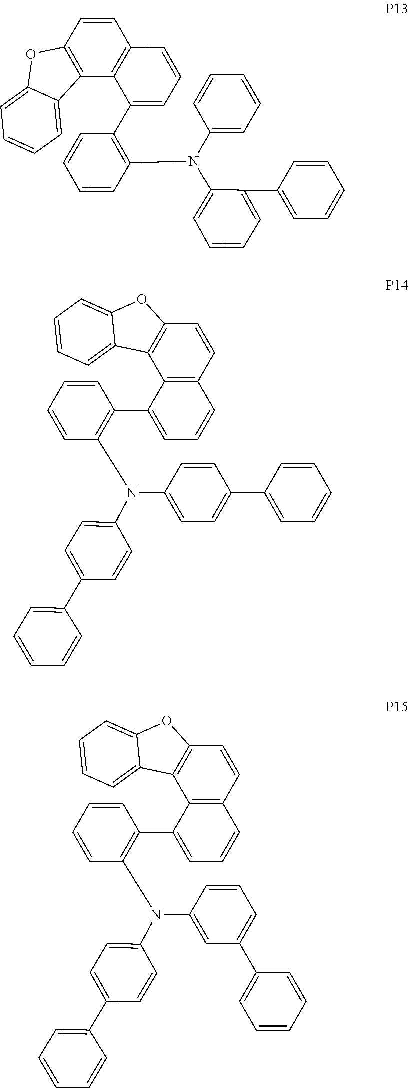 [SMILES: c1ccc(-c2ccccc2N(c2ccccc2)c2ccccc2-c2cccc3ccc4oc5ccccc5c4c23)cc1 ; c1ccc(-c2ccc(N(c3ccc(-c4ccccc4)cc3)c3ccccc3-c3cccc4ccc5oc6ccccc6c5c34)cc2)cc1 ; c1ccc(-c2ccc(N(c3cccc(-c4ccccc4)c3)c3ccccc3-c3cccc4ccc5oc6ccccc6c5c34)cc2)cc1]

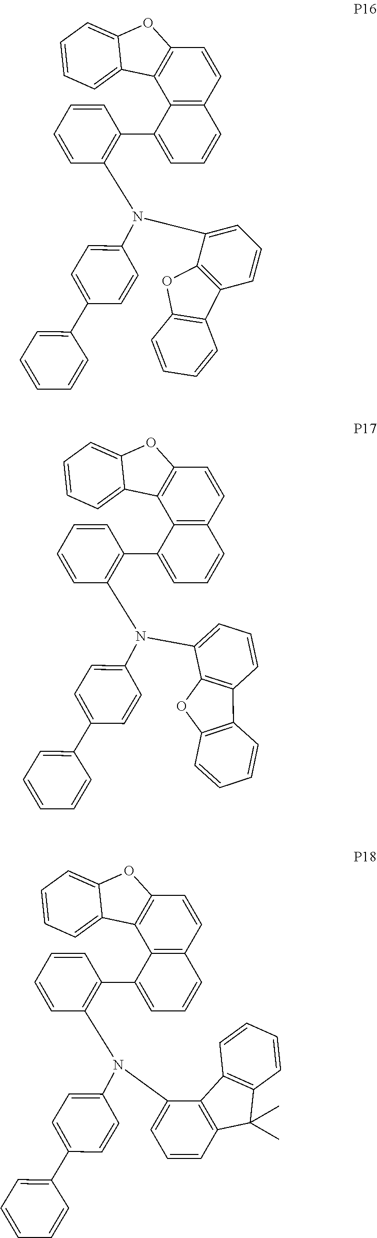 [SMILES: c1ccc(-c2ccc(N(c3ccccc3-c3cccc4ccc5oc6ccccc6c5c34)c3cccc4c3oc3ccccc34)cc2)cc1 ; c1ccc(-c2ccc(N(c3ccccc3-c3cccc4ccc5oc6ccccc6c5c34)c3cccc4c3oc3ccccc34)cc2)cc1 ; CC1(C)c2ccccc2-c2c(N(c3ccc(-c4ccccc4)cc3)c3ccccc3-c3cccc4ccc5oc6ccccc6c5c34)cccc21]

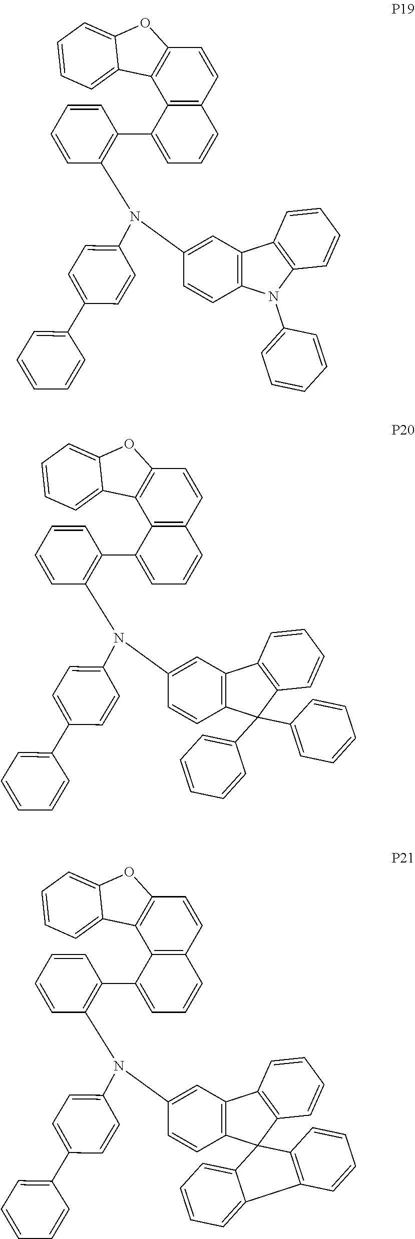 [SMILES: c1ccc(-c2ccc(N(c3ccc4c(c3)c3ccccc3n4-c3ccccc3)c3ccccc3-c3cccc4ccc5oc6ccccc6c5c34)cc2)cc1 ; c1ccc(-c2ccc(N(c3ccc4c(c3)-c3ccccc3C4(c3ccccc3)c3ccccc3)c3ccccc3-c3cccc4ccc5oc6ccccc6c5c34)cc2)cc1 ; c1ccc(-c2ccc(N(c3ccc4c(c3)-c3ccccc3C43c4ccccc4-c4ccccc43)c3ccccc3-c3cccc4ccc5oc6ccccc6c5c34)cc2)cc1]

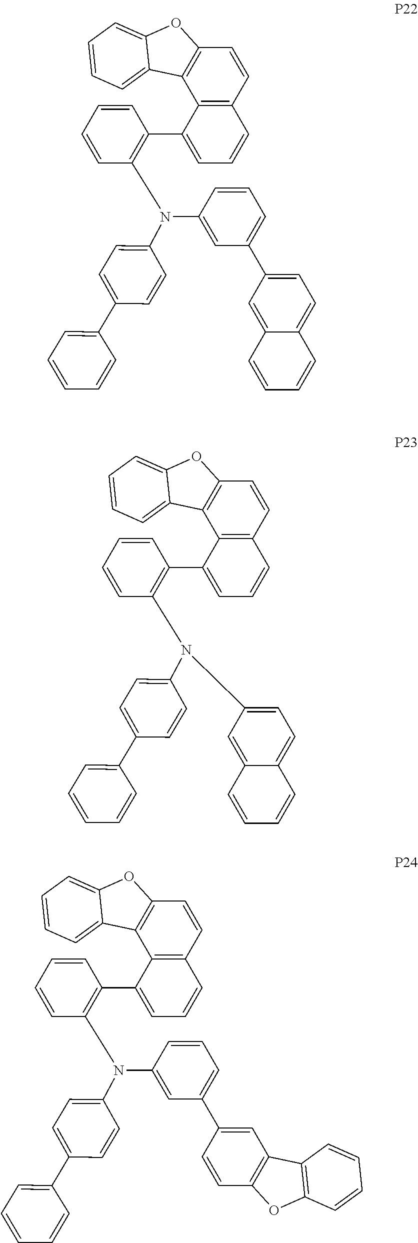 [SMILES: c1ccc(-c2ccc(N(c3cccc(-c4ccc5ccccc5c4)c3)c3ccccc3-c3cccc4ccc5oc6ccccc6c5c34)cc2)cc1 ; c1ccc(-c2ccc(N(c3ccc4ccccc4c3)c3ccccc3-c3cccc4ccc5oc6ccccc6c5c34)cc2)cc1 ; c1ccc(-c2ccc(N(c3cccc(-c4ccc5oc6ccccc6c5c4)c3)c3ccccc3-c3cccc4ccc5oc6ccccc6c5c34)cc2)cc1]

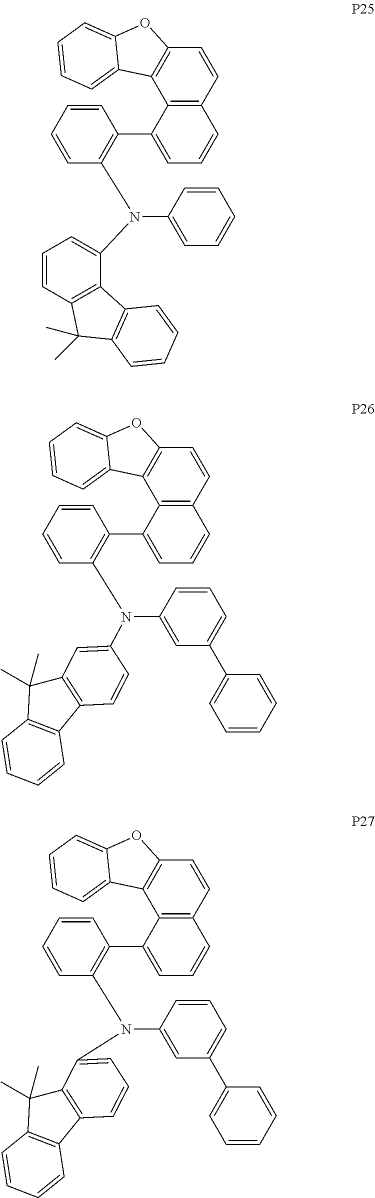 [SMILES: CC1(C)c2ccccc2-c2c(N(c3ccccc3)c3ccccc3-c3cccc4ccc5oc6ccccc6c5c34)cccc21 ; CC1(C)c2ccccc2-c2ccc(N(c3cccc(-c4ccccc4)c3)c3ccccc3-c3cccc4ccc5oc6ccccc6c5c34)cc21 ; CC1(C)c2ccccc2-c2cccc(N(c3cccc(-c4ccccc4)c3)c3ccccc3-c3cccc4ccc5oc6ccccc6c5c34)c21]

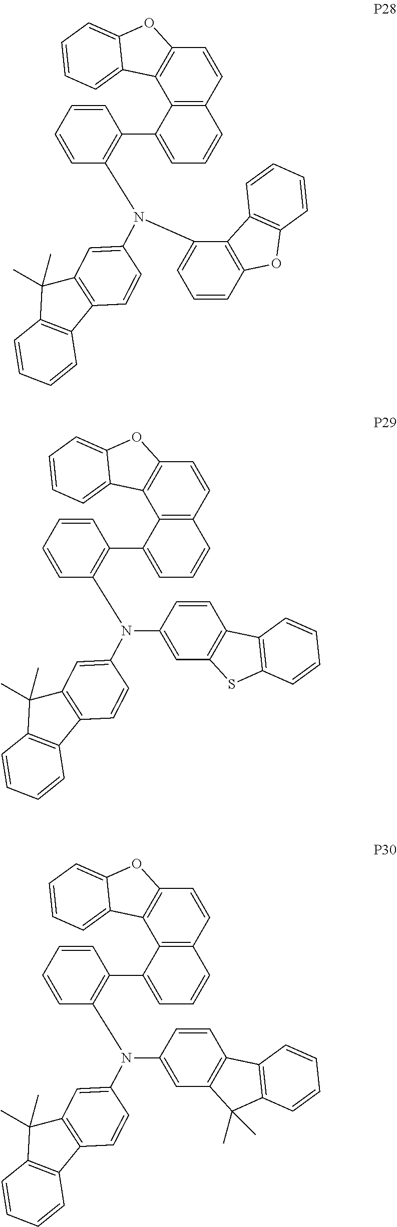 [SMILES: CC1(C)c2ccccc2-c2ccc(N(c3ccccc3-c3cccc4ccc5oc6ccccc6c5c34)c3cccc4oc5ccccc5c34)cc21 ; CC1(C)c2ccccc2-c2ccc(N(c3ccc4c(c3)sc3ccccc34)c3ccccc3-c3cccc4ccc5oc6ccccc6c5c34)cc21 ; CC1(C)c2ccccc2-c2ccc(N(c3ccc4c(c3)C(C)(C)c3ccccc3-4)c3ccccc3-c3cccc4ccc5oc6ccccc6c5c34)cc21]

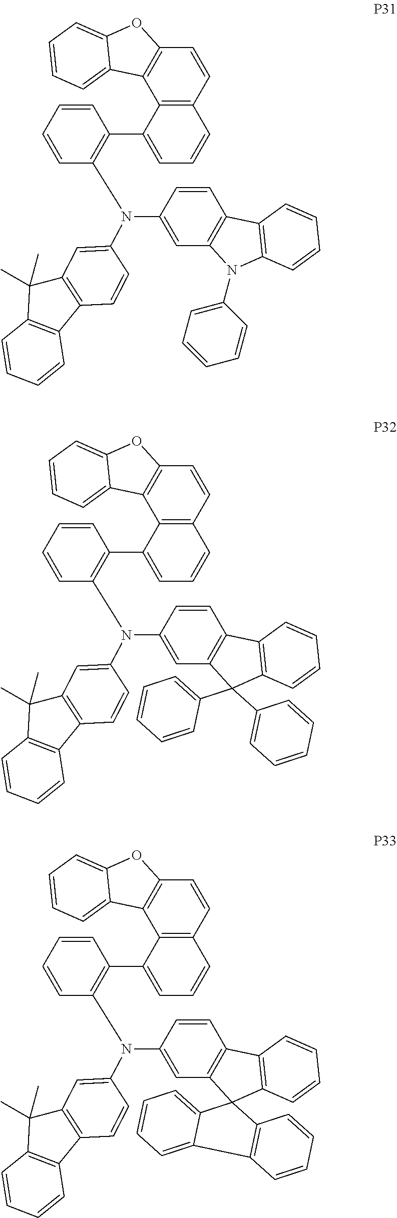 [SMILES: CC1(C)c2ccccc2-c2ccc(N(c3ccc4c5ccccc5n(-c5ccccc5)c4c3)c3ccccc3-c3cccc4ccc5oc6ccccc6c5c34)cc21 ; CC1(C)c2ccccc2-c2ccc(N(c3ccc4c(c3)C(c3ccccc3)(c3ccccc3)c3ccccc3-4)c3ccccc3-c3cccc4ccc5oc6ccccc6c5c34)cc21 ; CC1(C)c2ccccc2-c2ccc(N(c3ccc4c(c3)C3(c5ccccc5-c5ccccc53)c3ccccc3-4)c3ccccc3-c3cccc4ccc5oc6ccccc6c5c34)cc21]

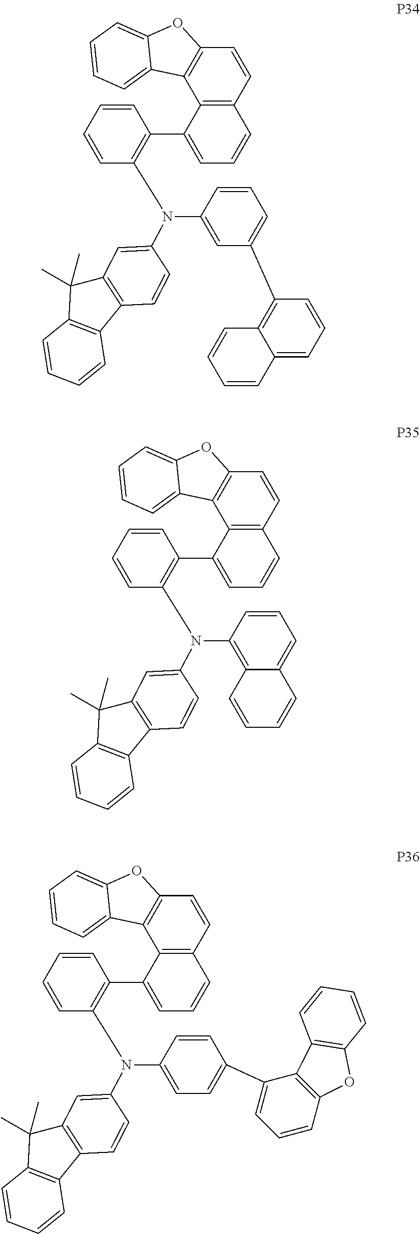 [SMILES: CC1(C)c2ccccc2-c2ccc(N(c3cccc(-c4cccc5ccccc45)c3)c3ccccc3-c3cccc4ccc5oc6ccccc6c5c34)cc21 ; CC1(C)c2ccccc2-c2ccc(N(c3ccccc3-c3cccc4ccc5oc6ccccc6c5c34)c3cccc4ccccc34)cc21 ; CC1(C)c2ccccc2-c2ccc(N(c3ccc(-c4cccc5oc6ccccc6c45)cc3)c3ccccc3-c3cccc4ccc5oc6ccccc6c5c34)cc21]

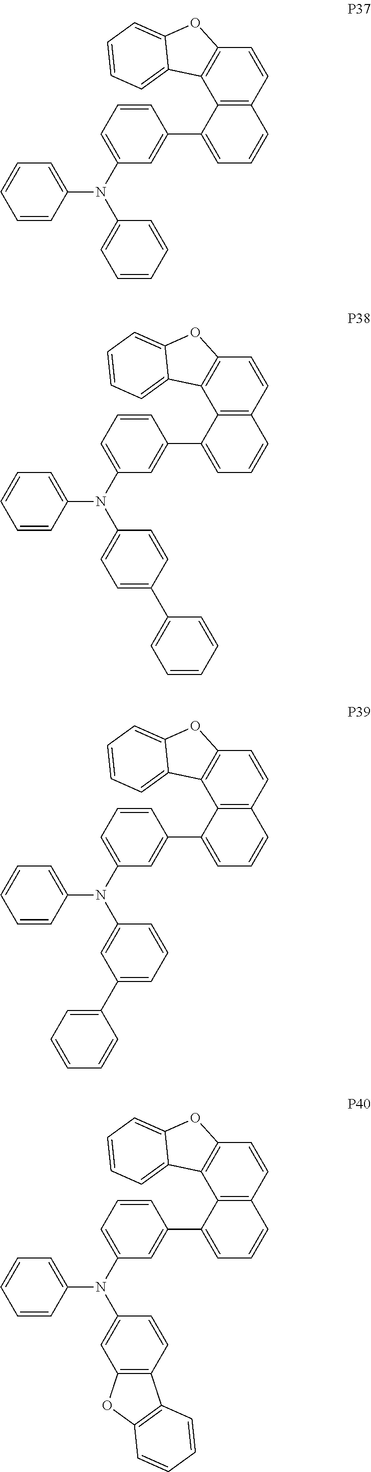 [SMILES: c1ccc(N(c2ccccc2)c2cccc(-c3cccc4ccc5oc6ccccc6c5c34)c2)cc1 ; c1ccc(-c2ccc(N(c3ccccc3)c3cccc(-c4cccc5ccc6oc7ccccc7c6c45)c3)cc2)cc1 ; c1ccc(-c2cccc(N(c3ccccc3)c3cccc(-c4cccc5ccc6oc7ccccc7c6c45)c3)c2)cc1 ; c1ccc(N(c2cccc(-c3cccc4ccc5oc6ccccc6c5c34)c2)c2ccc3c(c2)oc2ccccc23)cc1]

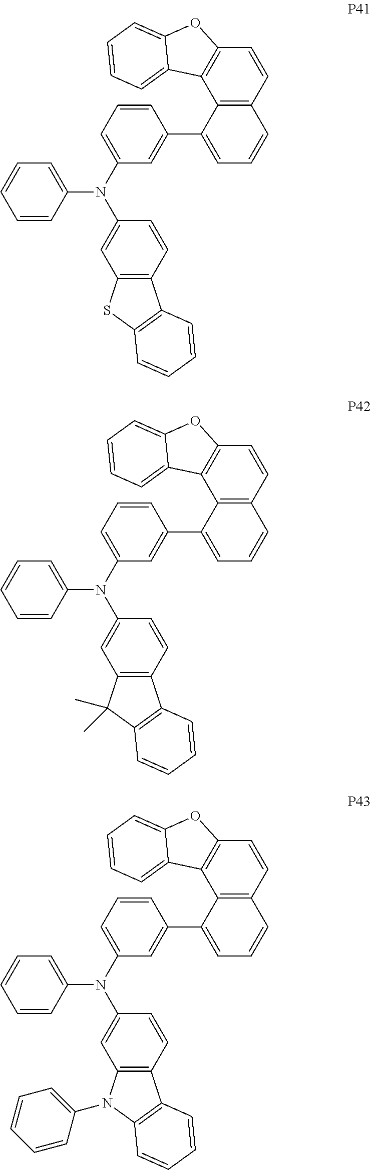 [SMILES: c1ccc(N(c2cccc(-c3cccc4ccc5oc6ccccc6c5c34)c2)c2ccc3c(c2)sc2ccccc23)cc1 ; CC1(C)c2ccccc2-c2ccc(N(c3ccccc3)c3cccc(-c4cccc5ccc6oc7ccccc7c6c45)c3)cc21 ; c1ccc(N(c2cccc(-c3cccc4ccc5oc6ccccc6c5c34)c2)c2ccc3c4ccccc4n(-c4ccccc4)c3c2)cc1]

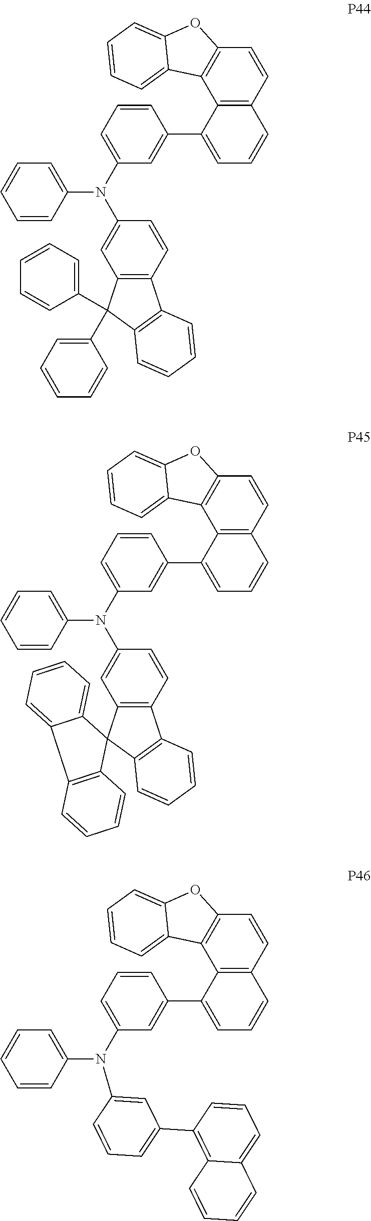 [SMILES: c1ccc(N(c2cccc(-c3cccc4ccc5oc6ccccc6c5c34)c2)c2ccc3c(c2)C(c2ccccc2)(c2ccccc2)c2ccccc2-3)cc1 ; c1ccc(N(c2cccc(-c3cccc4ccc5oc6ccccc6c5c34)c2)c2ccc3c(c2)C2(c4ccccc4-c4ccccc42)c2ccccc2-3)cc1 ; c1ccc(N(c2cccc(-c3cccc4ccccc34)c2)c2cccc(-c3cccc4ccc5oc6ccccc6c5c34)c2)cc1]

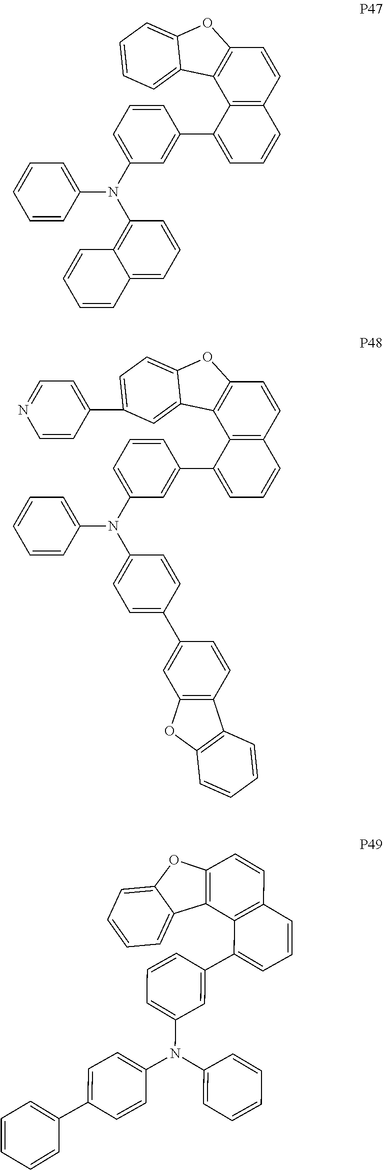 [SMILES: c1ccc(N(c2cccc(-c3cccc4ccc5oc6ccccc6c5c34)c2)c2cccc3ccccc23)cc1 ; c1ccc(N(c2ccc(-c3ccc4c(c3)oc3ccccc34)cc2)c2cccc(-c3cccc4ccc5oc6ccc(-c7ccncc7)cc6c5c34)c2)cc1 ; c1ccc(-c2ccc(N(c3ccccc3)c3cccc(-c4cccc5ccc6oc7ccccc7c6c45)c3)cc2)cc1]

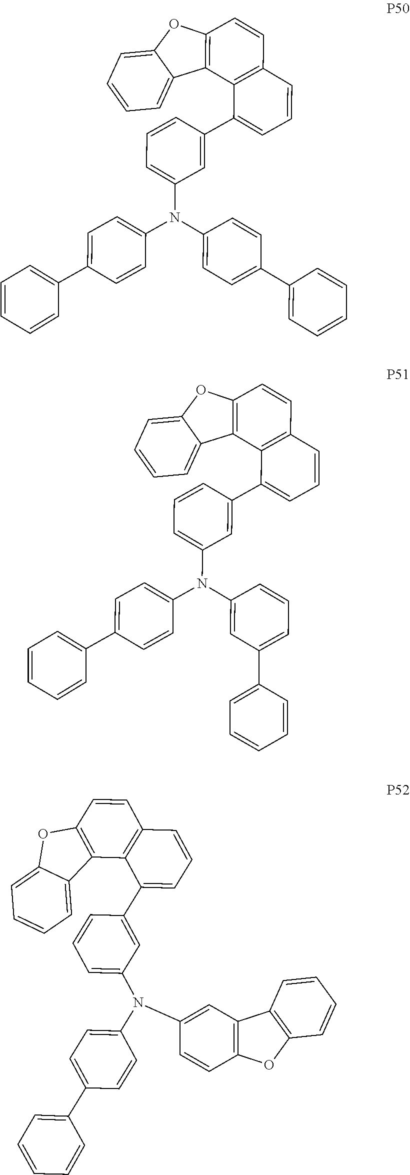 [SMILES: c1ccc(-c2ccc(N(c3ccc(-c4ccccc4)cc3)c3cccc(-c4cccc5ccc6oc7ccccc7c6c45)c3)cc2)cc1 ; c1ccc(-c2ccc(N(c3cccc(-c4ccccc4)c3)c3cccc(-c4cccc5ccc6oc7ccccc7c6c45)c3)cc2)cc1 ; c1ccc(-c2ccc(N(c3cccc(-c4cccc5ccc6oc7ccccc7c6c45)c3)c3ccc4oc5ccccc5c4c3)cc2)cc1]

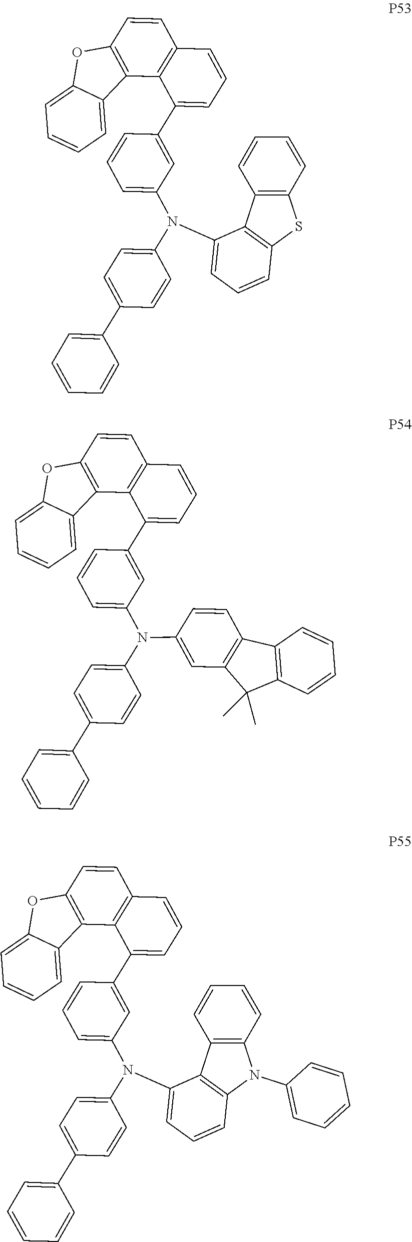

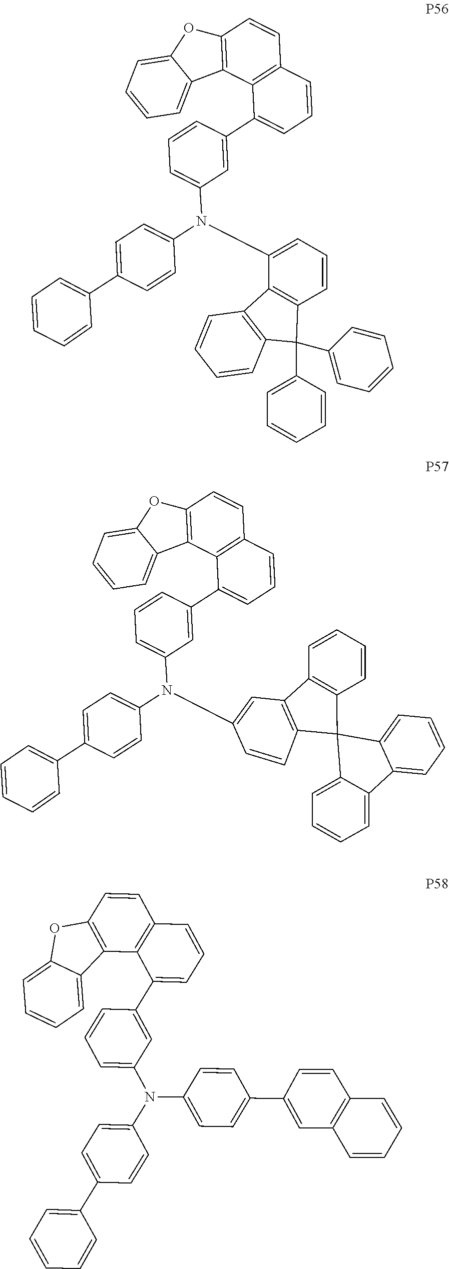 [SMILES: c1ccc(-c2ccc(N(c3cccc(-c4cccc5ccc6oc7ccccc7c6c45)c3)c3cccc4c3-c3ccccc3C4(c3ccccc3)c3ccccc3)cc2)cc1 ; c1ccc(-c2ccc(N(c3cccc(-c4cccc5ccc6oc7ccccc7c6c45)c3)c3ccc4c(c3)-c3ccccc3C43c4ccccc4-c4ccccc43)cc2)cc1 ; c1ccc(-c2ccc(N(c3ccc(-c4ccc5ccccc5c4)cc3)c3cccc(-c4cccc5ccc6oc7ccccc7c6c45)c3)cc2)cc1]

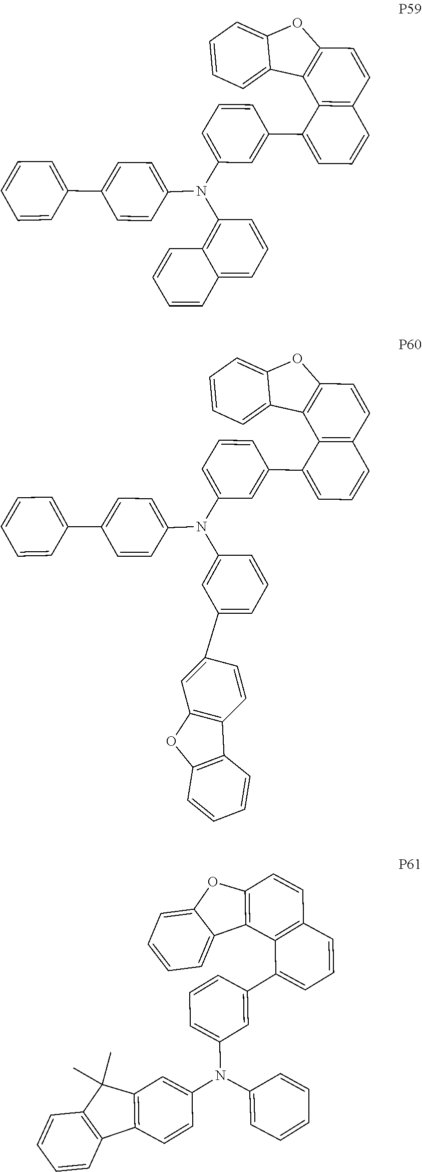 [SMILES: c1ccc(-c2ccc(N(c3cccc(-c4cccc5ccc6oc7ccccc7c6c45)c3)c3cccc4ccccc34)cc2)cc1 ; c1ccc(-c2ccc(N(c3cccc(-c4ccc5c(c4)oc4ccccc45)c3)c3cccc(-c4cccc5ccc6oc7ccccc7c6c45)c3)cc2)cc1 ; CC1(C)c2ccccc2-c2ccc(N(c3ccccc3)c3cccc(-c4cccc5ccc6oc7ccccc7c6c45)c3)cc21]

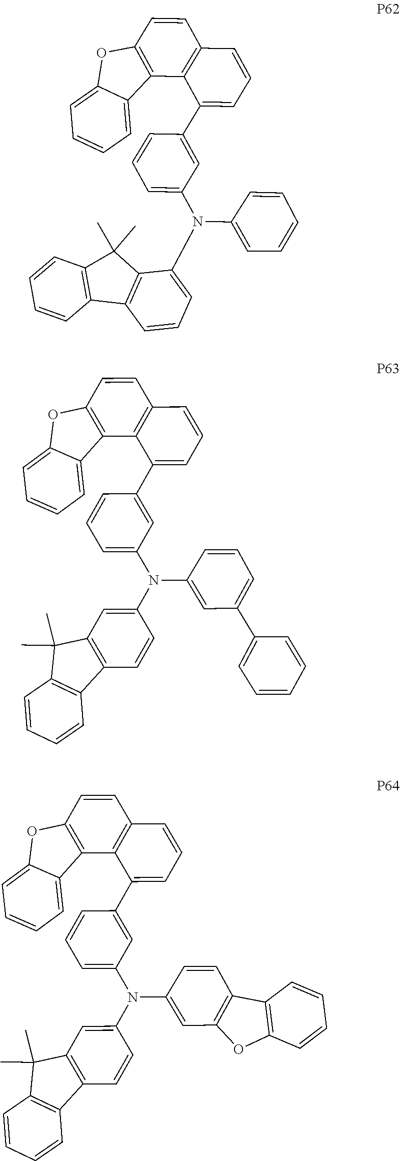 [SMILES: CC1(C)c2ccccc2-c2cccc(N(c3ccccc3)c3cccc(-c4cccc5ccc6oc7ccccc7c6c45)c3)c21 ; CC1(C)c2ccccc2-c2ccc(N(c3cccc(-c4ccccc4)c3)c3cccc(-c4cccc5ccc6oc7ccccc7c6c45)c3)cc21 ; CC1(C)c2ccccc2-c2ccc(N(c3cccc(-c4cccc5ccc6oc7ccccc7c6c45)c3)c3ccc4c(c3)oc3ccccc34)cc21]

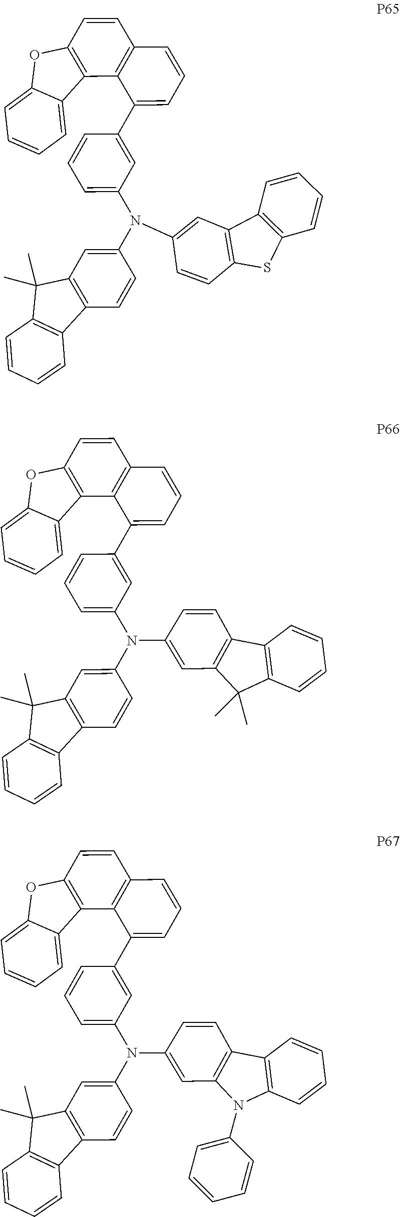 [SMILES: CC1(C)c2ccccc2-c2ccc(N(c3cccc(-c4cccc5ccc6oc7ccccc7c6c45)c3)c3ccc4sc5ccccc5c4c3)cc21 ; CC1(C)c2ccccc2-c2ccc(N(c3cccc(-c4cccc5ccc6oc7ccccc7c6c45)c3)c3ccc4c(c3)C(C)(C)c3ccccc3-4)cc21 ; CC1(C)c2ccccc2-c2ccc(N(c3cccc(-c4cccc5ccc6oc7ccccc7c6c45)c3)c3ccc4c5ccccc5n(-c5ccccc5)c4c3)cc21]

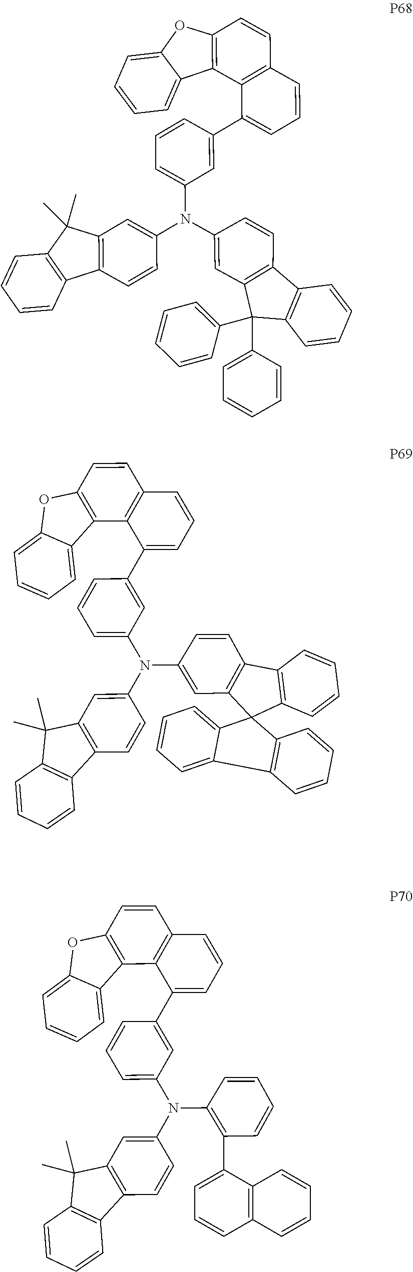 [SMILES: CC1(C)c2ccccc2-c2ccc(N(c3cccc(-c4cccc5ccc6oc7ccccc7c6c45)c3)c3ccc4c(c3)C(c3ccccc3)(c3ccccc3)c3ccccc3-4)cc21 ; CC1(C)c2ccccc2-c2ccc(N(c3cccc(-c4cccc5ccc6oc7ccccc7c6c45)c3)c3ccc4c(c3)C3(c5ccccc5-c5ccccc53)c3ccccc3-4)cc21 ; CC1(C)c2ccccc2-c2ccc(N(c3cccc(-c4cccc5ccc6oc7ccccc7c6c45)c3)c3ccccc3-c3cccc4ccccc34)cc21]

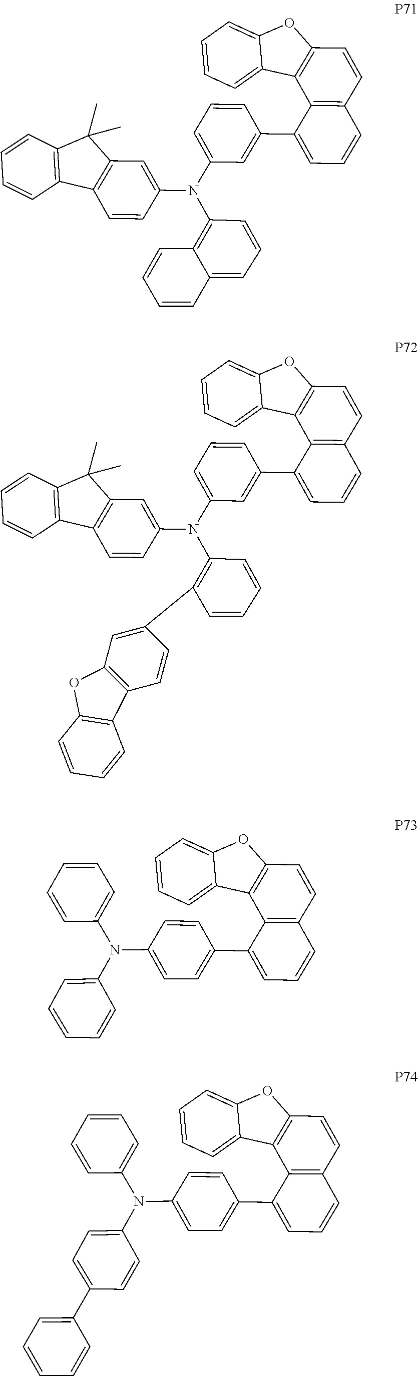 [SMILES: CC1(C)c2ccccc2-c2ccc(N(c3cccc(-c4cccc5ccc6oc7ccccc7c6c45)c3)c3cccc4ccccc34)cc21 ; CC1(C)c2ccccc2-c2ccc(N(c3cccc(-c4cccc5ccc6oc7ccccc7c6c45)c3)c3ccccc3-c3ccc4c(c3)oc3ccccc34)cc21 ; c1ccc(N(c2ccccc2)c2ccc(-c3cccc4ccc5oc6ccccc6c5c34)cc2)cc1 ; c1ccc(-c2ccc(N(c3ccccc3)c3ccc(-c4cccc5ccc6oc7ccccc7c6c45)cc3)cc2)cc1]

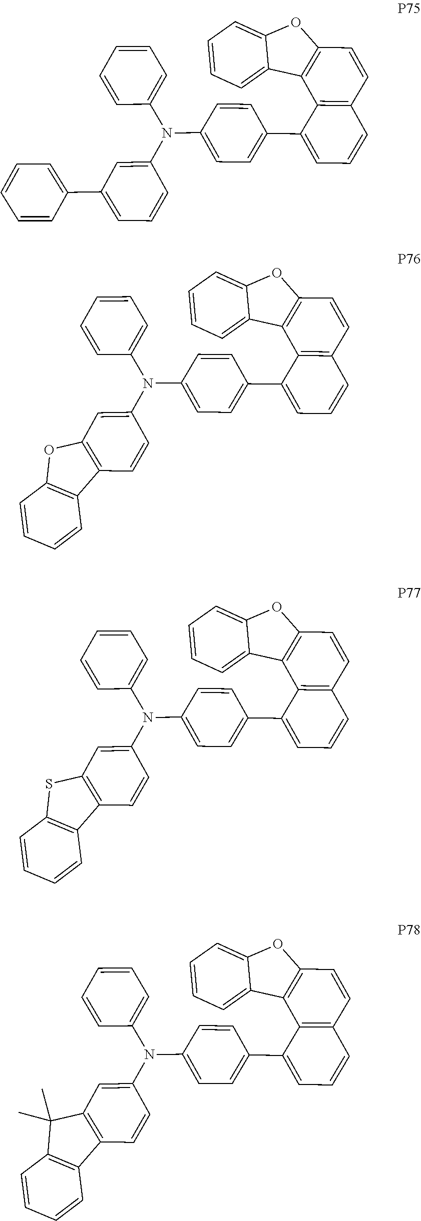 [SMILES: c1ccc(-c2cccc(N(c3ccccc3)c3ccc(-c4cccc5ccc6oc7ccccc7c6c45)cc3)c2)cc1 ; c1ccc(N(c2ccc(-c3cccc4ccc5oc6ccccc6c5c34)cc2)c2ccc3c(c2)oc2ccccc23)cc1 ; c1ccc(N(c2ccc(-c3cccc4ccc5oc6ccccc6c5c34)cc2)c2ccc3c(c2)sc2ccccc23)cc1 ; CC1(C)c2ccccc2-c2ccc(N(c3ccccc3)c3ccc(-c4cccc5ccc6oc7ccccc7c6c45)cc3)cc21]

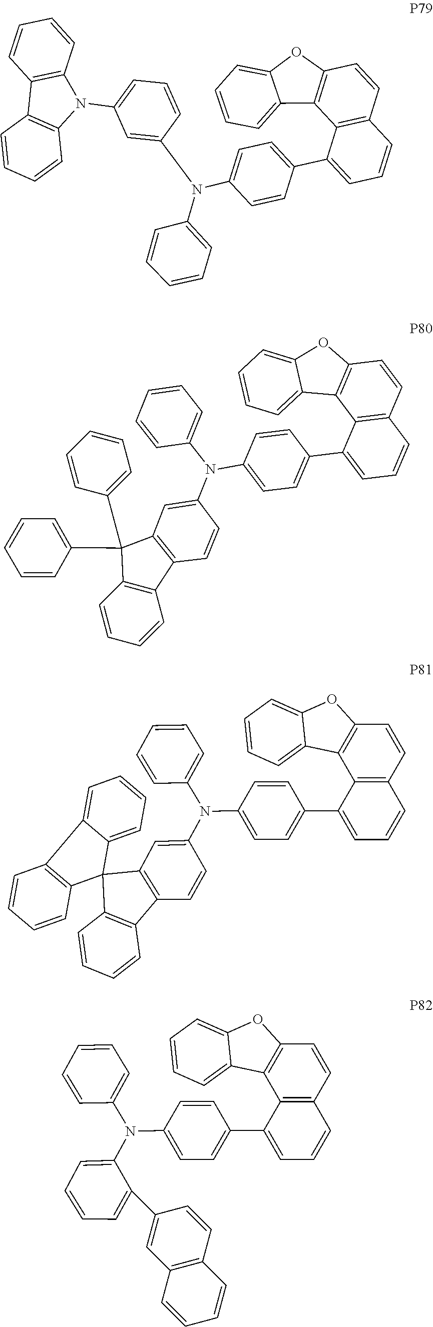 [SMILES: c1ccc(N(c2ccc(-c3cccc4ccc5oc6ccccc6c5c34)cc2)c2cccc(-n3c4ccccc4c4ccccc43)c2)cc1 ; c1ccc(N(c2ccc(-c3cccc4ccc5oc6ccccc6c5c34)cc2)c2ccc3c(c2)C(c2ccccc2)(c2ccccc2)c2ccccc2-3)cc1 ; c1ccc(N(c2ccc(-c3cccc4ccc5oc6ccccc6c5c34)cc2)c2ccc3c(c2)C2(c4ccccc4-c4ccccc42)c2ccccc2-3)cc1 ; c1ccc(N(c2ccc(-c3cccc4ccc5oc6ccccc6c5c34)cc2)c2ccccc2-c2ccc3ccccc3c2)cc1]

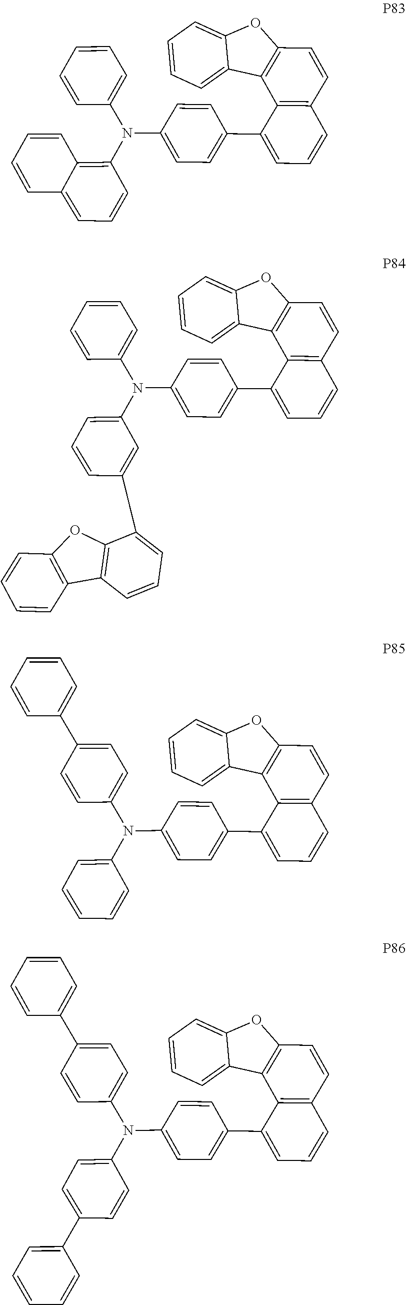

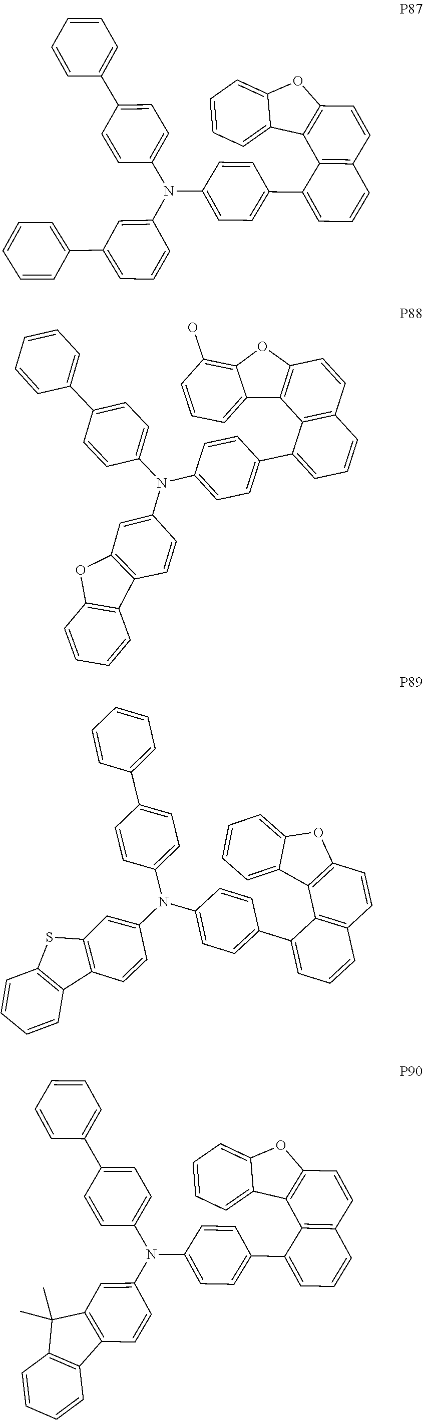

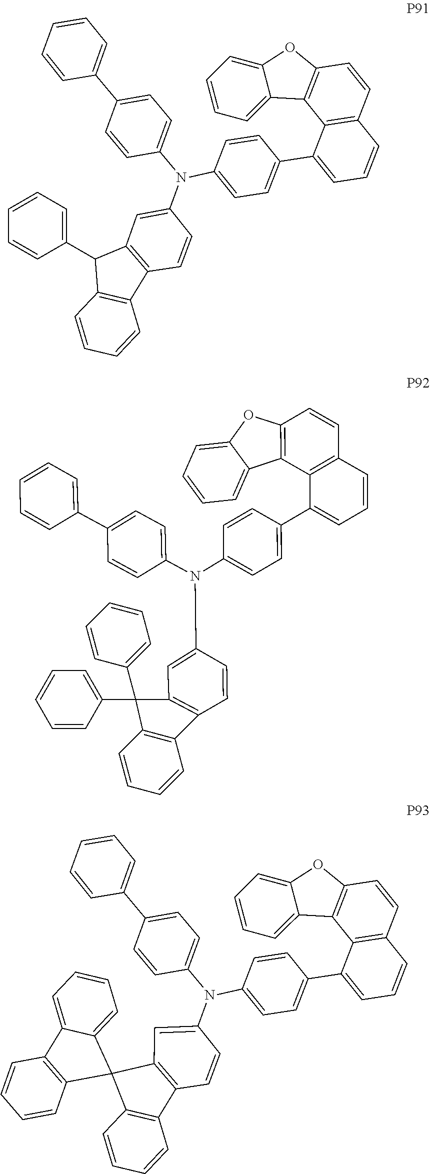 [SMILES: c1ccc(-c2ccc(N(c3ccc(-c4cccc5ccc6oc7ccccc7c6c45)cc3)c3ccc4c(c3)C(c3ccccc3)c3ccccc3-4)cc2)cc1 ; c1ccc(-c2ccc(N(c3ccc(-c4cccc5ccc6oc7ccccc7c6c45)cc3)c3ccc4c(c3)C(c3ccccc3)(c3ccccc3)c3ccccc3-4)cc2)cc1 ; c1ccc(-c2ccc(N(c3ccc(-c4cccc5ccc6oc7ccccc7c6c45)cc3)c3ccc4c(c3)C3(c5ccccc5-c5ccccc53)c3ccccc3-4)cc2)cc1]

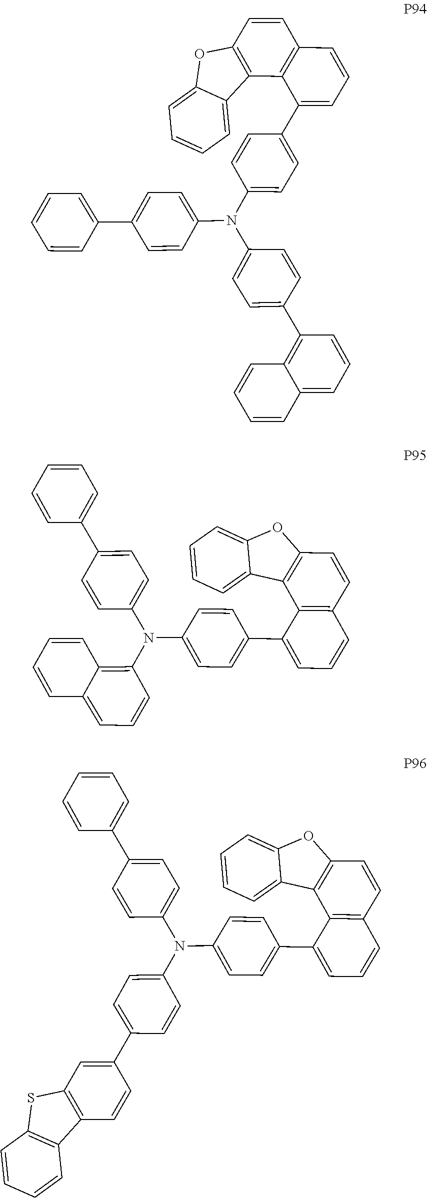 [SMILES: c1ccc(-c2ccc(N(c3ccc(-c4cccc5ccccc45)cc3)c3ccc(-c4cccc5ccc6oc7ccccc7c6c45)cc3)cc2)cc1 ; c1ccc(-c2ccc(N(c3ccc(-c4cccc5ccc6oc7ccccc7c6c45)cc3)c3cccc4ccccc34)cc2)cc1 ; c1ccc(-c2ccc(N(c3ccc(-c4ccc5c(c4)sc4ccccc45)cc3)c3ccc(-c4cccc5ccc6oc7ccccc7c6c45)cc3)cc2)cc1]

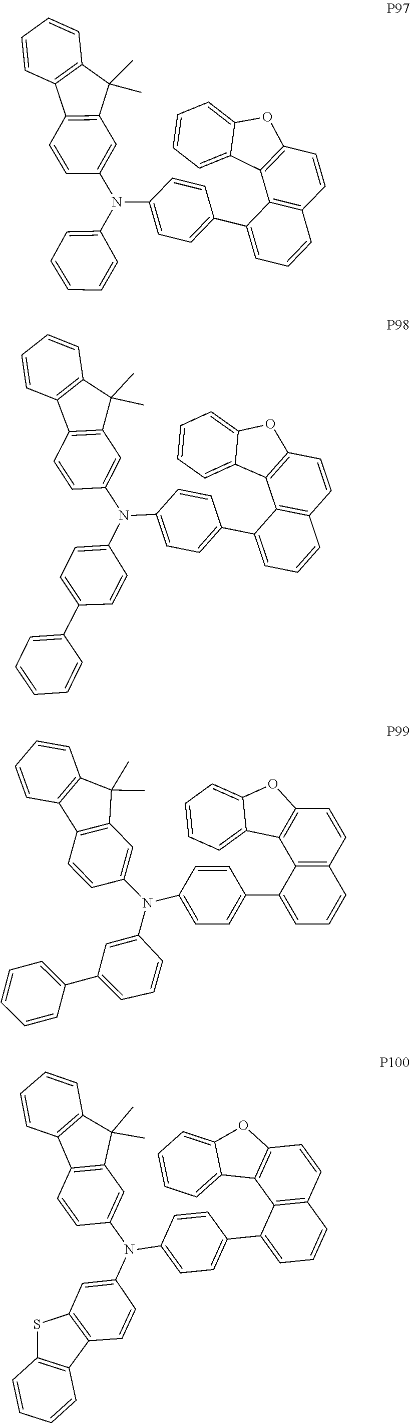

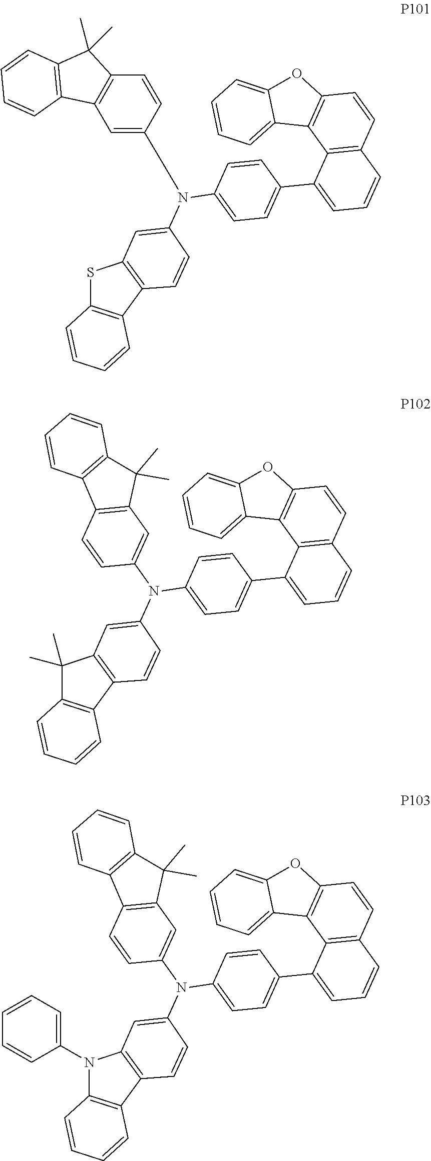 [SMILES: CC1(C)c2ccccc2-c2cc(N(c3ccc(-c4cccc5ccc6oc7ccccc7c6c45)cc3)c3ccc4c(c3)sc3ccccc34)ccc21 ; CC1(C)c2ccccc2-c2ccc(N(c3ccc(-c4cccc5ccc6oc7ccccc7c6c45)cc3)c3ccc4c(c3)C(C)(C)c3ccccc3-4)cc21 ; CC1(C)c2ccccc2-c2ccc(N(c3ccc(-c4cccc5ccc6oc7ccccc7c6c45)cc3)c3ccc4c5ccccc5n(-c5ccccc5)c4c3)cc21]

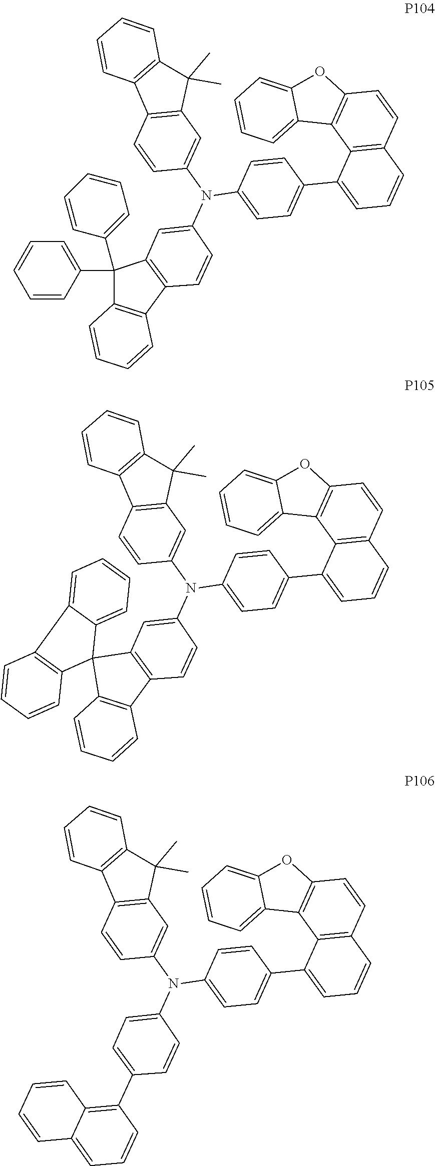 [SMILES: CC1(C)c2ccccc2-c2ccc(N(c3ccc(-c4cccc5ccc6oc7ccccc7c6c45)cc3)c3ccc4c(c3)C(c3ccccc3)(c3ccccc3)c3ccccc3-4)cc21 ; CC1(C)c2ccccc2-c2ccc(N(c3ccc(-c4cccc5ccc6oc7ccccc7c6c45)cc3)c3ccc4c(c3)C3(c5ccccc5-c5ccccc53)c3ccccc3-4)cc21 ; CC1(C)c2ccccc2-c2ccc(N(c3ccc(-c4cccc5ccccc45)cc3)c3ccc(-c4cccc5ccc6oc7ccccc7c6c45)cc3)cc21]

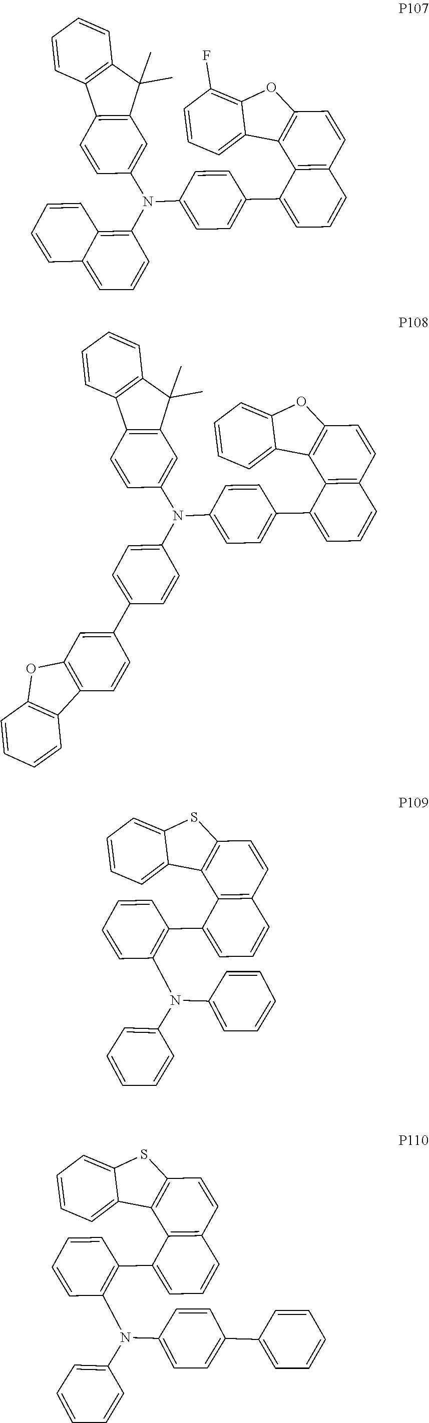

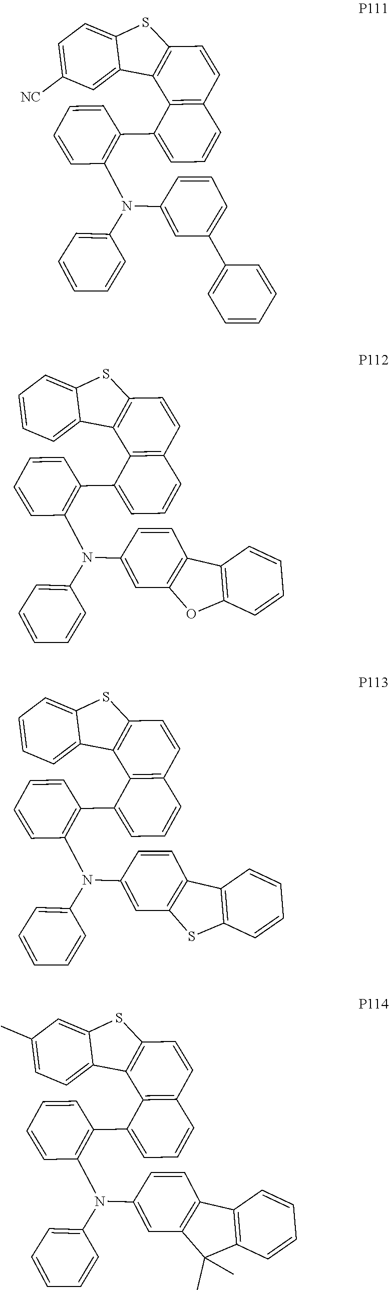 [SMILES: N#Cc1ccc2sc3ccc4cccc(-c5ccccc5N(c5ccccc5)c5cccc(-c6ccccc6)c5)c4c3c2c1 ; c1ccc(N(c2ccc3c(c2)oc2ccccc23)c2ccccc2-c2cccc3ccc4sc5ccccc5c4c23)cc1 ; c1ccc(N(c2ccc3c(c2)sc2ccccc23)c2ccccc2-c2cccc3ccc4sc5ccccc5c4c23)cc1 ; Cc1ccc2c(c1)sc1ccc3cccc(-c4ccccc4N(c4ccccc4)c4ccc5c(c4)C(C)(C)c4ccccc4-5)c3c12]

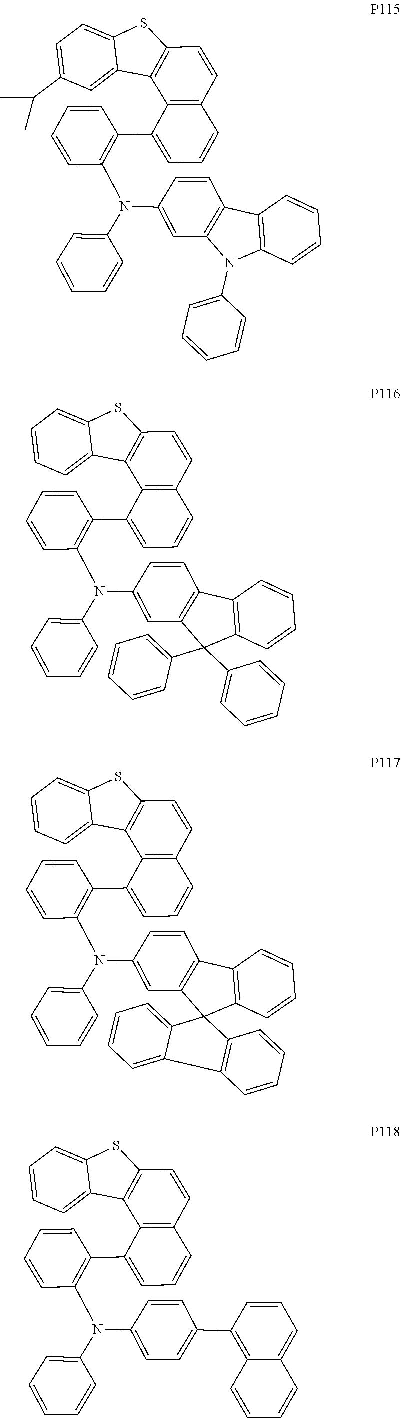 [SMILES: CC(C)c1ccc2sc3ccc4cccc(-c5ccccc5N(c5ccccc5)c5ccc6c7ccccc7n(-c7ccccc7)c6c5)c4c3c2c1 ; c1ccc(N(c2ccc3c(c2)C(c2ccccc2)(c2ccccc2)c2ccccc2-3)c2ccccc2-c2cccc3ccc4sc5ccccc5c4c23)cc1 ; c1ccc(N(c2ccc3c(c2)C2(c4ccccc4-c4ccccc42)c2ccccc2-3)c2ccccc2-c2cccc3ccc4sc5ccccc5c4c23)cc1 ; c1ccc(N(c2ccc(-c3cccc4ccccc34)cc2)c2ccccc2-c2cccc3ccc4sc5ccccc5c4c23)cc1]

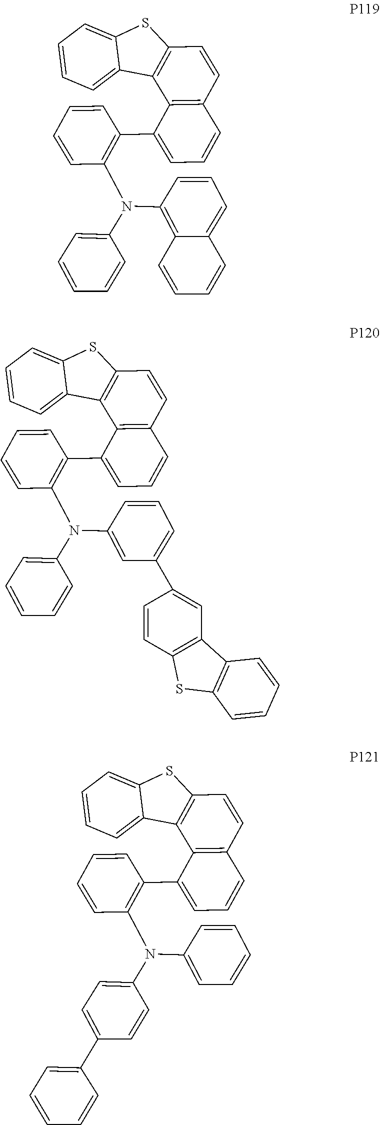 [SMILES: c1ccc(N(c2ccccc2-c2cccc3ccc4sc5ccccc5c4c23)c2cccc3ccccc23)cc1 ; c1ccc(N(c2cccc(-c3ccc4sc5ccccc5c4c3)c2)c2ccccc2-c2cccc3ccc4sc5ccccc5c4c23)cc1 ; c1ccc(-c2ccc(N(c3ccccc3)c3ccccc3-c3cccc4ccc5sc6ccccc6c5c34)cc2)cc1]

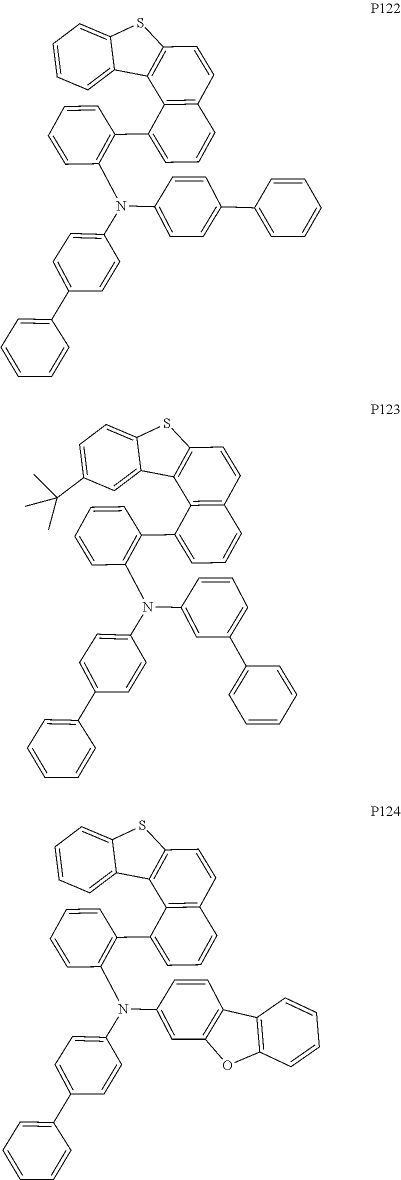 [SMILES: c1ccc(-c2ccc(N(c3ccc(-c4ccccc4)cc3)c3ccccc3-c3cccc4ccc5sc6ccccc6c5c34)cc2)cc1 ; CC(C)(C)c1ccc2sc3ccc4cccc(-c5ccccc5N(c5ccc(-c6ccccc6)cc5)c5cccc(-c6ccccc6)c5)c4c3c2c1 ; c1ccc(-c2ccc(N(c3ccc4c(c3)oc3ccccc34)c3ccccc3-c3cccc4ccc5sc6ccccc6c5c34)cc2)cc1]

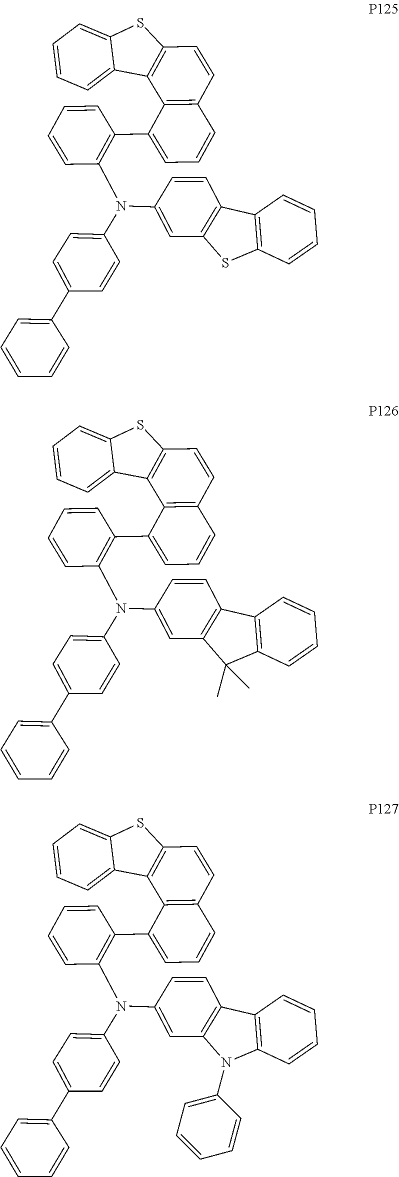 [SMILES: c1ccc(-c2ccc(N(c3ccc4c(c3)sc3ccccc34)c3ccccc3-c3cccc4ccc5sc6ccccc6c5c34)cc2)cc1 ; CC1(C)c2ccccc2-c2ccc(N(c3ccc(-c4ccccc4)cc3)c3ccccc3-c3cccc4ccc5sc6ccccc6c5c34)cc21 ; c1ccc(-c2ccc(N(c3ccc4c5ccccc5n(-c5ccccc5)c4c3)c3ccccc3-c3cccc4ccc5sc6ccccc6c5c34)cc2)cc1]

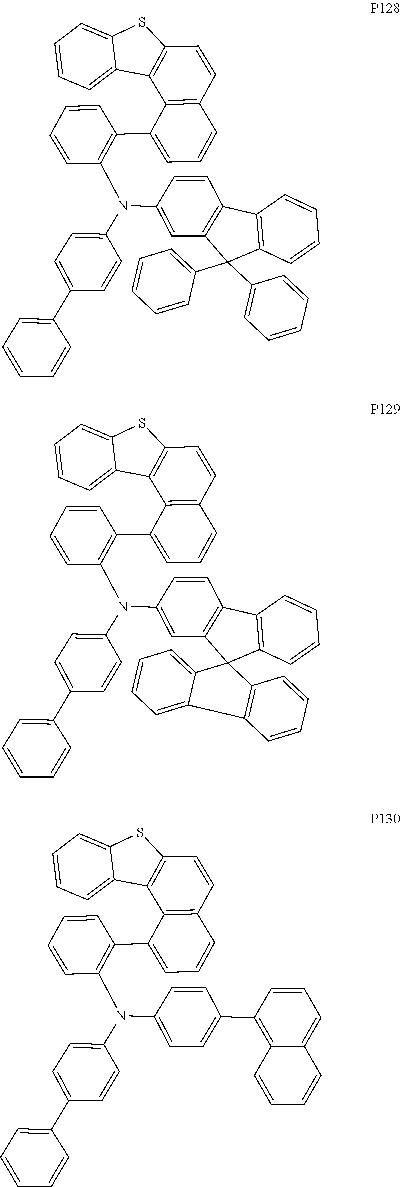 [SMILES: c1ccc(-c2ccc(N(c3ccc4c(c3)C(c3ccccc3)(c3ccccc3)c3ccccc3-4)c3ccccc3-c3cccc4ccc5sc6ccccc6c5c34)cc2)cc1 ; c1ccc(-c2ccc(N(c3ccc4c(c3)C3(c5ccccc5-c5ccccc53)c3ccccc3-4)c3ccccc3-c3cccc4ccc5sc6ccccc6c5c34)cc2)cc1 ; c1ccc(-c2ccc(N(c3ccc(-c4cccc5ccccc45)cc3)c3ccccc3-c3cccc4ccc5sc6ccccc6c5c34)cc2)cc1]

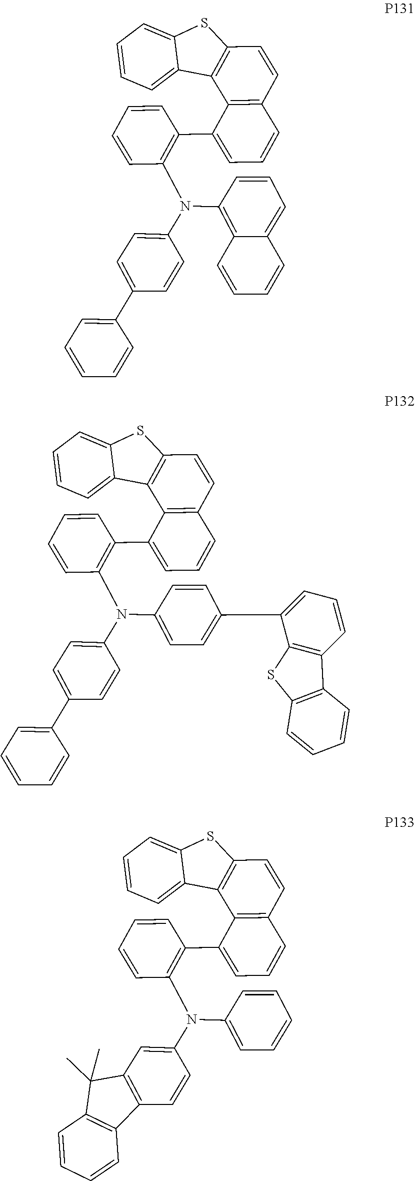 [SMILES: c1ccc(-c2ccc(N(c3ccccc3-c3cccc4ccc5sc6ccccc6c5c34)c3cccc4ccccc34)cc2)cc1 ; c1ccc(-c2ccc(N(c3ccc(-c4cccc5c4sc4ccccc45)cc3)c3ccccc3-c3cccc4ccc5sc6ccccc6c5c34)cc2)cc1 ; CC1(C)c2ccccc2-c2ccc(N(c3ccccc3)c3ccccc3-c3cccc4ccc5sc6ccccc6c5c34)cc21]

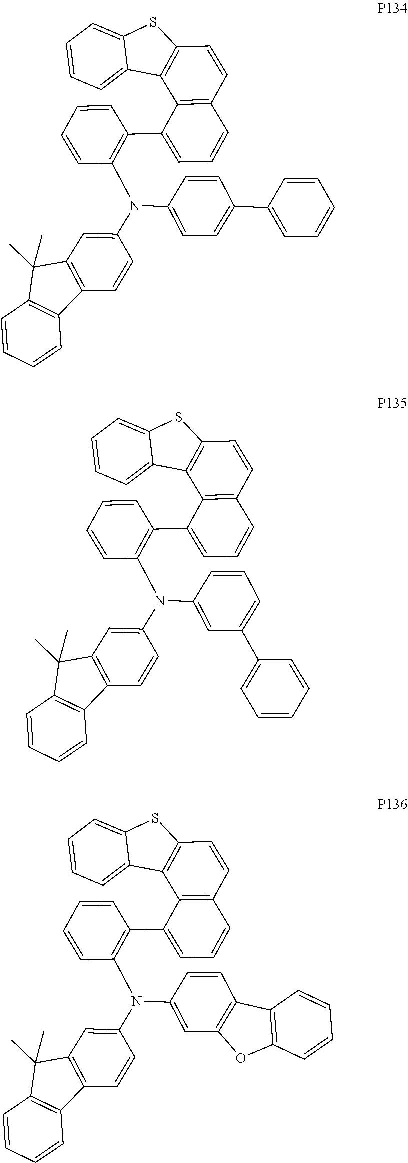 [SMILES: CC1(C)c2ccccc2-c2ccc(N(c3ccc(-c4ccccc4)cc3)c3ccccc3-c3cccc4ccc5sc6ccccc6c5c34)cc21 ; CC1(C)c2ccccc2-c2ccc(N(c3cccc(-c4ccccc4)c3)c3ccccc3-c3cccc4ccc5sc6ccccc6c5c34)cc21 ; CC1(C)c2ccccc2-c2ccc(N(c3ccc4c(c3)oc3ccccc34)c3ccccc3-c3cccc4ccc5sc6ccccc6c5c34)cc21]

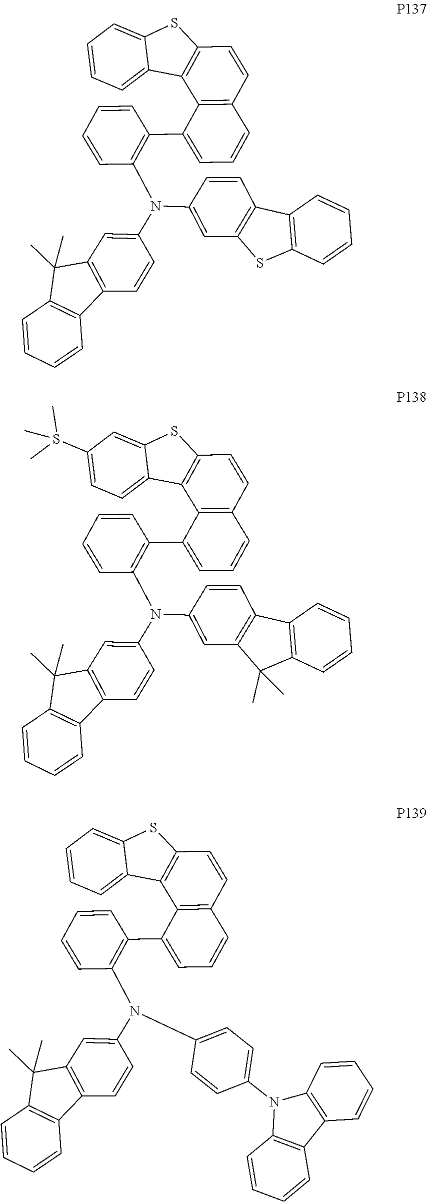

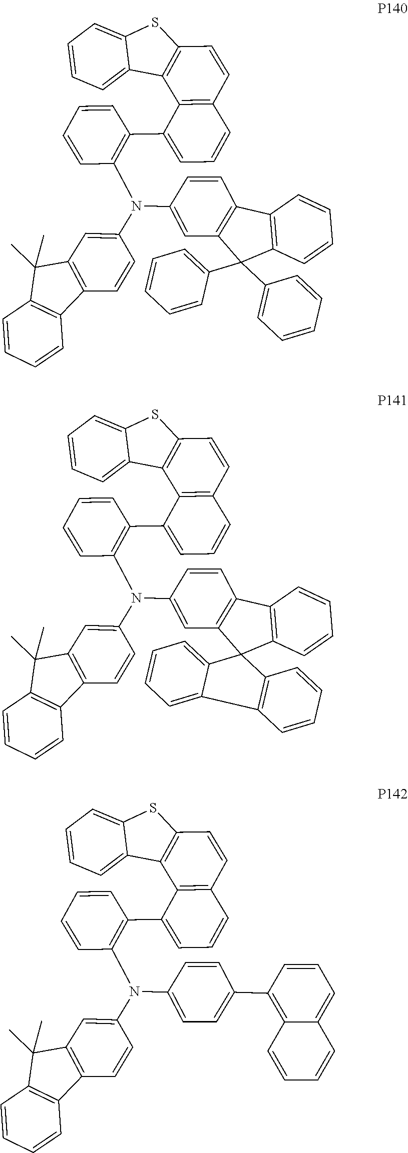 [SMILES: CC1(C)c2ccccc2-c2ccc(N(c3ccc4c(c3)C(c3ccccc3)(c3ccccc3)c3ccccc3-4)c3ccccc3-c3cccc4ccc5sc6ccccc6c5c34)cc21 ; CC1(C)c2ccccc2-c2ccc(N(c3ccc4c(c3)C3(c5ccccc5-c5ccccc53)c3ccccc3-4)c3ccccc3-c3cccc4ccc5sc6ccccc6c5c34)cc21 ; CC1(C)c2ccccc2-c2ccc(N(c3ccc(-c4cccc5ccccc45)cc3)c3ccccc3-c3cccc4ccc5sc6ccccc6c5c34)cc21]

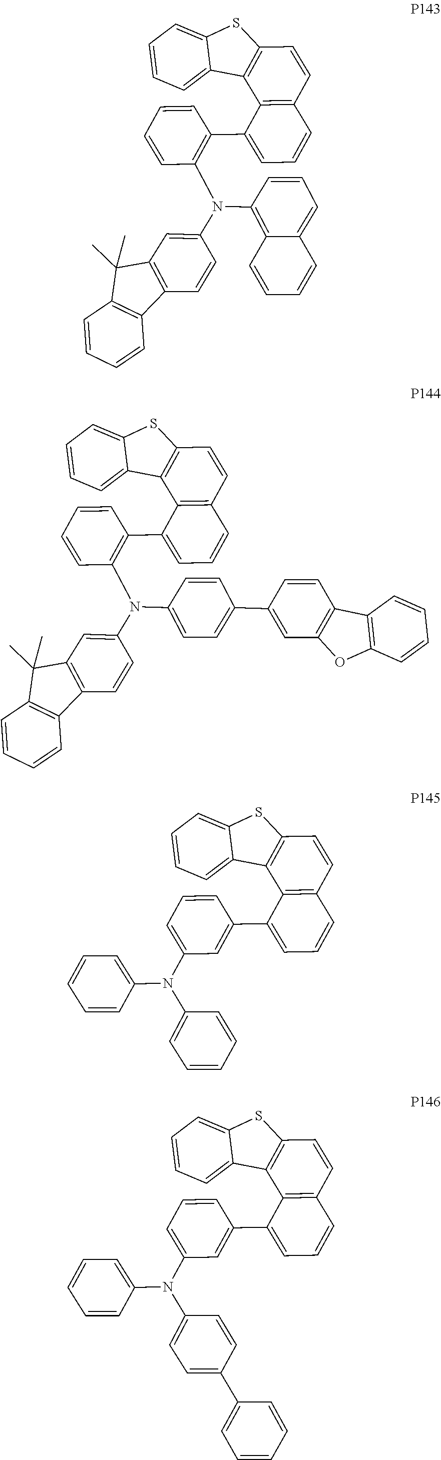 [SMILES: CC1(C)c2ccccc2-c2ccc(N(c3ccccc3-c3cccc4ccc5sc6ccccc6c5c34)c3cccc4ccccc34)cc21 ; CC1(C)c2ccccc2-c2ccc(N(c3ccc(-c4ccc5c(c4)oc4ccccc45)cc3)c3ccccc3-c3cccc4ccc5sc6ccccc6c5c34)cc21 ; c1ccc(N(c2ccccc2)c2cccc(-c3cccc4ccc5sc6ccccc6c5c34)c2)cc1 ; c1ccc(-c2ccc(N(c3ccccc3)c3cccc(-c4cccc5ccc6sc7ccccc7c6c45)c3)cc2)cc1]

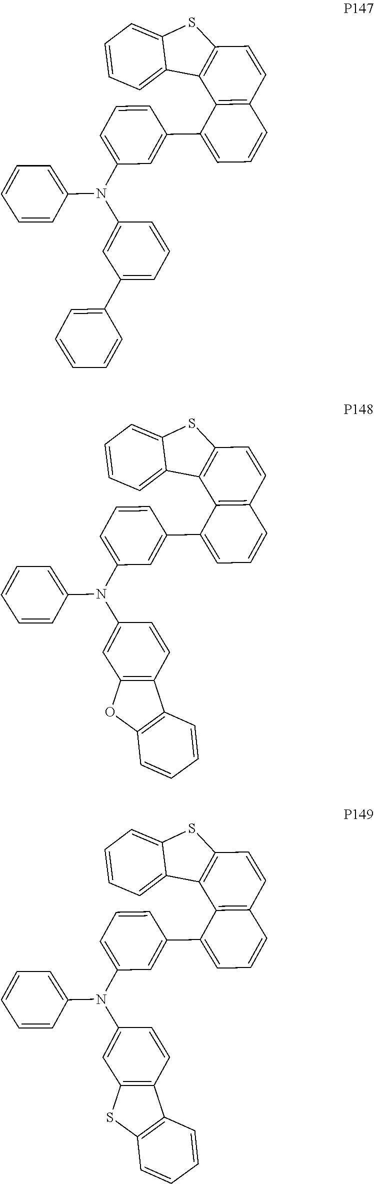 [SMILES: c1ccc(-c2cccc(N(c3ccccc3)c3cccc(-c4cccc5ccc6sc7ccccc7c6c45)c3)c2)cc1 ; c1ccc(N(c2cccc(-c3cccc4ccc5sc6ccccc6c5c34)c2)c2ccc3c(c2)oc2ccccc23)cc1 ; c1ccc(N(c2cccc(-c3cccc4ccc5sc6ccccc6c5c34)c2)c2ccc3c(c2)sc2ccccc23)cc1]

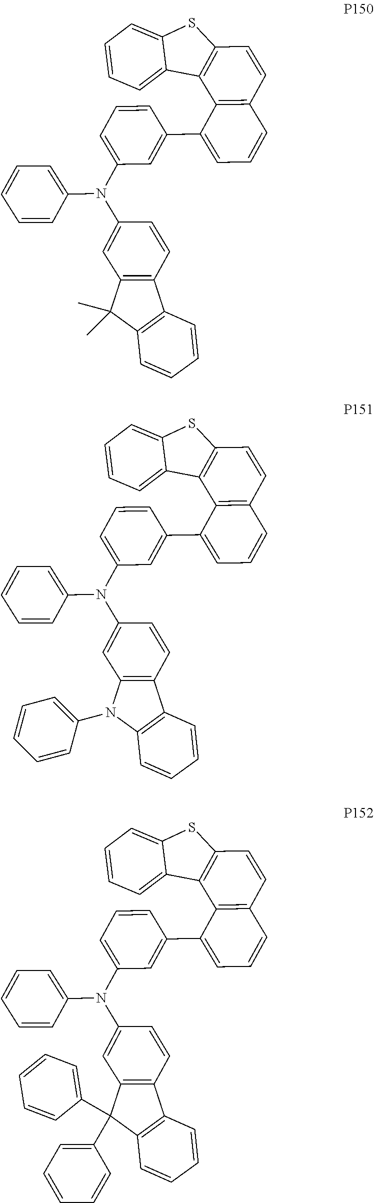 [SMILES: CC1(C)c2ccccc2-c2ccc(N(c3ccccc3)c3cccc(-c4cccc5ccc6sc7ccccc7c6c45)c3)cc21 ; c1ccc(N(c2cccc(-c3cccc4ccc5sc6ccccc6c5c34)c2)c2ccc3c4ccccc4n(-c4ccccc4)c3c2)cc1 ; c1ccc(N(c2cccc(-c3cccc4ccc5sc6ccccc6c5c34)c2)c2ccc3c(c2)C(c2ccccc2)(c2ccccc2)c2ccccc2-3)cc1]

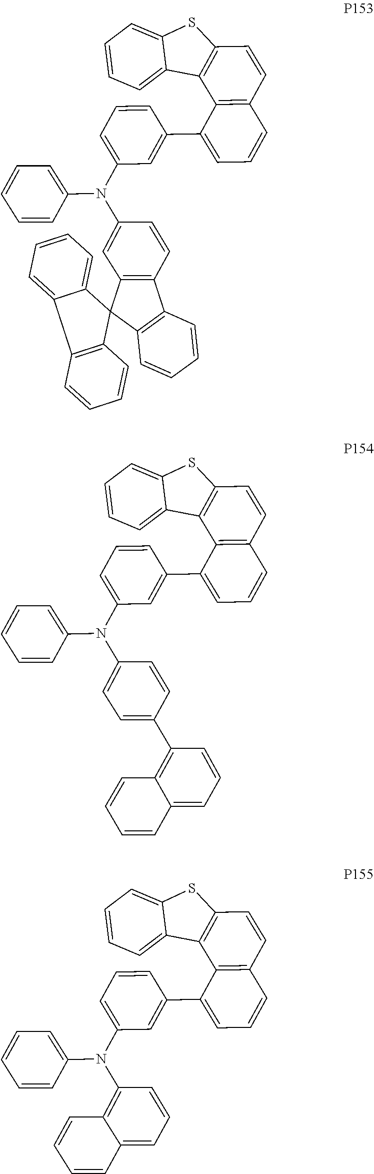 [SMILES: c1ccc(N(c2cccc(-c3cccc4ccc5sc6ccccc6c5c34)c2)c2ccc3c(c2)C2(c4ccccc4-c4ccccc42)c2ccccc2-3)cc1 ; c1ccc(N(c2ccc(-c3cccc4ccccc34)cc2)c2cccc(-c3cccc4ccc5sc6ccccc6c5c34)c2)cc1 ; c1ccc(N(c2cccc(-c3cccc4ccc5sc6ccccc6c5c34)c2)c2cccc3ccccc23)cc1]

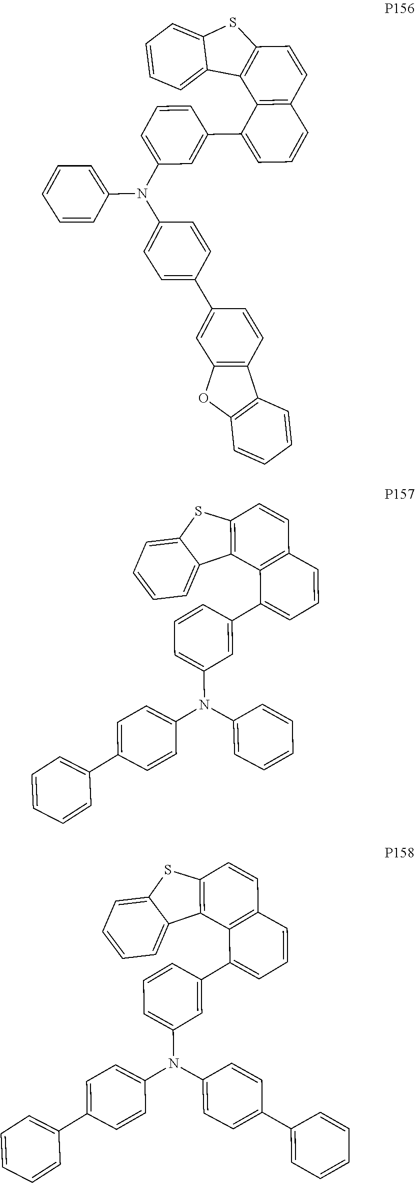

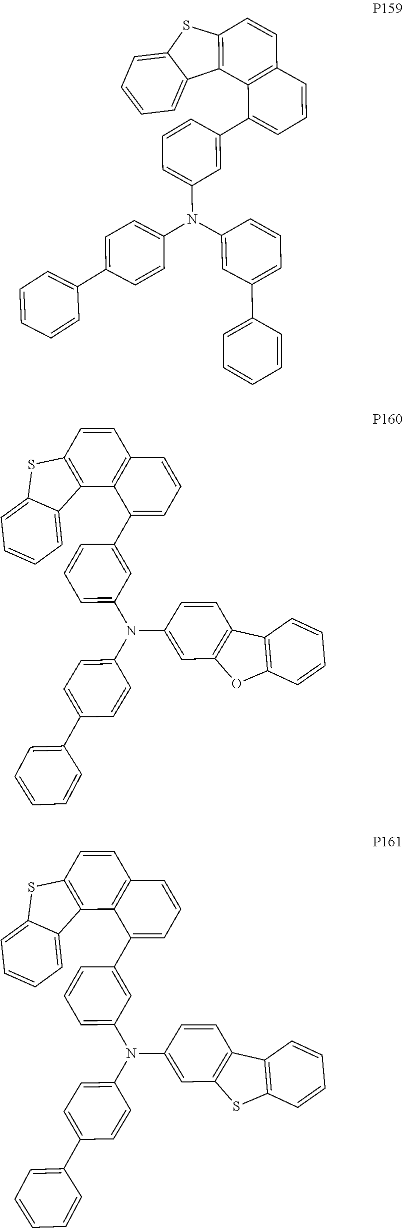 [SMILES: c1ccc(-c2ccc(N(c3cccc(-c4ccccc4)c3)c3cccc(-c4cccc5ccc6sc7ccccc7c6c45)c3)cc2)cc1 ; c1ccc(-c2ccc(N(c3cccc(-c4cccc5ccc6sc7ccccc7c6c45)c3)c3ccc4c(c3)oc3ccccc34)cc2)cc1 ; c1ccc(-c2ccc(N(c3cccc(-c4cccc5ccc6sc7ccccc7c6c45)c3)c3ccc4c(c3)sc3ccccc34)cc2)cc1]

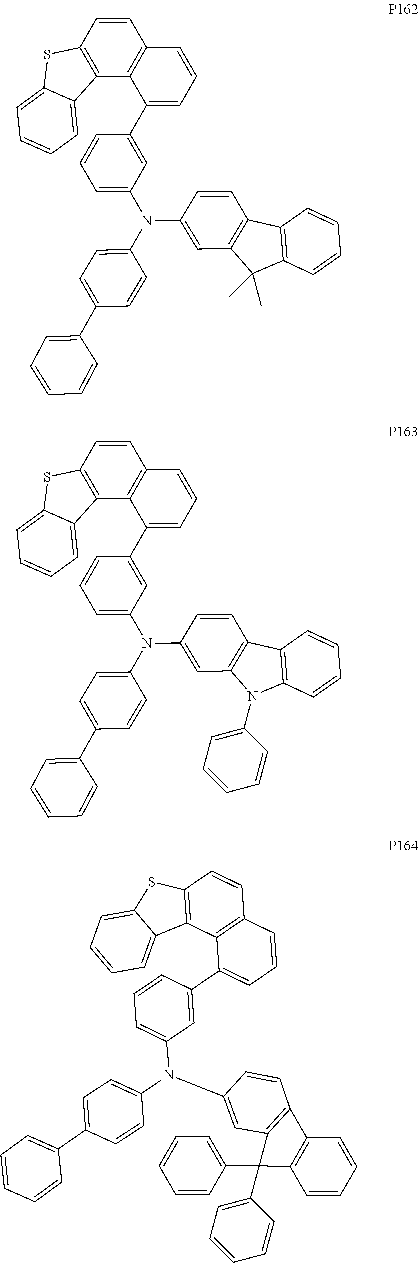 [SMILES: CC1(C)c2ccccc2-c2ccc(N(c3ccc(-c4ccccc4)cc3)c3cccc(-c4cccc5ccc6sc7ccccc7c6c45)c3)cc21 ; c1ccc(-c2ccc(N(c3cccc(-c4cccc5ccc6sc7ccccc7c6c45)c3)c3ccc4c5ccccc5n(-c5ccccc5)c4c3)cc2)cc1 ; c1ccc(-c2ccc(N(c3cccc(-c4cccc5ccc6sc7ccccc7c6c45)c3)c3ccc4c(c3)C(c3ccccc3)(c3ccccc3)c3ccccc3-4)cc2)cc1]

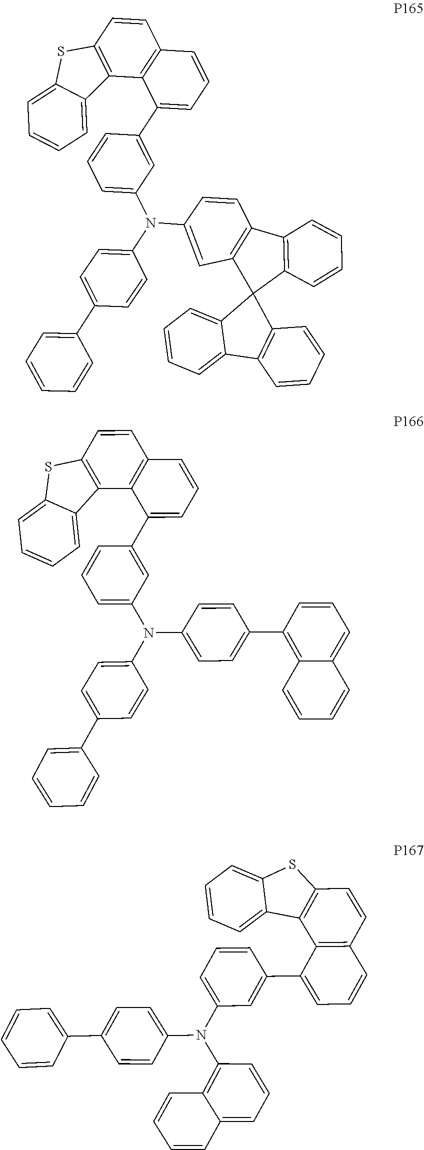

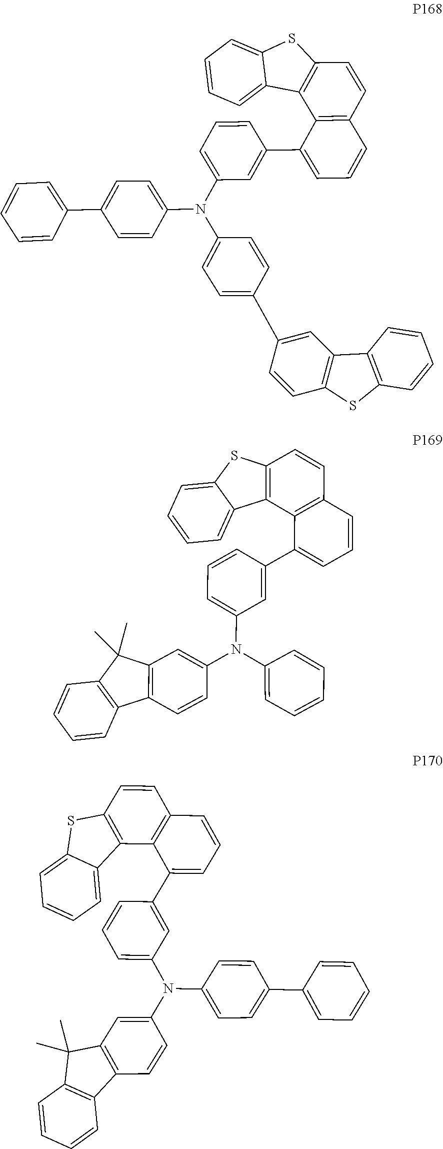 [SMILES: c1ccc(-c2ccc(N(c3ccc(-c4ccc5sc6ccccc6c5c4)cc3)c3cccc(-c4cccc5ccc6sc7ccccc7c6c45)c3)cc2)cc1 ; CC1(C)c2ccccc2-c2ccc(N(c3ccccc3)c3cccc(-c4cccc5ccc6sc7ccccc7c6c45)c3)cc21 ; CC1(C)c2ccccc2-c2ccc(N(c3ccc(-c4ccccc4)cc3)c3cccc(-c4cccc5ccc6sc7ccccc7c6c45)c3)cc21]

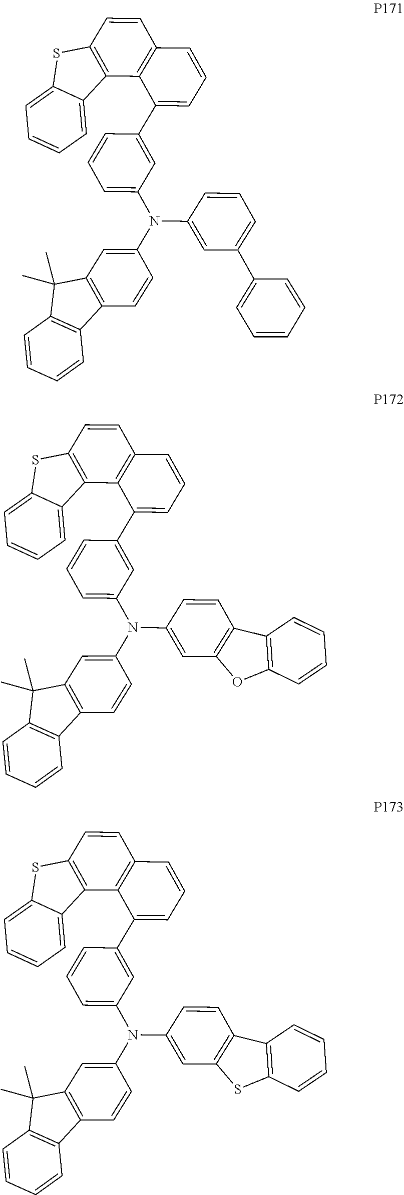 [SMILES: CC1(C)c2ccccc2-c2ccc(N(c3cccc(-c4ccccc4)c3)c3cccc(-c4cccc5ccc6sc7ccccc7c6c45)c3)cc21 ; CC1(C)c2ccccc2-c2ccc(N(c3cccc(-c4cccc5ccc6sc7ccccc7c6c45)c3)c3ccc4c(c3)oc3ccccc34)cc21 ; CC1(C)c2ccccc2-c2ccc(N(c3cccc(-c4cccc5ccc6sc7ccccc7c6c45)c3)c3ccc4c(c3)sc3ccccc34)cc21]

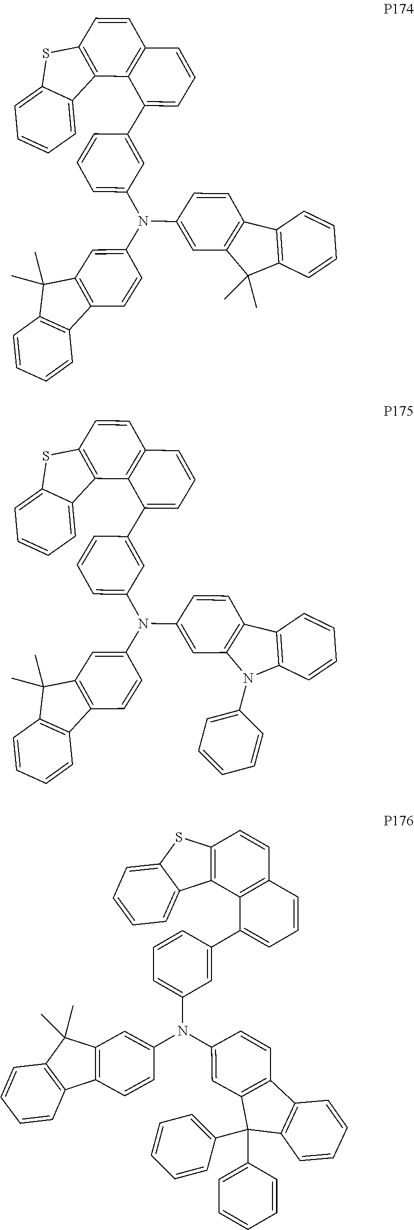 [SMILES: CC1(C)c2ccccc2-c2ccc(N(c3cccc(-c4cccc5ccc6sc7ccccc7c6c45)c3)c3ccc4c(c3)C(C)(C)c3ccccc3-4)cc21 ; CC1(C)c2ccccc2-c2ccc(N(c3cccc(-c4cccc5ccc6sc7ccccc7c6c45)c3)c3ccc4c5ccccc5n(-c5ccccc5)c4c3)cc21 ; CC1(C)c2ccccc2-c2ccc(N(c3cccc(-c4cccc5ccc6sc7ccccc7c6c45)c3)c3ccc4c(c3)C(c3ccccc3)(c3ccccc3)c3ccccc3-4)cc21]

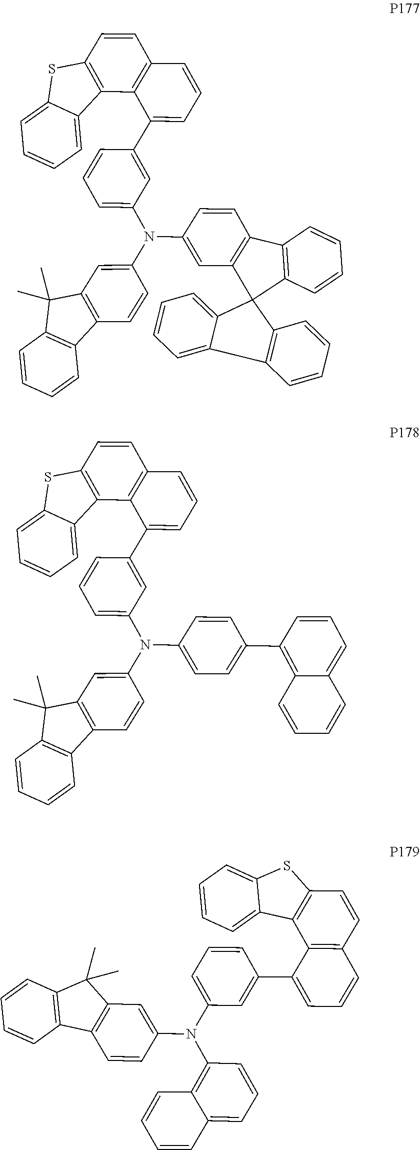 [SMILES: CC1(C)c2ccccc2-c2ccc(N(c3cccc(-c4cccc5ccc6sc7ccccc7c6c45)c3)c3ccc4c(c3)C3(c5ccccc5-c5ccccc53)c3ccccc3-4)cc21 ; CC1(C)c2ccccc2-c2ccc(N(c3ccc(-c4cccc5ccccc45)cc3)c3cccc(-c4cccc5ccc6sc7ccccc7c6c45)c3)cc21 ; CC1(C)c2ccccc2-c2ccc(N(c3cccc(-c4cccc5ccc6sc7ccccc7c6c45)c3)c3cccc4ccccc34)cc21]

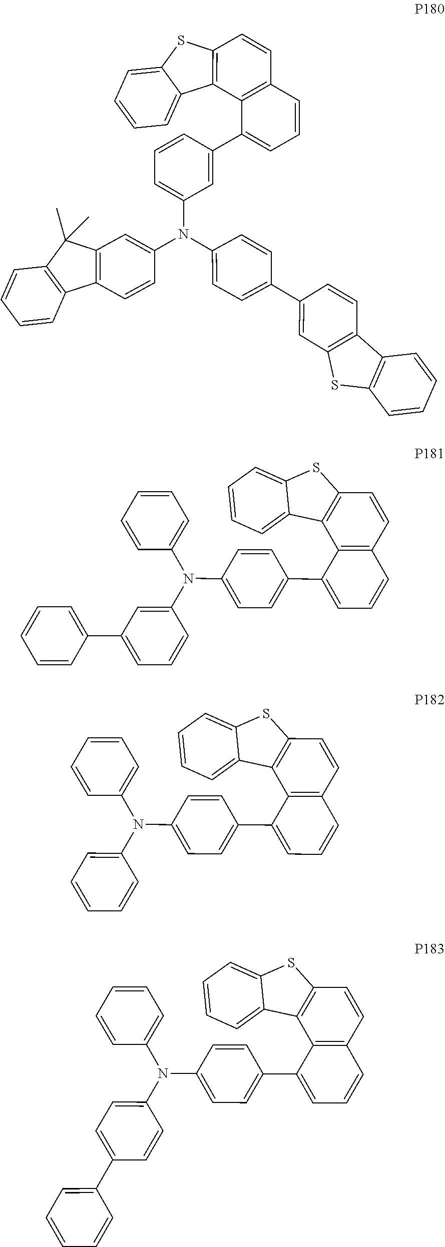 [SMILES: CC1(C)c2ccccc2-c2ccc(N(c3ccc(-c4ccc5c(c4)sc4ccccc45)cc3)c3cccc(-c4cccc5ccc6sc7ccccc7c6c45)c3)cc21 ; c1ccc(-c2cccc(N(c3ccccc3)c3ccc(-c4cccc5ccc6sc7ccccc7c6c45)cc3)c2)cc1 ; c1ccc(N(c2ccccc2)c2ccc(-c3cccc4ccc5sc6ccccc6c5c34)cc2)cc1 ; c1ccc(-c2ccc(N(c3ccccc3)c3ccc(-c4cccc5ccc6sc7ccccc7c6c45)cc3)cc2)cc1]

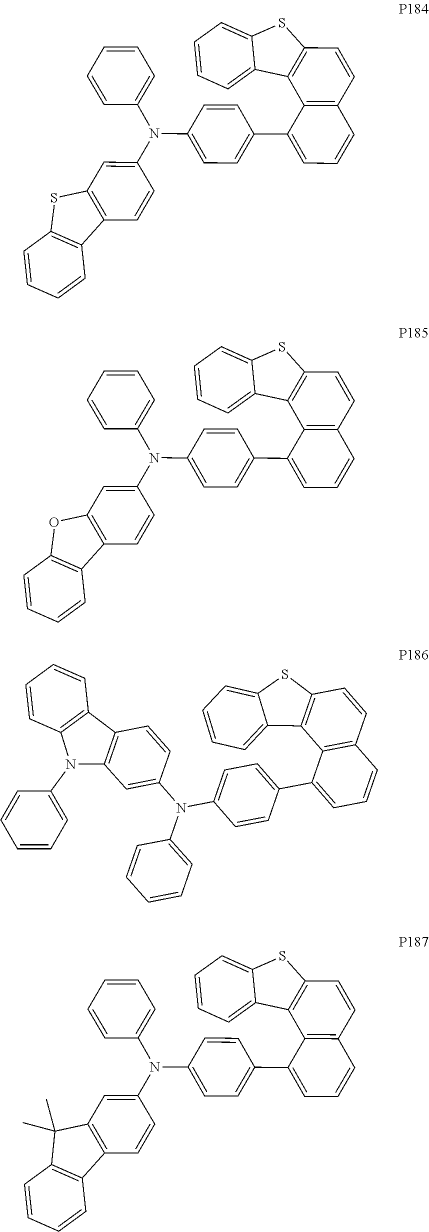

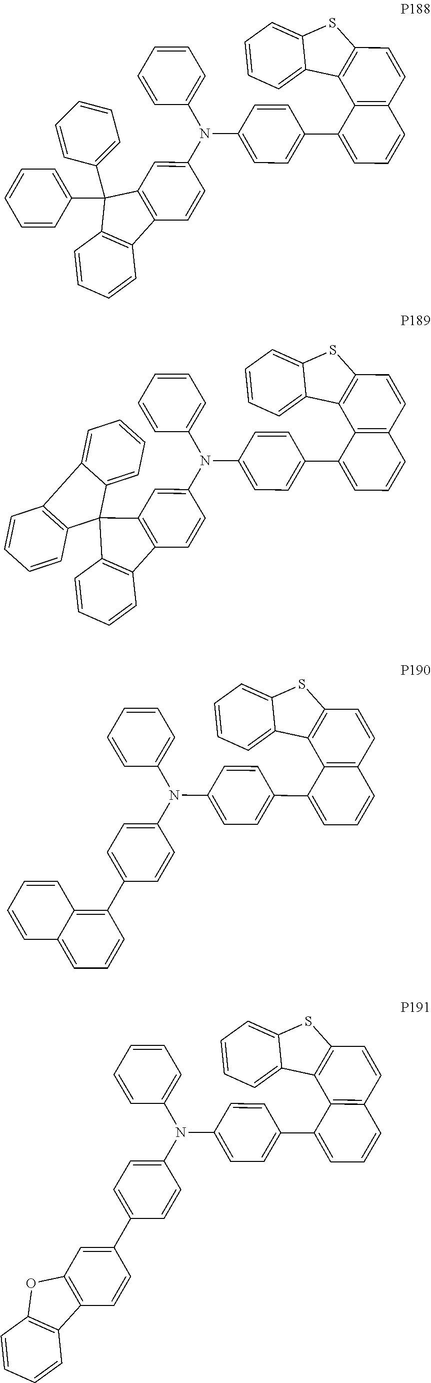 [SMILES: c1ccc(N(c2ccc(-c3cccc4ccc5sc6ccccc6c5c34)cc2)c2ccc3c(c2)C(c2ccccc2)(c2ccccc2)c2ccccc2-3)cc1 ; c1ccc(N(c2ccc(-c3cccc4ccc5sc6ccccc6c5c34)cc2)c2ccc3c(c2)C2(c4ccccc4-c4ccccc42)c2ccccc2-3)cc1 ; c1ccc(N(c2ccc(-c3cccc4ccccc34)cc2)c2ccc(-c3cccc4ccc5sc6ccccc6c5c34)cc2)cc1 ; c1ccc(N(c2ccc(-c3ccc4c(c3)oc3ccccc34)cc2)c2ccc(-c3cccc4ccc5sc6ccccc6c5c34)cc2)cc1]

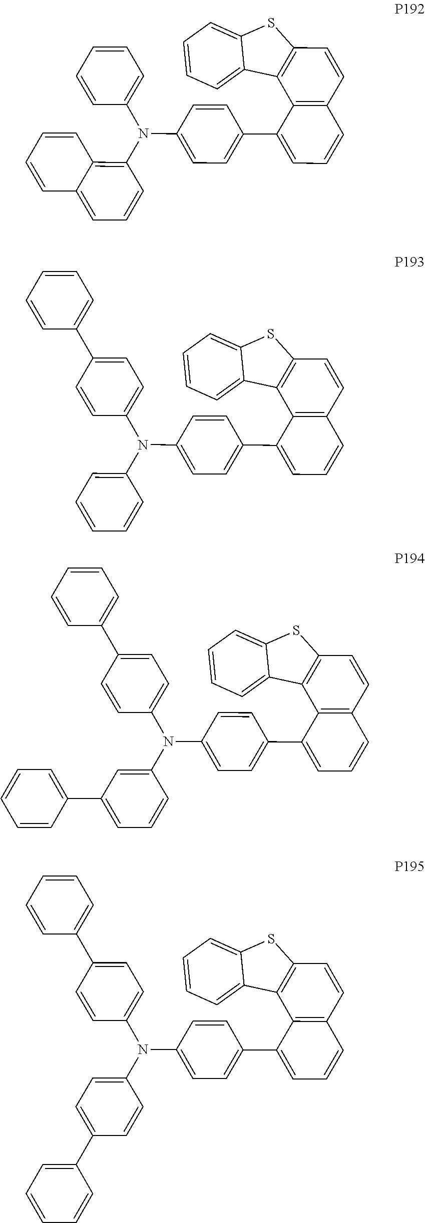

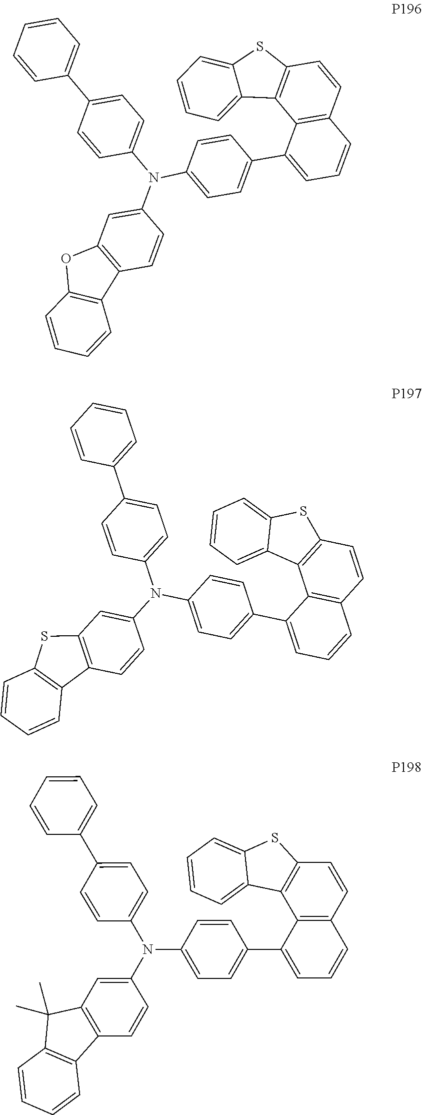

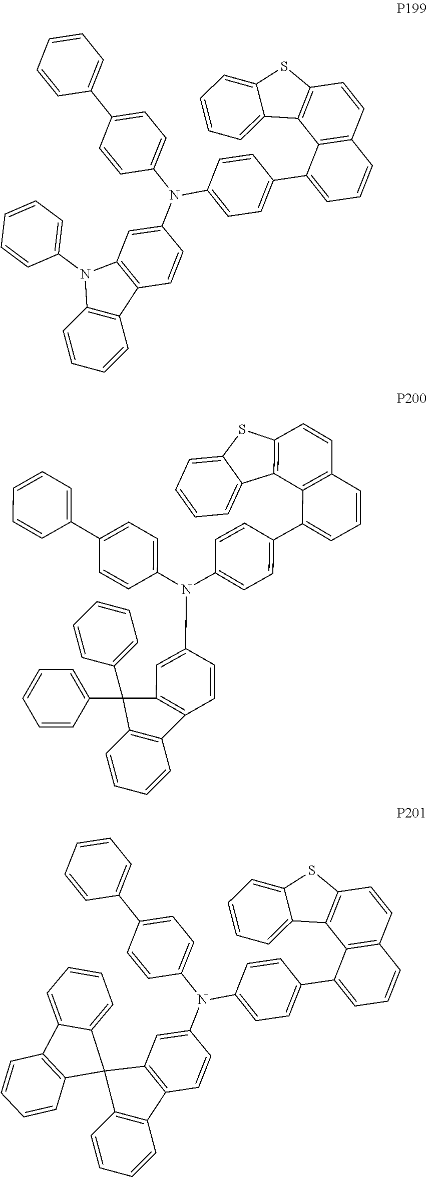 [SMILES: c1ccc(-c2ccc(N(c3ccc(-c4cccc5ccc6sc7ccccc7c6c45)cc3)c3ccc4c5ccccc5n(-c5ccccc5)c4c3)cc2)cc1 ; c1ccc(-c2ccc(N(c3ccc(-c4cccc5ccc6sc7ccccc7c6c45)cc3)c3ccc4c(c3)C(c3ccccc3)(c3ccccc3)c3ccccc3-4)cc2)cc1 ; c1ccc(-c2ccc(N(c3ccc(-c4cccc5ccc6sc7ccccc7c6c45)cc3)c3ccc4c(c3)C3(c5ccccc5-c5ccccc53)c3ccccc3-4)cc2)cc1]

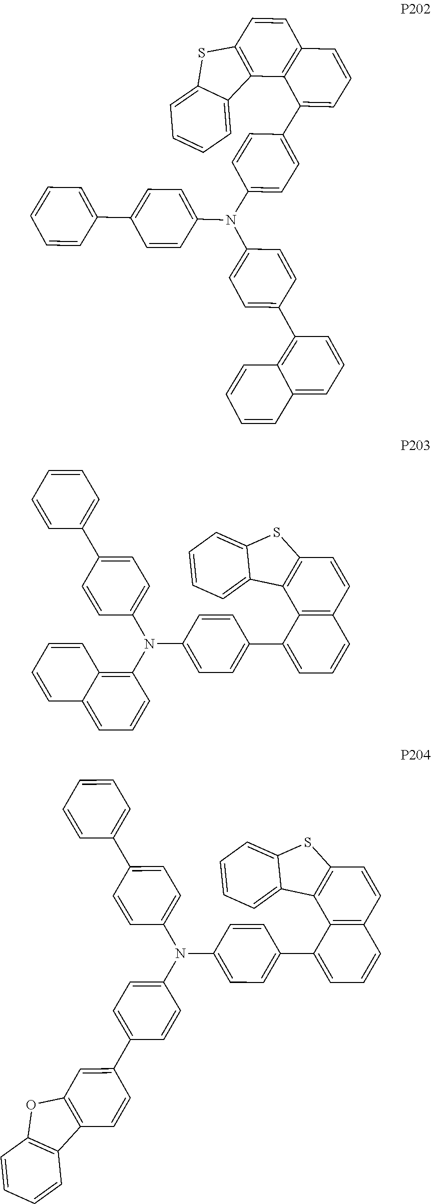 [SMILES: c1ccc(-c2ccc(N(c3ccc(-c4cccc5ccccc45)cc3)c3ccc(-c4cccc5ccc6sc7ccccc7c6c45)cc3)cc2)cc1 ; c1ccc(-c2ccc(N(c3ccc(-c4cccc5ccc6sc7ccccc7c6c45)cc3)c3cccc4ccccc34)cc2)cc1 ; c1ccc(-c2ccc(N(c3ccc(-c4ccc5c(c4)oc4ccccc45)cc3)c3ccc(-c4cccc5ccc6sc7ccccc7c6c45)cc3)cc2)cc1]

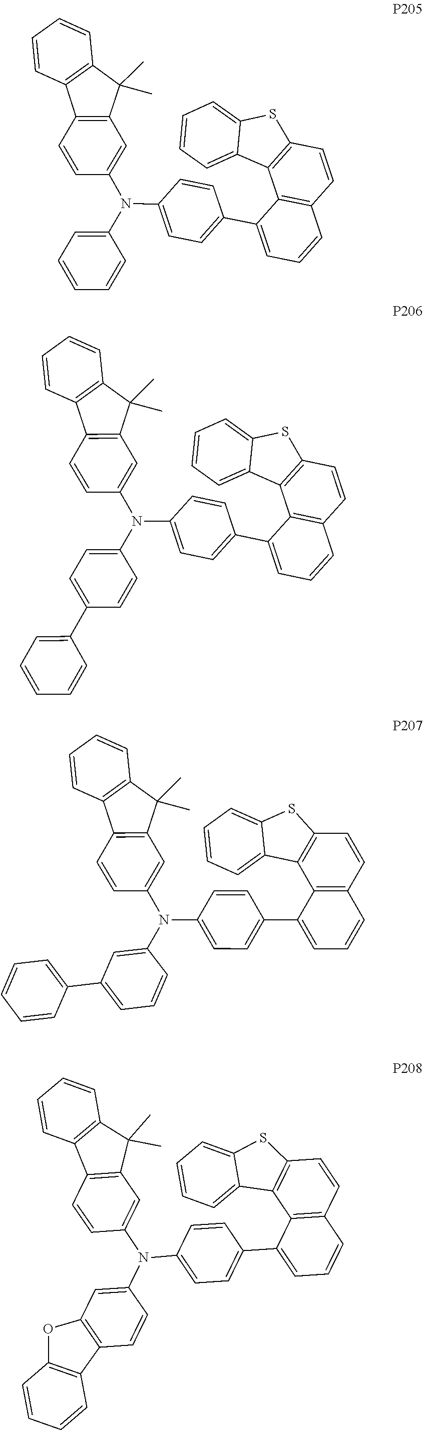 [SMILES: CC1(C)c2ccccc2-c2ccc(N(c3ccccc3)c3ccc(-c4cccc5ccc6sc7ccccc7c6c45)cc3)cc21 ; CC1(C)c2ccccc2-c2ccc(N(c3ccc(-c4ccccc4)cc3)c3ccc(-c4cccc5ccc6sc7ccccc7c6c45)cc3)cc21 ; CC1(C)c2ccccc2-c2ccc(N(c3ccc(-c4cccc5ccc6sc7ccccc7c6c45)cc3)c3cccc(-c4ccccc4)c3)cc21 ; CC1(C)c2ccccc2-c2ccc(N(c3ccc(-c4cccc5ccc6sc7ccccc7c6c45)cc3)c3ccc4c(c3)oc3ccccc34)cc21]

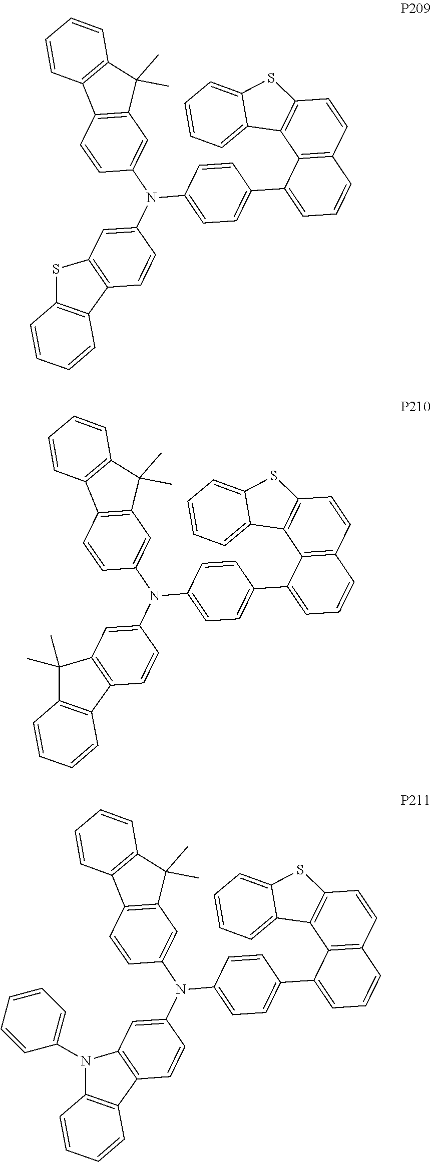 [SMILES: CC1(C)c2ccccc2-c2ccc(N(c3ccc(-c4cccc5ccc6sc7ccccc7c6c45)cc3)c3ccc4c(c3)sc3ccccc34)cc21 ; CC1(C)c2ccccc2-c2ccc(N(c3ccc(-c4cccc5ccc6sc7ccccc7c6c45)cc3)c3ccc4c(c3)C(C)(C)c3ccccc3-4)cc21 ; CC1(C)c2ccccc2-c2ccc(N(c3ccc(-c4cccc5ccc6sc7ccccc7c6c45)cc3)c3ccc4c5ccccc5n(-c5ccccc5)c4c3)cc21]

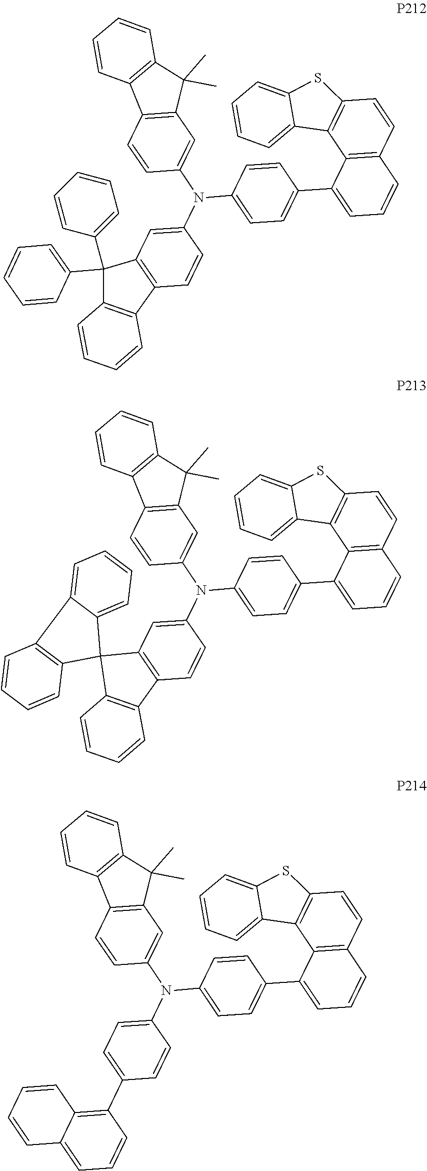

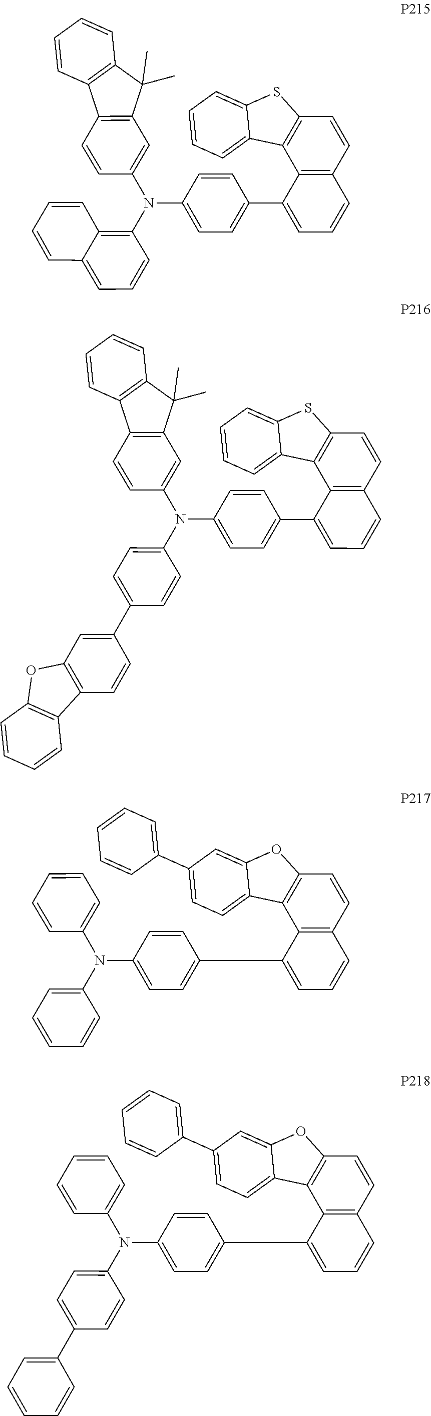 [SMILES: CC1(C)c2ccccc2-c2ccc(N(c3ccc(-c4cccc5ccc6sc7ccccc7c6c45)cc3)c3cccc4ccccc34)cc21 ; CC1(C)c2ccccc2-c2ccc(N(c3ccc(-c4ccc5c(c4)oc4ccccc45)cc3)c3ccc(-c4cccc5ccc6sc7ccccc7c6c45)cc3)cc21 ; c1ccc(-c2ccc3c(c2)oc2ccc4cccc(-c5ccc(N(c6ccccc6)c6ccccc6)cc5)c4c23)cc1 ; c1ccc(-c2ccc(N(c3ccccc3)c3ccc(-c4cccc5ccc6oc7cc(-c8ccccc8)ccc7c6c45)cc3)cc2)cc1]

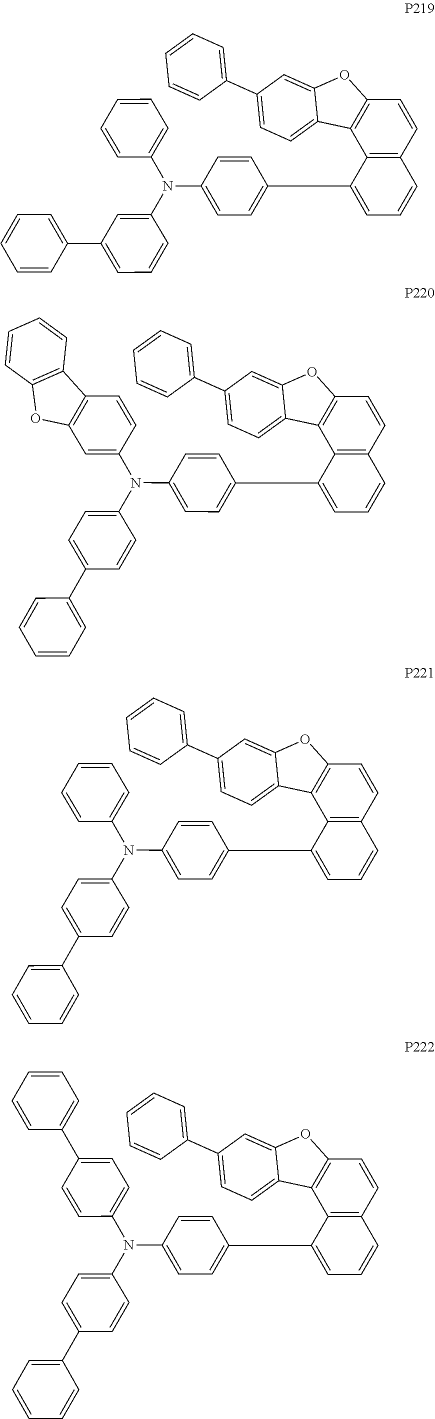 [SMILES: c1ccc(-c2cccc(N(c3ccccc3)c3ccc(-c4cccc5ccc6oc7cc(-c8ccccc8)ccc7c6c45)cc3)c2)cc1 ; c1ccc(-c2ccc(N(c3ccc(-c4cccc5ccc6oc7cc(-c8ccccc8)ccc7c6c45)cc3)c3ccc4c(c3)oc3ccccc34)cc2)cc1 ; c1ccc(-c2ccc(N(c3ccccc3)c3ccc(-c4cccc5ccc6oc7cc(-c8ccccc8)ccc7c6c45)cc3)cc2)cc1 ; c1ccc(-c2ccc(N(c3ccc(-c4ccccc4)cc3)c3ccc(-c4cccc5ccc6oc7cc(-c8ccccc8)ccc7c6c45)cc3)cc2)cc1]

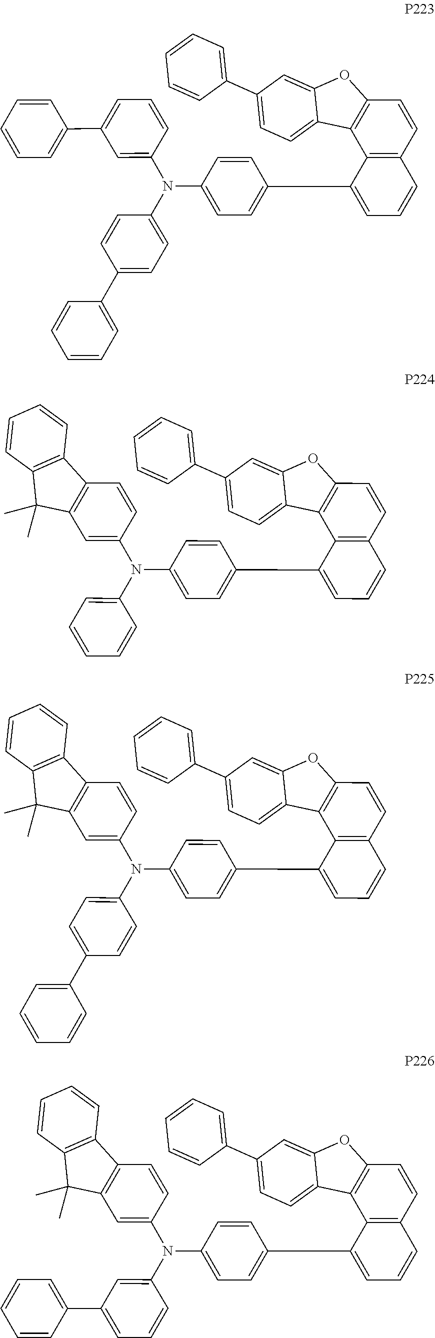

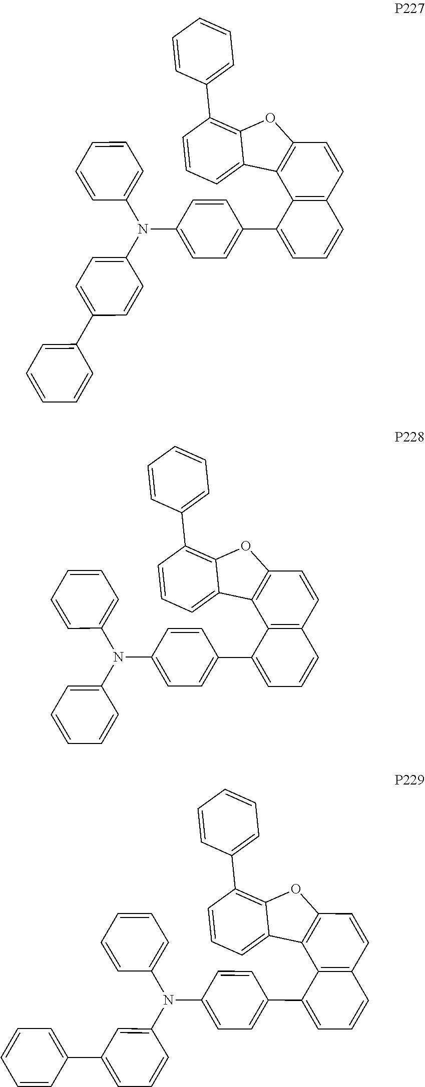 [SMILES: c1ccc(-c2ccc(N(c3ccccc3)c3ccc(-c4cccc5ccc6oc7c(-c8ccccc8)cccc7c6c45)cc3)cc2)cc1 ; c1ccc(-c2cccc3c2oc2ccc4cccc(-c5ccc(N(c6ccccc6)c6ccccc6)cc5)c4c23)cc1 ; c1ccc(-c2cccc(N(c3ccccc3)c3ccc(-c4cccc5ccc6oc7c(-c8ccccc8)cccc7c6c45)cc3)c2)cc1]

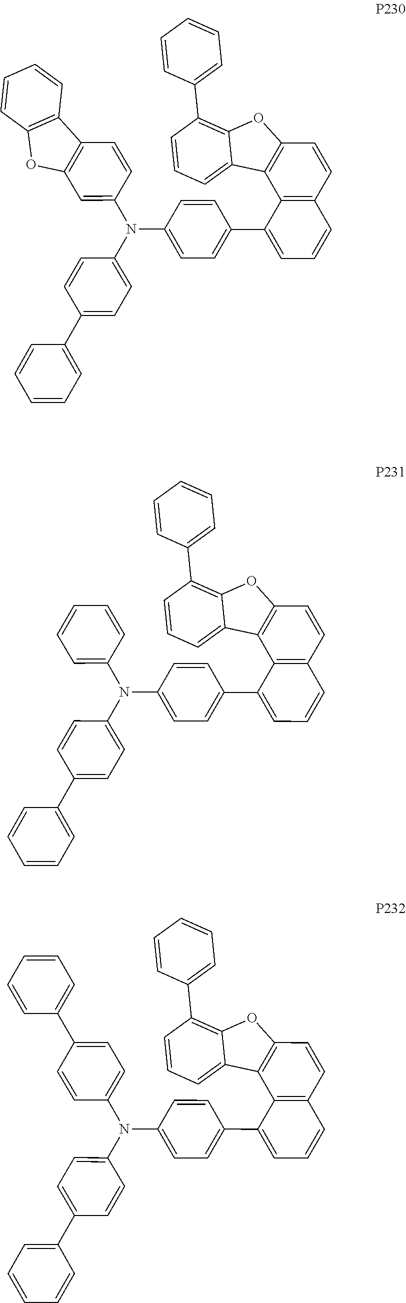 [SMILES: c1ccc(-c2ccc(N(c3ccc(-c4cccc5ccc6oc7c(-c8ccccc8)cccc7c6c45)cc3)c3ccc4c(c3)oc3ccccc34)cc2)cc1 ; c1ccc(-c2ccc(N(c3ccccc3)c3ccc(-c4cccc5ccc6oc7c(-c8ccccc8)cccc7c6c45)cc3)cc2)cc1 ; c1ccc(-c2ccc(N(c3ccc(-c4ccccc4)cc3)c3ccc(-c4cccc5ccc6oc7c(-c8ccccc8)cccc7c6c45)cc3)cc2)cc1]

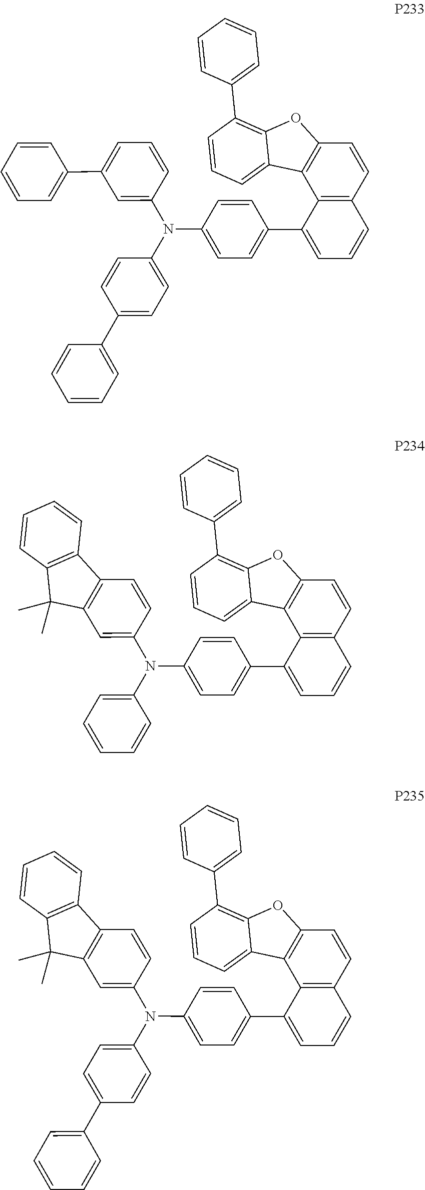 [SMILES: c1ccc(-c2ccc(N(c3ccc(-c4cccc5ccc6oc7c(-c8ccccc8)cccc7c6c45)cc3)c3cccc(-c4ccccc4)c3)cc2)cc1 ; CC1(C)c2ccccc2-c2ccc(N(c3ccccc3)c3ccc(-c4cccc5ccc6oc7c(-c8ccccc8)cccc7c6c45)cc3)cc21 ; CC1(C)c2ccccc2-c2ccc(N(c3ccc(-c4ccccc4)cc3)c3ccc(-c4cccc5ccc6oc7c(-c8ccccc8)cccc7c6c45)cc3)cc21]

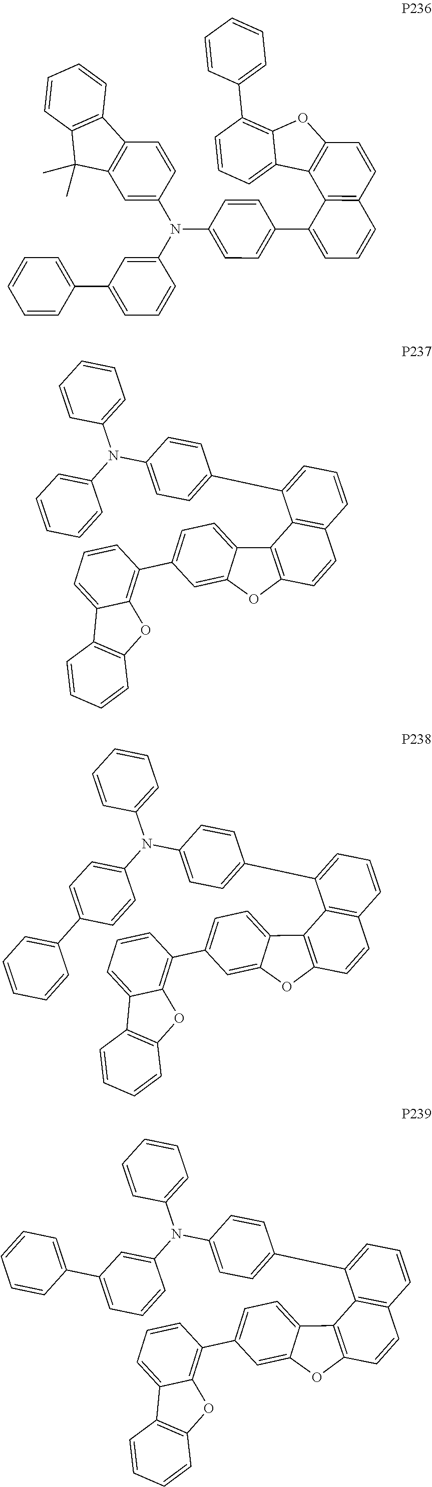

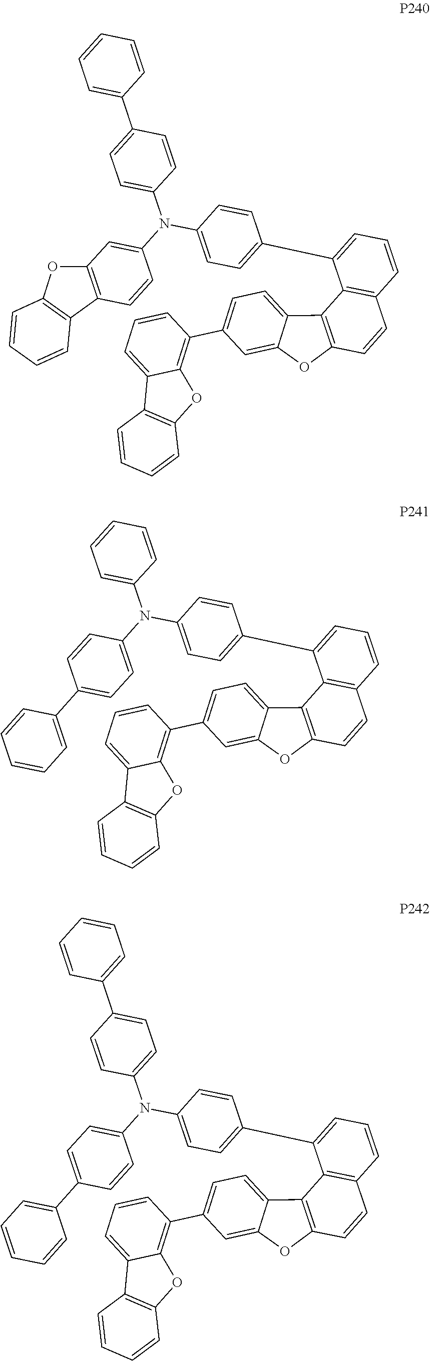 [SMILES: c1ccc(-c2ccc(N(c3ccc(-c4cccc5ccc6oc7cc(-c8cccc9c8oc8ccccc89)ccc7c6c45)cc3)c3ccc4c(c3)oc3ccccc34)cc2)cc1 ; c1ccc(-c2ccc(N(c3ccccc3)c3ccc(-c4cccc5ccc6oc7cc(-c8cccc9c8oc8ccccc89)ccc7c6c45)cc3)cc2)cc1 ; c1ccc(-c2ccc(N(c3ccc(-c4ccccc4)cc3)c3ccc(-c4cccc5ccc6oc7cc(-c8cccc9c8oc8ccccc89)ccc7c6c45)cc3)cc2)cc1]

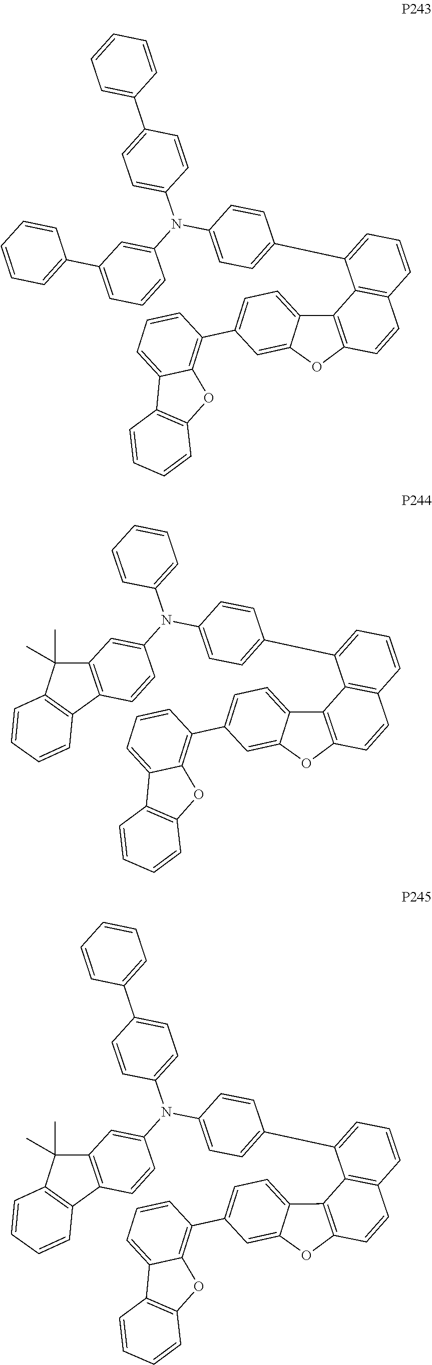 [SMILES: c1ccc(-c2ccc(N(c3ccc(-c4cccc5ccc6oc7cc(-c8cccc9c8oc8ccccc89)ccc7c6c45)cc3)c3cccc(-c4ccccc4)c3)cc2)cc1 ; CC1(C)c2ccccc2-c2ccc(N(c3ccccc3)c3ccc(-c4cccc5ccc6oc7cc(-c8cccc9c8oc8ccccc89)ccc7c6c45)cc3)cc21 ; CC1(C)c2ccccc2-c2ccc(N(c3ccc(-c4ccccc4)cc3)c3ccc(-c4cccc5ccc6oc7cc(-c8cccc9c8oc8ccccc89)ccc7c6c45)cc3)cc21]

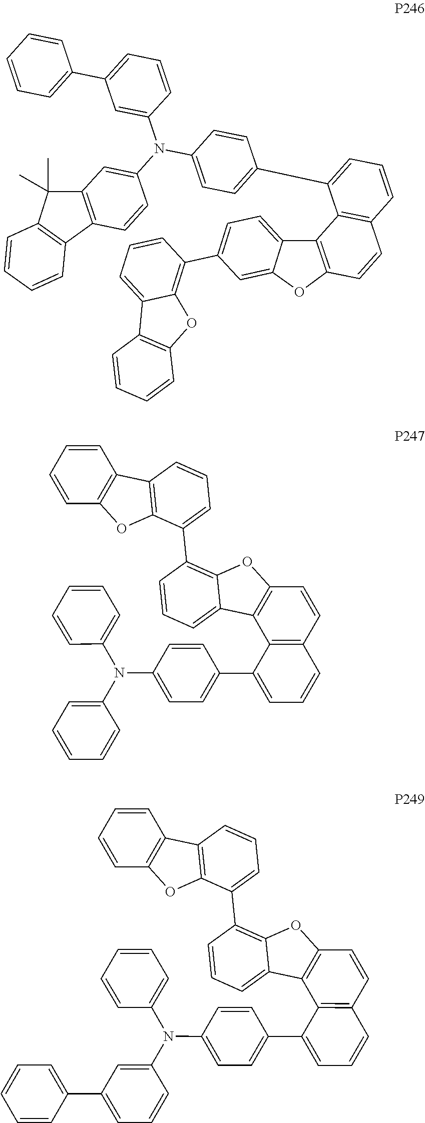 [SMILES: CC1(C)c2ccccc2-c2ccc(N(c3ccc(-c4cccc5ccc6oc7cc(-c8cccc9c8oc8ccccc89)ccc7c6c45)cc3)c3cccc(-c4ccccc4)c3)cc21 ; c1ccc(N(c2ccccc2)c2ccc(-c3cccc4ccc5oc6c(-c7cccc8c7oc7ccccc78)cccc6c5c34)cc2)cc1 ; c1ccc(-c2cccc(N(c3ccccc3)c3ccc(-c4cccc5ccc6oc7c(-c8cccc9c8oc8ccccc89)cccc7c6c45)cc3)c2)cc1]

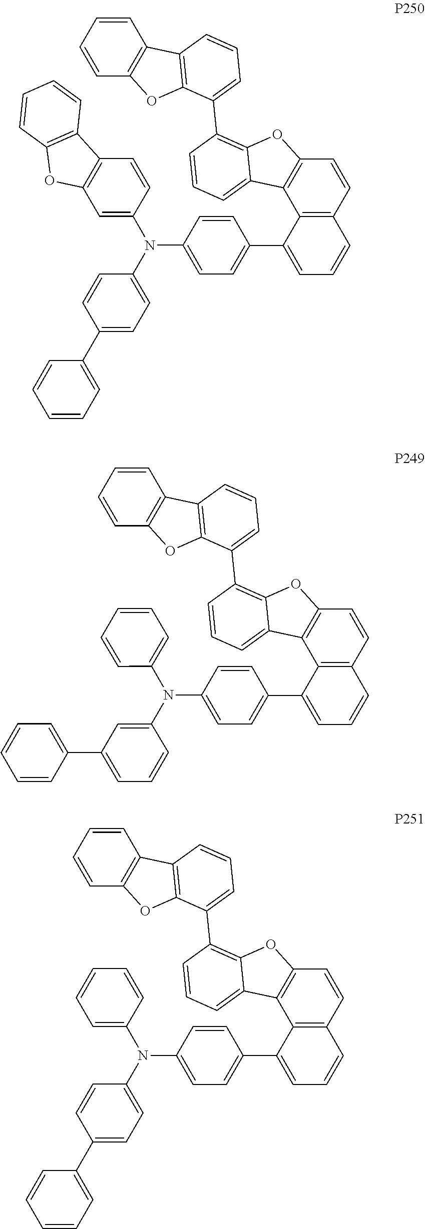 [SMILES: c1ccc(-c2ccc(N(c3ccc(-c4cccc5ccc6oc7c(-c8cccc9c8oc8ccccc89)cccc7c6c45)cc3)c3ccc4c(c3)oc3ccccc34)cc2)cc1 ; c1ccc(-c2cccc(N(c3ccccc3)c3ccc(-c4cccc5ccc6oc7c(-c8cccc9c8oc8ccccc89)cccc7c6c45)cc3)c2)cc1 ; c1ccc(-c2ccc(N(c3ccccc3)c3ccc(-c4cccc5ccc6oc7c(-c8cccc9c8oc8ccccc89)cccc7c6c45)cc3)cc2)cc1]

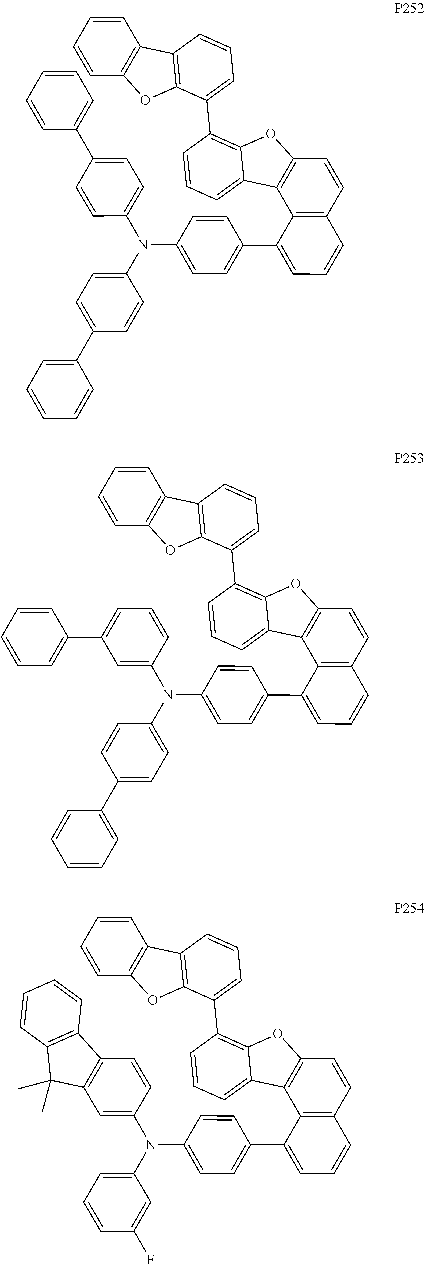 [SMILES: c1ccc(-c2ccc(N(c3ccc(-c4ccccc4)cc3)c3ccc(-c4cccc5ccc6oc7c(-c8cccc9c8oc8ccccc89)cccc7c6c45)cc3)cc2)cc1 ; c1ccc(-c2ccc(N(c3ccc(-c4cccc5ccc6oc7c(-c8cccc9c8oc8ccccc89)cccc7c6c45)cc3)c3cccc(-c4ccccc4)c3)cc2)cc1 ; CC1(C)c2ccccc2-c2ccc(N(c3ccc(-c4cccc5ccc6oc7c(-c8cccc9c8oc8ccccc89)cccc7c6c45)cc3)c3cccc(F)c3)cc21]

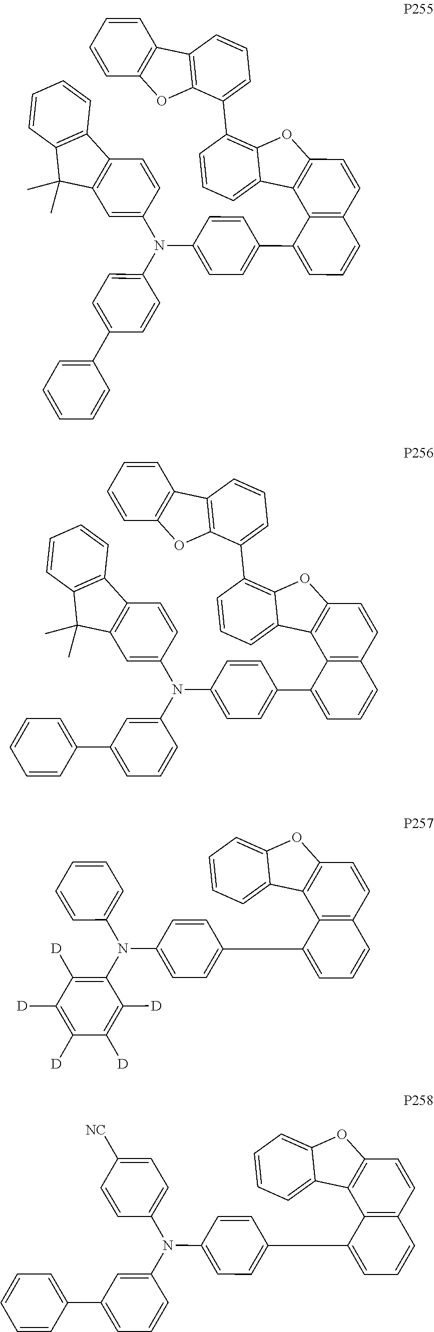 [SMILES: CC1(C)c2ccccc2-c2ccc(N(c3ccc(-c4ccccc4)cc3)c3ccc(-c4cccc5ccc6oc7c(-c8cccc9c8oc8ccccc89)cccc7c6c45)cc3)cc21 ; CC1(C)c2ccccc2-c2ccc(N(c3ccc(-c4cccc5ccc6oc7c(-c8cccc9c8oc8ccccc89)cccc7c6c45)cc3)c3cccc(-c4ccccc4)c3)cc21 ; [2H]c1c([2H])c([2H])c(N(c2ccccc2)c2ccc(-c3cccc4ccc5oc6ccccc6c5c34)cc2)c([2H])c1[2H] ; N#Cc1ccc(N(c2ccc(-c3cccc4ccc5oc6ccccc6c5c34)cc2)c2cccc(-c3ccccc3)c2)cc1]

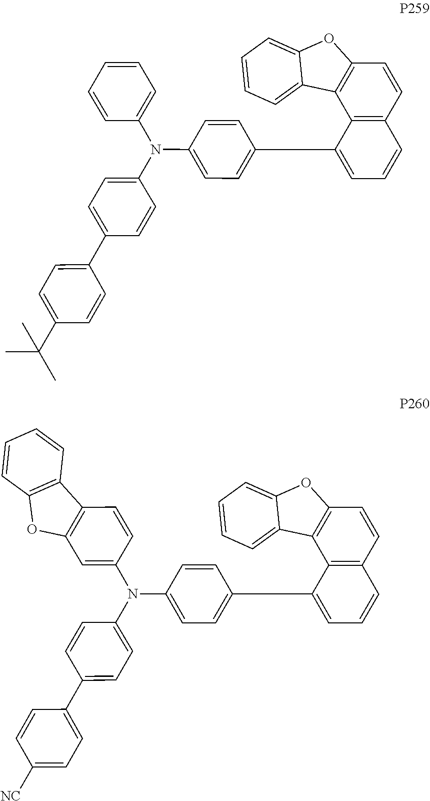 [SMILES: CC(C)(C)c1ccc(-c2ccc(N(c3ccccc3)c3ccc(-c4cccc5ccc6oc7ccccc7c6c45)cc3)cc2)cc1 ; N#Cc1ccc(-c2ccc(N(c3ccc(-c4cccc5ccc6oc7ccccc7c6c45)cc3)c3ccc4c(c3)oc3ccccc34)cc2)cc1]

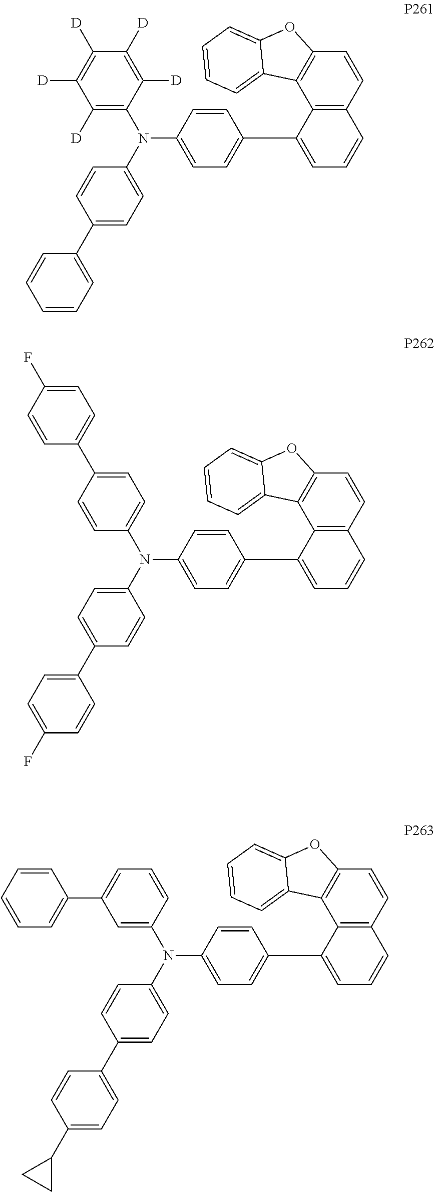 [SMILES: [2H]c1c([2H])c([2H])c(N(c2ccc(-c3ccccc3)cc2)c2ccc(-c3cccc4ccc5oc6ccccc6c5c34)cc2)c([2H])c1[2H] ; Fc1ccc(-c2ccc(N(c3ccc(-c4ccc(F)cc4)cc3)c3ccc(-c4cccc5ccc6oc7ccccc7c6c45)cc3)cc2)cc1 ; c1ccc(-c2cccc(N(c3ccc(-c4ccc(C5CC5)cc4)cc3)c3ccc(-c4cccc5ccc6oc7ccccc7c6c45)cc3)c2)cc1]

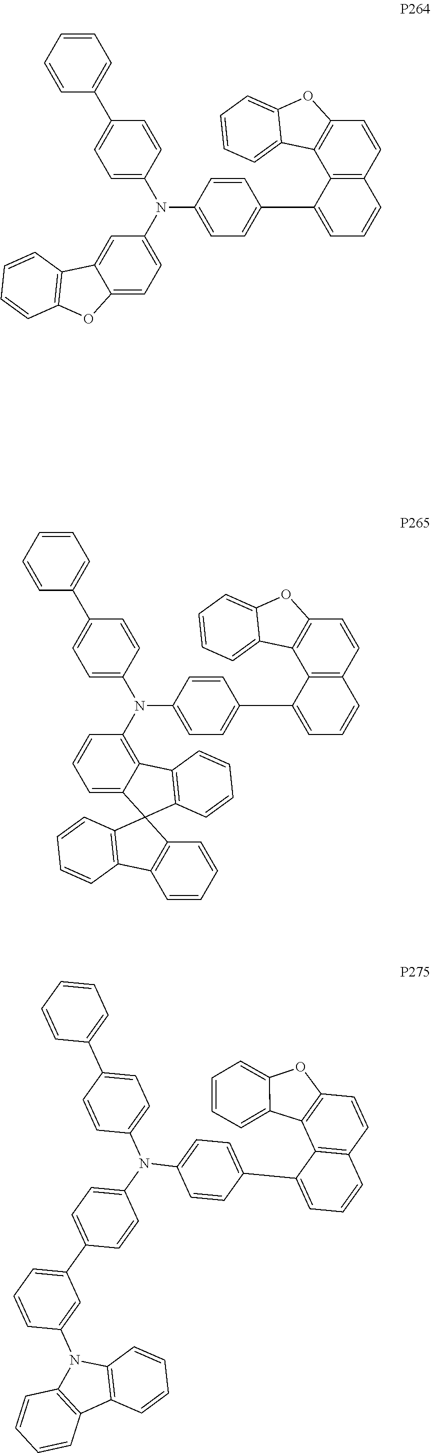 [SMILES: c1ccc(-c2ccc(N(c3ccc(-c4cccc5ccc6oc7ccccc7c6c45)cc3)c3ccc4oc5ccccc5c4c3)cc2)cc1 ; c1ccc(-c2ccc(N(c3ccc(-c4cccc5ccc6oc7ccccc7c6c45)cc3)c3cccc4c3-c3ccccc3C43c4ccccc4-c4ccccc43)cc2)cc1 ; c1ccc(-c2ccc(N(c3ccc(-c4cccc(-n5c6ccccc6c6ccccc65)c4)cc3)c3ccc(-c4cccc5ccc6oc7ccccc7c6c45)cc3)cc2)cc1]

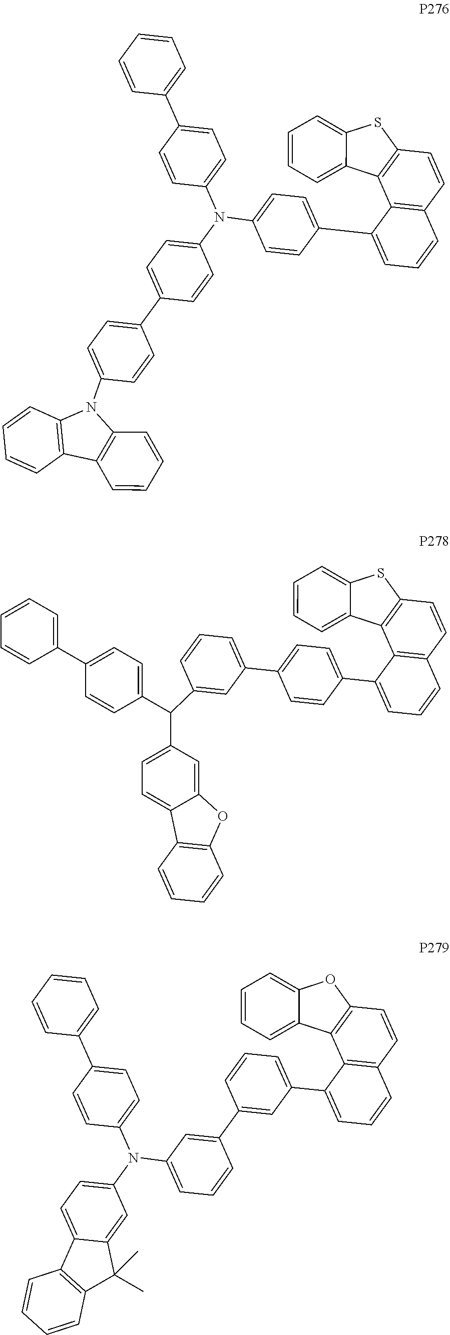 [SMILES: c1ccc(-c2ccc(N(c3ccc(-c4ccc(-n5c6ccccc6c6ccccc65)cc4)cc3)c3ccc(-c4cccc5ccc6sc7ccccc7c6c45)cc3)cc2)cc1 ; c1ccc(-c2ccc(C(c3cccc(-c4ccc(-c5cccc6ccc7sc8ccccc8c7c56)cc4)c3)c3ccc4c(c3)oc3ccccc34)cc2)cc1 ; CC1(C)c2ccccc2-c2ccc(N(c3ccc(-c4ccccc4)cc3)c3cccc(-c4cccc(-c5cccc6ccc7oc8ccccc8c7c56)c4)c3)cc21]

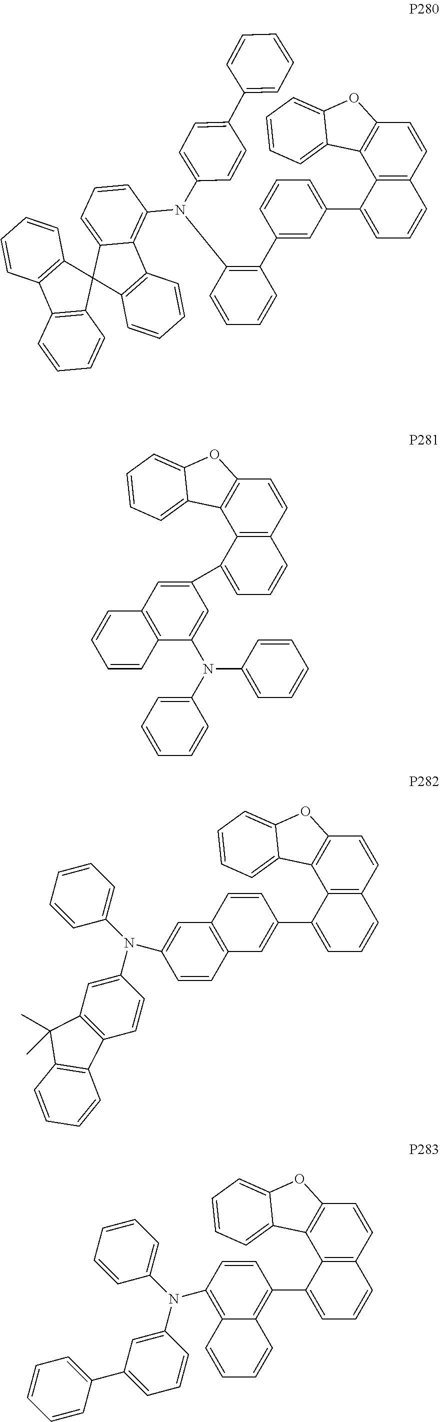 [SMILES: c1ccc(-c2ccc(N(c3ccccc3-c3cccc(-c4cccc5ccc6oc7ccccc7c6c45)c3)c3cccc4c3-c3ccccc3C43c4ccccc4-c4ccccc43)cc2)cc1 ; c1ccc(N(c2ccccc2)c2cc(-c3cccc4ccc5oc6ccccc6c5c34)cc3ccccc23)cc1 ; CC1(C)c2ccccc2-c2ccc(N(c3ccccc3)c3ccc4cc(-c5cccc6ccc7oc8ccccc8c7c56)ccc4c3)cc21 ; c1ccc(-c2cccc(N(c3ccccc3)c3ccc(-c4cccc5ccc6oc7ccccc7c6c45)c4ccccc34)c2)cc1]

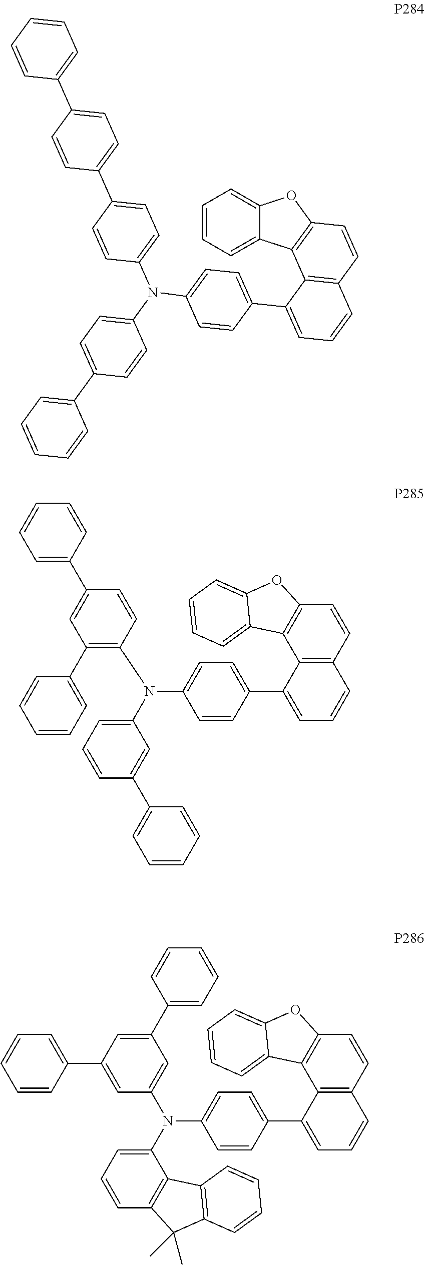

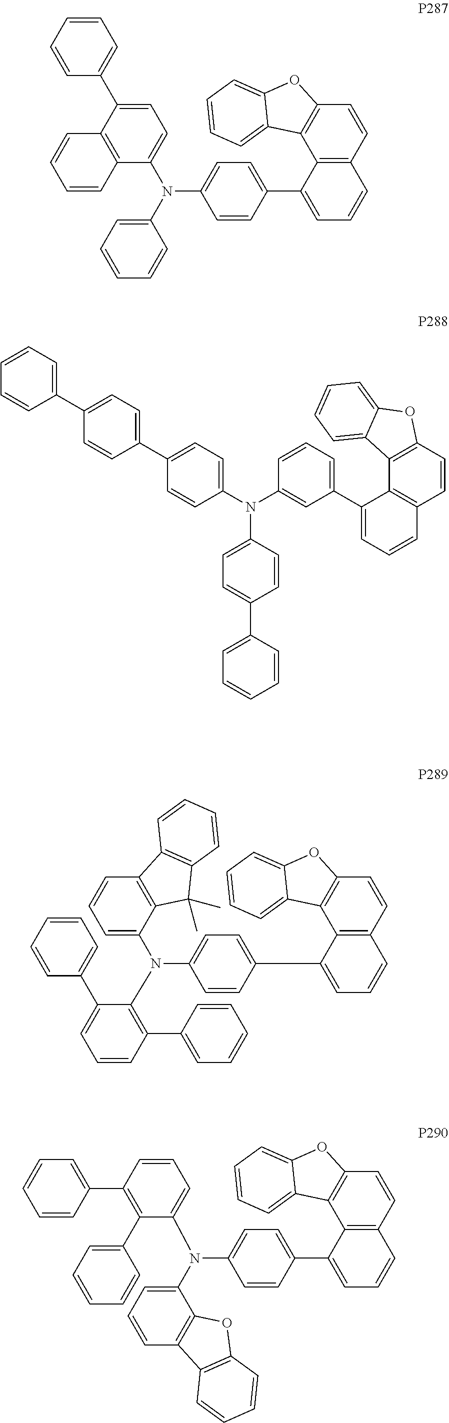 [SMILES: c1ccc(-c2ccc(N(c3ccccc3)c3ccc(-c4cccc5ccc6oc7ccccc7c6c45)cc3)c3ccccc23)cc1 ; c1ccc(-c2ccc(-c3ccc(N(c4ccc(-c5ccccc5)cc4)c4cccc(-c5cccc6ccc7oc8ccccc8c7c56)c4)cc3)cc2)cc1 ; CC1(C)c2ccccc2-c2cccc(N(c3ccc(-c4cccc5ccc6oc7ccccc7c6c45)cc3)c3c(-c4ccccc4)cccc3-c3ccccc3)c21 ; c1ccc(-c2cccc(N(c3ccc(-c4cccc5ccc6oc7ccccc7c6c45)cc3)c3cccc4c3oc3ccccc34)c2-c2ccccc2)cc1]

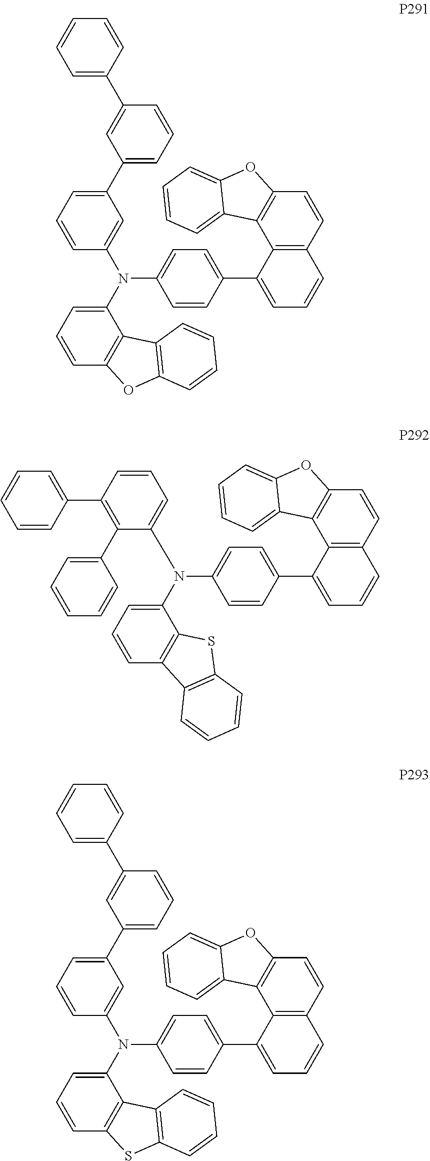 [SMILES: c1ccc(-c2cccc(-c3cccc(N(c4ccc(-c5cccc6ccc7oc8ccccc8c7c56)cc4)c4cccc5oc6ccccc6c45)c3)c2)cc1 ; c1ccc(-c2cccc(N(c3ccc(-c4cccc5ccc6oc7ccccc7c6c45)cc3)c3cccc4c3sc3ccccc34)c2-c2ccccc2)cc1 ; c1ccc(-c2cccc(-c3cccc(N(c4ccc(-c5cccc6ccc7oc8ccccc8c7c56)cc4)c4cccc5sc6ccccc6c45)c3)c2)cc1]

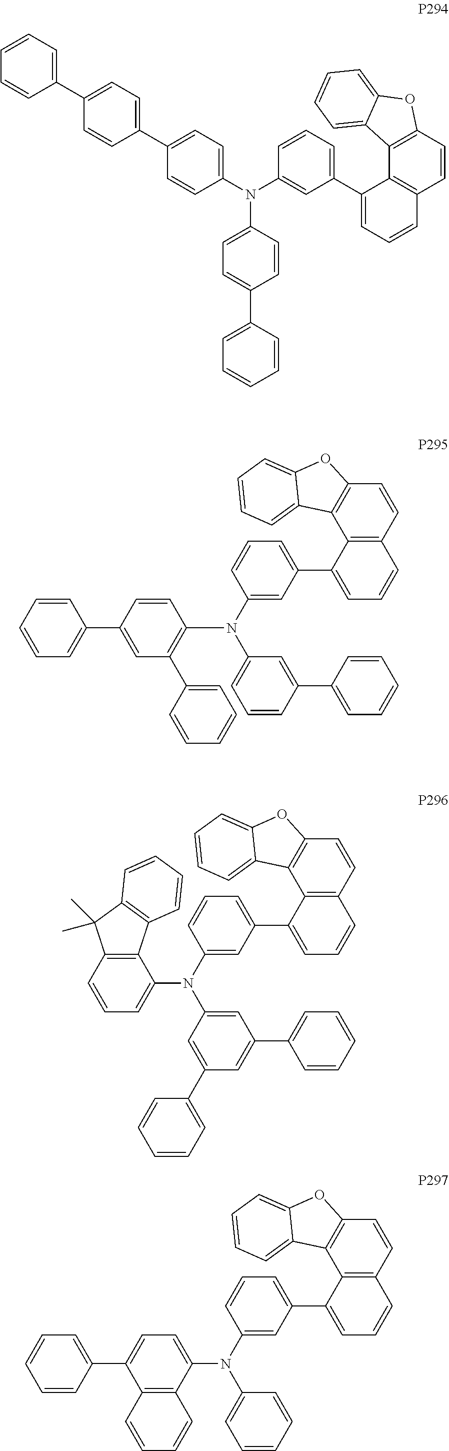 [SMILES: c1ccc(-c2ccc(-c3ccc(N(c4ccc(-c5ccccc5)cc4)c4cccc(-c5cccc6ccc7oc8ccccc8c7c56)c4)cc3)cc2)cc1 ; c1ccc(-c2cccc(N(c3cccc(-c4cccc5ccc6oc7ccccc7c6c45)c3)c3ccc(-c4ccccc4)cc3-c3ccccc3)c2)cc1 ; CC1(C)c2ccccc2-c2c(N(c3cc(-c4ccccc4)cc(-c4ccccc4)c3)c3cccc(-c4cccc5ccc6oc7ccccc7c6c45)c3)cccc21 ; c1ccc(-c2ccc(N(c3ccccc3)c3cccc(-c4cccc5ccc6oc7ccccc7c6c45)c3)c3ccccc23)cc1]

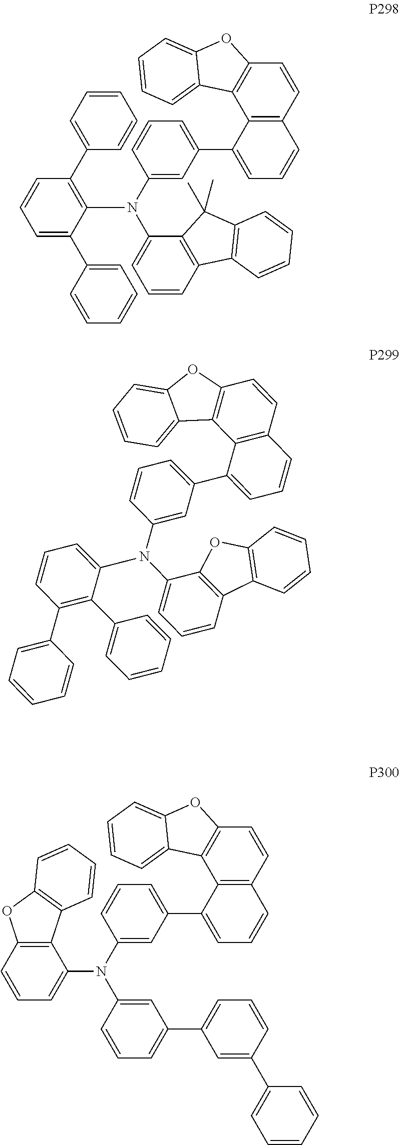 [SMILES: CC1(C)c2ccccc2-c2cccc(N(c3cccc(-c4cccc5ccc6oc7ccccc7c6c45)c3)c3c(-c4ccccc4)cccc3-c3ccccc3)c21 ; c1ccc(-c2cccc(N(c3cccc(-c4cccc5ccc6oc7ccccc7c6c45)c3)c3cccc4c3oc3ccccc34)c2-c2ccccc2)cc1 ; c1ccc(-c2cccc(-c3cccc(N(c4cccc(-c5cccc6ccc7oc8ccccc8c7c56)c4)c4cccc5oc6ccccc6c45)c3)c2)cc1]

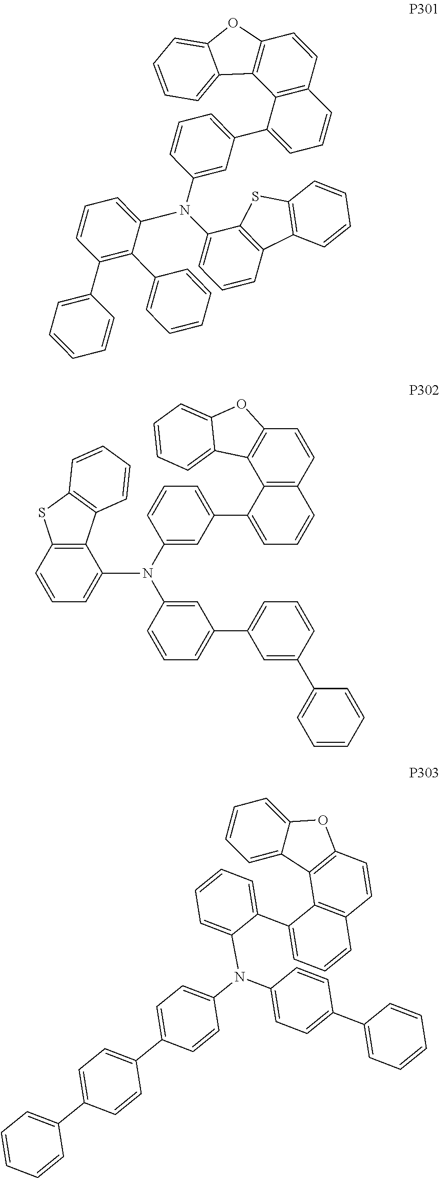 [SMILES: c1ccc(-c2cccc(N(c3cccc(-c4cccc5ccc6oc7ccccc7c6c45)c3)c3cccc4c3sc3ccccc34)c2-c2ccccc2)cc1 ; c1ccc(-c2cccc(-c3cccc(N(c4cccc(-c5cccc6ccc7oc8ccccc8c7c56)c4)c4cccc5sc6ccccc6c45)c3)c2)cc1 ; c1ccc(-c2ccc(-c3ccc(N(c4ccc(-c5ccccc5)cc4)c4ccccc4-c4cccc5ccc6oc7ccccc7c6c45)cc3)cc2)cc1]

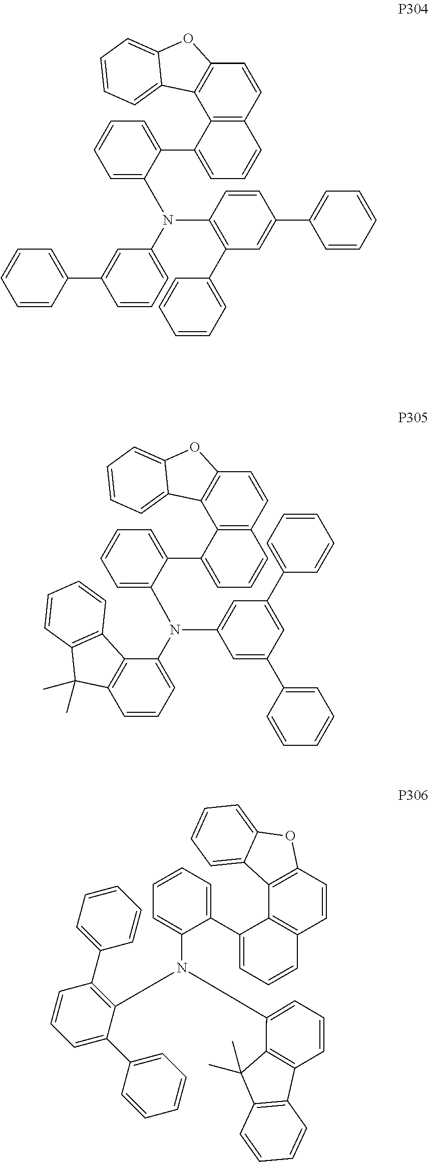 [SMILES: c1ccc(-c2cccc(N(c3ccc(-c4ccccc4)cc3-c3ccccc3)c3ccccc3-c3cccc4ccc5oc6ccccc6c5c34)c2)cc1 ; CC1(C)c2ccccc2-c2c(N(c3cc(-c4ccccc4)cc(-c4ccccc4)c3)c3ccccc3-c3cccc4ccc5oc6ccccc6c5c34)cccc21 ; CC1(C)c2ccccc2-c2cccc(N(c3ccccc3-c3cccc4ccc5oc6ccccc6c5c34)c3c(-c4ccccc4)cccc3-c3ccccc3)c21]

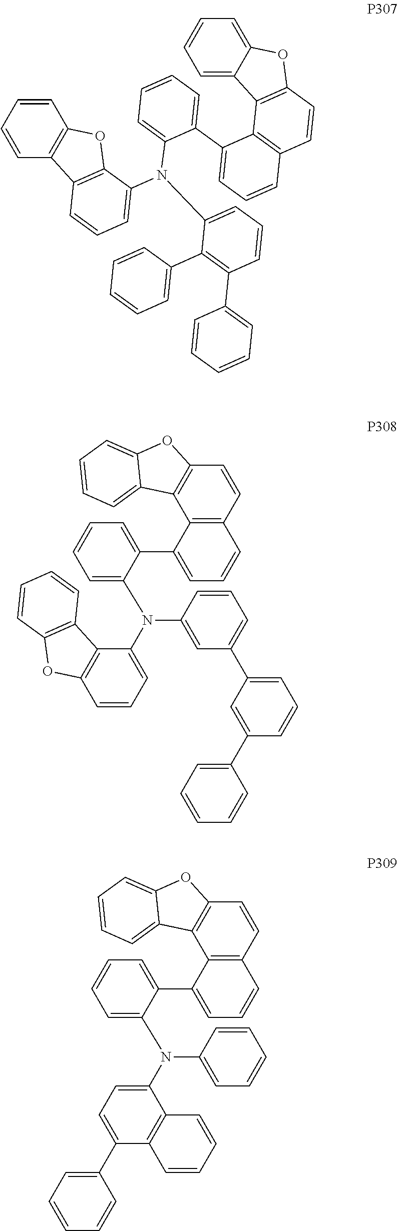 [SMILES: c1ccc(-c2cccc(N(c3ccccc3-c3cccc4ccc5oc6ccccc6c5c34)c3cccc4c3oc3ccccc34)c2-c2ccccc2)cc1 ; c1ccc(-c2cccc(-c3cccc(N(c4ccccc4-c4cccc5ccc6oc7ccccc7c6c45)c4cccc5oc6ccccc6c45)c3)c2)cc1 ; c1ccc(-c2ccc(N(c3ccccc3)c3ccccc3-c3cccc4ccc5oc6ccccc6c5c34)c3ccccc23)cc1]

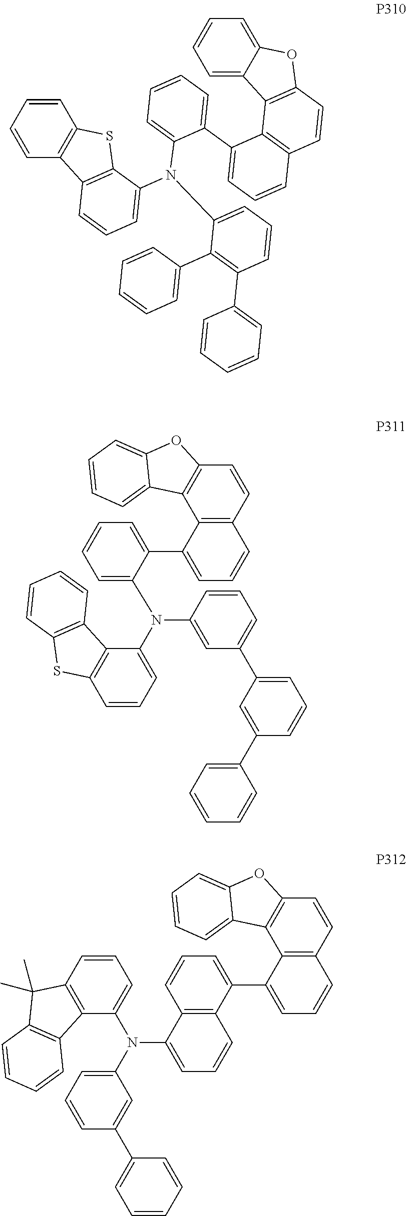 [SMILES: c1ccc(-c2cccc(N(c3ccccc3-c3cccc4ccc5oc6ccccc6c5c34)c3cccc4c3sc3ccccc34)c2-c2ccccc2)cc1 ; c1ccc(-c2cccc(-c3cccc(N(c4ccccc4-c4cccc5ccc6oc7ccccc7c6c45)c4cccc5sc6ccccc6c45)c3)c2)cc1 ; CC1(C)c2ccccc2-c2c(N(c3cccc(-c4ccccc4)c3)c3cccc4c(-c5cccc6ccc7oc8ccccc8c7c56)cccc34)cccc21]

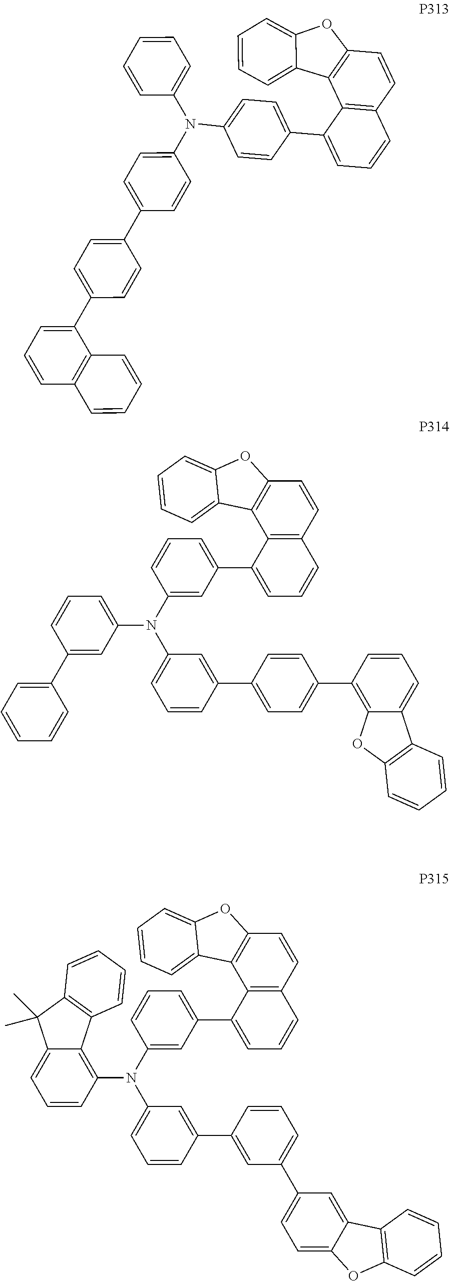 [SMILES: c1ccc(N(c2ccc(-c3ccc(-c4cccc5ccccc45)cc3)cc2)c2ccc(-c3cccc4ccc5oc6ccccc6c5c34)cc2)cc1 ; c1ccc(-c2cccc(N(c3cccc(-c4ccc(-c5cccc6c5oc5ccccc56)cc4)c3)c3cccc(-c4cccc5ccc6oc7ccccc7c6c45)c3)c2)cc1 ; CC1(C)c2ccccc2-c2c(N(c3cccc(-c4cccc(-c5ccc6oc7ccccc7c6c5)c4)c3)c3cccc(-c4cccc5ccc6oc7ccccc7c6c45)c3)cccc21]

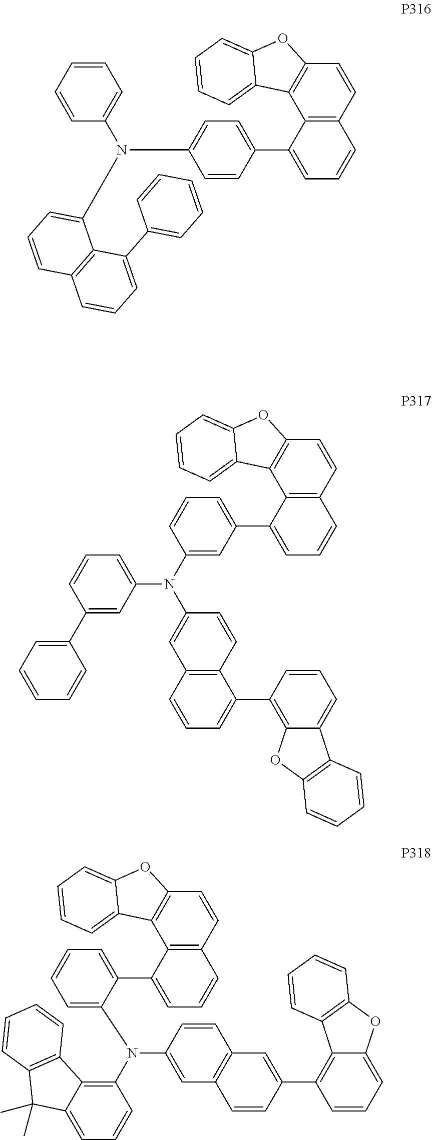 [SMILES: c1ccc(-c2cccc3cccc(N(c4ccccc4)c4ccc(-c5cccc6ccc7oc8ccccc8c7c56)cc4)c23)cc1 ; c1ccc(-c2cccc(N(c3cccc(-c4cccc5ccc6oc7ccccc7c6c45)c3)c3ccc4c(-c5cccc6c5oc5ccccc56)cccc4c3)c2)cc1 ; CC1(C)c2ccccc2-c2c(N(c3ccc4cc(-c5cccc6oc7ccccc7c56)ccc4c3)c3ccccc3-c3cccc4ccc5oc6ccccc6c5c34)cccc21]

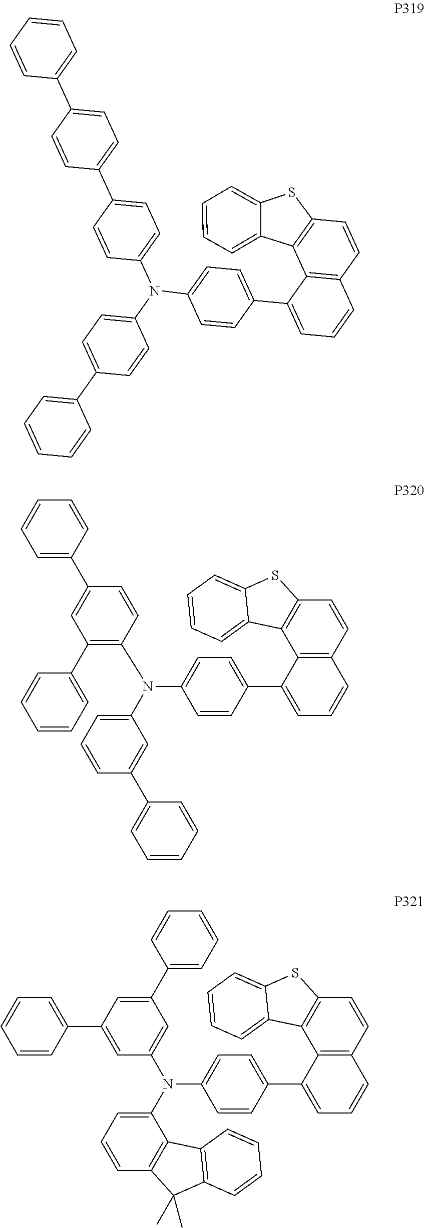

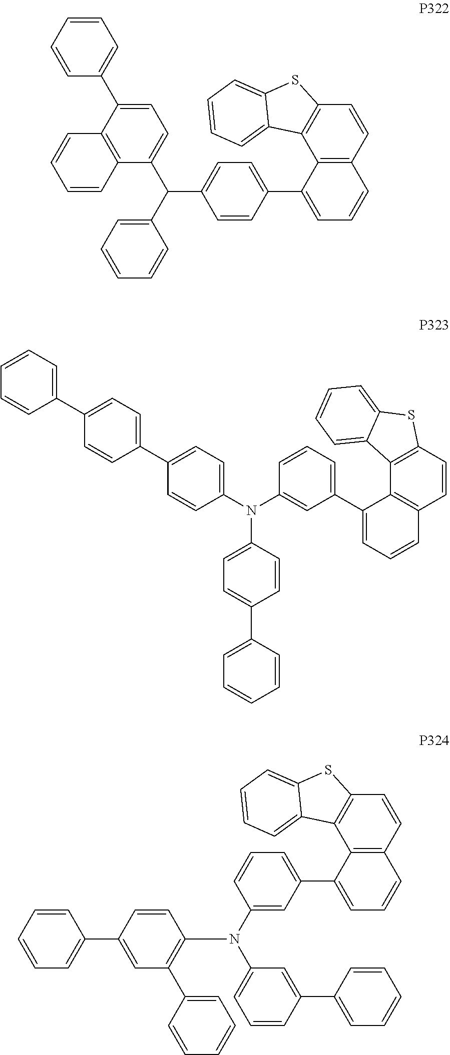 [SMILES: c1ccc(-c2ccc(C(c3ccccc3)c3ccc(-c4cccc5ccc6sc7ccccc7c6c45)cc3)c3ccccc23)cc1 ; c1ccc(-c2ccc(-c3ccc(N(c4ccc(-c5ccccc5)cc4)c4cccc(-c5cccc6ccc7sc8ccccc8c7c56)c4)cc3)cc2)cc1 ; c1ccc(-c2cccc(N(c3cccc(-c4cccc5ccc6sc7ccccc7c6c45)c3)c3ccc(-c4ccccc4)cc3-c3ccccc3)c2)cc1]

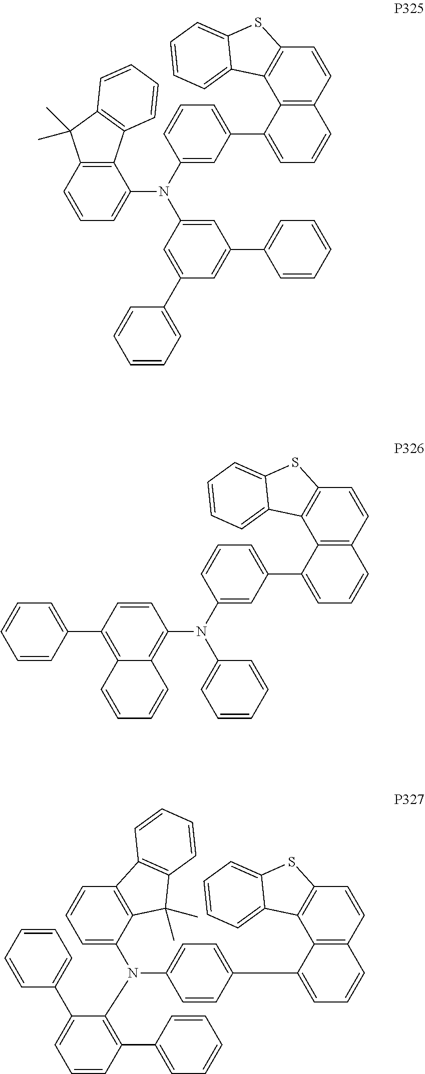 [SMILES: CC1(C)c2ccccc2-c2c(N(c3cc(-c4ccccc4)cc(-c4ccccc4)c3)c3cccc(-c4cccc5ccc6sc7ccccc7c6c45)c3)cccc21 ; c1ccc(-c2ccc(N(c3ccccc3)c3cccc(-c4cccc5ccc6sc7ccccc7c6c45)c3)c3ccccc23)cc1 ; CC1(C)c2ccccc2-c2cccc(N(c3ccc(-c4cccc5ccc6sc7ccccc7c6c45)cc3)c3c(-c4ccccc4)cccc3-c3ccccc3)c21]

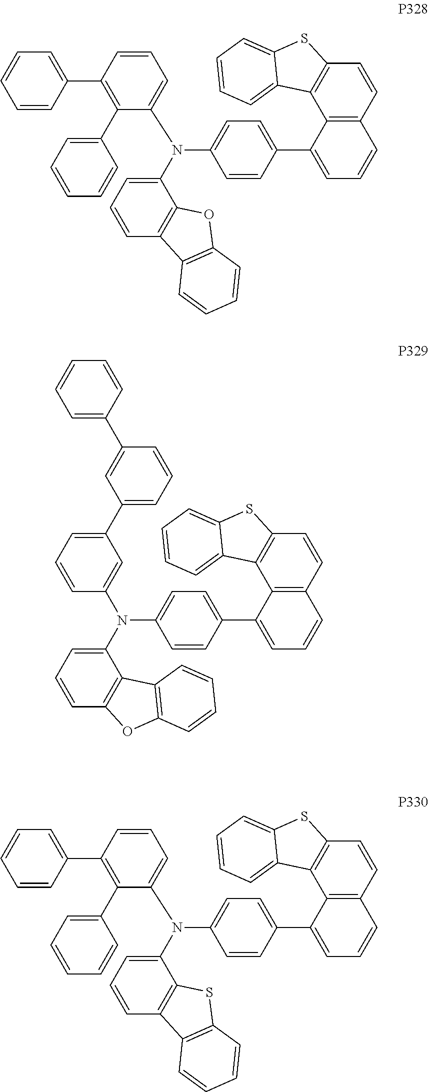 [SMILES: c1ccc(-c2cccc(N(c3ccc(-c4cccc5ccc6sc7ccccc7c6c45)cc3)c3cccc4c3oc3ccccc34)c2-c2ccccc2)cc1 ; c1ccc(-c2cccc(-c3cccc(N(c4ccc(-c5cccc6ccc7sc8ccccc8c7c56)cc4)c4cccc5oc6ccccc6c45)c3)c2)cc1 ; c1ccc(-c2cccc(N(c3ccc(-c4cccc5ccc6sc7ccccc7c6c45)cc3)c3cccc4c3sc3ccccc34)c2-c2ccccc2)cc1]

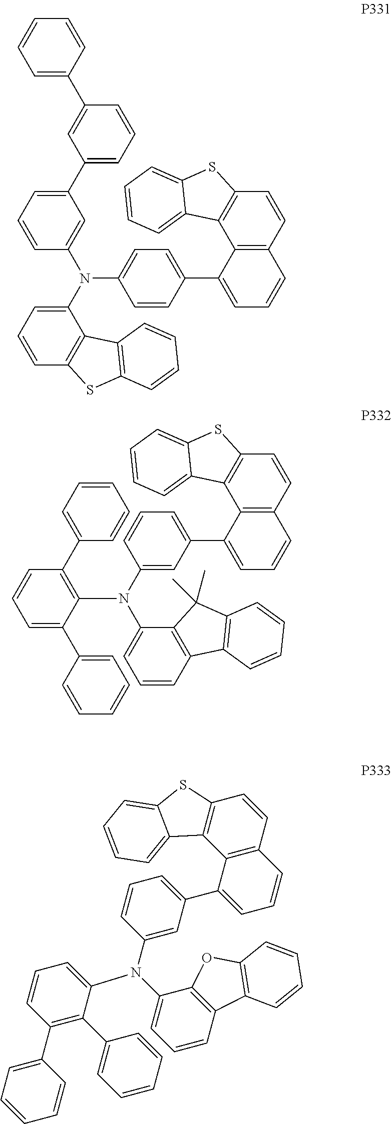

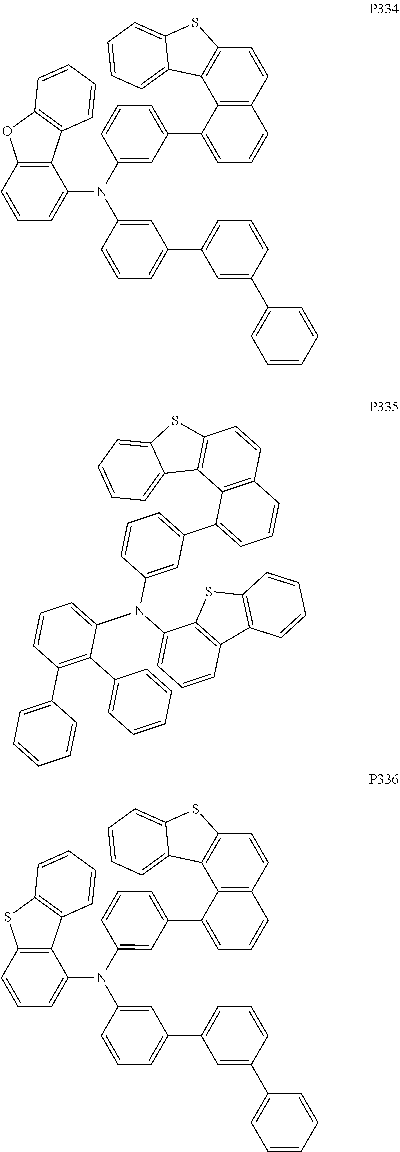 [SMILES: c1ccc(-c2cccc(-c3cccc(N(c4cccc(-c5cccc6ccc7sc8ccccc8c7c56)c4)c4cccc5oc6ccccc6c45)c3)c2)cc1 ; c1ccc(-c2cccc(N(c3cccc(-c4cccc5ccc6sc7ccccc7c6c45)c3)c3cccc4c3sc3ccccc34)c2-c2ccccc2)cc1 ; c1ccc(-c2cccc(-c3cccc(N(c4cccc(-c5cccc6ccc7sc8ccccc8c7c56)c4)c4cccc5sc6ccccc6c45)c3)c2)cc1]

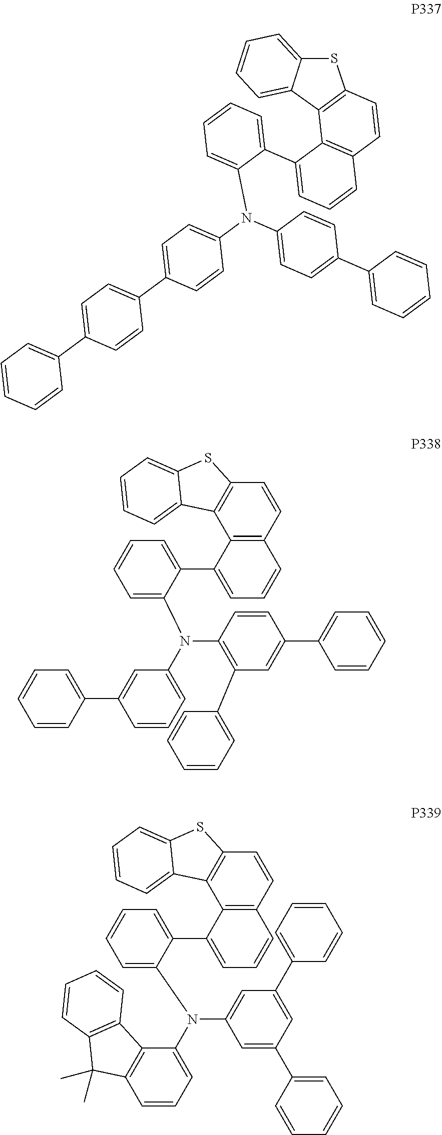 [SMILES: c1ccc(-c2ccc(-c3ccc(N(c4ccc(-c5ccccc5)cc4)c4ccccc4-c4cccc5ccc6sc7ccccc7c6c45)cc3)cc2)cc1 ; c1ccc(-c2cccc(N(c3ccc(-c4ccccc4)cc3-c3ccccc3)c3ccccc3-c3cccc4ccc5sc6ccccc6c5c34)c2)cc1 ; CC1(C)c2ccccc2-c2c(N(c3cc(-c4ccccc4)cc(-c4ccccc4)c3)c3ccccc3-c3cccc4ccc5sc6ccccc6c5c34)cccc21]

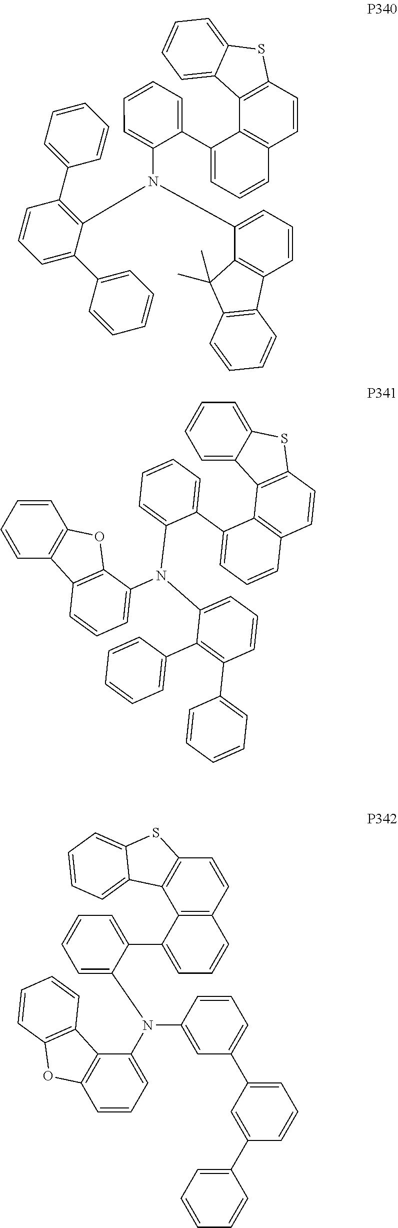

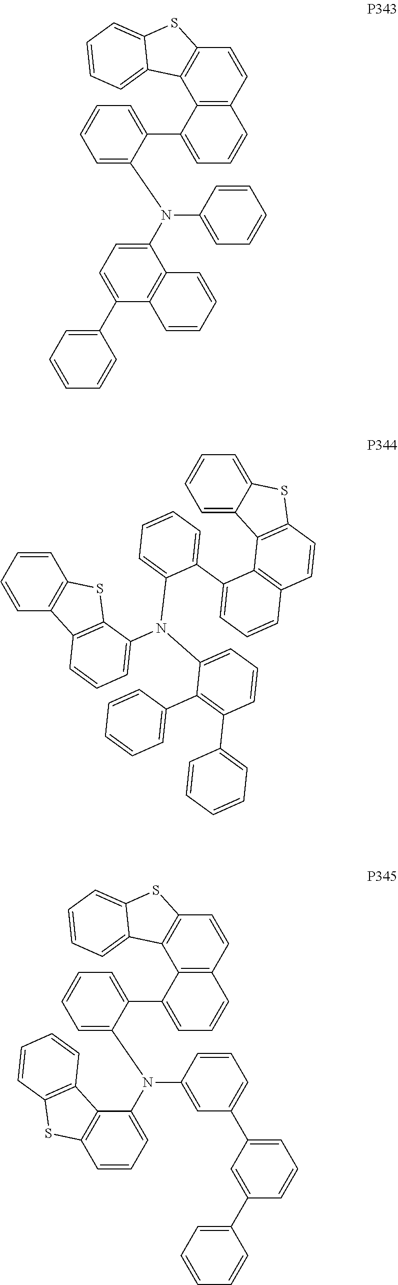 [SMILES: c1ccc(-c2ccc(N(c3ccccc3)c3ccccc3-c3cccc4ccc5sc6ccccc6c5c34)c3ccccc23)cc1 ; c1ccc(-c2cccc(N(c3ccccc3-c3cccc4ccc5sc6ccccc6c5c34)c3cccc4c3sc3ccccc34)c2-c2ccccc2)cc1 ; c1ccc(-c2cccc(-c3cccc(N(c4ccccc4-c4cccc5ccc6sc7ccccc7c6c45)c4cccc5sc6ccccc6c45)c3)c2)cc1]

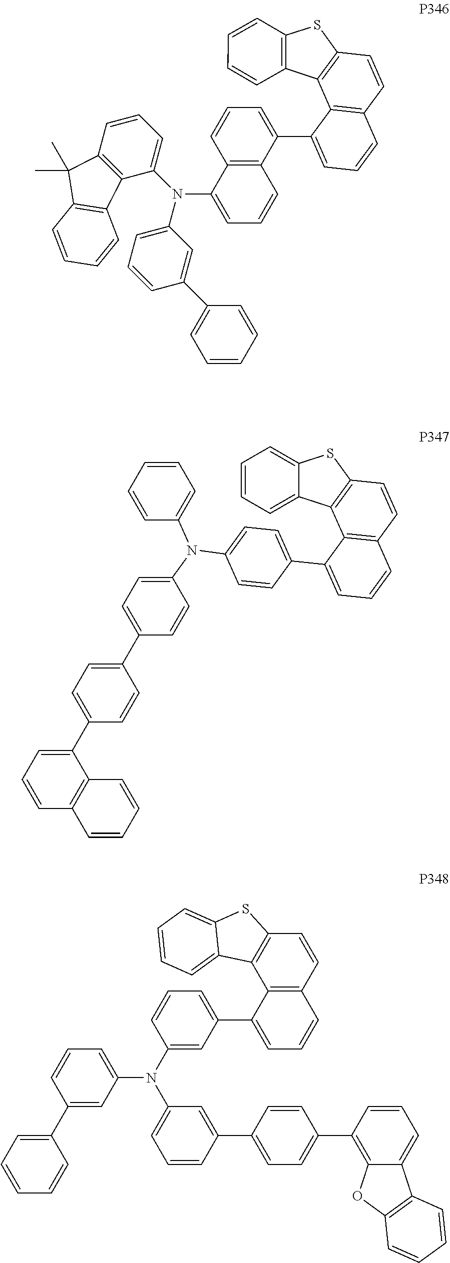 [SMILES: CC1(C)c2ccccc2-c2c(N(c3cccc(-c4ccccc4)c3)c3cccc4c(-c5cccc6ccc7sc8ccccc8c7c56)cccc34)cccc21 ; c1ccc(N(c2ccc(-c3ccc(-c4cccc5ccccc45)cc3)cc2)c2ccc(-c3cccc4ccc5sc6ccccc6c5c34)cc2)cc1 ; c1ccc(-c2cccc(N(c3cccc(-c4ccc(-c5cccc6c5oc5ccccc56)cc4)c3)c3cccc(-c4cccc5ccc6sc7ccccc7c6c45)c3)c2)cc1]

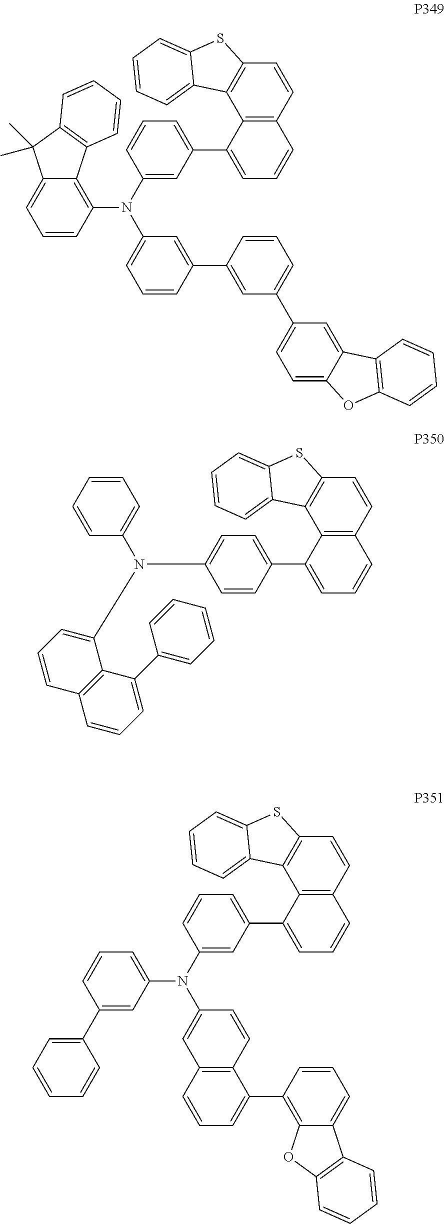 [SMILES: CC1(C)c2ccccc2-c2c(N(c3cccc(-c4cccc(-c5ccc6oc7ccccc7c6c5)c4)c3)c3cccc(-c4cccc5ccc6sc7ccccc7c6c45)c3)cccc21 ; c1ccc(-c2cccc3cccc(N(c4ccccc4)c4ccc(-c5cccc6ccc7sc8ccccc8c7c56)cc4)c23)cc1 ; c1ccc(-c2cccc(N(c3cccc(-c4cccc5ccc6sc7ccccc7c6c45)c3)c3ccc4c(-c5cccc6c5oc5ccccc56)cccc4c3)c2)cc1]

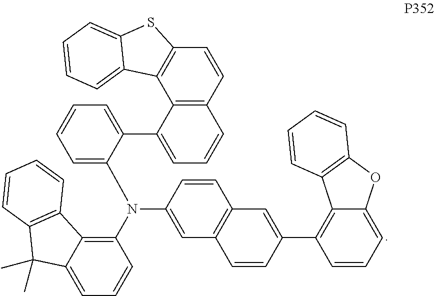 [SMILES: CC1(C)c2ccccc2-c2c(N(c3ccc4cc(-c5cccc6oc7ccccc7c56)ccc4c3)c3ccccc3-c3cccc4ccc5sc6ccccc6c5c34)cccc21]

The present application has no specific limitation on a synthesis method of the nitrogen-containing compound, and those skilled in the art can determine a suitable synthesis method according to the preparation methods provided in the synthesis examples of the nitrogen-containing compound of the present application. In other words, a preparation method of the nitrogen-containing compound is exemplarily provided in the synthesis examples of the present disclosure, and the raw materials used can be obtained commercially or by methods well known in the art. Those skilled in the art can prepare any of the nitrogen-containing compounds provided in the present application according to these exemplary preparation methods, and all specific preparation methods for preparing the nitrogen-containing compounds will not be described in detail here, which should not be construed as a limitation to the present application.

The present application also provides an electronic element, including: an anode and a cathode that are arranged oppositely, and a functional layer arranged between the anode and the cathode, wherein the functional layer includes the nitrogen-containing compound of the present application.

Optionally, the functional layer may include an HTL, and the HTL may include the nitrogen-containing compound. The nitrogen-containing compound provided in the present application can be used for an HTL of an OLED to improve the light-emitting efficiency and life span of the OLED.

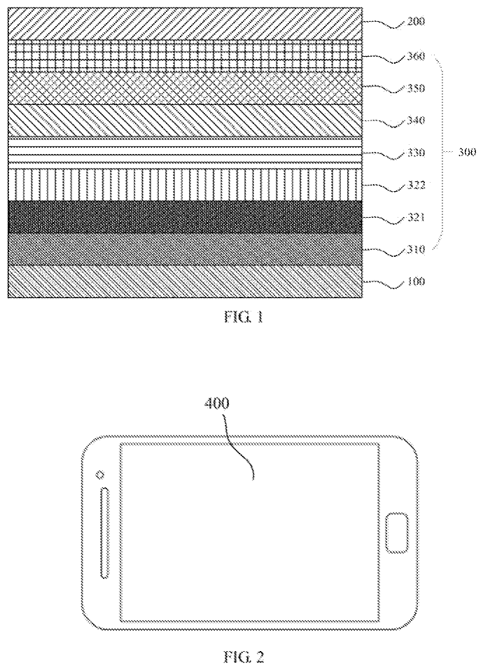

In an embodiment of the present application, the electronic element is an OLED. As shown in FIG. 1, the OLED may include an anode 100, an HTL 321, an EBL 322, an organic electroluminescent layer 330, an ETL 350, and a cathode 200 that are successively stacked.

Optionally, the anode 100 is preferably made of a material with a large work function that facilitates the injection of holes into the functional layer. Specific examples of the anode material may include: metals such as nickel, platinum, vanadium, chromium, copper, zinc, and gold or alloys thereof; metal oxides such as zinc oxide, indium oxide, indium tin oxide (ITO), and indium zinc oxide (IZO); a recombination of a metal and an oxide such as ZnO:Al or SnO₂:Sb; or conductive polymers such as poly(3-methylthiophene), poly[3,4-(ethylene-1,2-dioxy)thiophene] (PEDT), polypyrrole (PPy), and polyaniline (PANI); but are not limited thereto. Preferably, a transparent electrode with ITO is adopted as the anode.

Optionally, the HTL 321 may include the nitrogen-containing compound of the present application.

Optionally, materials for the EBL 322 can be EBL materials such as carbazole polymers and carbazole-linked triarylamine compounds well known in the art, which are not repeated here. For example, the EBL can be EB-01.

Optionally, the organic electroluminescent layer 330 is prepared from a single light-emitting material, or may include a host material and a guest material. Optionally, the organic electroluminescent layer 330 may include a host material and a guest material, wherein holes and electrons injected into the organic electroluminescent layer 330 can be recombined in the organic electroluminescent layer 330 to form excitons, the excitons transfer energy to the host material, and then the host material transfers energy to the guest material, such that the guest material can emit light.

The host material of the organic electroluminescent layer 330 is a metal chelate compound, a bisstyryl derivative, an aromatic amine derivative, a dibenzofuran derivative, or the like, which is not particularly limited in the present application. In an embodiment of the present application, the host material of the organic electroluminescent layer 330 is BH-01.

The guest material of the organic electroluminescent layer 330 is a compound with a condensed aryl ring or a derivative thereof, a compound with a heteroaryl ring or a derivative thereof, an aromatic amine derivative, or the like, which is not particularly limited in the present application. In an embodiment of the present application, the guest material of the organic electroluminescent layer 330 is BD-01.

The ETL 350 may have a single-layer structure or a multi-layer structure, which may include one or more electron transport materials. The electron transport materials may be benzimidazole derivatives, oxadiazole derivatives, quinoxaline derivatives, or other electron transport materials, which are not particularly limited in the present application. For example, the ETL 350 may include ET-06 and LiQ.

Optionally, the cathode 200 is made of a material with a small work function that facilitates the injection of electrons into the functional layer. Specific examples of the cathode material may include, but are not limited to: metals such as magnesium, calcium, sodium, potassium, titanium, indium, yttrium, lithium, gadolinium, aluminum, argentum, tin, and lead or alloys thereof: or multi-layer materials such as LiF/Al, Liq/Al, LiO₂/Al, LiF/Ca, LiF/Al, and BaF₂/Ca. Preferably, a metal electrode with magnesium (Mg) and argentum (Ag) is adopted as the cathode.

Optionally, as shown in FIG. 1, an HIL 310 is further arranged between the anode 100 and the HTL 321 to enhance the ability to inject holes into the HTL 321. The HIL 310 can be made of a benzidine derivative, a starburst arylamine compound, a phthalocyanine derivative, or another material, which is not particularly limited in the present application. For example, the HIL 310 may include F4-TCNQ.

Optionally, as shown in FIG. 1, an EIL 360 is further arranged between the cathode 200 and the ETL 350 to enhance the ability to inject electrons into the ETL 350. The EIL 360 may include an inorganic material such as an alkali metal sulfide and an alkali metal halide, or may include a complex of an alkali metal and an organic substance. In an embodiment of the present application, the EIL 360 may include Yb.

Optionally, an HBL 340 may or may not be arranged between the organic electroluminescent layer 330 and the ETL 350, and a material for the HBL 340 is well known in the art and will not be repeated here.

In an embodiment of the present application, an electronic device is also provided, and the electronic device includes the electronic element described above.

For example, as shown in FIG. 2, the electronic device is a first electronic device 400, and the first electronic device 400 includes the OLED described above. The first electronic device 400 is a display device, a lighting device, an optical communication device, or another electronic device, including but not limited to computer screen, mobile phone screen, television set, electronic paper, emergency light, and optical module.

The present application will be further described in detail below through examples. However, the following examples are only illustrations of the present application, and do not limit the present application.

### SYNTHESIS EXAMPLES

### 1. Synthesis of an Intermediate IM 1-1

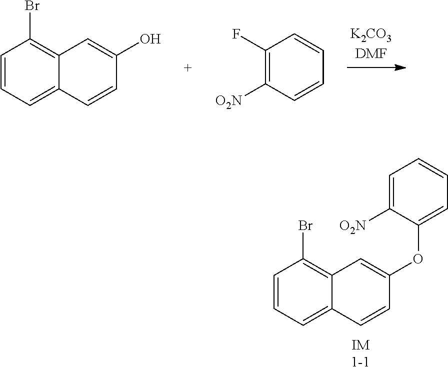 [SMILES: Oc1ccc2cccc(Br)c2c1 ; O=[N+]([O-])c1ccccc1F ; O=[N+]([O-])c1ccccc1Oc1ccc2cccc(Br)c2c1]

Under the protection of N₂, 1-hydroxy-7-bromonaphthalene (80 g, 359 mmol), 2-fluoronitrobenzene (42.2 g, 299 mmol), potassium carbonate (K₂CO₃) (82.6 g, 598 mmol), and dimethylformamide (DMF) (640 mL) were added to a 1,000 mL three-necked flask, a resulting mixture was heated to reflux at 130° C. and stirred for 7 h, then the heating was stopped, and 1,000 mL of ethyl acetate was added; a resulting system was washed with water until neutral, dried with anhydrous magnesium sulfate, and filtered; a resulting organic phase was concentrated, and a concentrate was passed by a silica gel column with petroleum ether (PE):ethyl acetate (v/v)=20:1 to obtain a pale yellow solid IM 1-1 (93.5 g, yield: 91%);

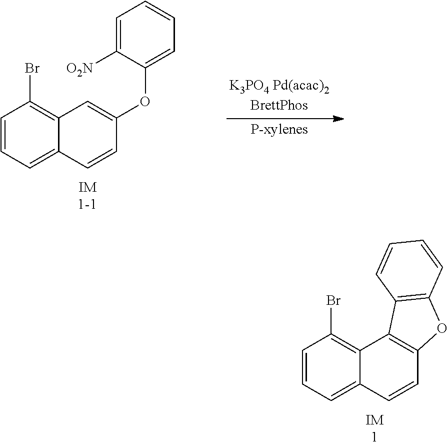 [SMILES: O=[N+]([O-])c1ccccc1Oc1ccc2cccc(Br)c2c1 ; Brc1cccc2ccc3oc4ccccc4c3c12]

Under the protection of N₂, the IM 1-1 (93.5 g, 272 mmol), potassium phosphate (K₃PO₄) (172 g, 815 mmol), BrettPhos (1.46 g, 2.7 mmol), palladium 2,4-glutarate (Pd(acac)₂) (0.41 g, 1.36 mmol), and p-xylene (720 mL) were added to a 1,000 mL three-necked flask, a resulting mixture was heated to 150° C. to 160° C. to allow a reaction for 24 h, and the heating was stopped; a resulting system was cooled to room temperature, washed with water until neutral, dried with anhydrous magnesium sulfate, and filtered; and a resulting organic phase was concentrated, and a concentrate was passed by a silica gel column with PE:ethyl acetate (v/v)=10:1 to obtain a white solid IM 1 (70.2 g, yield: 87%).

### 2. Synthesis of an Intermediate IM 2

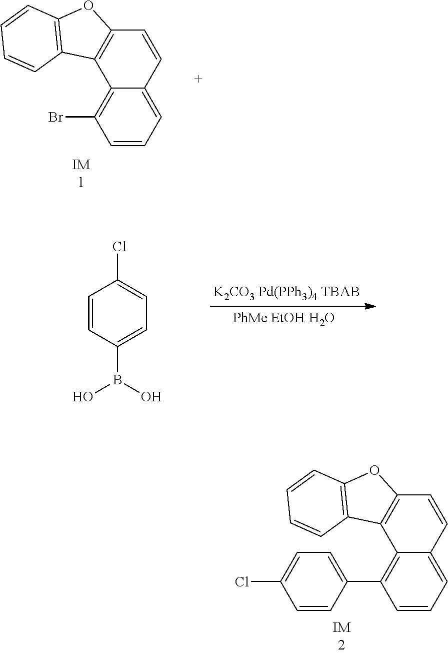 [SMILES: Brc1cccc2ccc3oc4ccccc4c3c12 ; OB(O)c1ccc(Cl)cc1 ; Clc1ccc(-c2cccc3ccc4oc5ccccc5c4c23)cc1]

Under the protection of N₂, the IM 1 (70.2 g, 236 mmol), 4-chlorophenylboronic acid (36.8 g, 236 mmol), potassium carbonate (65.3 g, 473 mmol), tetrakis(triphenylphosphine)palladium (Pd(PPh₃)₄) (2.73 g, 2.36 mmol), tetrabutylammonium bromide (TBAB) (1.5 g, 4.7 mmol), toluene (PhMe) (420 mL), absolute ethanol (210 mL), and water (70 mL) were added to a 500 mL three-necked flask, a resulting mixture was heated to reflux at 80° C. and stirred for 24 h, and then the reaction was stopped; a resulting reaction system was cooled to room temperature, washed with water until neutral, dried with anhydrous magnesium sulfate, and filtered; and a resulting organic phase was concentrated, and a concentrate was passed by a silica gel column with dichloromethane (DCM):n-heptane (v/v)=1:3 to obtain a white solid IM 2 (69.1 g, yield: 89%).

IM X was synthesized with reference to the synthesis method of IM 2, except that a raw material 1 was used instead of 4-chlorophenylboronic acid. The main raw materials used and the synthesized intermediates and yields thereof were shown in Table 1:

TABLE 1

| Raw material 1 | IMX | Yield/% |
|---|---|---|
| 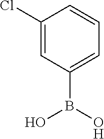 | 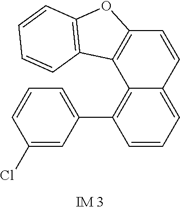 | 85 |
| 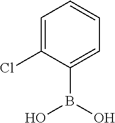 | 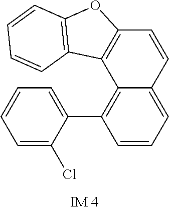 | 72 |
| 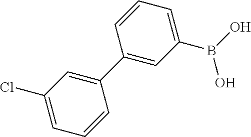 [SMILES: OB(O)c1cccc(-c2cccc(Cl)c2)c1] | 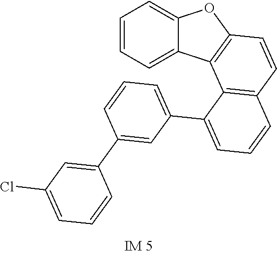 | 69 |
| 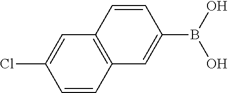 [SMILES: OB(O)c1ccc2cc(Cl)ccc2c1] | 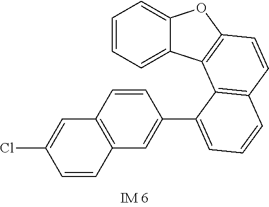 | 71 |
| 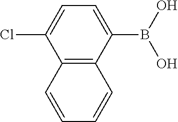 [SMILES: OB(O)c1ccc(Cl)c2ccccc12] | 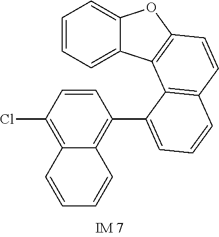 | 68% |
| 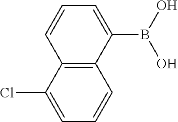 [SMILES: OB(O)c1cccc2c(Cl)cccc12] | 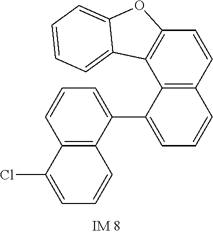 | 67% |

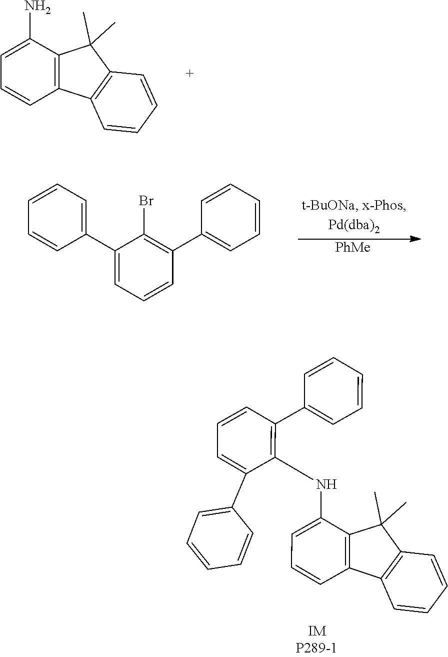 [SMILES: CC1(C)c2ccccc2-c2cccc(N)c21 ; Brc1c(-c2ccccc2)cccc1-c1ccccc1 ; "PhMe" ; CC1(C)c2ccccc2-c2cccc(Nc3c(-c4ccccc4)cccc3-c3ccccc3)c21]

### 3. Synthesis of an Intermediate IM P289-1

Under the protection of N₂, 1-amino-9,9-dimethylfluorene (3.13 g, 15 mmol), 2′-bromo-1,1′:3′,1′-terphenyl (4.6 g, 15 mmol), and toluene (50 mL) were added to a 100 mL three-necked flask, a resulting mixture was heated to reflux at 108° C. and stirred until a clear solution was obtained, and then the clear solution was cooled to 70° C. to 80° C.; sodium tert-butoxide (t-BuONa) (2.2 g, 22.8 mmol), 2-dicyclohexylphosphino-2,4,6-triisopropylbiphenyl (x-Phos) (0.12 g, 0.3 mmol), and bis(dibenzylideneacetone)palladium (Pd(dba)₂) (0.14 g, 0.15 mmol) were added, a resulting mixture was heated to reflux at 108° C. for 5 h, and then the heating was stopped; and a resulting reaction system was cooled to room temperature, washed with water until neutral, dried with anhydrous magnesium sulfate, and filtered; and a resulting organic phase was concentrated, and a concentrate was passed through a silica gel column with DCM:n-heptane (v/v)=1:5 to obtain a white solid IM P289-1 (4 g, yield: 61%).

The intermediates listed in Table 2 were each synthesized with reference to the synthesis method of IM P289-1, except that a raw material 2 was used instead of 1-amino-9,9-dimethylfluorene and a raw material 3 was used instead of 2′-bromo-1,1′:3′,1′-terphenyl. The main raw materials used and the synthesized intermediates and yields thereof were shown in Table 2:

TABLE 2

| Raw material 2 | Raw material 3 | Intermediate | Yield/% |
|---|---|---|---|
| 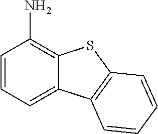 [SMILES: Nc1cccc2c1sc1ccccc12] | 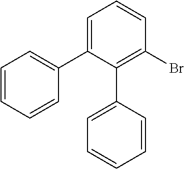 [SMILES: Brc1cccc(-c2ccccc2)c1-c1ccccc1] | 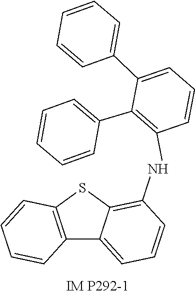 [SMILES: c1ccc(-c2cccc(Nc3cccc4c3sc3ccccc34)c2-c2ccccc2)cc1] | 54 |
| 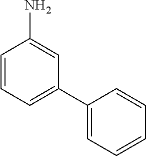 [SMILES: Nc1cccc(-c2ccccc2)c1] | 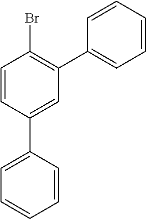 [SMILES: Brc1ccc(-c2ccccc2)cc1-c1ccccc1] | 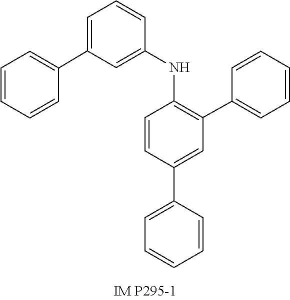 [SMILES: c1ccc(-c2cccc(Nc3ccc(-c4ccccc4)cc3-c3ccccc3)c2)cc1] | 65 |
| | 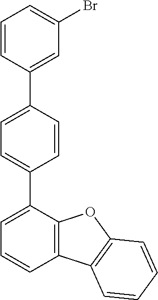 [SMILES: Brc1cccc(-c2ccc(-c3cccc4c3oc3ccccc34)cc2)c1] | 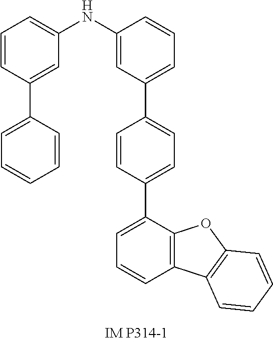 [SMILES: c1ccc(-c2cccc(Nc3cccc(-c4ccc(-c5cccc6c5oc5ccccc56)cc4)c3)c2)cc1] | 78 |
| | 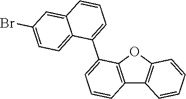 [SMILES: Brc1ccc2c(-c3cccc4c3oc3ccccc34)cccc2c1] | 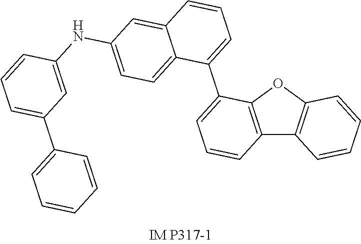 [SMILES: c1ccc(-c2cccc(Nc3ccc4c(-c5cccc6c5oc5ccccc56)cccc4c3)c2)cc1] | 56 |
| 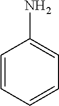 | 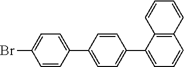 [SMILES: Brc1ccc(-c2ccc(-c3cccc4ccccc34)cc2)cc1] | 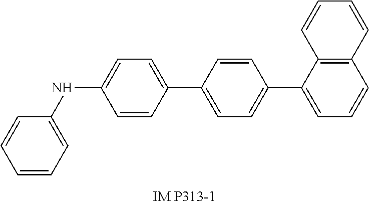 [SMILES: c1ccc(Nc2ccc(-c3ccc(-c4cccc5ccccc45)cc3)cc2)cc1] | 74 |
| | 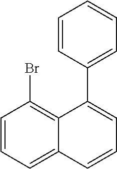 [SMILES: Brc1cccc2cccc(-c3ccccc3)c12] | 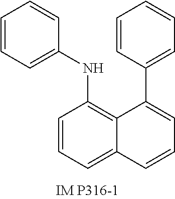 [SMILES: c1ccc(Nc2cccc3cccc(-c4ccccc4)c23)cc1] | 81 |
| 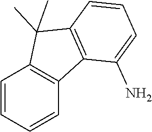 [SMILES: CC1(C)c2ccccc2-c2c(N)cccc21] | 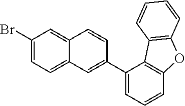 [SMILES: Brc1ccc2cc(-c3cccc4oc5ccccc5c34)ccc2c1] | 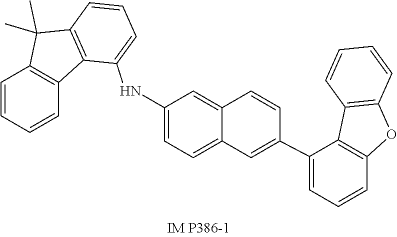 [SMILES: CC1(C)c2ccccc2-c2c(Nc3ccc4cc(-c5cccc6oc7ccccc7c56)ccc4c3)cccc21] | 53 |

### 4. Synthesis of a Compound P1

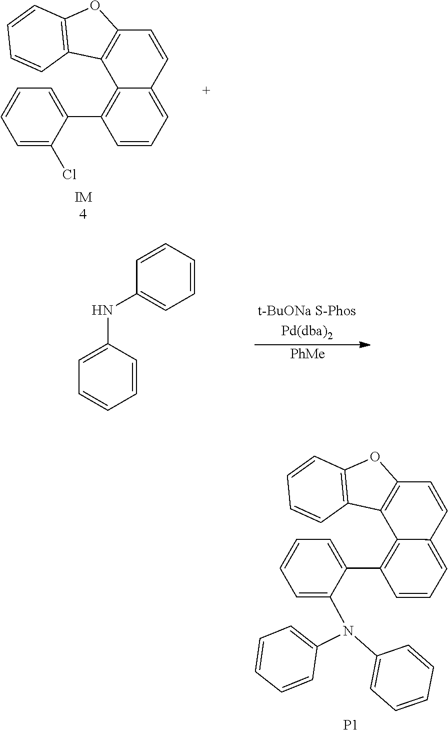

Under the protection of N₂, IM 4 (5 g, 15 mmol), diphenylamine (DPA) (2.6 g, 15 mmol), and toluene (50 mL) were added to a 100 mL three-necked flask, a resulting mixture was heated to reflux at 108° C. and stirred until a clear solution was obtained, and then the clear solution was cooled to 70° C. to 80° C.; sodium tert-butoxide (2.2 g, 22.8 mmol), 2-dicyclohexylphosphino-2′,6′-dimethoxybiphenyl (S-Phos) (0.12 g, 0.3 mmol), and bis(dibenzylideneacetone)palladium (Pd(dba)₂) (0.14 g, 0.15 mmol) were added, a resulting mixture was heated to reflux at 108° C. for 3 h, and then the heating was stopped; and a resulting reaction system was cooled to room temperature, washed with water until neutral, dried with anhydrous magnesium sulfate, and filtered; and a resulting organic phase was concentrated, and a concentrate was passed through a silica gel column with DCM:n-heptane (v/v)=1:3 to obtain a white solid compound P1 (4 g, yield: 57%, and MS: (m/z)=462.18 [M+H]⁺).

The compounds listed in Table 3 were each synthesized with reference to the synthesis method of the compound P1, except that a raw material 4 was used instead of the IM 4 and a raw material 5 was used instead of the DPA. The main raw materials used and the synthesized compounds and yields and MS data thereof were shown in Table 3:

TABLE 3

| | | | | MS (m/z)/ |
|---|---|---|---|---|
| Raw material 4 | Raw material 5 | Compound | Yield/% | [M + H] |
| 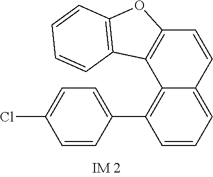 [SMILES: Clc1ccc(-c2cccc3ccc4oc5ccccc5c4c23)cc1] | 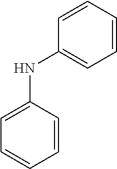 [SMILES: c1ccc(Nc2ccccc2)cc1] | 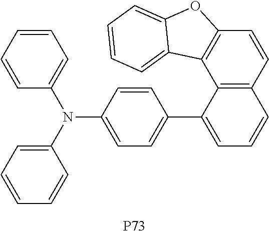 [SMILES: "P73" ; c1ccc(N(c2ccccc2)c2ccc(-c3cccc4ccc5oc6ccccc6c5c34)cc2)cc1] | 86 | 462.28 |
| | 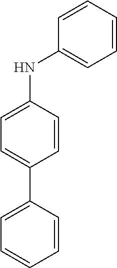 [SMILES: c1ccc(Nc2ccc(-c3ccccc3)cc2)cc1] | 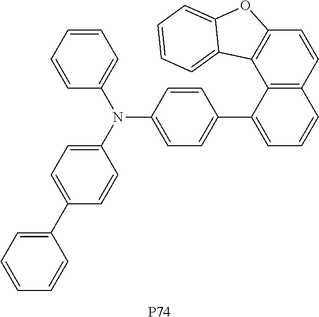 [SMILES: "P74" ; c1ccc(-c2ccc(N(c3ccccc3)c3ccc(-c4cccc5ccc6oc7ccccc7c6c45)cc3)cc2)cc1] | 84 | 538.21 |
| | 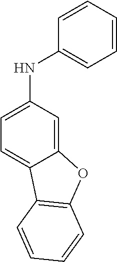 [SMILES: c1ccc(Nc2ccc3c(c2)oc2ccccc23)cc1] | 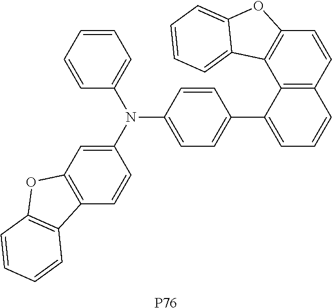 [SMILES: "P76" ; c1ccc(N(c2ccc(-c3cccc4ccc5oc6ccccc6c5c34)cc2)c2ccc3c(c2)oc2ccccc23)cc1] | 83 | 552.19 |
| | 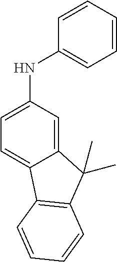 [SMILES: CC1(C)c2ccccc2-c2ccc(Nc3ccccc3)cc21] | 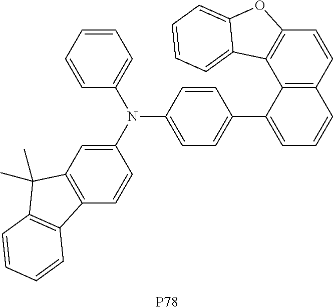 [SMILES: "P78" ; CC1(C)c2ccccc2-c2ccc(N(c3ccccc3)c3ccc(-c4cccc5ccc6oc7ccccc7c6c45)cc3)cc21] | 82 | 578.24 |
| | 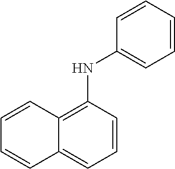 [SMILES: c1ccc(Nc2cccc3ccccc23)cc1] | 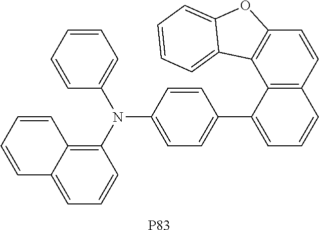 [SMILES: "P83" ; c1ccc(N(c2ccc(-c3cccc4ccc5oc6ccccc6c5c34)cc2)c2cccc3ccccc23)cc1] | 79 | 512.19 |
| | 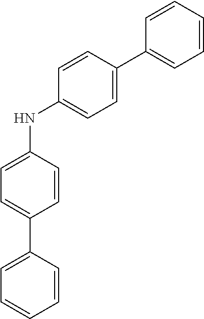 [SMILES: c1ccc(-c2ccc(Nc3ccc(-c4ccccc4)cc3)cc2)cc1] | 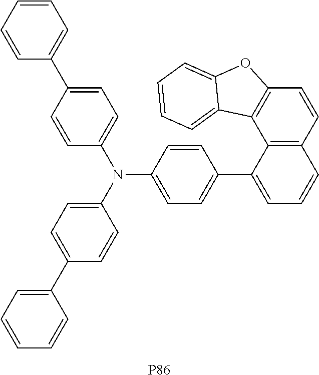 [SMILES: "P86" ; c1ccc(-c2ccc(N(c3ccc(-c4ccccc4)cc3)c3ccc(-c4cccc5ccc6oc7ccccc7c6c45)cc3)cc2)cc1] | 81 | 614.24 |
| | 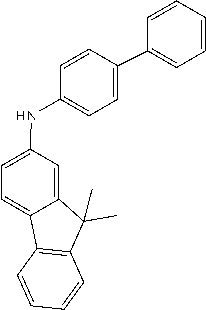 [SMILES: CC1(C)c2ccccc2-c2ccc(Nc3ccc(-c4ccccc4)cc3)cc21] | 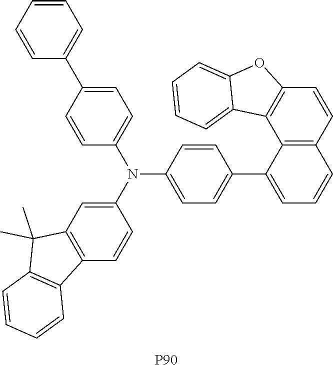 [SMILES: "P90" ; CC1(C)c2ccccc2-c2ccc(N(c3ccc(-c4ccccc4)cc3)c3ccc(-c4cccc5ccc6oc7ccccc7c6c45)cc3)cc21] | 81 | 654.27 |
| | 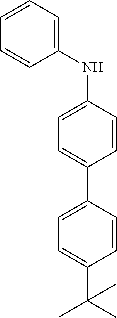 [SMILES: CC(C)(C)c1ccc(-c2ccc(Nc3ccccc3)cc2)cc1] | 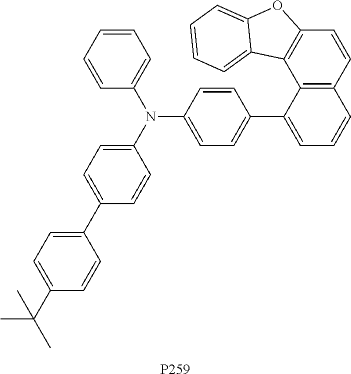 [SMILES: CC(C)(C)c1ccc(-c2ccc(N(c3ccccc3)c3ccc(-c4cccc5ccc6oc7ccccc7c6c45)cc3)cc2)cc1 ; "P259"] | 79 | 594.27 |
| | 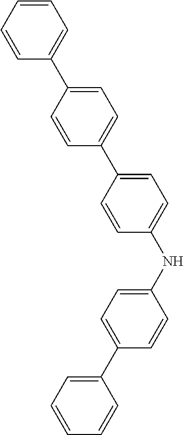 [SMILES: c1ccc(-c2ccc(Nc3ccc(-c4ccc(-c5ccccc5)cc4)cc3)cc2)cc1] | 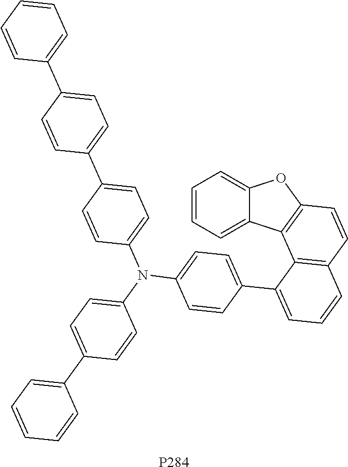 [SMILES: c1ccc(-c2ccc(-c3ccc(N(c4ccc(-c5ccccc5)cc4)c4ccc(-c5cccc6ccc7oc8ccccc8c7c56)cc4)cc3)cc2)cc1 ; "P284"] | 72 | 670.27 |
| | 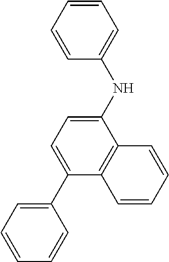 [SMILES: c1ccc(Nc2ccc(-c3ccccc3)c3ccccc23)cc1] | 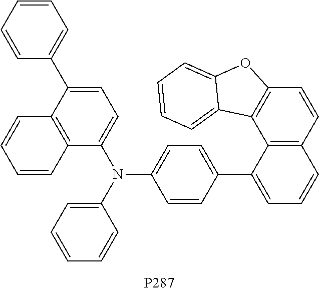 [SMILES: c1ccc(-c2ccc(N(c3ccccc3)c3ccc(-c4cccc5ccc6oc7ccccc7c6c45)cc3)c3ccccc23)cc1 ; "P287"] | 74 | 588,22 |
| | 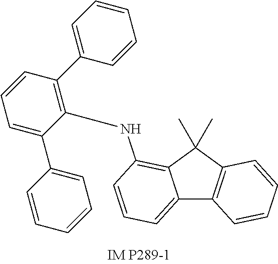 [SMILES: CC1(C)c2ccccc2-c2cccc(Nc3c(-c4ccccc4)cccc3-c3ccccc3)c21] | 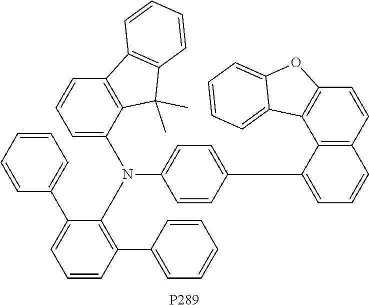 | 45 | 730.30 |
| | 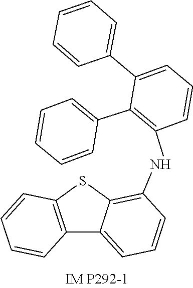 [SMILES: c1ccc(-c2cccc(Nc3cccc4c3sc3ccccc34)c2-c2ccccc2)cc1] | 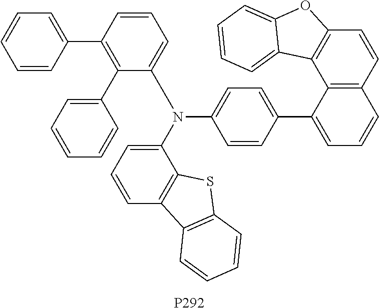 | 46 | 720.23 |
| | 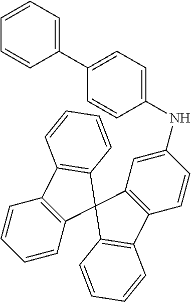 [SMILES: c1ccc(-c2ccc(Nc3ccc4c(c3)C3(c5ccccc5-c5ccccc53)c3ccccc3-4)cc2)cc1] | 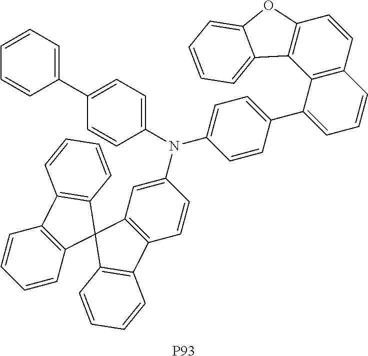 [SMILES: "P93" ; c1ccc(-c2ccc(N(c3ccc(-c4cccc5ccc6oc7ccccc7c6c45)cc3)c3ccc4c(c3)C3(c5ccccc5-c5ccccc53)c3ccccc3-4)cc2)cc1] | 69 | 776 29 |
| | 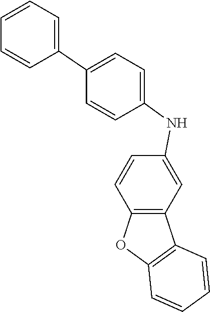 [SMILES: c1ccc(-c2ccc(Nc3ccc4oc5ccccc5c4c3)cc2)cc1] | 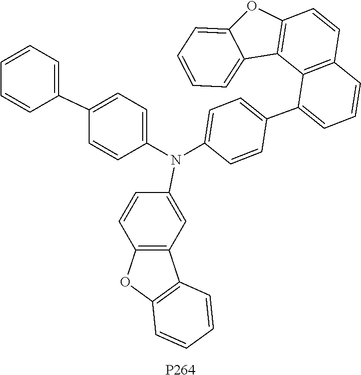 | 75 | 628.22 |
| | 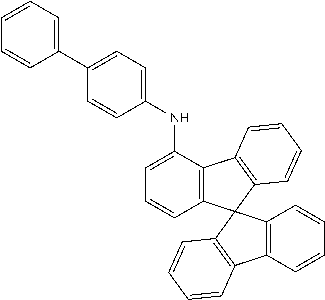 [SMILES: c1ccc(-c2ccc(Nc3cccc4c3-c3ccccc3C43c4ccccc4-c4ccccc43)cc2)cc1] | 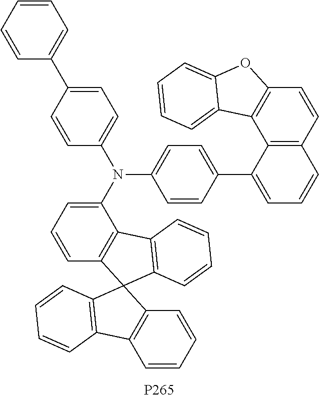 | 74 | 776.29 |
| | 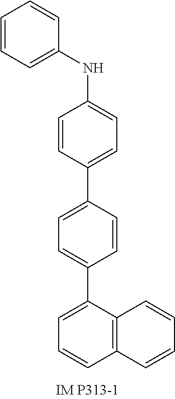 [SMILES: c1ccc(Nc2ccc(-c3ccc(-c4cccc5ccccc45)cc3)cc2)cc1] | 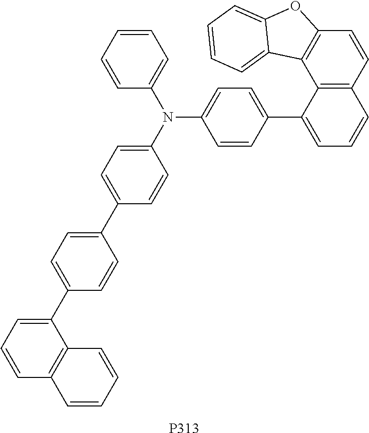 [SMILES: c1ccc(N(c2ccc(-c3ccc(-c4cccc5ccccc45)cc3)cc2)c2ccc(-c3cccc4ccc5oc6ccccc6c5c34)cc2)cc1 ; "P313"] | 64 | 664.26 |
| | 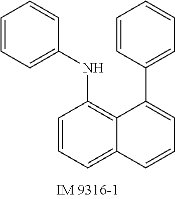 [SMILES: c1ccc(Nc2cccc3cccc(-c4ccccc4)c23)cc1] | 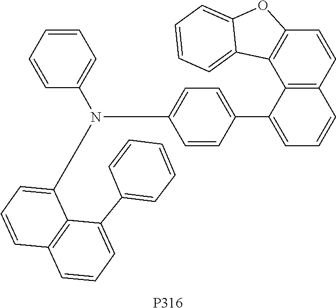 [SMILES: c1ccc(-c2cccc3cccc(N(c4ccccc4)c4ccc(-c5cccc6ccc7oc8ccccc8c7c56)cc4)c23)cc1 ; "P316"] | 67 | 588.22. |
| 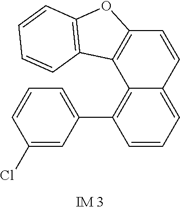 [SMILES: Clc1cccc(-c2cccc3ccc4oc5ccccc5c4c23)c1] | 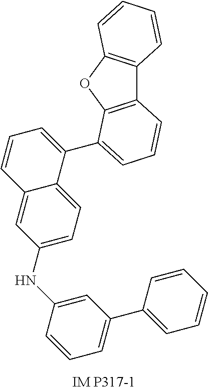 [SMILES: c1ccc(-c2cccc(Nc3ccc4c(-c5cccc6c5oc5ccccc56)cccc4c3)c2)cc1] | 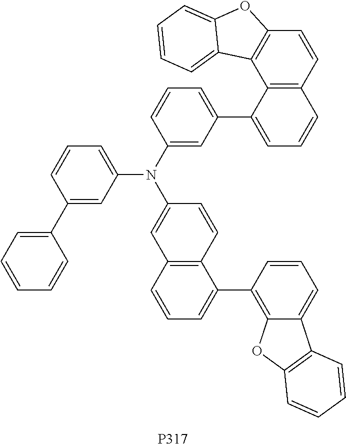 [SMILES: c1ccc(-c2cccc(N(c3cccc(-c4cccc5ccc6oc7ccccc7c6c45)c3)c3ccc4c(-c5cccc6c5oc5ccccc56)cccc4c3)c2)cc1 ; "P317"] | 64 | 754.27 |
| | 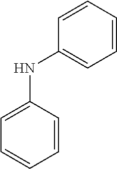 [SMILES: c1ccc(Nc2ccccc2)cc1] | 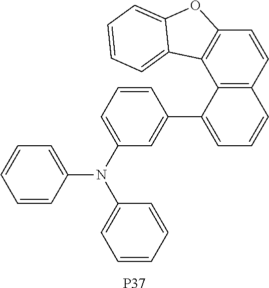 | 84 | 462.18 |
| | 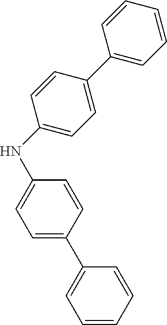 [SMILES: c1ccc(-c2ccc(Nc3ccc(-c4ccccc4)cc3)cc2)cc1] | 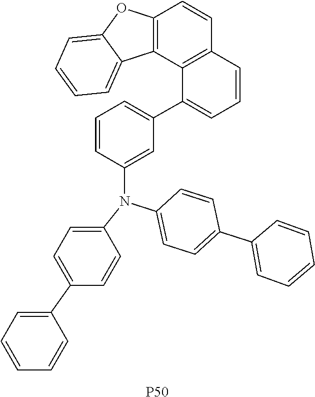 [SMILES: "P50" ; c1ccc(-c2ccc(N(c3ccc(-c4ccccc4)cc3)c3cccc(-c4cccc5ccc6oc7ccccc7c6c45)c3)cc2)cc1] | 72 | 614.24 |
| | 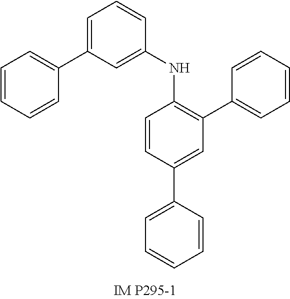 [SMILES: c1ccc(-c2cccc(Nc3ccc(-c4ccccc4)cc3-c3ccccc3)c2)cc1] | 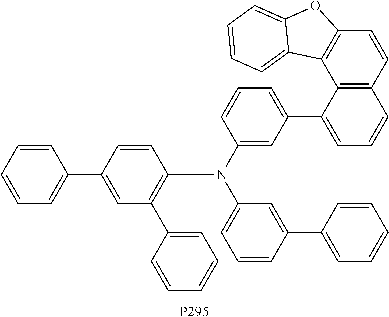 | 76 | 690.27 |
| | 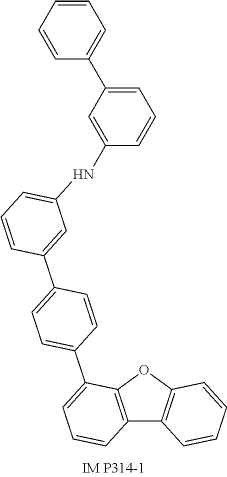 [SMILES: c1ccc(-c2cccc(Nc3cccc(-c4ccc(-c5cccc6c5oc5ccccc56)cc4)c3)c2)cc1] | 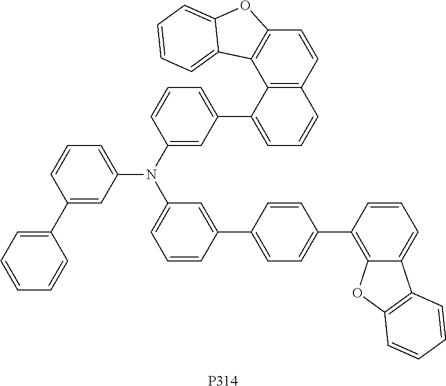 [SMILES: c1ccc(-c2cccc(N(c3cccc(-c4ccc(-c5cccc6c5oc5ccccc56)cc4)c3)c3cccc(-c4cccc5ccc6oc7ccccc7c6c45)c3)c2)cc1 ; "P314"] | 57 | 780.28 |
| 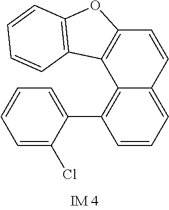 [SMILES: Clc1ccccc1-c1cccc2ccc3oc4ccccc4c3c12] | 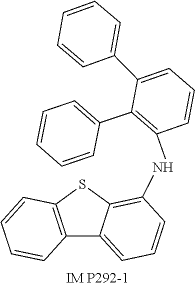 [SMILES: c1ccc(-c2cccc(Nc3cccc4c3sc3ccccc34)c2-c2ccccc2)cc1] | 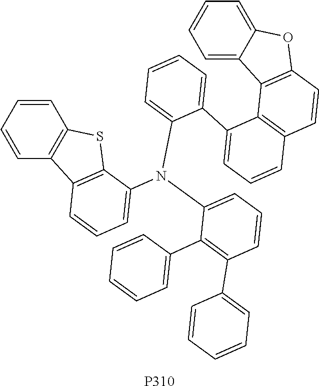 [SMILES: c1ccc(-c2cccc(N(c3ccccc3-c3cccc4ccc5oc6ccccc6c5c34)c3cccc4c3sc3ccccc34)c2-c2ccccc2)cc1 ; "P310"] | 56 | 720.23 |
| | 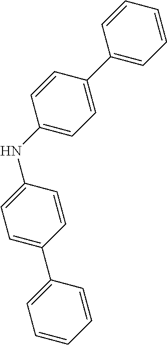 [SMILES: c1ccc(-c2ccc(Nc3ccc(-c4ccccc4)cc3)cc2)cc1] | 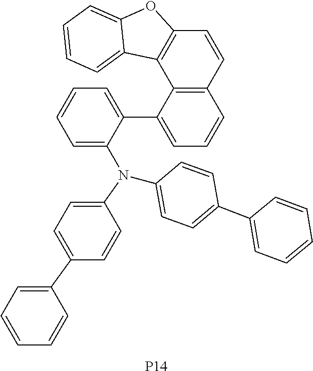 | 67 | 614.24 |
| 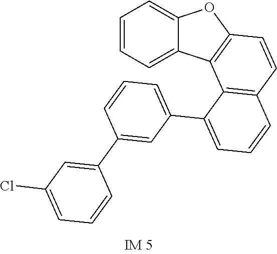 [SMILES: Clc1cccc(-c2cccc(-c3cccc4ccc5oc6ccccc6c5c34)c2)c1] | 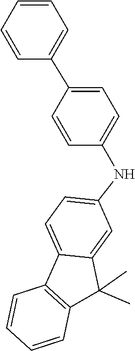 [SMILES: CC1(C)c2ccccc2-c2ccc(Nc3ccc(-c4ccccc4)cc3)cc21] | 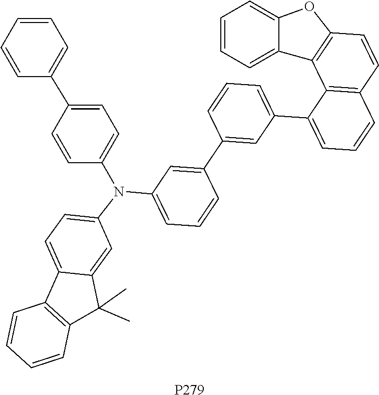 [SMILES: CC1(C)c2ccccc2-c2ccc(N(c3ccc(-c4ccccc4)cc3)c3cccc(-c4cccc(-c5cccc6ccc7oc8ccccc8c7c56)c4)c3)cc21 ; "P279"] | 68 | 730.30 |
| 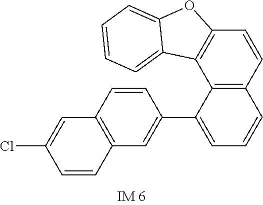 [SMILES: Clc1ccc2cc(-c3cccc4ccc5oc6ccccc6c5c34)ccc2c1] | 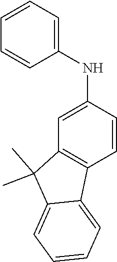 [SMILES: CC1(C)c2ccccc2-c2ccc(Nc3ccccc3)cc21] | 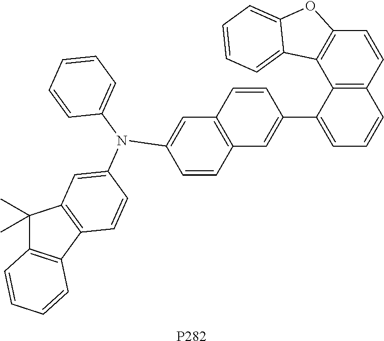 [SMILES: CC1(C)c2ccccc2-c2ccc(N(c3ccccc3)c3ccc4cc(-c5cccc6ccc7oc8ccccc8c7c56)ccc4c3)cc21 ; "P282"] | 65 | 628.26 |
| 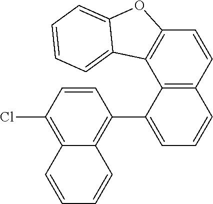 [SMILES: Clc1ccc(-c2cccc3ccc4oc5ccccc5c4c23)c2ccccc12] | 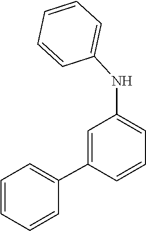 [SMILES: c1ccc(Nc2cccc(-c3ccccc3)c2)cc1] | 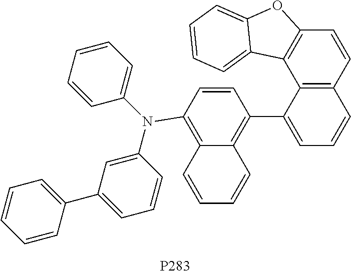 [SMILES: c1ccc(-c2cccc(N(c3ccccc3)c3ccc(-c4cccc5ccc6oc7ccccc7c6c45)c4ccccc34)c2)cc1 ; "P283"] | 68 | 588.22 |
| 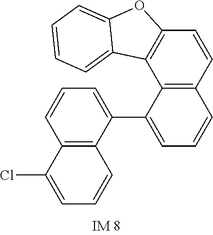 [SMILES: Clc1cccc2c(-c3cccc4ccc5oc6ccccc6c5c34)cccc12] | 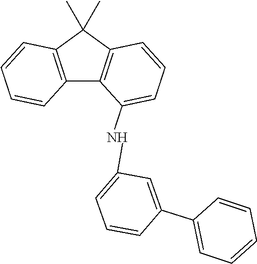 | 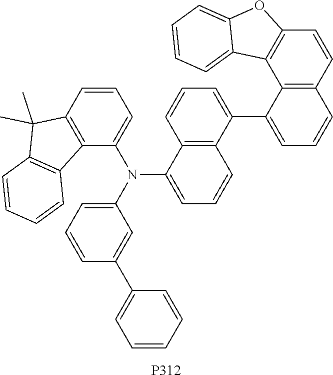 | 67 | 704.29 |

### 5. Synthesis of an Intermediate IM 9-2

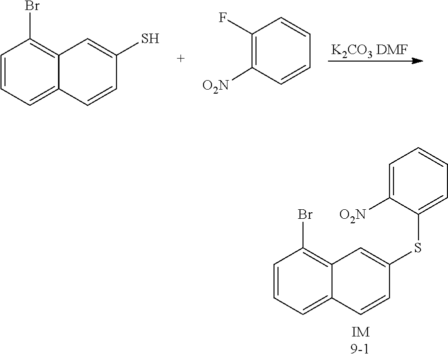

Under the protection of N₂, 1-thiol-7-bromonaphthalene (85.8 g, 359 mmol), 2-fluoronitrobenzene (42.2 g, 299 mmol), potassium carbonate (82.6 g, 598 mmol), and DMF (640 mL) were added to a 1,000 mL three-necked flask, a resulting mixture was heated to reflux at 130° C. and stirred for 7 h, then the heating was stopped, and 1,000 mL of ethyl acetate was added; a resulting system was washed with water until neutral, dried with anhydrous magnesium sulfate, and filtered; a resulting organic phase was concentrated, and a concentrate was passed by a silica gel column with PE:ethyl acetate (v/v)=20:1 to obtain a yellow solid IM 9-1 (118.9 g. yield: 92%)

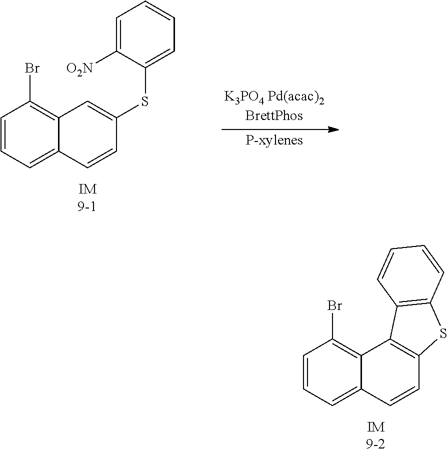 [SMILES: O=[N+]([O-])c1ccccc1Sc1ccc2cccc(Br)c2c1 ; Brc1cccc2ccc3sc4ccccc4c3c12]

Under the protection of N₂, the IM 9-1 (97.9 g, 272 mmol), potassium phosphate (172 g, 815 mmol), BrettPhos (1.46 g, 2.7 mmol), palladium 2,4-glutarate (0.41 g, 1.36 mmol), and xylene (720 mL) were added to a 1,000 mL three-necked flask, a resulting mixture was heated to 150° C. to 160° C. to allow a reaction for 24 h, and the heating was stopped; a resulting system was cooled to room temperature, washed with water until neutral, dried with anhydrous magnesium sulfate, and filtered; and a resulting organic phase was concentrated, and a concentrate was passed by a silica gel column with PE:ethyl acetate (v/v)=10:1 to obtain a white solid IM 9-2 (74.9 g, yield: 88%).

### 6. Synthesis of an Intermediate IM 9

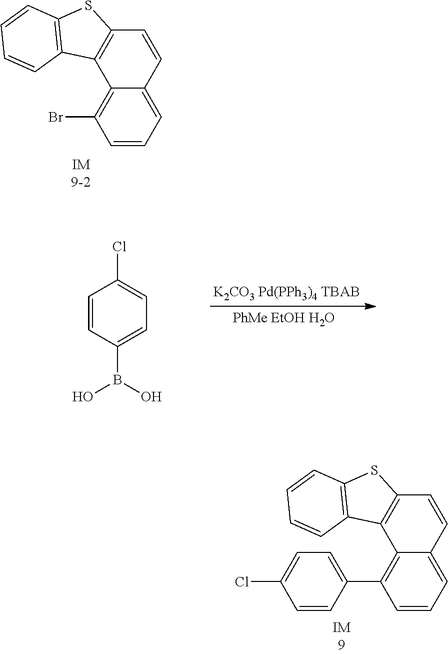 [SMILES: Brc1cccc2ccc3sc4ccccc4c3c12 ; OB(O)c1ccc(Cl)cc1 ; Clc1ccc(-c2cccc3ccc4sc5ccccc5c4c23)cc1]

Under the protection of N₂, the IM 9-2 (73.9 g, 236 mmol), 4-chlorophenylboronic acid (36.8 g, 236 mmol), potassium carbonate (65.3 g, 473 mmol), tetrakis(triphenylphosphine)palladium (2.73 g, 2.36 mmol), TBAB (1.5 g, 4.7 mmol), toluene (420 mL), absolute ethanol (210 mL), and water (70 mL) were added to a 500 mL three-necked flask, a resulting mixture was heated to reflux at 80° C. and stirred for 24 h, and then the reaction was stopped; a resulting reaction system was cooled to room temperature, washed with water until neutral, dried with anhydrous magnesium sulfate, and filtered; and a resulting organic phase was concentrated, and a concentrate was passed by a silica gel column with DCM:n-heptane (v/v)=1:3 to obtain a white solid IM 9 (69.1 g, yield: 85%).

The intermediates listed in Table 4 were each synthesized with reference to the synthesis method of IM 9, except that a raw material 6 was used instead of 4-chlorophenylboronic acid. The main raw materials used and the synthesized intermediates and yields thereof were shown in Table 4:

TABLE 4

| Raw material 6 | Intermediate | Yield/% |
|---|---|---|
| 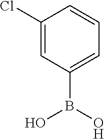 | 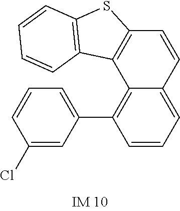 [SMILES: Clc1cccc(-c2cccc3ccc4sc5ccccc5c4c23)c1] | 85 |
| 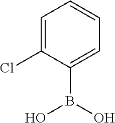 | 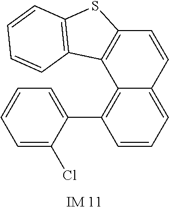 [SMILES: Clc1ccccc1-c1cccc2ccc3sc4ccccc4c3c12] | 72 |
| 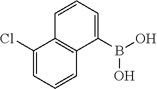 [SMILES: OB(O)c1cccc2c(Cl)cccc12] | 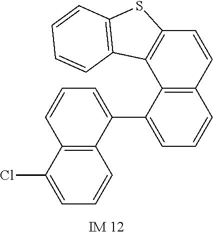 [SMILES: Clc1cccc2c(-c3cccc4ccc5sc6ccccc6c5c34)cccc12] | 73 |

### 7. Synthesis of a Compound P109

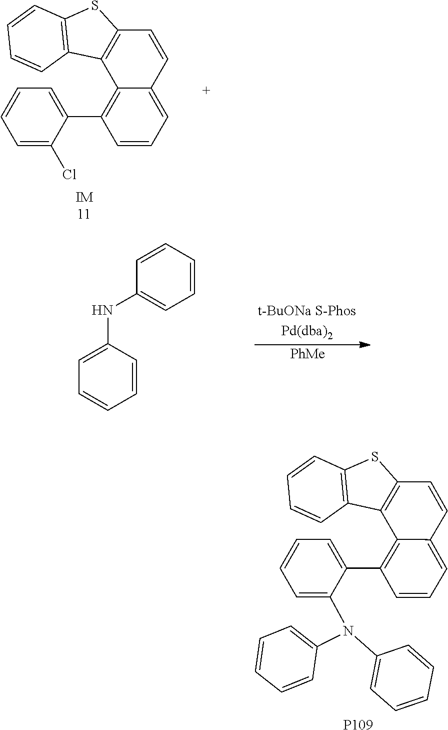 [SMILES: Clc1ccccc1-c1cccc2ccc3sc4ccccc4c3c12 ; c1ccc(Nc2ccccc2)cc1 ; "PhMe" ; "P109" ; c1ccc(N(c2ccccc2)c2ccccc2-c2cccc3ccc4sc5ccccc5c4c23)cc1]

Under the protection of N₂, IM 11 (5.17 g, 15 mmol), DPA (2.6 g, 15 mmol), and toluene (50 mL) were added to a 100 mL three-necked flask, a resulting mixture was heated to reflux at 108° C. and stirred until a clear solution was obtained, and then the clear solution was cooled to 70° C. to 80° C.; sodium tert-butoxide (2.2 g, 22.8 mmol), 2-dicyclohexylphosphino-2′,6′-dimethoxybiphenyl (0.12 g, 0.3 mmol), and bis(dibenzylideneacetone)palladium (0.14 g, 0.15 mmol) were added, a resulting mixture was heated to reflux at 108° C. for 3 h, and then the heating was stopped; and a resulting reaction system was cooled to room temperature, washed with water until neutral, dried with anhydrous magnesium sulfate, and filtered; and a resulting organic phase was concentrated, and a concentrate was passed by a silica gel column with DCM:n-heptane (v/v)=1:3 to obtain a white solid compound P109 (4 g, yield: 56%, and MS: (m/z)=478.16 [M+H]⁺).

The compounds listed in Table 5 were each synthesized with reference to the synthesis method of the compound P109, except that a raw material 7 was used instead of the IM 8 and a raw material 8 was used instead of the DPA. The main raw materials used and the synthesized compounds and yields and MS data thereof were shown in Table 5.

TABLE 5

| | | | | MS (m/z)/ |
|---|---|---|---|---|
| Raw material 7 | Raw material 8 | Compound | Yield/% | [M + H]⁺ |
| 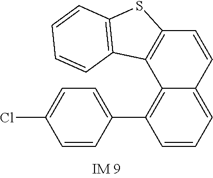 [SMILES: Clc1ccc(-c2cccc3ccc4sc5ccccc5c4c23)cc1] | 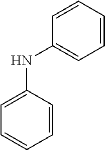 [SMILES: c1ccc(Nc2ccccc2)cc1] | 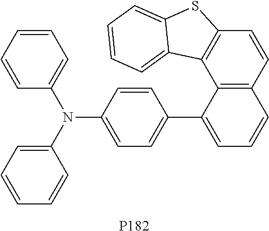 [SMILES: "P182" ; c1ccc(N(c2ccccc2)c2ccc(-c3cccc4ccc5sc6ccccc6c5c34)cc2)cc1] | 80 | 478.16 |
| | 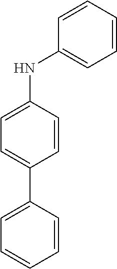 [SMILES: c1ccc(Nc2ccc(-c3ccccc3)cc2)cc1] | 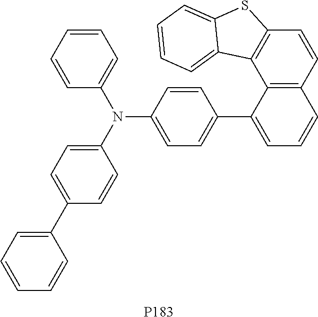 [SMILES: "P183" ; c1ccc(-c2ccc(N(c3ccccc3)c3ccc(-c4cccc5ccc6sc7ccccc7c6c45)cc3)cc2)cc1] | 79 | 554.19 |
| | 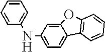 [SMILES: c1ccc(Nc2ccc3c(c2)oc2ccccc23)cc1] | 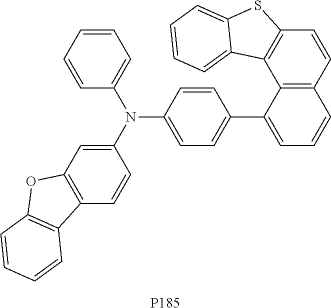 [SMILES: "P185" ; c1ccc(N(c2ccc(-c3cccc4ccc5sc6ccccc6c5c34)cc2)c2ccc3c(c2)oc2ccccc23)cc1] | 82 | 568.17 |
| | 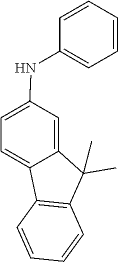 [SMILES: CC1(C)c2ccccc2-c2ccc(Nc3ccccc3)cc21] | 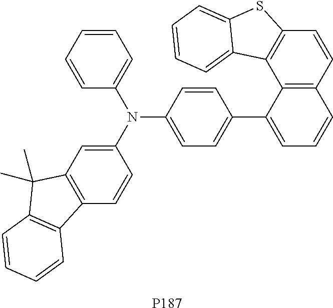 [SMILES: "P187" ; CC1(C)c2ccccc2-c2ccc(N(c3ccccc3)c3ccc(-c4cccc5ccc6sc7ccccc7c6c45)cc3)cc21] | 84 | 594.22 |
| | 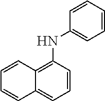 [SMILES: c1ccc(Nc2cccc3ccccc23)cc1] | 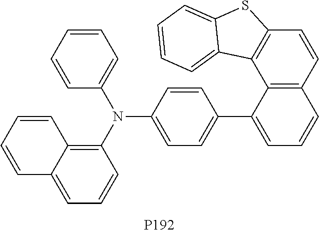 [SMILES: "P192" ; c1ccc(N(c2ccc(-c3cccc4ccc5sc6ccccc6c5c34)cc2)c2cccc3ccccc23)cc1] | 76 | 527.17 |
| | 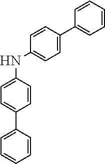 [SMILES: c1ccc(-c2ccc(Nc3ccc(-c4ccccc4)cc3)cc2)cc1] | 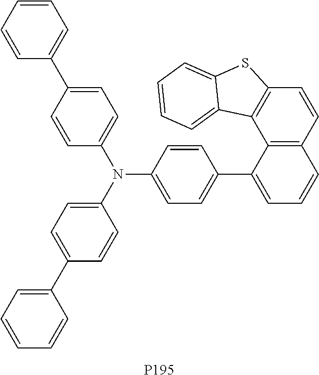 [SMILES: "P195" ; c1ccc(-c2ccc(N(c3ccc(-c4ccccc4)cc3)c3ccc(-c4cccc5ccc6sc7ccccc7c6c45)cc3)cc2)cc1] | 77 | 630.22 |
| | 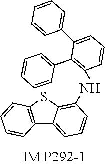 [SMILES: c1ccc(-c2cccc(Nc3cccc4c3sc3ccccc34)c2-c2ccccc2)cc1] | 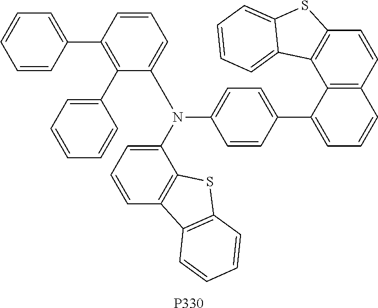 | 68 | 736.21 |
| | 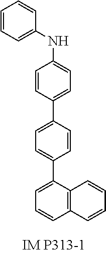 [SMILES: c1ccc(Nc2ccc(-c3ccc(-c4cccc5ccccc45)cc3)cc2)cc1] | 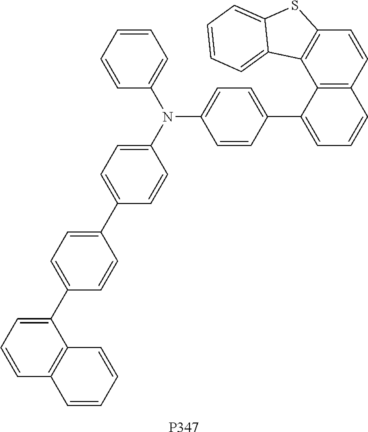 [SMILES: c1ccc(N(c2ccc(-c3ccc(-c4cccc5ccccc45)cc3)cc2)c2ccc(-c3cccc4ccc5sc6ccccc6c5c34)cc2)cc1 ; "P347"] | 82 | 680.23 |
| | 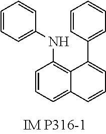 [SMILES: c1ccc(Nc2cccc3cccc(-c4ccccc4)c23)cc1] | 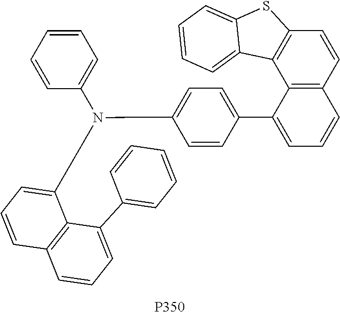 [SMILES: c1ccc(-c2cccc3cccc(N(c4ccccc4)c4ccc(-c5cccc6ccc7sc8ccccc8c7c56)cc4)c23)cc1 ; "P350"] | 85 | 604.20 |
| 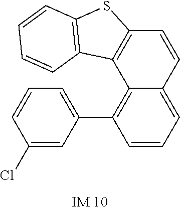 [SMILES: Clc1cccc(-c2cccc3ccc4sc5ccccc5c4c23)c1] | 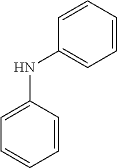 [SMILES: c1ccc(Nc2ccccc2)cc1] | 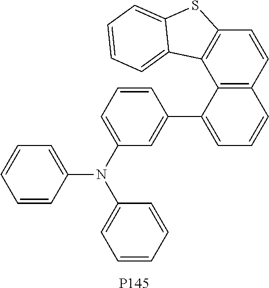 [SMILES: "P145" ; c1ccc(N(c2ccccc2)c2cccc(-c3cccc4ccc5sc6ccccc6c5c34)c2)cc1] | 76 | 478.16 |
| 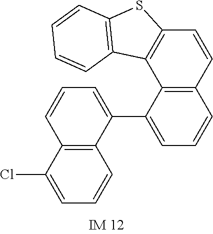 [SMILES: Clc1cccc2c(-c3cccc4ccc5sc6ccccc6c5c34)cccc12] | 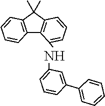 [SMILES: CC1(C)c2ccccc2-c2c(Nc3cccc(-c4ccccc4)c3)cccc21] | 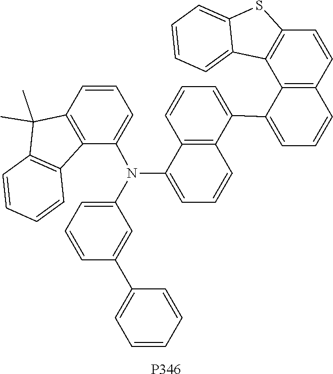 | 72 | 720.26 |

### 8. Synthesis of a Compound P2

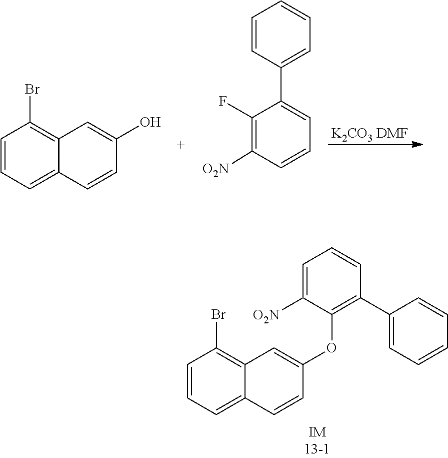

Under the protection of N₂, 1-hydroxy-7-bromonaphthalene (40 g, 179 mmol), 2-fluoro-3-nitrobiphenyl (32.5 g, 149 mmol), potassium carbonate (41 g, 299 mmol), and DMF (320 mL) were added to a 500 mL three-necked flask, a resulting mixture was heated to reflux at 130° C. and stirred for 7 h, then the heating was stopped, and 500 mL of ethyl acetate was added; a resulting system was washed with water until neutral, dried with anhydrous magnesium sulfate, and filtered; a resulting organic phase was concentrated, and a concentrate was passed by a silica gel column with PE:ethyl acetate (v/v)=20:1 to obtain a pale yellow solid IM 13-1 (46.5 g, yield: 75%).

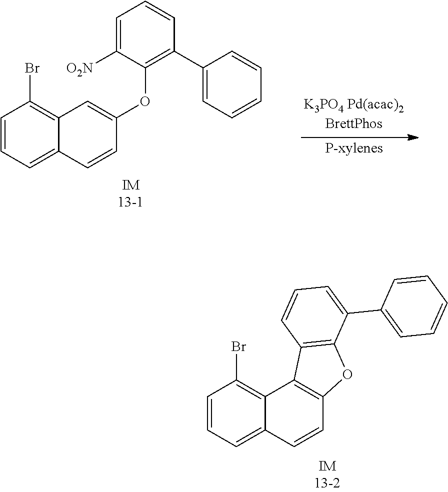 [SMILES: O=[N+]([O-])c1cccc(-c2ccccc2)c1Oc1ccc2cccc(Br)c2c1 ; Brc1cccc2ccc3oc4c(-c5ccccc5)cccc4c3c12]

Under the protection of N₂, the IM 13-1 (40 g, 95 mmol), potassium phosphate (60 g, 285 mmol), BrettPhos (0.51 g, 0.95 mmol), palladium 2,4-glutarate (0.15 g, 0.48 mmol), and xylene (320 mL) were added to a 500 mL three-necked flask, a resulting mixture was heated to 150° C. to 160° C. for 24 h, and the heating was stopped; a resulting system was cooled to room temperature, washed with water until neutral, dried with anhydrous magnesium sulfate, and filtered; and a resulting organic phase was concentrated, and a concentrate was passed by a silica gel column with PE:ethyl acetate (v/v)=10:1 to obtain a white solid IM 13-2 (30 g, yield: 85%).

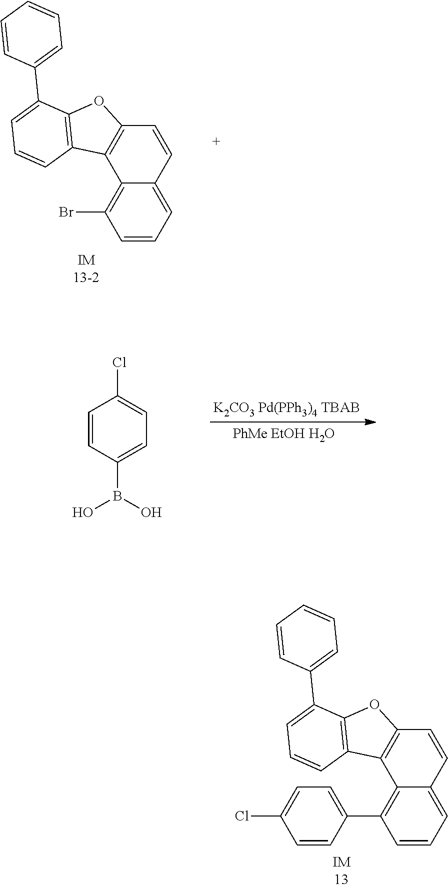 [SMILES: Brc1cccc2ccc3oc4c(-c5ccccc5)cccc4c3c12 ; OB(O)c1ccc(Cl)cc1 ; Clc1ccc(-c2cccc3ccc4oc5c(-c6ccccc6)cccc5c4c23)cc1]

Under the protection of N₂, the IM 13-2 (30 g, 80 mmol), 4-chlorophenylboronic acid (12.6 g, 80 mmol), potassium carbonate (22 g, 160 mmol), tetrakis(triphenylphosphine)palladium (0.93 g, 0.8 mmol), TBAB (0.51 g, 1.6 mmol), toluene (180 mL), absolute ethanol (90 mL), and water (30 mL) were added to a 500 mL three-necked flask, and a resulting mixture was heated to reflux at 80° C. and stirred for 24 h; a resulting reaction system was cooled to room temperature, washed with water until neutral, dried with anhydrous magnesium sulfate, and filtered; and a resulting organic phase was concentrated, and a concentrate was passed by a silica gel column with DCM:n-heptane (v/v)=1:4 to obtain a white solid IM 13 (25 g, yield: 79%).

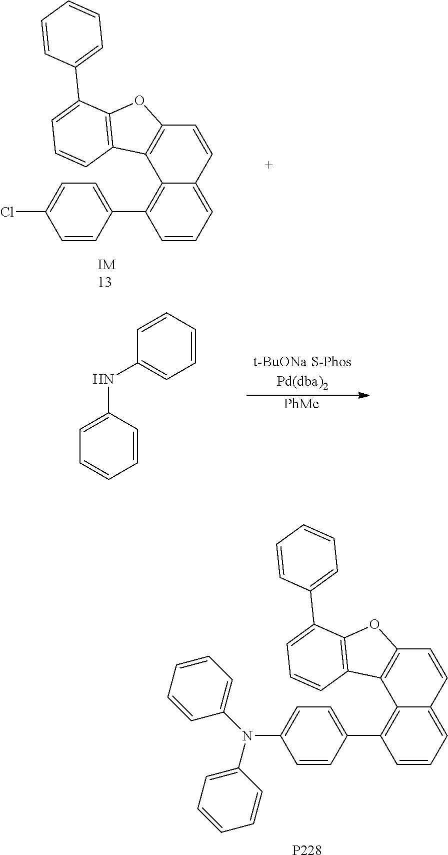 [SMILES: Clc1ccc(-c2cccc3ccc4oc5c(-c6ccccc6)cccc5c4c23)cc1 ; "P228" ; c1ccc(Nc2ccccc2)cc1 ; c1ccc(-c2cccc3c2oc2ccc4cccc(-c5ccc(N(c6ccccc6)c6ccccc6)cc5)c4c23)cc1 ; "PhMe"]

Under the protection of N₂, IM 13 (10 g, 24.7 mmol), DPA (4.2 g, 24.7 mmol), and toluene (80 mL) were added to a 250 mL three-necked flask, a resulting mixture was heated to reflux at 108° C. and stirred until a clear solution was obtained, and then the clear solution was cooled to 70° C. to 80° C.; sodium tert-butoxide (3.6 g, 37 mmol), 2-dicyclohexylphosphino-2′,6′-dimethoxybiphenyl (0.20 g, 0.49 mmol), and bis(dibenzylideneacetone)palladium (0.23 g, 0.25 mmol) were added, a resulting mixture was heated to reflux at 108° C. for 4 h, and then the heating was stopped; and a resulting reaction system was cooled to room temperature, washed with water until neutral, dried with anhydrous magnesium sulfate, and filtered; and a resulting organic phase was concentrated, and a concentrate was passed by a silica gel column with DCM:n-heptane (v/v)=1:4 to obtain a white solid compound P228 (7.2 g, yield: 54%, and MS: (m/z)=538.21 [M+H]⁺).

Nuclear magnetic resonance (NMR) data of some compounds were shown in Table 6 below:

TABLE 6

| Compound | NMR data |
|---|---|
| P73 | ¹H-NMR(CD₂Cl₂, 400 MHz) δ(ppm): 8.56(d, 1H), 8.26(d, 1H), 8.02(d, 1H), |
| | 7.89-7.83(m, 4H), 7.74-7.79(m 2H), 7.55(t, 4H), 7.48(d, 2H), 7.16(d, 2H), |
| | 7.09(t, 2H), 6.94(d, 4H). |
| P86 | ¹H-NMR(CD₂Cl₂, 400 MHz) δ(ppm): 8.56(d, 1H), 8.36(d, 1H), 8.12(d, 1H), |
| | 7.89-7.80(m, 12H), 7.72-7.70(m, 8H), 7.48(d, 2H), 7.16(d, 2H), 7.06(d, 4H). |
| P182 | ¹H-NMR(CD₂Cl₂, 400 MHz) δ(ppm): 8.57(d, 1H), 8.27(d, 1H), 8.01 (d, 1H), |
| | 7.90-7.81 (m, 4H), 7.73-7.78(m, 2H), 7.54(t, 4H), 7.49(d, 2H), 7.14(d, 2H), |
| | 7.010(t, 2H), 6.96(d, 4H). |

### Fabrication and Evaluation of OLEDs

### Example 1

Blue Light-Emitting OLED

An anode was produced by the following process: An ITO substrate with a thickness of 1,500 Å (manufactured by Corning) was cut into a size of 40 mm×40 mm×0.7 mm, then the substrate was processed through photolithography into an experimental substrate with cathode, anode, and insulating layer patterns, and the experimental substrate was subjected to a surface treatment with ultraviolet (UV)-ozone and O₂:N₂ plasma to increase a work function of the anode (experimental substrate) and remove scums.

F4-TCNQ was vacuum-deposited on the experimental substrate (anode) to form an HIL with a thickness of 100 Å. The compound P1 was deposited on the HIL to form an HTL with a thickness of 1,230 Å.

EB-01 was vacuum-deposited on the HTL to form an EBL with a thickness of 100 Å.

BH-01 and BD-01 were co-deposited on the EBL in a ratio of 98%:2% to form an organic blue light-emitting layer (EML) with a thickness of 220 Å.

ET-06 and LiQ were deposited on the organic blue light-emitting layer (EML) in a film thickness ratio of 1:1 to form an ETL with a thickness of 350 Å, ytterbium (Yb) was deposited on the ETL to form an EIL with a thickness of 10 Å, and then magnesium (Mg) and argentum (Ag) were vacuum-deposited on the EIL in a film thickness ratio of 1:10 to form a cathode with a thickness of 140 Å.

In addition, CP-5 was deposited on the cathode to form an organic capping layer (CPL) with a thickness of 650 Å, thereby completing the fabrication of the OLED.

### Examples 2 to 42

OLEDs were each fabricated by the same method as in Example 1, except that the compounds shown in Table 8 below were each used instead of the compound P1 in the formation of the HTL.

### Comparative Examples 1 to 3

OLEDs were each fabricated by the same method as in Example 1, except that compounds A, B, and C were each used instead of the compound P1 in the formation of the HTL.

Structures of the main materials used in the above examples and comparative examples were shown in Table 7 below:

TABLE 7

| | |
|---|---|
| 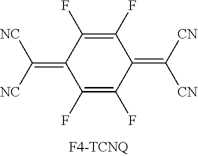 | 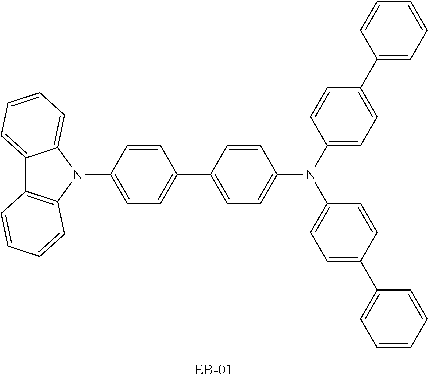 [SMILES: c1ccc(-c2ccc(N(c3ccc(-c4ccccc4)cc3)c3ccc(-c4ccc(-n5c6ccccc6c6ccccc65)cc4)cc3)cc2)cc1] |
| 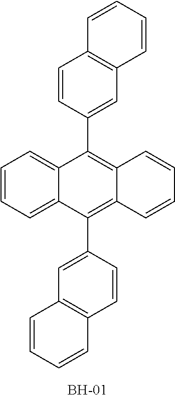 | 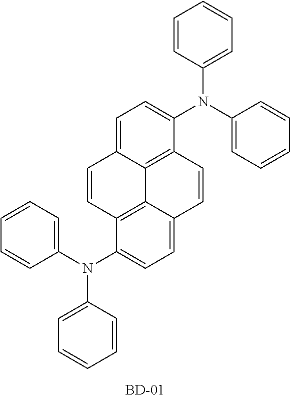 [SMILES: c1ccc(N(c2ccccc2)c2ccc3ccc4c(N(c5ccccc5)c5ccccc5)ccc5ccc2c3c54)cc1] |
| 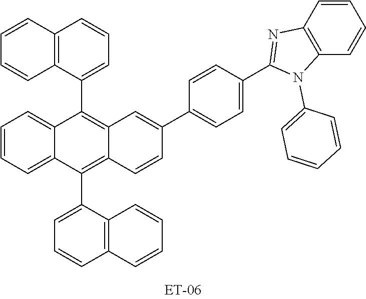 | 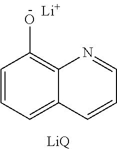 |
| 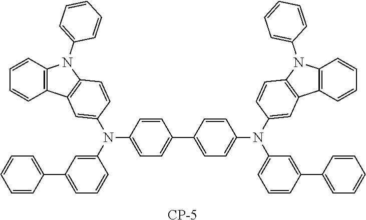 | 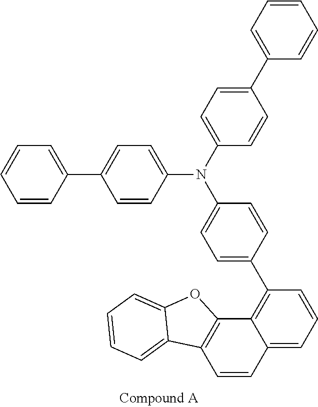 [SMILES: c1ccc(-c2ccc(N(c3ccc(-c4ccccc4)cc3)c3ccc(-c4cccc5ccc6c7ccccc7oc6c45)cc3)cc2)cc1] |
| 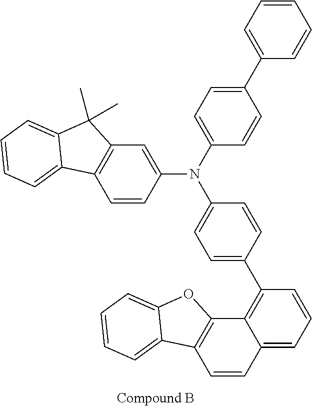 | 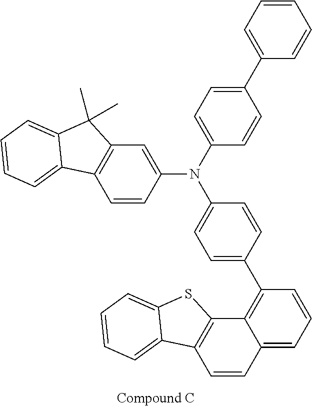 [SMILES: CC1(C)c2ccccc2-c2ccc(N(c3ccc(-c4ccccc4)cc3)c3ccc(-c4cccc5ccc6c7ccccc7sc6c45)cc3)cc21] |

The OLEDs fabricated above were subjected to performance analysis at 15 mA/cm², and results were shown in Table 8 below:

TABLE 8

| | | | | | | External | T95 |
|---|---|---|---|---|---|---|---|
| | | | Light-emitting | Power | Chromaticity | quantum | life |
| | | Driving | efficiency | efficiency | coordinate | efficiency | span |
| Example | HTL | voltage (V) | (Cd/A) | (lm/W) | CIE-x, CIE-y | (EQE, %) | (h) |
| Example 1 | Compound P1 | 3.94 | 6.59 | 5.25 | 0.14, 0.05 | 13.56 | 317 |
| Example 2 | Compound P14 | 3.86 | 6.72 | 5.47 | 0.14, 0.05 | 13.82 | 263 |
| Example 3 | Compound P37 | 3.92 | 6.55 | 5.25 | 0.14, 0.05 | 13.47 | 328 |
| Example 4 | Compound P50 | 3.82 | 6.50 | 5.35 | 0.14, 0.05 | 13.37 | 272 |
| Example 5 | Compound P73 | 3.85 | 6.51 | 5.31 | 0.14, 0.05 | 13.39 | 250 |
| Example 6 | Compound P74 | 3.81 | 6.53 | 5.38 | 0.14, 0.05 | 13.43 | 293 |
| Example 7 | Compound P76 | 3.87 | 6.76 | 5.49 | 0.14, 0.05 | 13.91 | 309 |
| Example 8 | Compound P78 | 3.83 | 6.60 | 5.41 | 0.14, 0.05 | 13.58 | 324 |
| Example 9 | Compound P83 | 3.81 | 6.61 | 5.45 | 0.14, 0.05 | 13.60 | 311 |
| Example 10 | Compound P86 | 3.90 | 6.50 | 5.24 | 0.14, 0.05 | 13.37 | 250 |
| Example 11 | Compound P90 | 3.91 | 6.73 | 5.41 | 0.14, 0.05 | 13.84 | 291 |
| Example 12 | Compound P93 | 3.82 | 6.73 | 5.53 | 0.14, 0.05 | 13.84 | 279 |
| Example 13 | Compound P264 | 3.89 | 6.69 | 5.40 | 0.14, 0.05 | 13.76 | 320 |
| Example 14 | Compound P265 | 3.92 | 6.66 | 5.34 | 0.14, 0.05 | 13.70 | 320 |
| Example 15 | Compound P109 | 3.82 | 6.64 | 5.46 | 0.14, 0.05 | 13.66 | 259 |
| Example 16 | Compound P145 | 3.92 | 6.68 | 5.35 | 0.14, 0.05 | 13.74 | 251 |
| Example 17 | Compound P182 | 3.95 | 6.77 | 5.38 | 0.14, 0.05 | 13.93 | 255 |
| Example 18 | Compound P183 | 3.82 | 6.59 | 5.42 | 0.14, 0.05 | 13.56 | 256 |
| Example 19 | Compound P185 | 3.94 | 6.64 | 5.29 | 0.14, 0.05 | 13.66 | 281 |
| Example 20 | Compound P187 | 3.94 | 6.78 | 5.41 | 0.14, 0.05 | 13.95 | 300 |
| Example 21 | Compound P192 | 3.94 | 6.76 | 5.39 | 0.14, 0.05 | 13.91 | 319 |
| Example 22 | Compound P195 | 3.86 | 6.65 | 5.41 | 0.14, 0.05 | 13.68 | 329 |
| Example 23 | Compound P228 | 3.83 | 6.69 | 5.49 | 0.14, 0.05 | 13.76 | 316 |
| Example 24 | Compound P259 | 3.89 | 6.51 | 5.23 | 0.14, 0.05 | 13.35 | 295 |
| Example 25 | Compound P279 | 3.91 | 6.53 | 5.26 | 0.14, 0.05 | 13.43 | 308 |
| Example 26 | Compound P282 | 3.92 | 6.68 | 5.35 | 0.14, 0.05 | 13.74 | 294 |
| Example 27 | Compound P283 | 3.81 | 6.57 | 5.42 | 0.14, 0.05 | 13.51 | 267 |
| Example 28 | Compound P284 | 3.82 | 6.53 | 5.37 | 0.14, 0.05 | 13.43 | 279 |
| Example 29 | Compound P287 | 3.82 | 6.78 | 5.58 | 0.14, 0.05 | 13.95 | 312 |
| Example 30 | Compound P289 | 3.85 | 6.77 | 5.52 | 0.14, 0.05 | 13.93 | 274 |
| Example 31 | Compound P292 | 3.93 | 6.80 | 5.44 | 0.14, 0.05 | 13.99 | 303 |
| Example 32 | Compound P295 | 3.91 | 6.77 | 5.44 | 0.14, 0.05 | 13.93 | 265 |
| Example 33 | Compound P310 | 3.93 | 6.71 | 5.36 | 0.14, 0.05 | 13.80 | 295 |
| Example 34 | Compound P312 | 3.85 | 6.76 | 5.52 | 0.14, 0.05 | 13.91 | 256 |
| Example 35 | Compound P313 | 3.87 | 6.54 | 5.31 | 0.14, 0.05 | 13.45 | 263 |
| Example 36 | Compound P314 | 3.81 | 6.52 | 5.38 | 0.14, 0.05 | 13.41 | 292 |
| Example 37 | Compound P316 | 3.92 | 6.59 | 5.28 | 0.14, 0.05 | 13.56 | 311 |
| Example 38 | Compound P317 | 3.93 | 6.53 | 5.22 | 0.14, 0.05 | 13.43 | 328 |
| Example 39 | Compound P330 | 3.92 | 6.53 | 5.23 | 0.14, 0.05 | 13.43 | 314 |
| Example 40 | Compound P346 | 3.83 | 6.61 | 5.42 | 0.14, 0.05 | 13.60 | 251 |
| Example 41 | Compound P347 | 3 92 | 6.54 | 5.24 | 0.14, 0.05 | 13.45 | 324 |
| Example 42 | Compound P350 | 3.83 | 6.61 | 5.42 | 0.14, 0.05 | 13.60 | 289 |
| Comparative | Compound A | 4.13 | 5.45 | 4.15 | 0.14, 0.05 | 11.21 | 202 |
| Example 1 | | | | | | | |
| Comparative | Compound B | 4.06 | 5.65 | 4.37 | 0.14, 0.05 | 11.62 | 209 |
| Example 2 | | | | | | | |
| Comparative | Compound C | 4.08 | 5.41 | 4.17 | 0.14, 0.05 | 11.13 | 192 |
| Example 3 | | | | | | | |

According to the results in Table 8, compared with the OLEDs fabricated with a known compound as an HTL (Comparative Examples 1 to 3), the OLEDs fabricated with the nitrogen-containing compound of the present application as an HTL (Examples 1 to 42) have a driving voltage reduced by at least 0.11 V, a light-emitting efficiency (Cd/A) increased by at least 15.04%, an EQE increased by at least 14.89%, and a life span increased by at least 19.62% (the highest life span can be increased by 137 h). Therefore, when used in an HTL, the nitrogen-containing compound of the present application can improve the light-emitting efficiency and service life of the OLED.

Preferred embodiments of the present application are described above in detail with reference to the accompanying drawings, but the present application is not limited to specific details in the above embodiments. Various simple variations can be made to the technical solutions of the present application without departing from the technical ideas of the present application, and these simple variations fall within the protection scope of the present application. In addition, it should be noted that the various specific technical features described in the above-mentioned specific embodiments can be combined in any suitable manner unless they conflict with each other. In order to avoid unnecessary repetition, various additional possible combinations are not described in the present application. In addition, various different embodiments of the present application can also be combined arbitrarily, as long as the spirit of the present application is not violated, which should also be regarded as a content disclosed in the present application.

1.-17. (canceled)

18. A nitrogen-containing compound with a structure shown in formula 1: 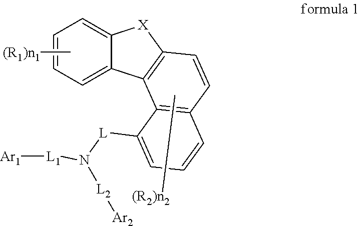
wherein X is selected from O and S;
R₁ and R₂ are the same or different, and are each independently selected from the group consisting of deuterium, cyano, fluorine, alkyl with 1 to 5 carbon atoms, trimethylsilyl, phenyl, naphthyl, biphenyl, and pyridyl;
n₁ represents the number of R₁, and n₁ is selected from 0, 1, 2, 3, and 4; and when n₁ is greater than 1, any two R₁ are the same or different;
n₂ represents the number of R₂, and n₂ is selected from 0, 1, 2, 3, 4, and 5; and when n₂ is greater than 1, any two R₂ are the same or different;
L is selected from substituted or unsubstituted arylene with 6 to 30 carbon atoms, and the arylene is phenylene, naphthylene, and biphenylene;
L₁ and L₂ are the same or different, and are each independently selected from the group consisting of a single bond, substituted or unsubstituted arylene with 6 to 12 carbon atoms, and substituted or unsubstituted heteroarylene with 3 to 12 carbon atoms;
Ar₁ and Ar₂ are the same or different, and are each independently selected from the group consisting of substituted or unsubstituted aryl with 6 to 30 carbon atoms and substituted or unsubstituted heteroaryl with 3 to 30 carbon atoms; and
substituents on Ar₁ and Ar₂ are the same or different, and are each independently selected from the group consisting of deuterium, fluorine, cyano, alkyl with 1 to 5 carbon atoms, cycloalkyl with 3 to 6 carbon atoms, aryl with 6 to 12 carbon atoms, heteroaryl with 12 to 18 carbon atoms, and trimethylsilyl;
substituents on L, L₁, and L₂ are each independently selected from the group consisting of deuterium, fluorine, cyano, alkyl with 1 to 5 carbon atoms, and phenyl.

19. The nitrogen-containing compound according to claim 18, wherein L is selected from the group consisting of the following groups: 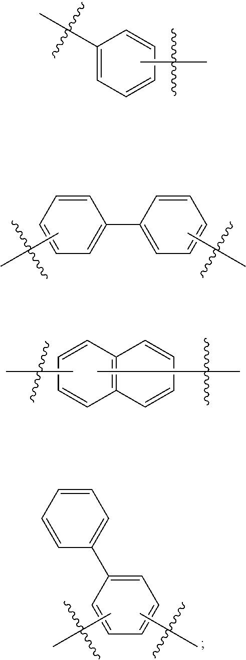
and
L₁ and L₂ are each independently selected from the group consisting of a single bond and the following groups:

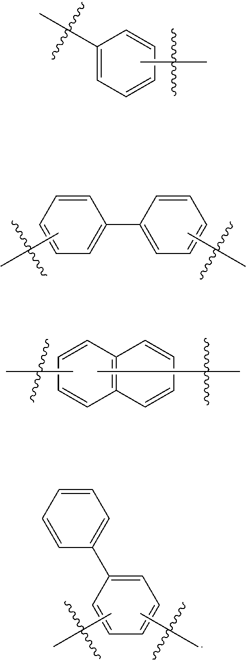

20. The nitrogen-containing compound according to claim 18, wherein Ar₁ and Ar₂ are each independently selected from the group consisting of substituted or unsubstituted aryl with 6 to 25 carbon atoms and substituted or unsubstituted heteroaryl with 12 to 18 carbon atoms.

21. The nitrogen-containing compound according to claim 18, wherein Ar₁ and Ar₂ are each independently a substituted or unsubstituted group W; an unsubstituted group W is selected from the group consisting of the following groups: 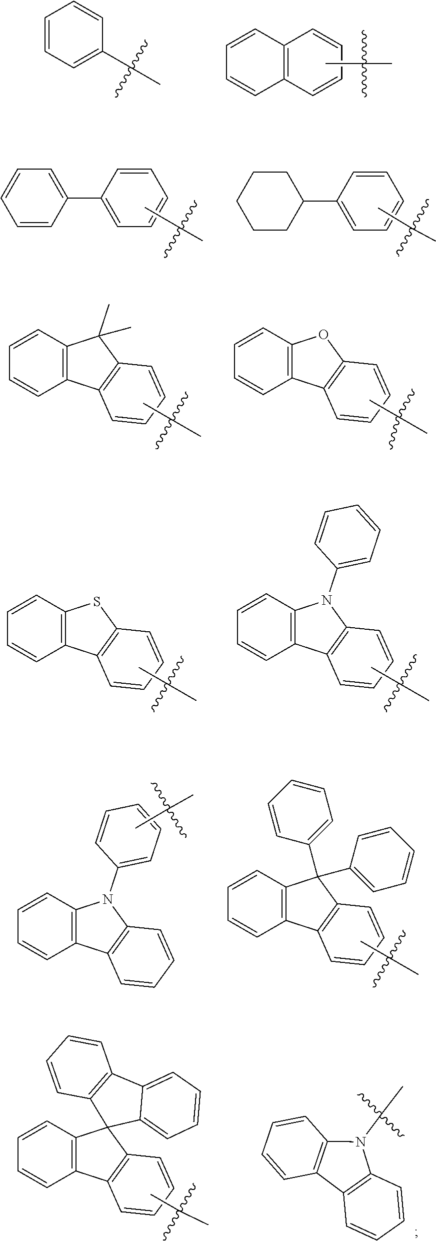
when the group W is substituted, a substituent on the group W is selected from the group consisting of deuterium, fluorine, cyano, trimethylsilyl, methyl, ethyl, isopropyl, tert-butyl, cyclopropyl, cyclohexyl, phenyl, naphthyl, biphenyl, dibenzofuranyl, dibenzothienyl, and carbazolyl; and when there are two or more substituents on the group W, the two or more substituents are the same or different.

22. The nitrogen-containing compound according to claim 18, wherein Ar₁ and Ar₂ are each independently a substituted or unsubstituted group W′; an unsubstituted group W′ is elected from the group consisting of the following groups: 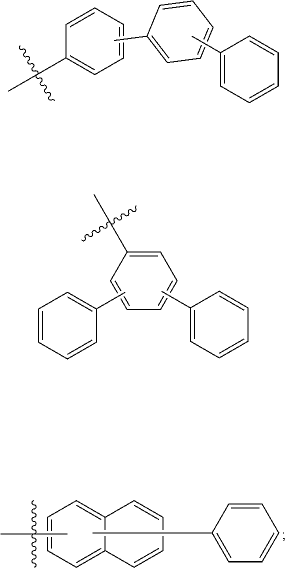
when the group W′ is substituted, a substituent on the group W′ is selected from the group consisting of deuterium, fluorine, cyano, trimethylsilyl, methyl, ethyl, isopropyl, tert-butyl, cyclopropyl, cyclohexyl, phenyl, naphthyl, biphenyl, dibenzofuranyl, dibenzothienyl, and carbazolyl; and when there are two or more substituents on the group W′, the two or more substituents are the same or different.

23. The nitrogen-containing compound according to claim 18, wherein Ar₁ and Ar₂ are each independently selected from the group consisting of the following groups:

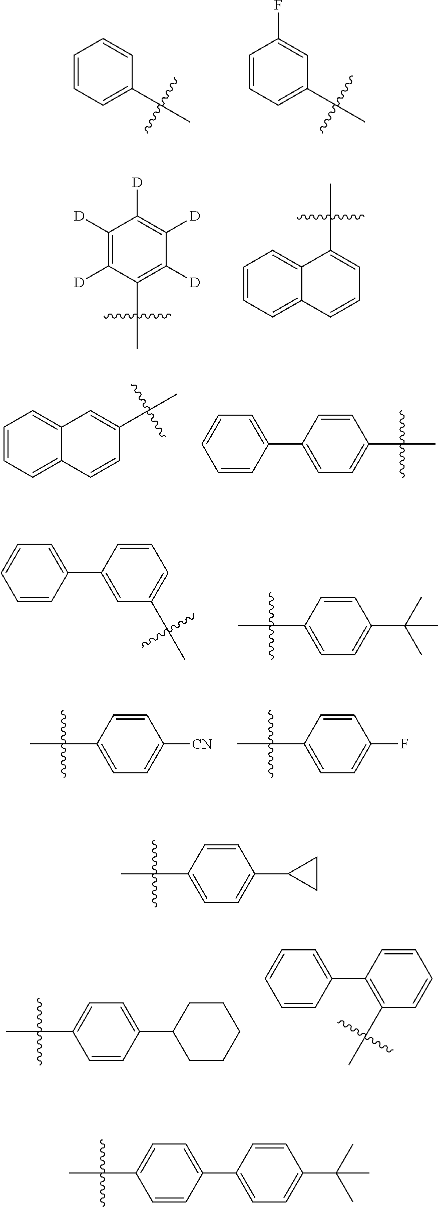

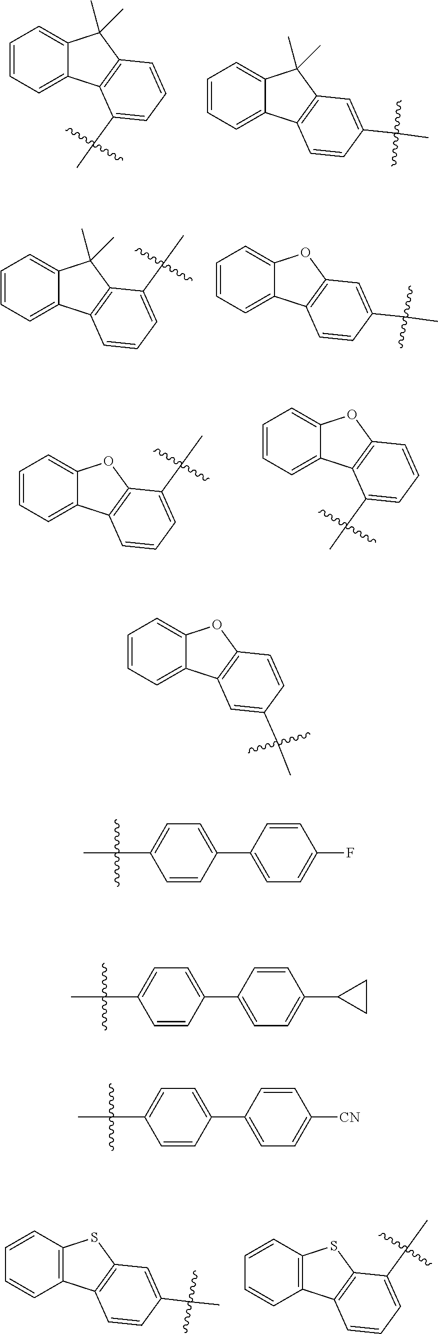

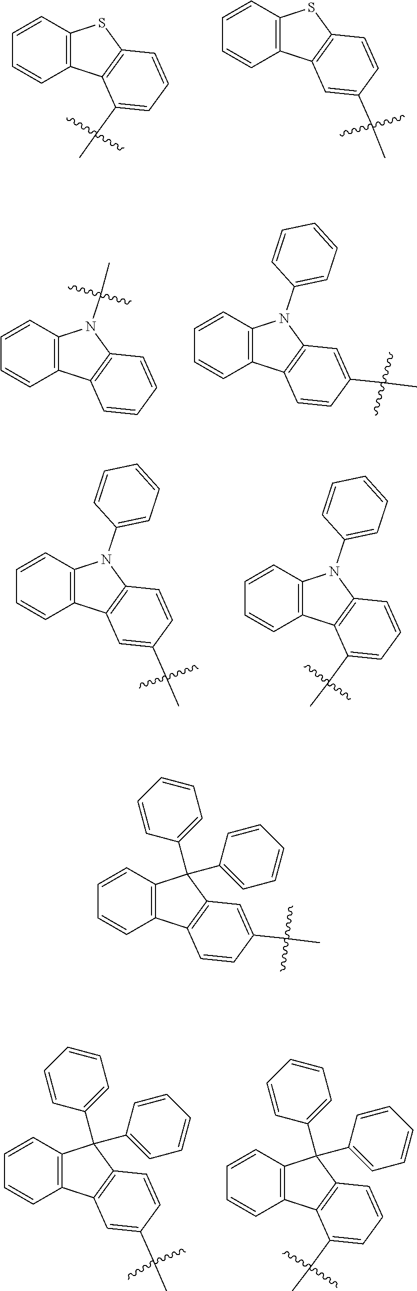

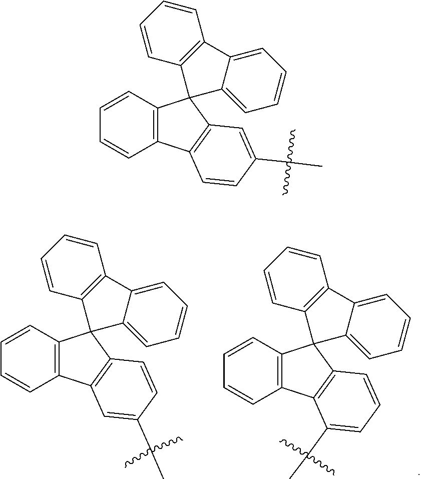 [SMILES: *c1ccc2c(c1)C1(c3ccccc3-c3ccccc31)c1ccccc1-2 ; *c1ccc2c(c1)-c1ccccc1C21c2ccccc2-c2ccccc21 ; *c1cccc2c1-c1ccccc1C21c2ccccc2-c2ccccc21]

24. The nitrogen-containing compound according to claim 18, wherein Ar₁ and Ar₂ are each independently selected from the group consisting of the following groups:

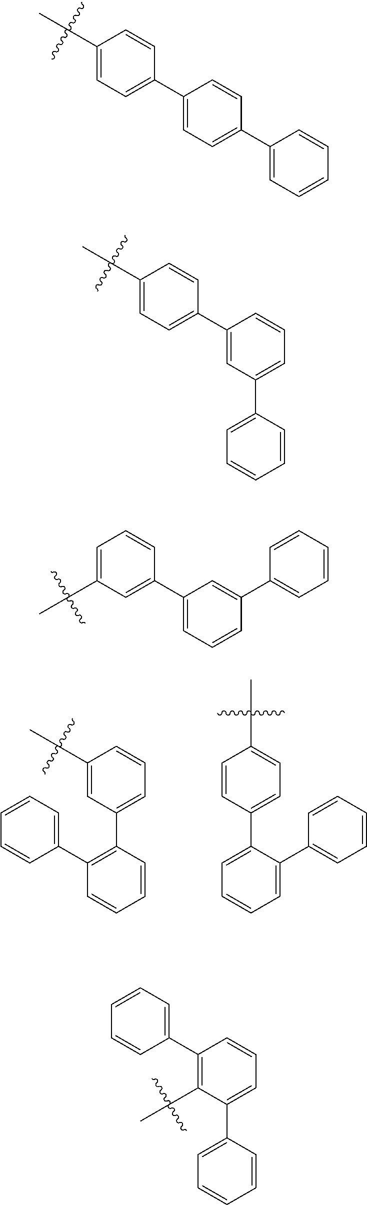 [SMILES: *c1ccc(-c2ccc(-c3ccccc3)cc2)cc1 ; *c1ccc(-c2cccc(-c3ccccc3)c2)cc1 ; *c1cccc(-c2cccc(-c3ccccc3)c2)c1 ; *c1cccc(-c2ccccc2-c2ccccc2)c1 ; *c1ccc(-c2ccccc2-c2ccccc2)cc1 ; *c1c(-c2ccccc2)cccc1-c1ccccc1]

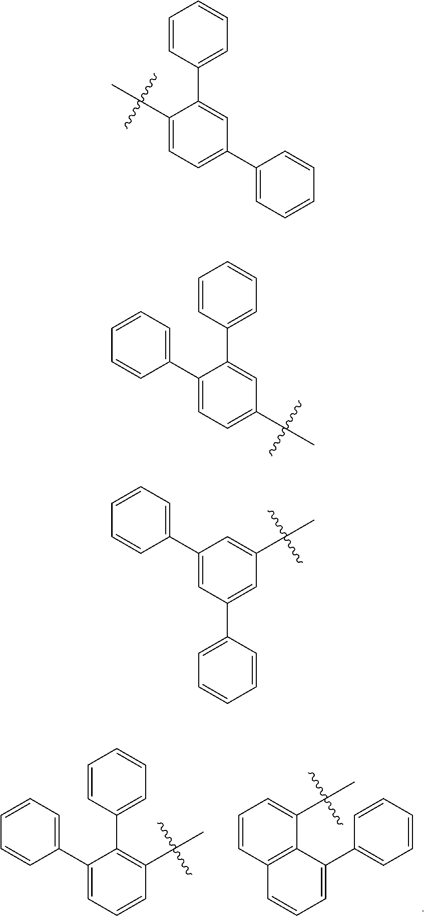 [SMILES: *c1ccc(-c2ccccc2)cc1-c1ccccc1 ; *c1ccc(-c2ccccc2)c(-c2ccccc2)c1 ; *c1cc(-c2ccccc2)cc(-c2ccccc2)c1 ; *c1cccc(-c2ccccc2)c1-c1ccccc1 ; *c1cccc2cccc(-c3ccccc3)c12]

25. The nitrogen-containing compound according to claim 18, wherein the nitrogen-containing compound is selected from the group consisting of the following compounds:

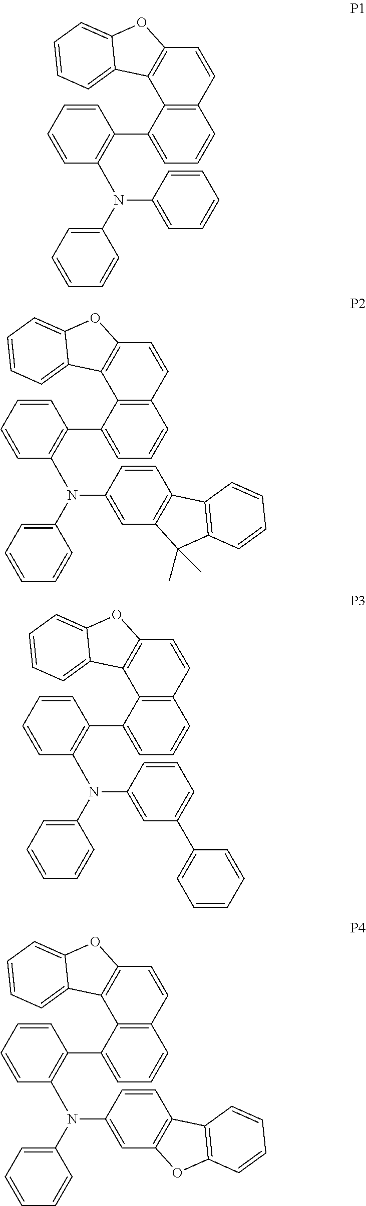

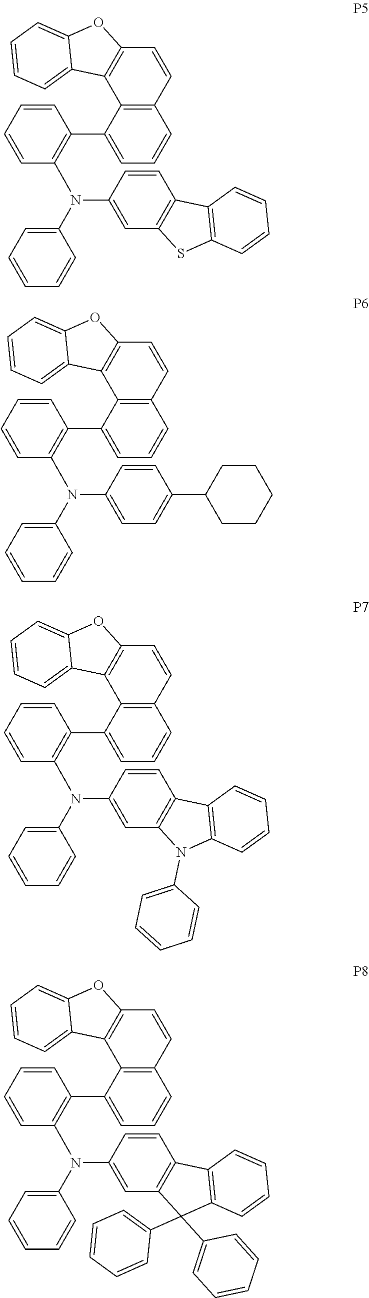

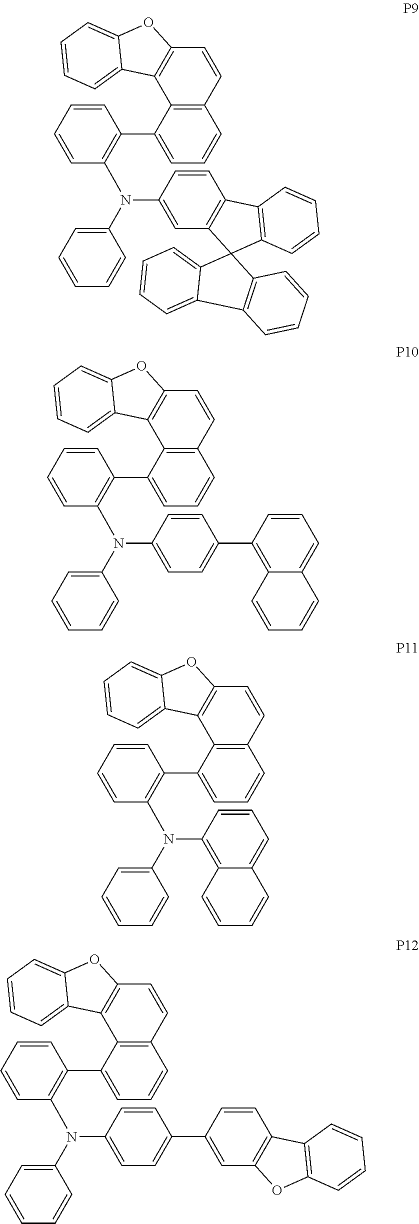

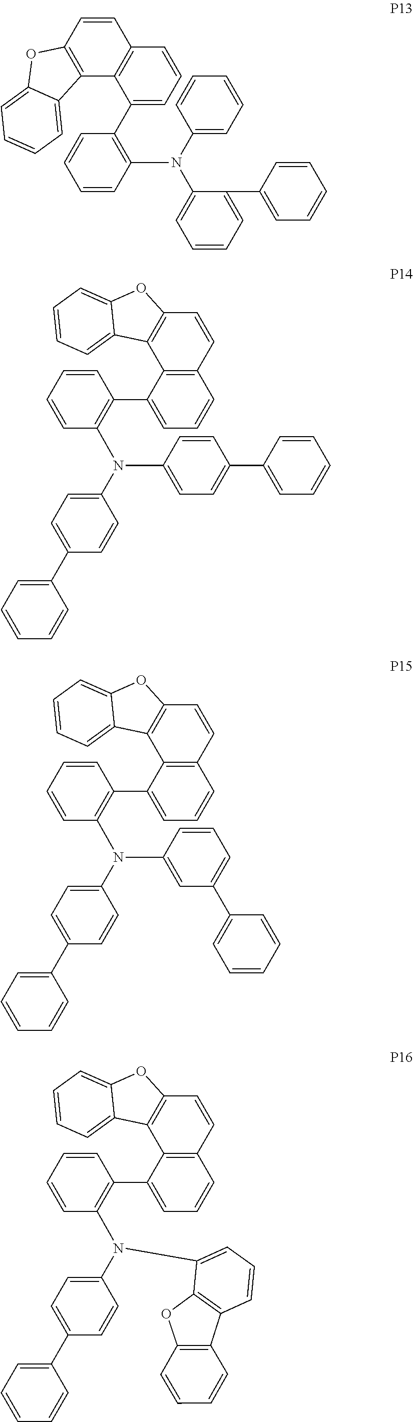 [SMILES: c1ccc(-c2ccccc2N(c2ccccc2)c2ccccc2-c2cccc3ccc4oc5ccccc5c4c23)cc1 ; c1ccc(-c2ccc(N(c3ccc(-c4ccccc4)cc3)c3ccccc3-c3cccc4ccc5oc6ccccc6c5c34)cc2)cc1 ; c1ccc(-c2ccc(N(c3cccc(-c4ccccc4)c3)c3ccccc3-c3cccc4ccc5oc6ccccc6c5c34)cc2)cc1 ; c1ccc(-c2ccc(N(c3ccccc3-c3cccc4ccc5oc6ccccc6c5c34)c3cccc4c3oc3ccccc34)cc2)cc1]

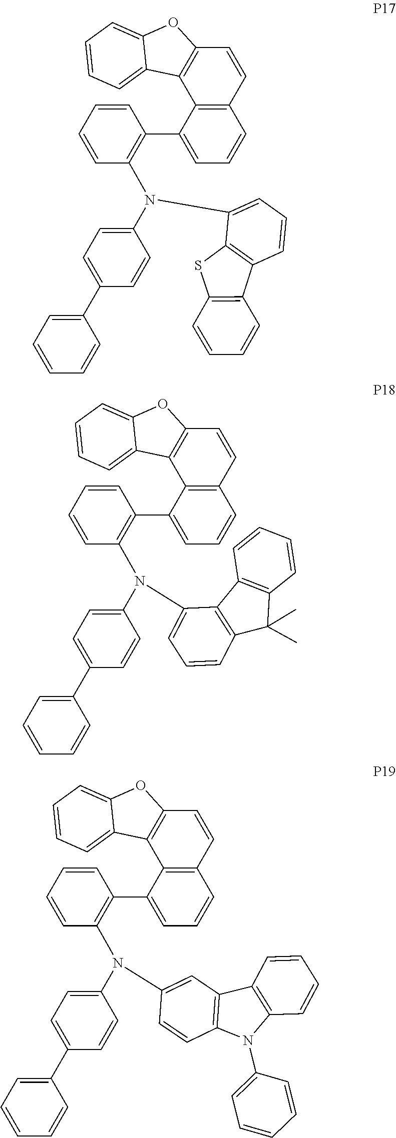

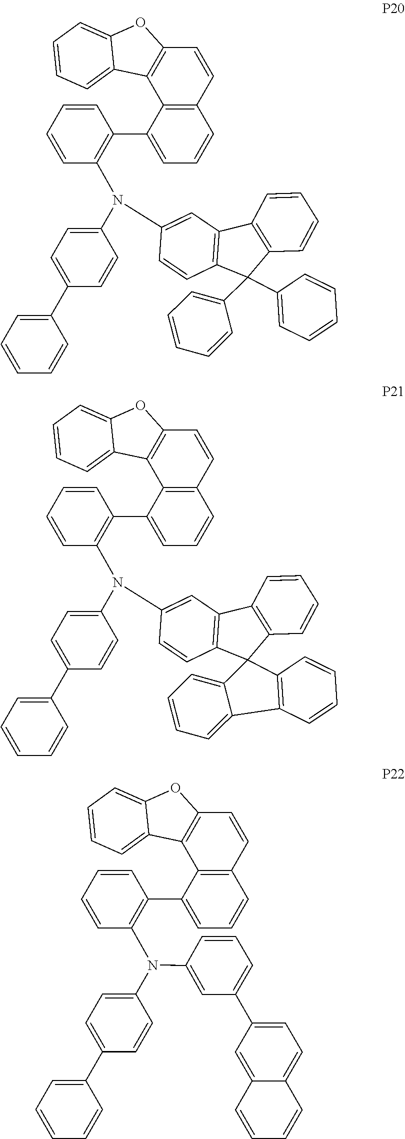

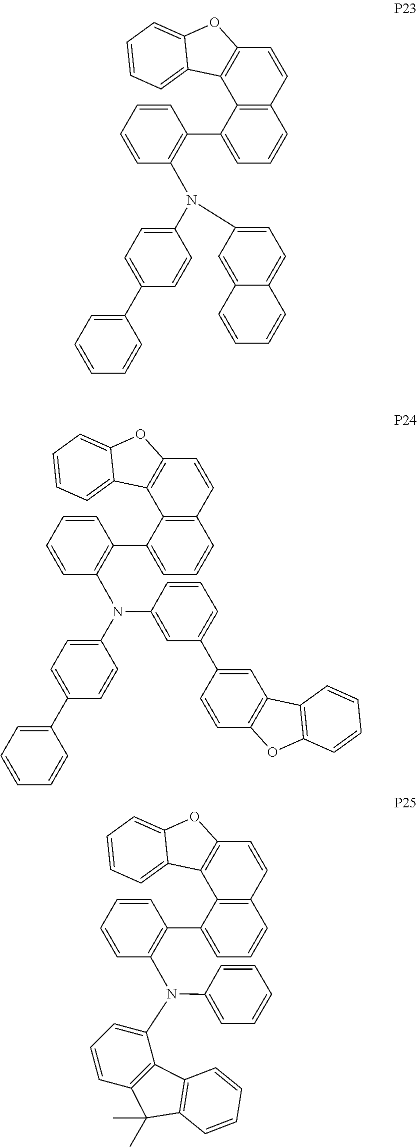 [SMILES: c1ccc(-c2ccc(N(c3ccc4ccccc4c3)c3ccccc3-c3cccc4ccc5oc6ccccc6c5c34)cc2)cc1 ; c1ccc(-c2ccc(N(c3cccc(-c4ccc5oc6ccccc6c5c4)c3)c3ccccc3-c3cccc4ccc5oc6ccccc6c5c34)cc2)cc1 ; CC1(C)c2ccccc2-c2c(N(c3ccccc3)c3ccccc3-c3cccc4ccc5oc6ccccc6c5c34)cccc21]

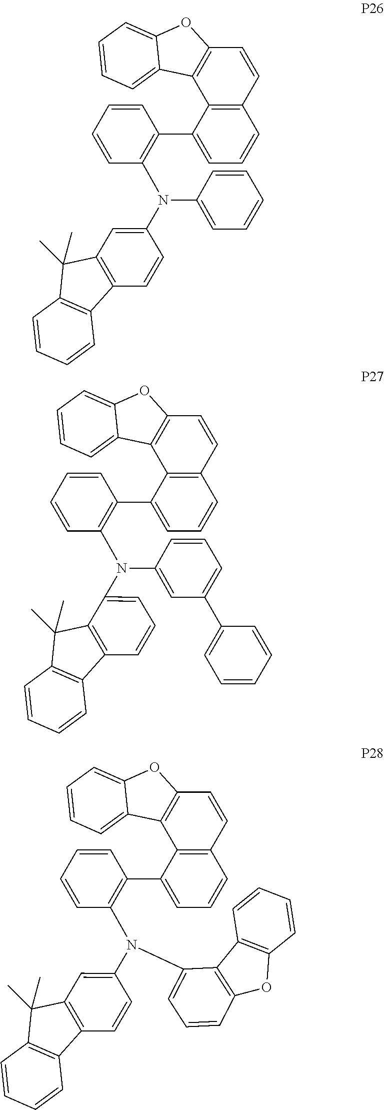

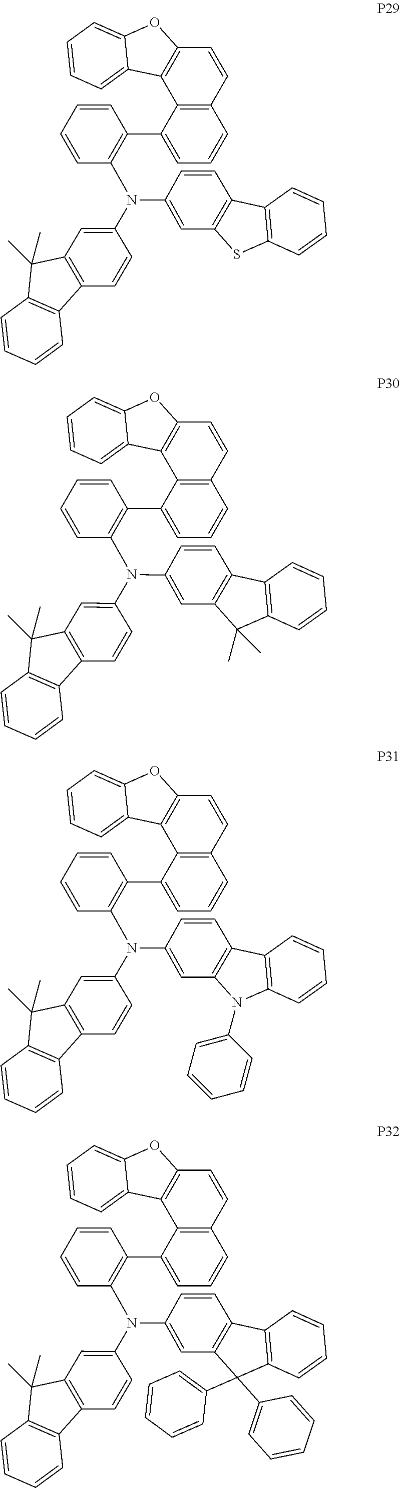 [SMILES: CC1(C)c2ccccc2-c2ccc(N(c3ccc4c(c3)sc3ccccc34)c3ccccc3-c3cccc4ccc5oc6ccccc6c5c34)cc21 ; CC1(C)c2ccccc2-c2ccc(N(c3ccc4c(c3)C(C)(C)c3ccccc3-4)c3ccccc3-c3cccc4ccc5oc6ccccc6c5c34)cc21 ; CC1(C)c2ccccc2-c2ccc(N(c3ccc4c5ccccc5n(-c5ccccc5)c4c3)c3ccccc3-c3cccc4ccc5oc6ccccc6c5c34)cc21 ; CC1(C)c2ccccc2-c2ccc(N(c3ccc4c(c3)C(c3ccccc3)(c3ccccc3)c3ccccc3-4)c3ccccc3-c3cccc4ccc5oc6ccccc6c5c34)cc21]

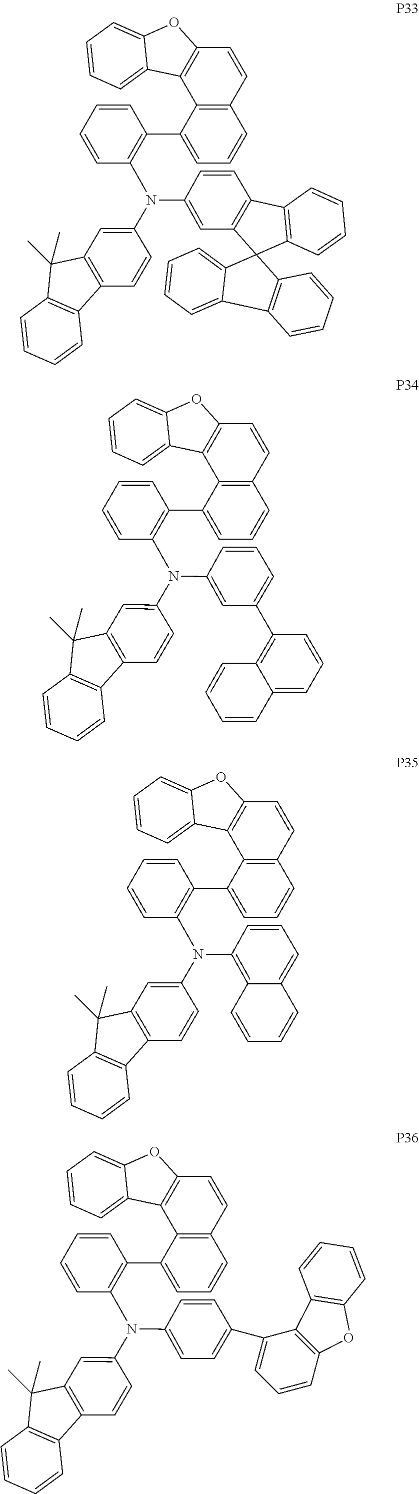

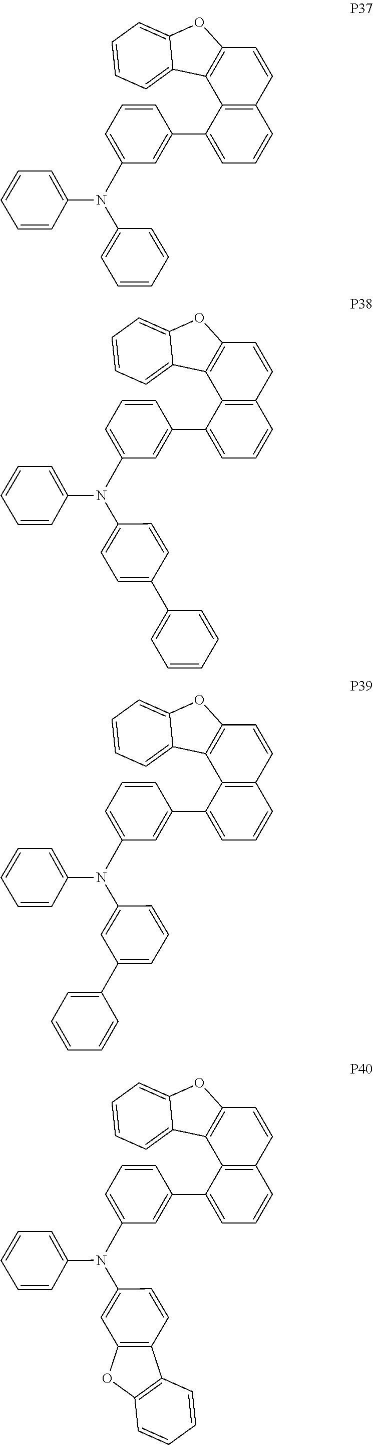

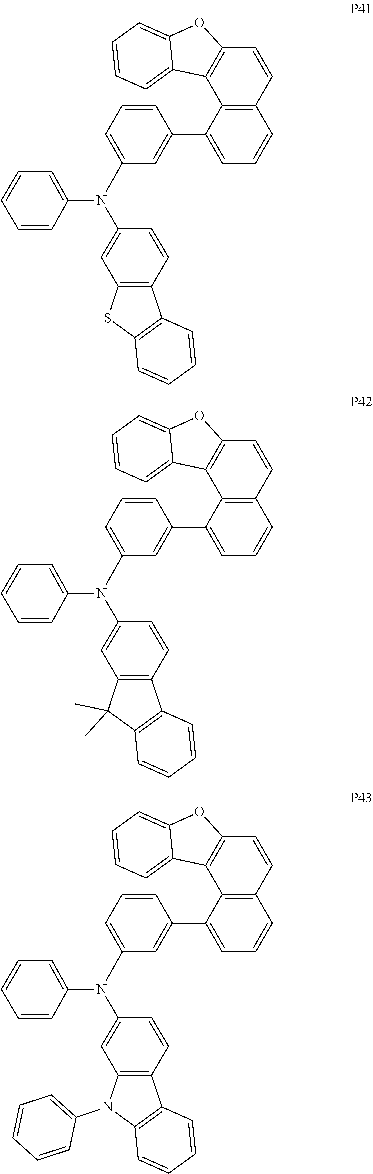

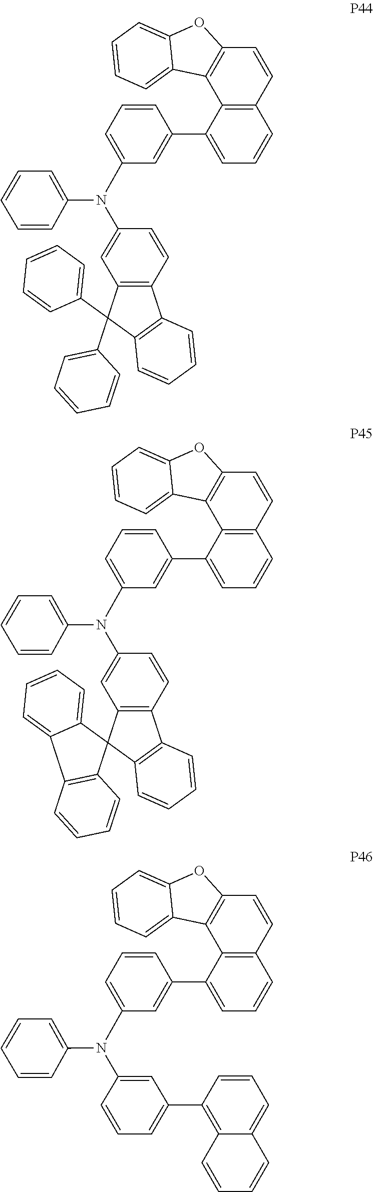 [SMILES: c1ccc(N(c2cccc(-c3cccc4ccc5oc6ccccc6c5c34)c2)c2ccc3c(c2)C(c2ccccc2)(c2ccccc2)c2ccccc2-3)cc1 ; c1ccc(N(c2cccc(-c3cccc4ccc5oc6ccccc6c5c34)c2)c2ccc3c(c2)C2(c4ccccc4-c4ccccc42)c2ccccc2-3)cc1 ; c1ccc(N(c2cccc(-c3cccc4ccccc34)c2)c2cccc(-c3cccc4ccc5oc6ccccc6c5c34)c2)cc1]

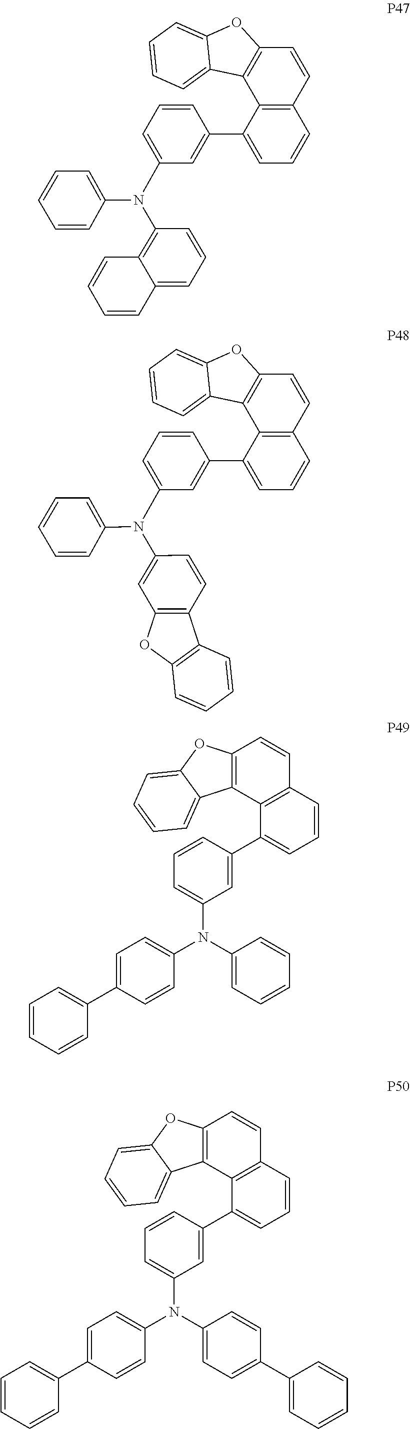

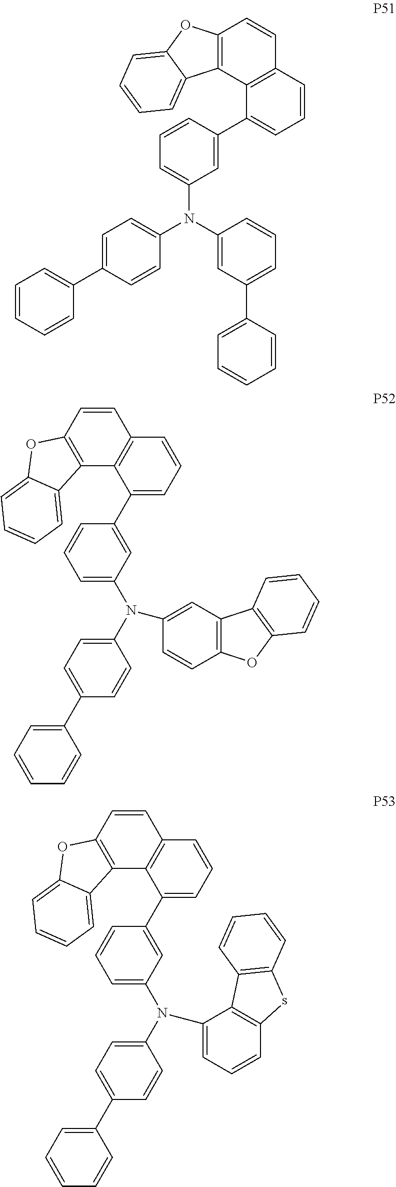

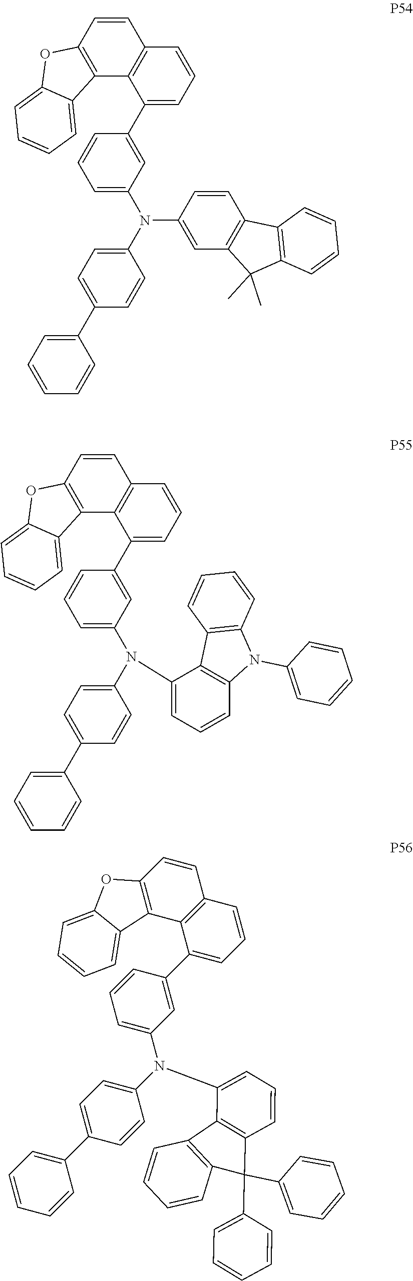 [SMILES: CC1(C)c2ccccc2-c2ccc(N(c3ccc(-c4ccccc4)cc3)c3cccc(-c4cccc5ccc6oc7ccccc7c6c45)c3)cc21 ; c1ccc(-c2ccc(N(c3cccc(-c4cccc5ccc6oc7ccccc7c6c45)c3)c3cccc4c3c3ccccc3n4-c3ccccc3)cc2)cc1 ; c1ccc(-c2ccc(N(c3cccc(-c4cccc5ccc6oc7ccccc7c6c45)c3)c3cccc4c3-c3ccccc3C4(c3ccccc3)c3ccccc3)cc2)cc1]

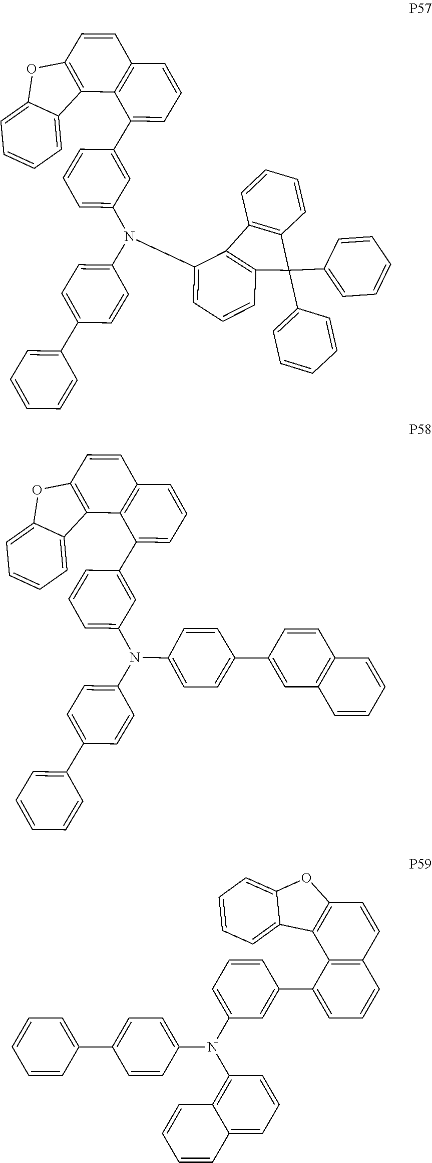 [SMILES: c1ccc(-c2ccc(N(c3cccc(-c4cccc5ccc6oc7ccccc7c6c45)c3)c3cccc4c3-c3ccccc3C4(c3ccccc3)c3ccccc3)cc2)cc1 ; c1ccc(-c2ccc(N(c3ccc(-c4ccc5ccccc5c4)cc3)c3cccc(-c4cccc5ccc6oc7ccccc7c6c45)c3)cc2)cc1 ; c1ccc(-c2ccc(N(c3cccc(-c4cccc5ccc6oc7ccccc7c6c45)c3)c3cccc4ccccc34)cc2)cc1]

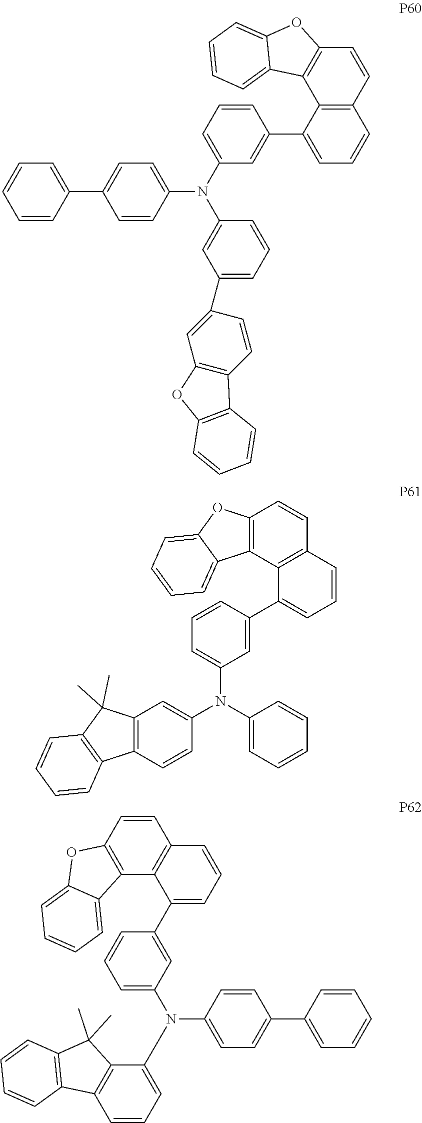

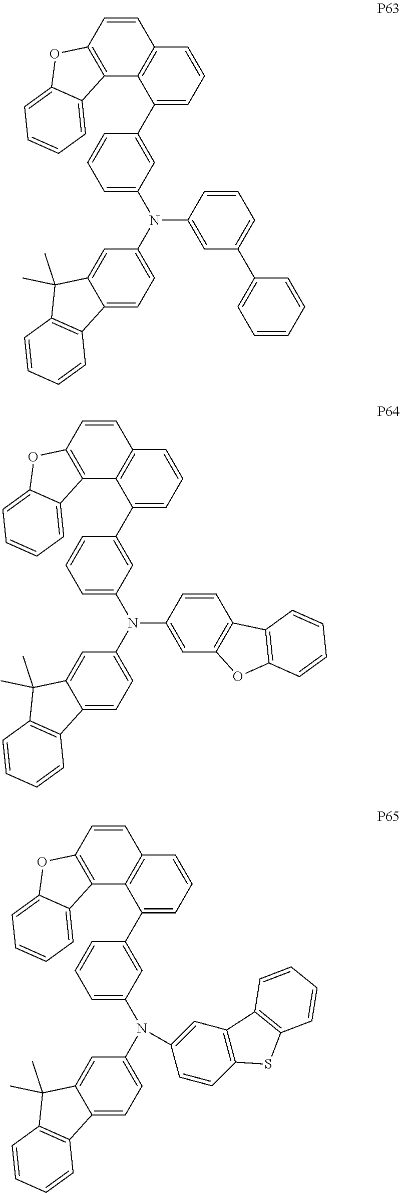

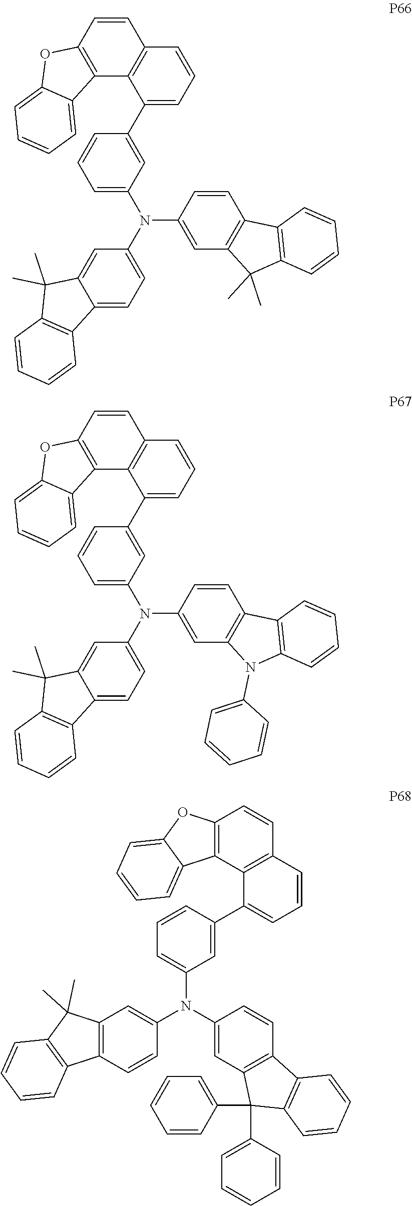

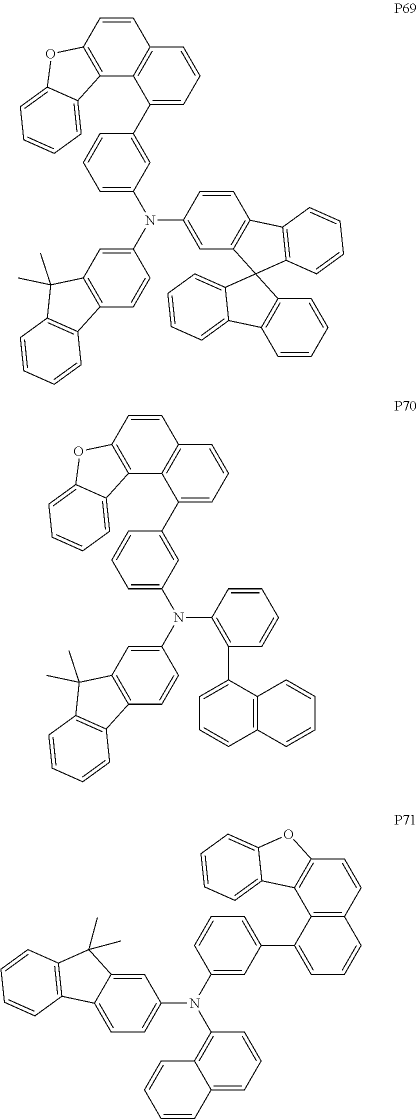 [SMILES: CC1(C)c2ccccc2-c2ccc(N(c3cccc(-c4cccc5ccc6oc7ccccc7c6c45)c3)c3ccc4c(c3)C3(c5ccccc5-c5ccccc53)c3ccccc3-4)cc21 ; CC1(C)c2ccccc2-c2ccc(N(c3cccc(-c4cccc5ccc6oc7ccccc7c6c45)c3)c3ccccc3-c3cccc4ccccc34)cc21 ; CC1(C)c2ccccc2-c2ccc(N(c3cccc(-c4cccc5ccc6oc7ccccc7c6c45)c3)c3cccc4ccccc34)cc21]

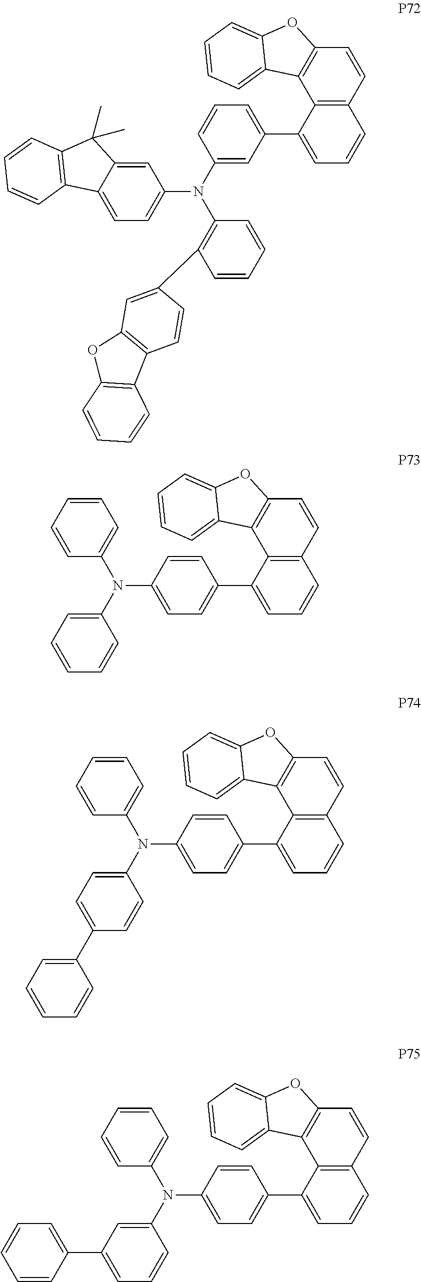 [SMILES: CC1(C)c2ccccc2-c2ccc(N(c3cccc(-c4cccc5ccc6oc7ccccc7c6c45)c3)c3ccccc3-c3ccc4c(c3)oc3ccccc34)cc21 ; c1ccc(N(c2ccccc2)c2ccc(-c3cccc4ccc5oc6ccccc6c5c34)cc2)cc1 ; c1ccc(-c2ccc(N(c3ccccc3)c3ccc(-c4cccc5ccc6oc7ccccc7c6c45)cc3)cc2)cc1 ; c1ccc(-c2cccc(N(c3ccccc3)c3ccc(-c4cccc5ccc6oc7ccccc7c6c45)cc3)c2)cc1]

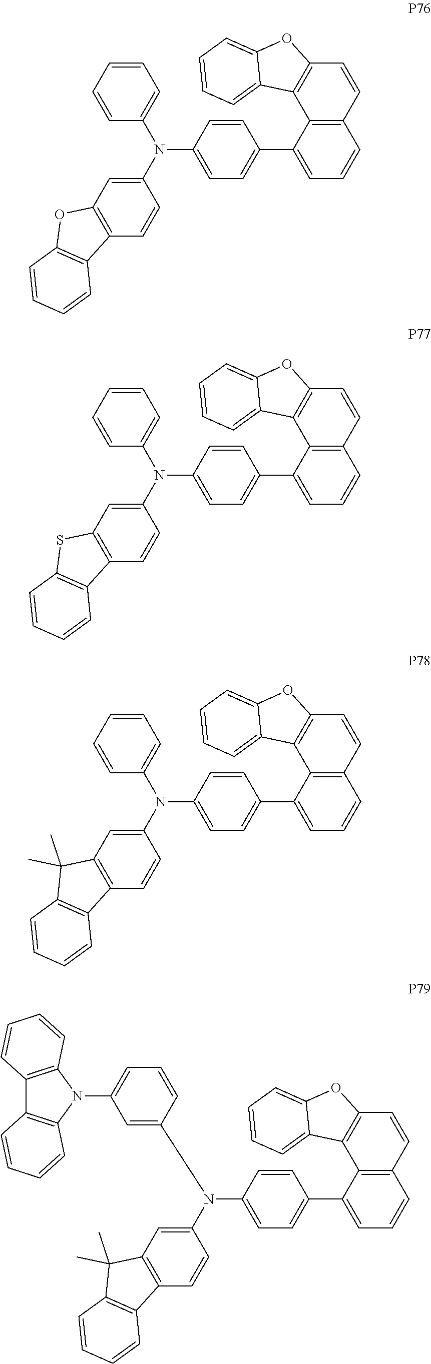 [SMILES: c1ccc(N(c2ccc(-c3cccc4ccc5oc6ccccc6c5c34)cc2)c2ccc3c(c2)oc2ccccc23)cc1 ; c1ccc(N(c2ccc(-c3cccc4ccc5oc6ccccc6c5c34)cc2)c2ccc3c(c2)sc2ccccc23)cc1 ; CC1(C)c2ccccc2-c2ccc(N(c3ccccc3)c3ccc(-c4cccc5ccc6oc7ccccc7c6c45)cc3)cc21 ; CC1(C)c2ccccc2-c2ccc(N(c3ccc(-c4cccc5ccc6oc7ccccc7c6c45)cc3)c3cccc(-n4c5ccccc5c5ccccc54)c3)cc21]

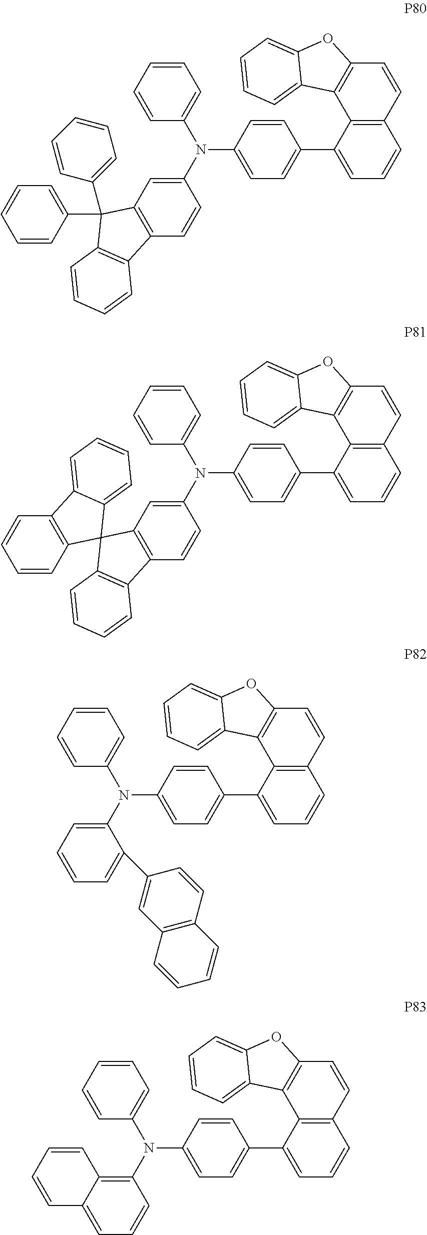 [SMILES: c1ccc(N(c2ccc(-c3cccc4ccc5oc6ccccc6c5c34)cc2)c2ccc3c(c2)C(c2ccccc2)(c2ccccc2)c2ccccc2-3)cc1 ; c1ccc(N(c2ccc(-c3cccc4ccc5oc6ccccc6c5c34)cc2)c2ccc3c(c2)C2(c4ccccc4-c4ccccc42)c2ccccc2-3)cc1 ; c1ccc(N(c2ccc(-c3cccc4ccc5oc6ccccc6c5c34)cc2)c2ccccc2-c2ccc3ccccc3c2)cc1 ; c1ccc(N(c2ccc(-c3cccc4ccc5oc6ccccc6c5c34)cc2)c2cccc3ccccc23)cc1]

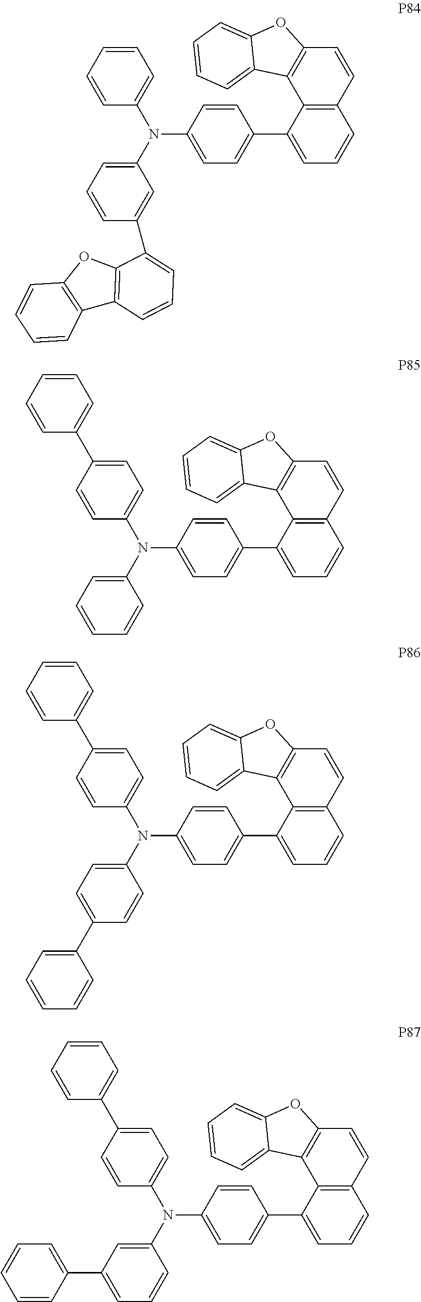

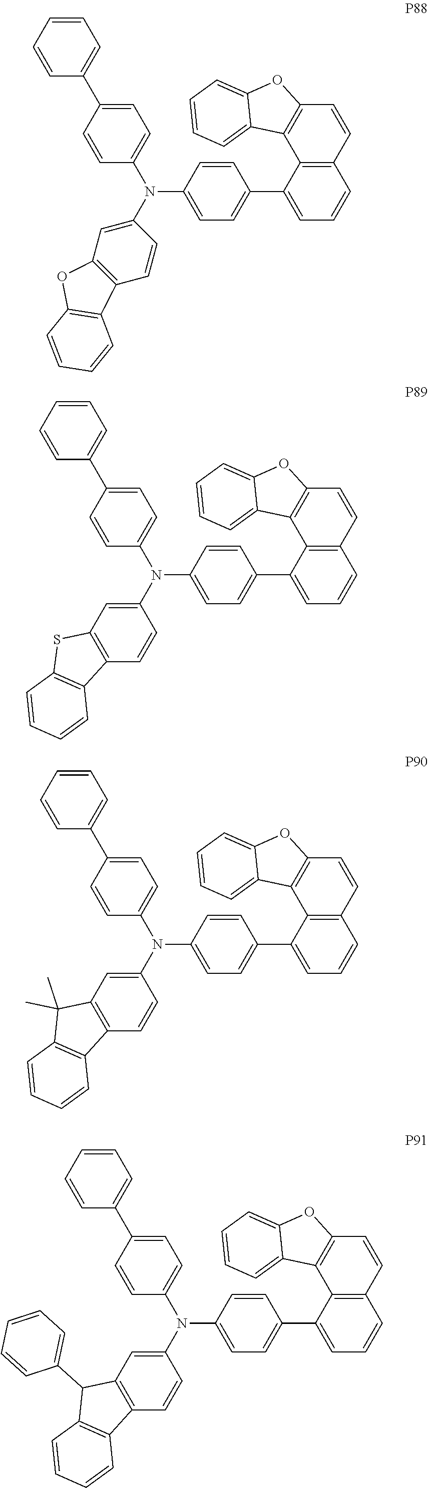

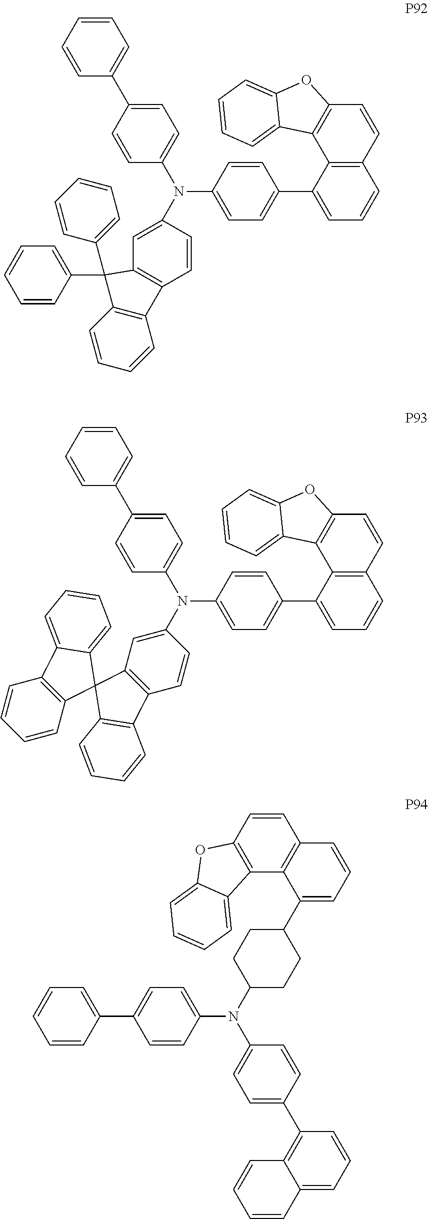 [SMILES: c1ccc(-c2ccc(N(c3ccc(-c4cccc5ccc6oc7ccccc7c6c45)cc3)c3ccc4c(c3)C(c3ccccc3)(c3ccccc3)c3ccccc3-4)cc2)cc1 ; c1ccc(-c2ccc(N(c3ccc(-c4cccc5ccc6oc7ccccc7c6c45)cc3)c3ccc4c(c3)C3(c5ccccc5-c5ccccc53)c3ccccc3-4)cc2)cc1 ; c1ccc(-c2ccc(N(c3ccc(-c4cccc5ccccc45)cc3)C3CCC(c4cccc5ccc6oc7ccccc7c6c45)CC3)cc2)cc1]

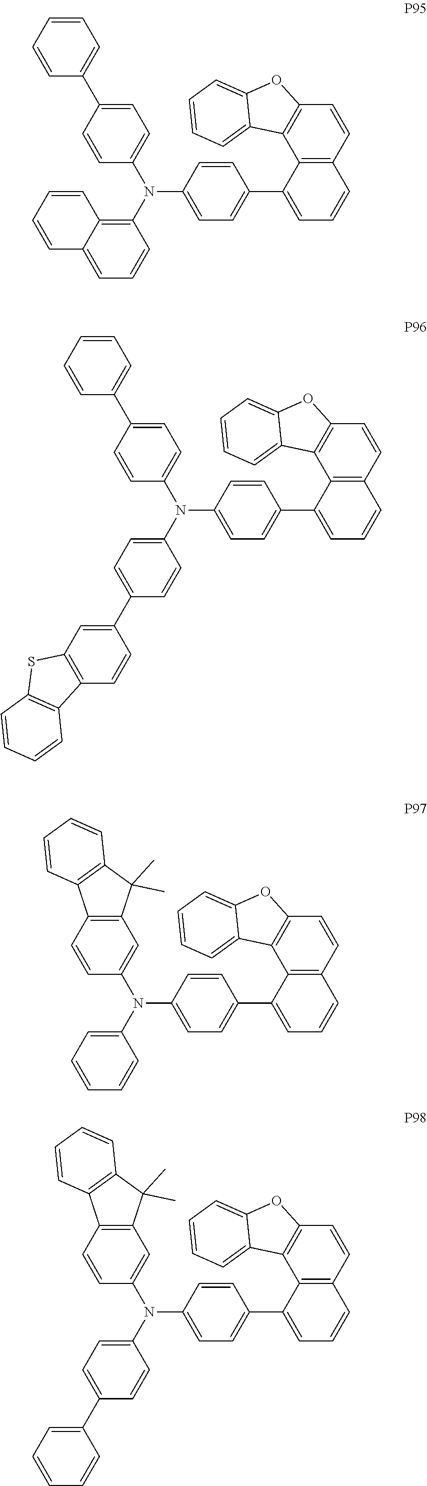

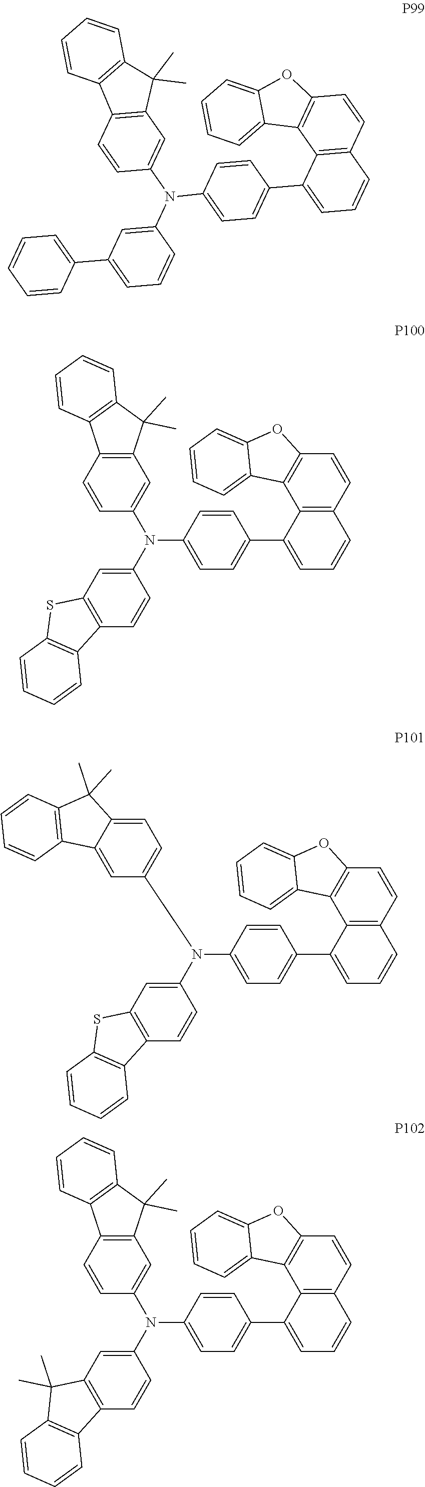

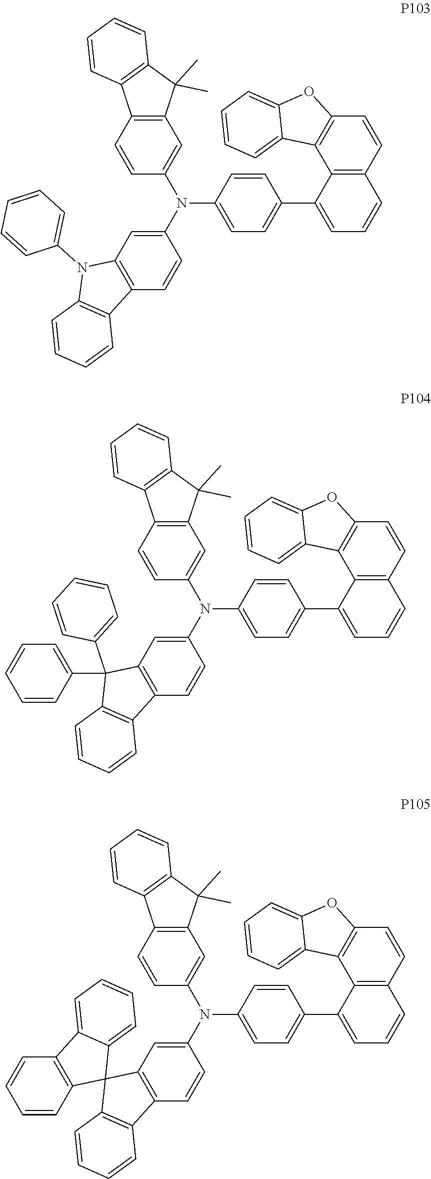 [SMILES: CC1(C)c2ccccc2-c2ccc(N(c3ccc(-c4cccc5ccc6oc7ccccc7c6c45)cc3)c3ccc4c5ccccc5n(-c5ccccc5)c4c3)cc21 ; CC1(C)c2ccccc2-c2ccc(N(c3ccc(-c4cccc5ccc6oc7ccccc7c6c45)cc3)c3ccc4c(c3)C(c3ccccc3)(c3ccccc3)c3ccccc3-4)cc21 ; CC1(C)c2ccccc2-c2ccc(N(c3ccc(-c4cccc5ccc6oc7ccccc7c6c45)cc3)c3ccc4c(c3)C3(c5ccccc5-c5ccccc53)c3ccccc3-4)cc21]

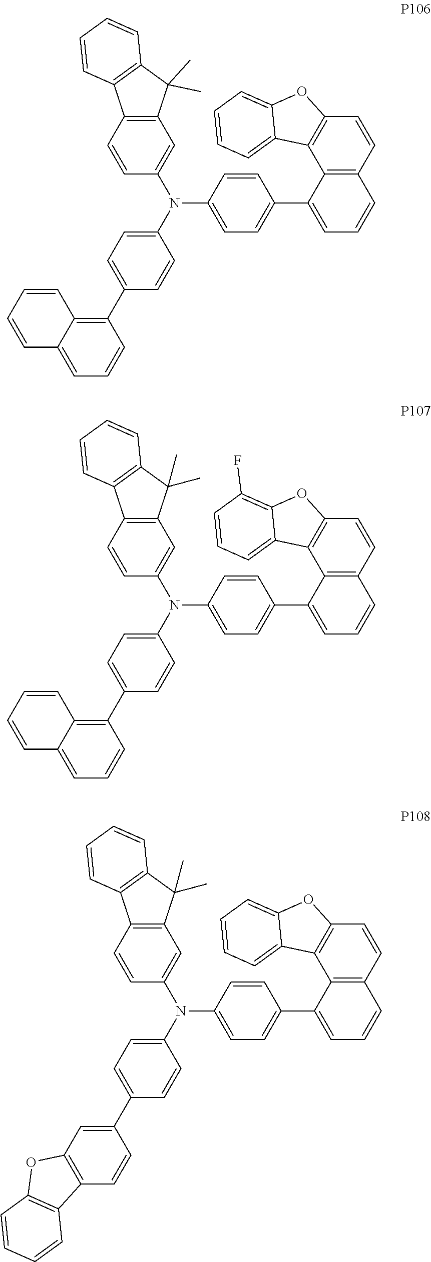

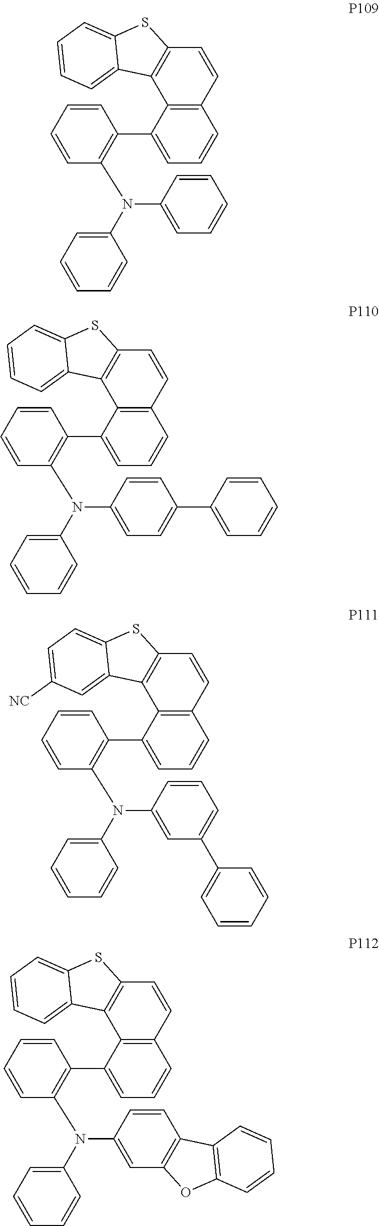

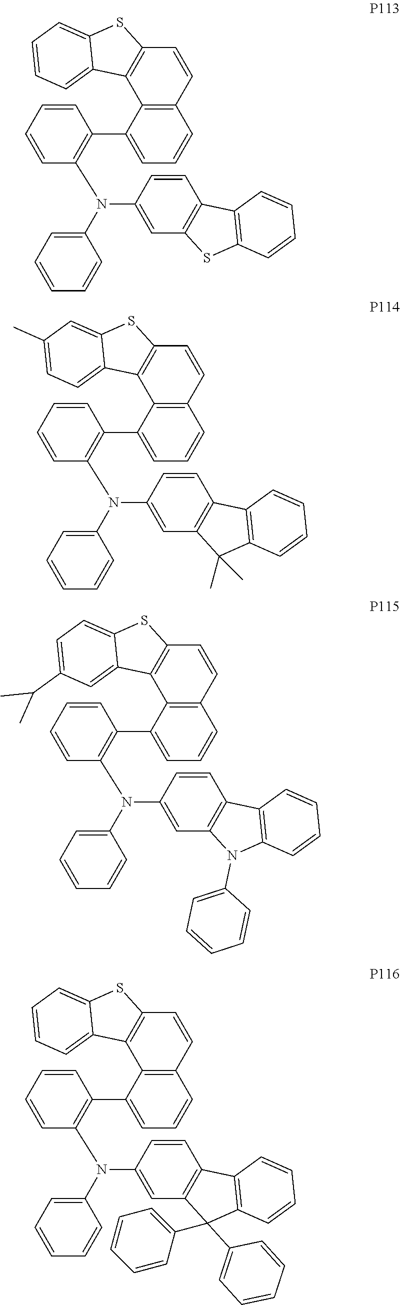

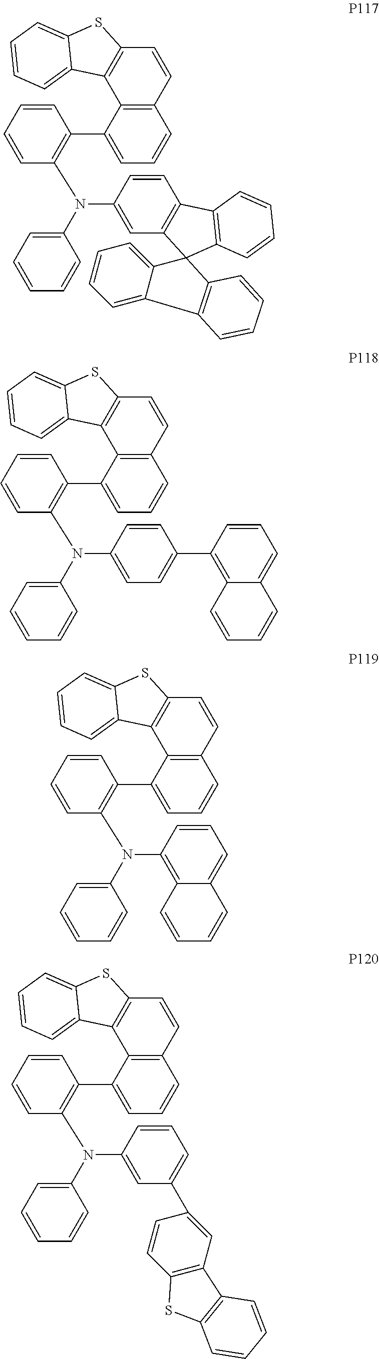

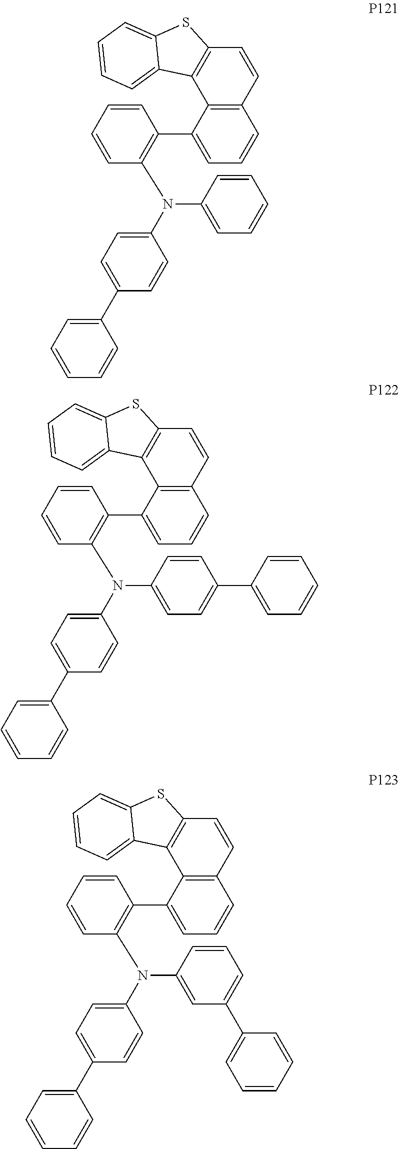

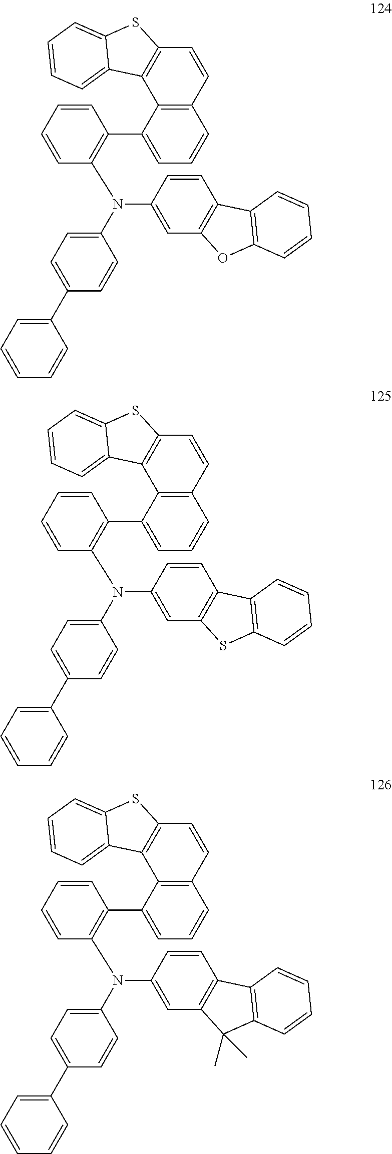

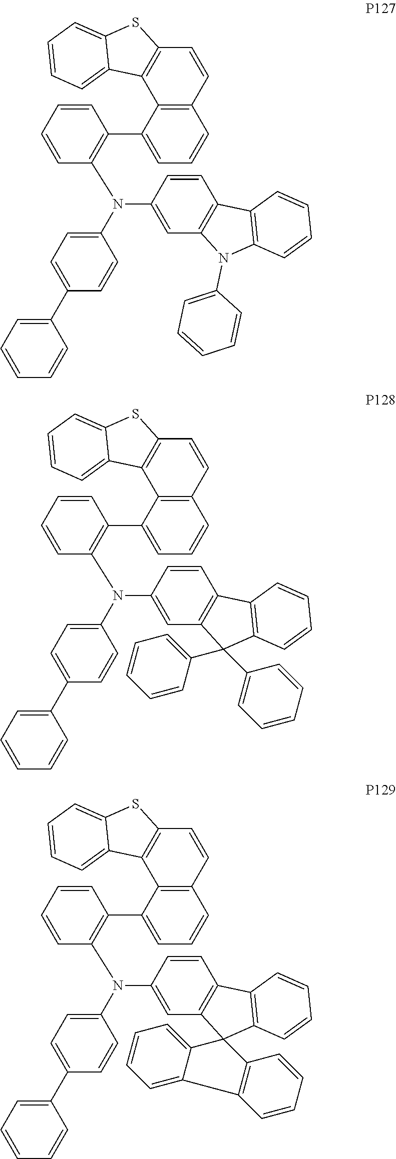

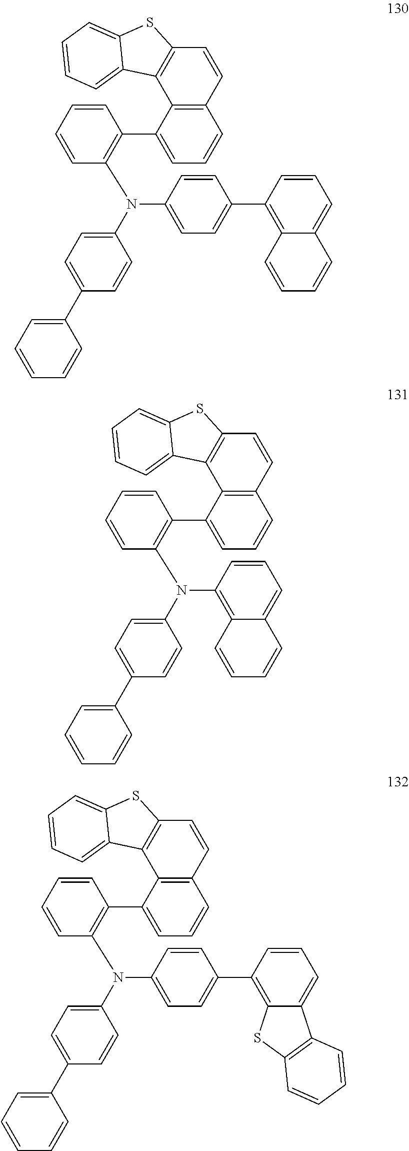

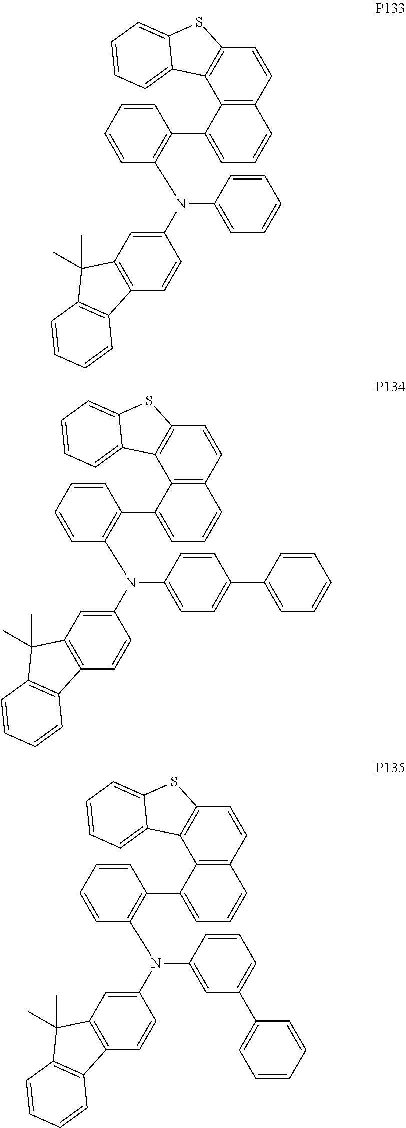

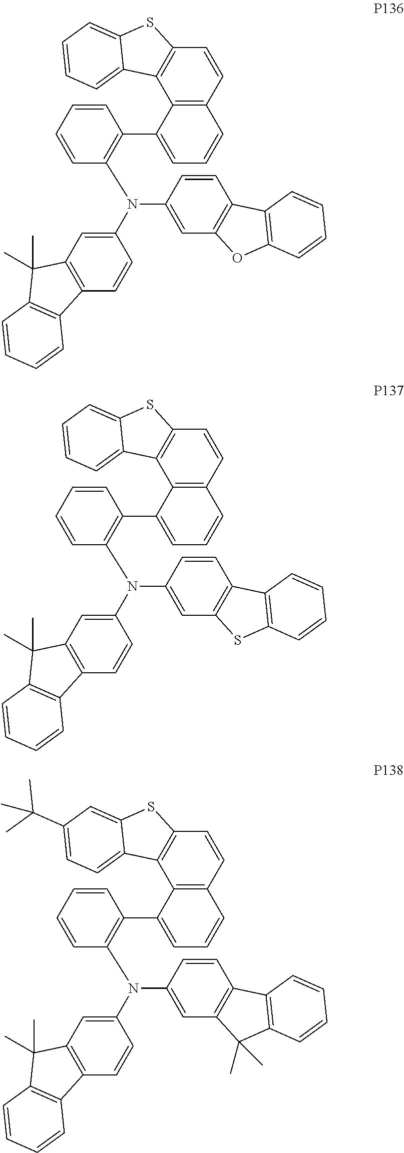

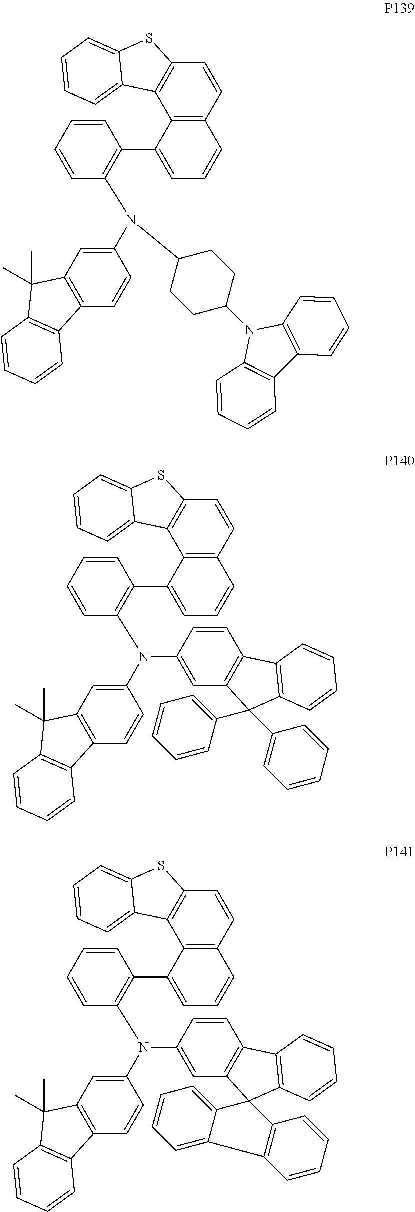 [SMILES: CC1(C)c2ccccc2-c2ccc(N(c3ccccc3-c3cccc4ccc5sc6ccccc6c5c34)C3CCC(n4c5ccccc5c5ccccc54)CC3)cc21 ; CC1(C)c2ccccc2-c2ccc(N(c3ccc4c(c3)C(c3ccccc3)(c3ccccc3)c3ccccc3-4)c3ccccc3-c3cccc4ccc5sc6ccccc6c5c34)cc21 ; CC1(C)c2ccccc2-c2ccc(N(c3ccc4c(c3)C3(c5ccccc5-c5ccccc53)c3ccccc3-4)c3ccccc3-c3cccc4ccc5sc6ccccc6c5c34)cc21]

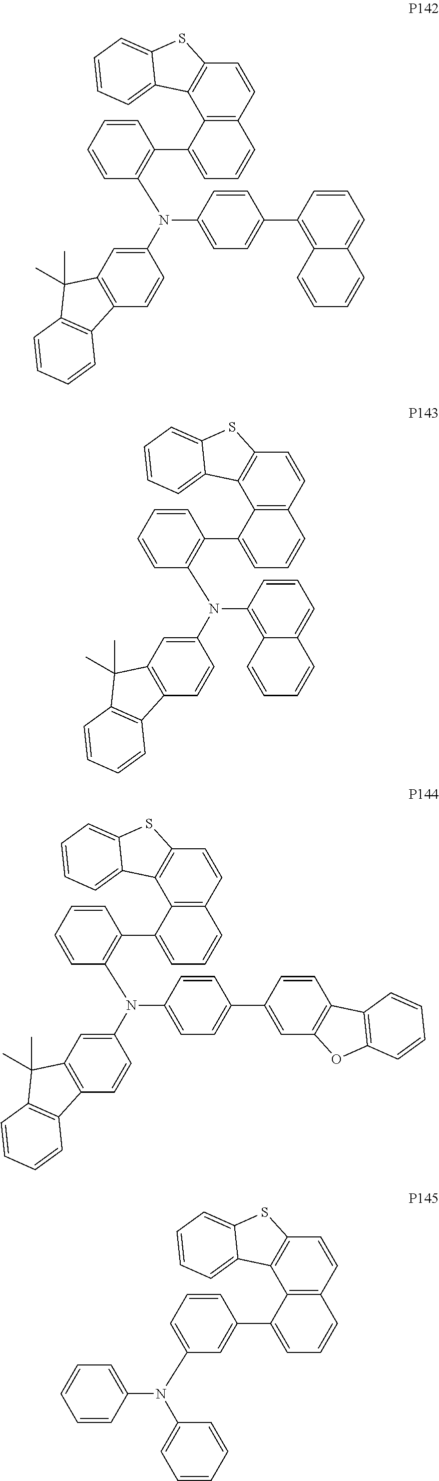 [SMILES: CC1(C)c2ccccc2-c2ccc(N(c3ccc(-c4cccc5ccccc45)cc3)c3ccccc3-c3cccc4ccc5sc6ccccc6c5c34)cc21 ; CC1(C)c2ccccc2-c2ccc(N(c3ccccc3-c3cccc4ccc5sc6ccccc6c5c34)c3cccc4ccccc34)cc21 ; CC1(C)c2ccccc2-c2ccc(N(c3ccc(-c4ccc5c(c4)oc4ccccc45)cc3)c3ccccc3-c3cccc4ccc5sc6ccccc6c5c34)cc21 ; c1ccc(N(c2ccccc2)c2cccc(-c3cccc4ccc5sc6ccccc6c5c34)c2)cc1]

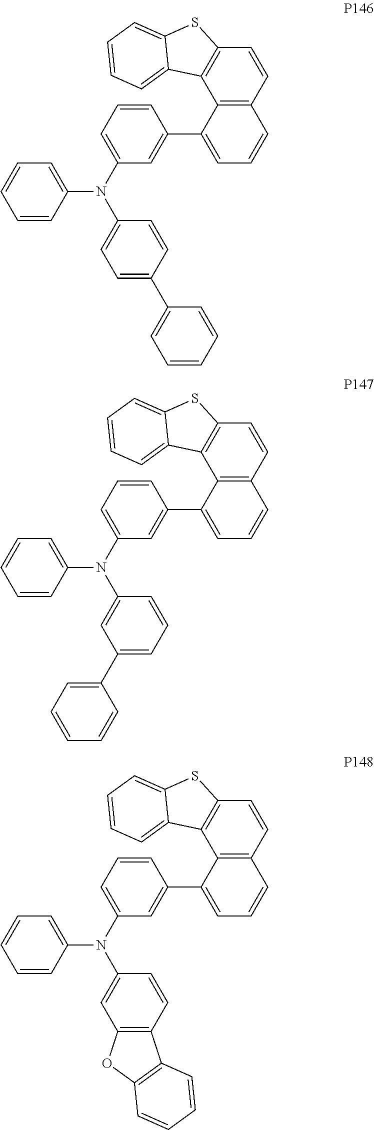

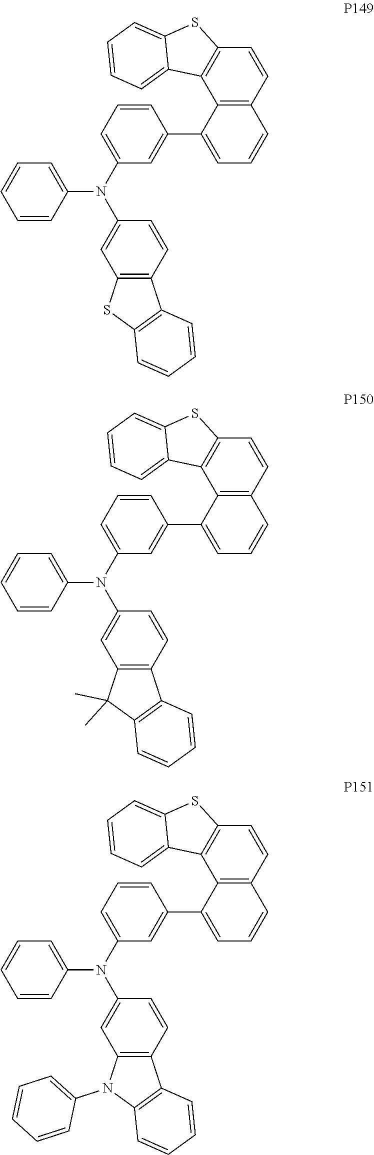

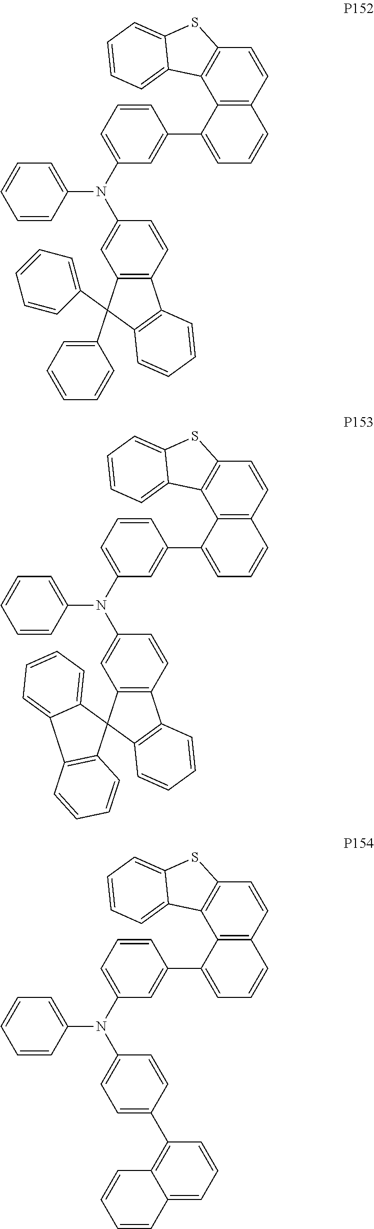 [SMILES: c1ccc(N(c2cccc(-c3cccc4ccc5sc6ccccc6c5c34)c2)c2ccc3c(c2)C(c2ccccc2)(c2ccccc2)c2ccccc2-3)cc1 ; c1ccc(N(c2cccc(-c3cccc4ccc5sc6ccccc6c5c34)c2)c2ccc3c(c2)C2(c4ccccc4-c4ccccc42)c2ccccc2-3)cc1 ; c1ccc(N(c2ccc(-c3cccc4ccccc34)cc2)c2cccc(-c3cccc4ccc5sc6ccccc6c5c34)c2)cc1]

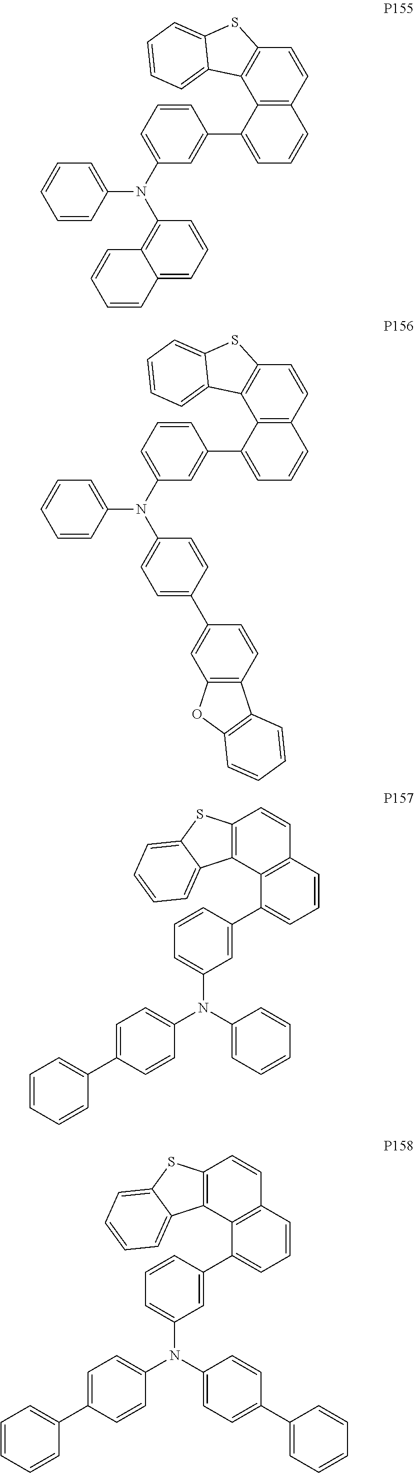

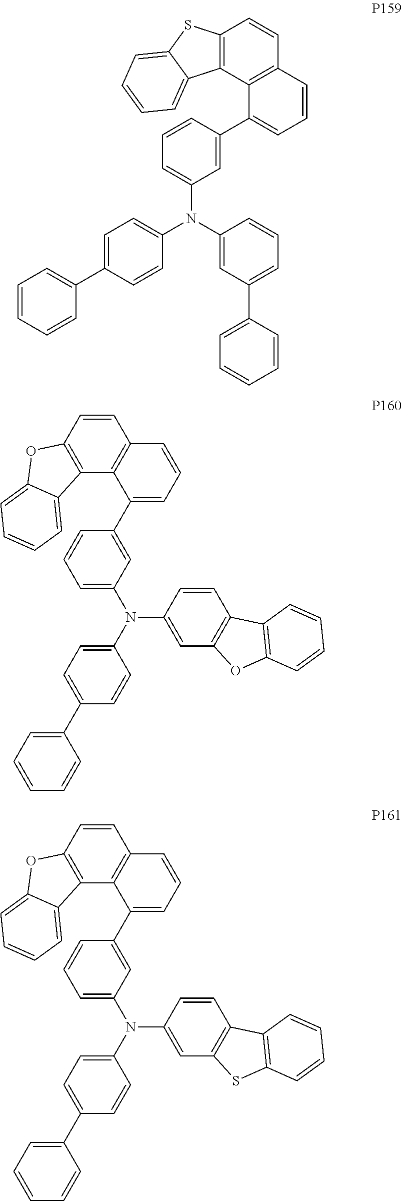

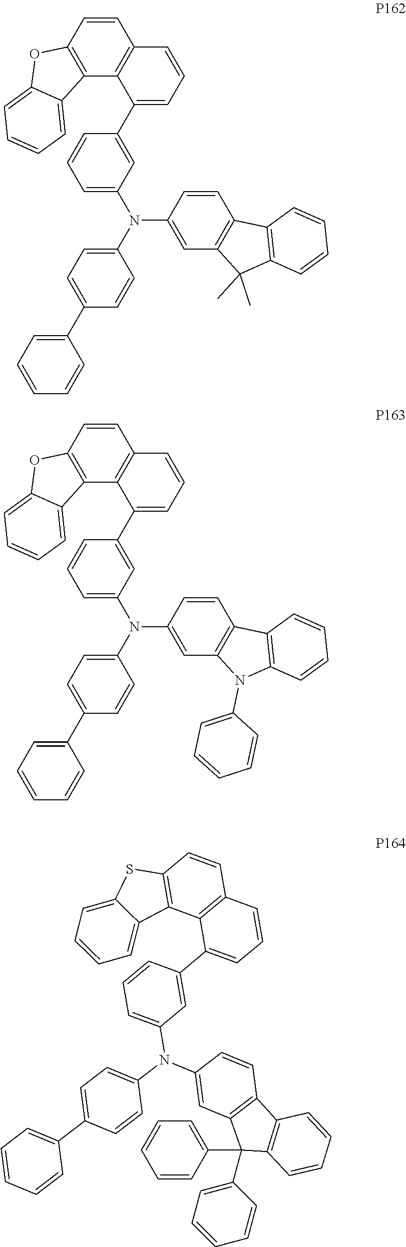

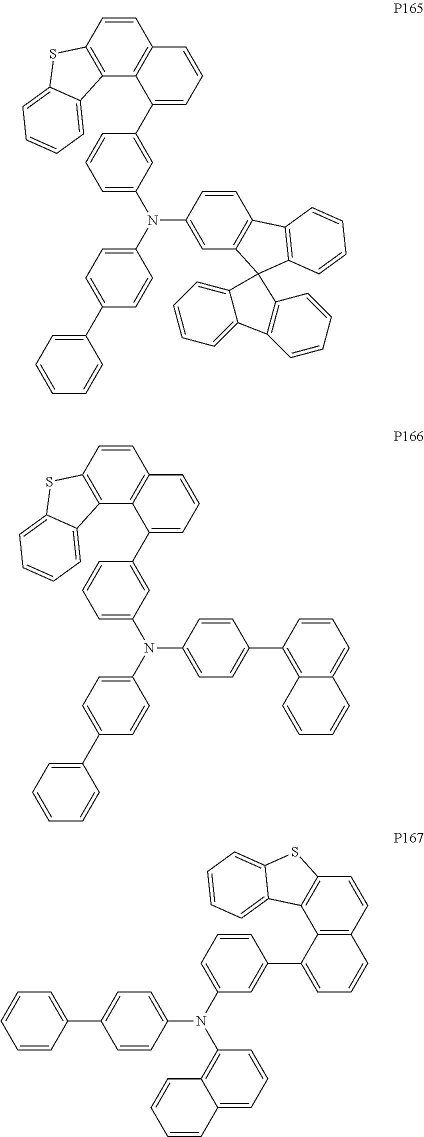 [SMILES: c1ccc(-c2ccc(N(c3cccc(-c4cccc5ccc6sc7ccccc7c6c45)c3)c3ccc4c(c3)C3(c5ccccc5-c5ccccc53)c3ccccc3-4)cc2)cc1 ; c1ccc(-c2ccc(N(c3ccc(-c4cccc5ccccc45)cc3)c3cccc(-c4cccc5ccc6sc7ccccc7c6c45)c3)cc2)cc1 ; c1ccc(-c2ccc(N(c3cccc(-c4cccc5ccc6sc7ccccc7c6c45)c3)c3cccc4ccccc34)cc2)cc1]

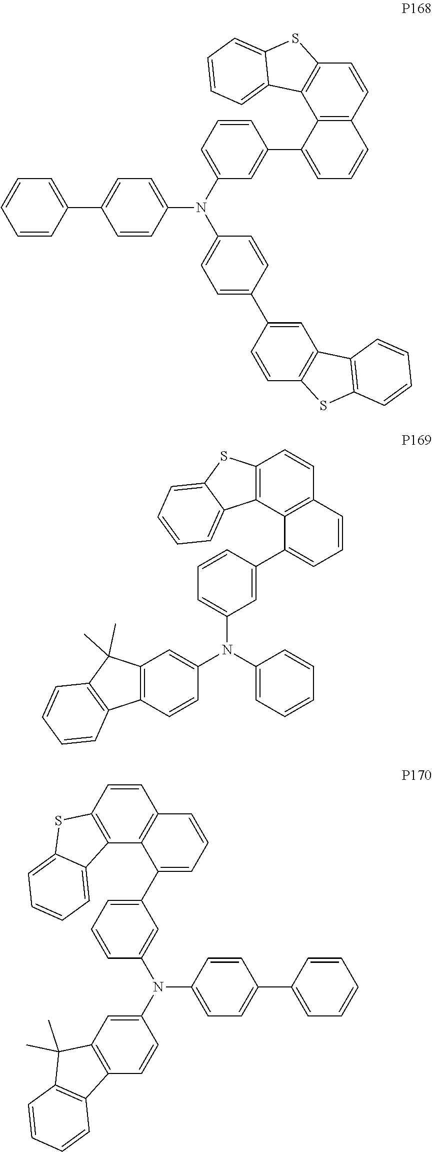

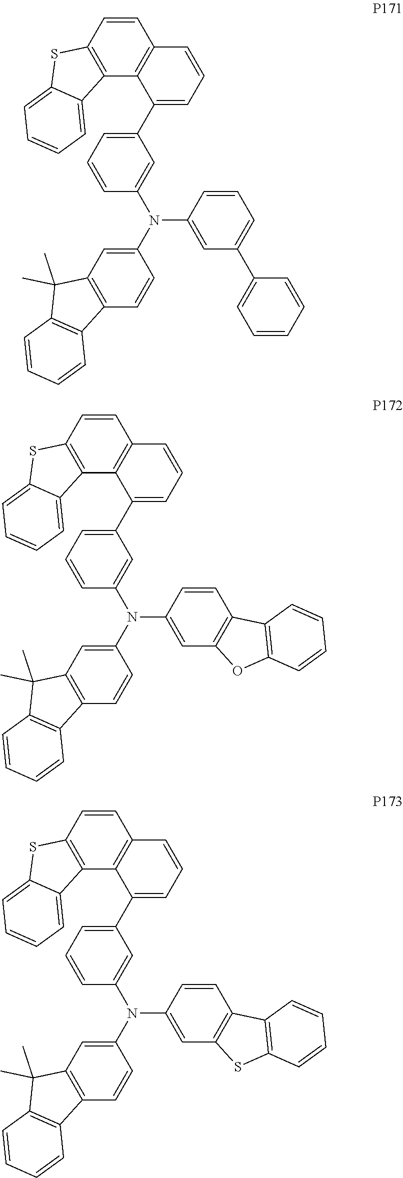

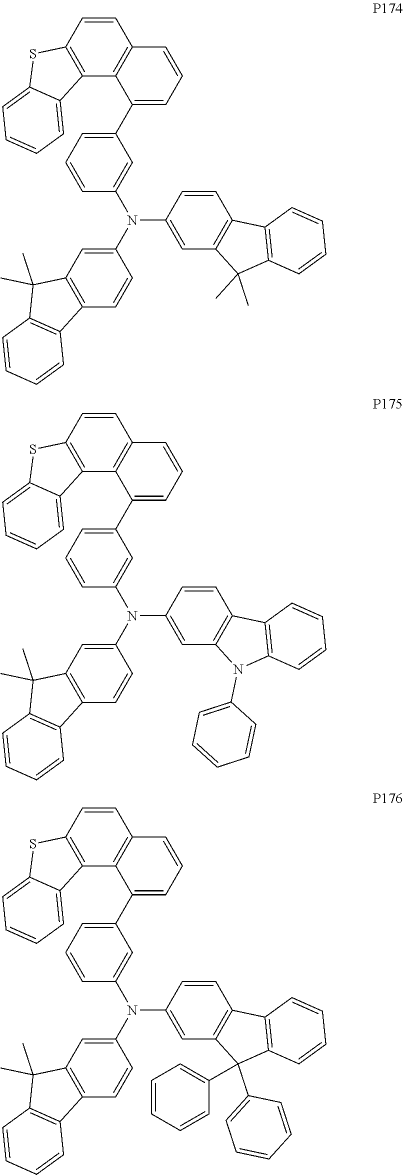 [SMILES: CC1(C)c2ccccc2-c2ccc(N(c3cccc(-c4cccc5ccc6sc7ccccc7c6c45)c3)c3ccc4c(c3)C(C)(C)c3ccccc3-4)cc21 ; CC1(C)c2ccccc2-c2ccc(N(c3cccc(-c4cccc5ccc6sc7ccccc7c6c45)c3)c3ccc4c5ccccc5n(-c5ccccc5)c4c3)cc21 ; CC1(C)c2ccccc2-c2ccc(N(c3cccc(-c4cccc5ccc6sc7ccccc7c6c45)c3)c3ccc4c(c3)C(c3ccccc3)(c3ccccc3)c3ccccc3-4)cc21]

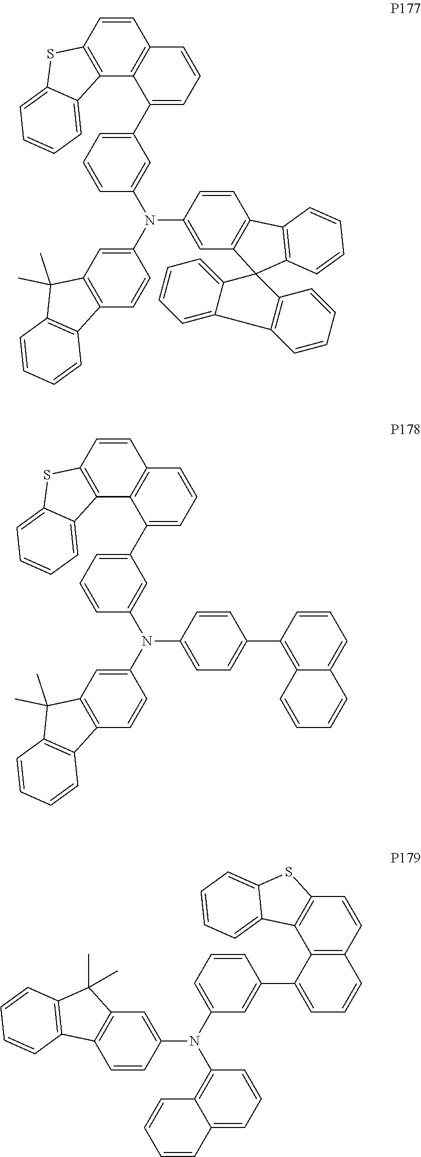 [SMILES: CC1(C)c2ccccc2-c2ccc(N(c3cccc(-c4cccc5ccc6sc7ccccc7c6c45)c3)c3ccc4c(c3)C3(c5ccccc5-c5ccccc53)c3ccccc3-4)cc21 ; CC1(C)c2ccccc2-c2ccc(N(c3ccc(-c4cccc5ccccc45)cc3)c3cccc(-c4cccc5ccc6sc7ccccc7c6c45)c3)cc21 ; CC1(C)c2ccccc2-c2ccc(N(c3cccc(-c4cccc5ccc6sc7ccccc7c6c45)c3)c3cccc4ccccc34)cc21]

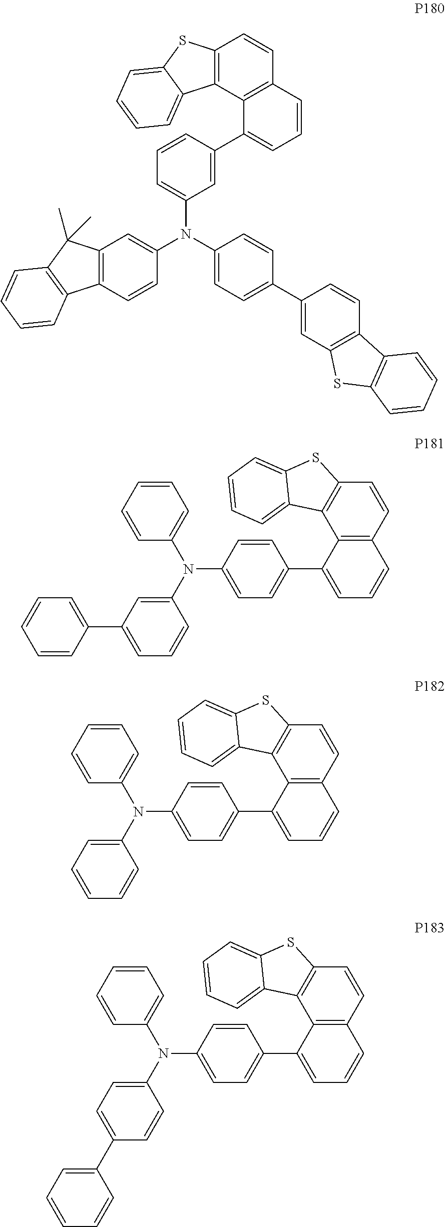 [SMILES: CC1(C)c2ccccc2-c2ccc(N(c3ccc(-c4ccc5c(c4)sc4ccccc45)cc3)c3cccc(-c4cccc5ccc6sc7ccccc7c6c45)c3)cc21 ; c1ccc(-c2cccc(N(c3ccccc3)c3ccc(-c4cccc5ccc6sc7ccccc7c6c45)cc3)c2)cc1 ; c1ccc(N(c2ccccc2)c2ccc(-c3cccc4ccc5sc6ccccc6c5c34)cc2)cc1 ; c1ccc(-c2ccc(N(c3ccccc3)c3ccc(-c4cccc5ccc6sc7ccccc7c6c45)cc3)cc2)cc1]

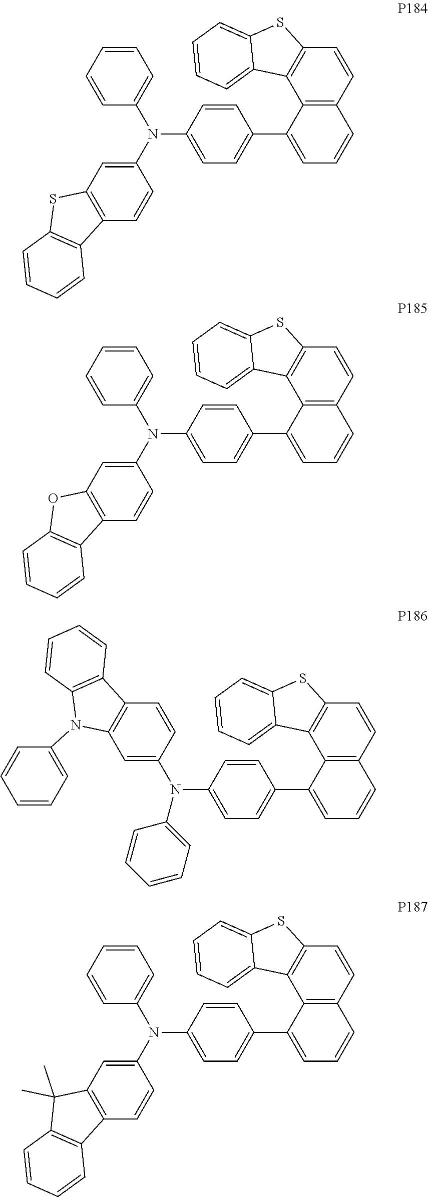

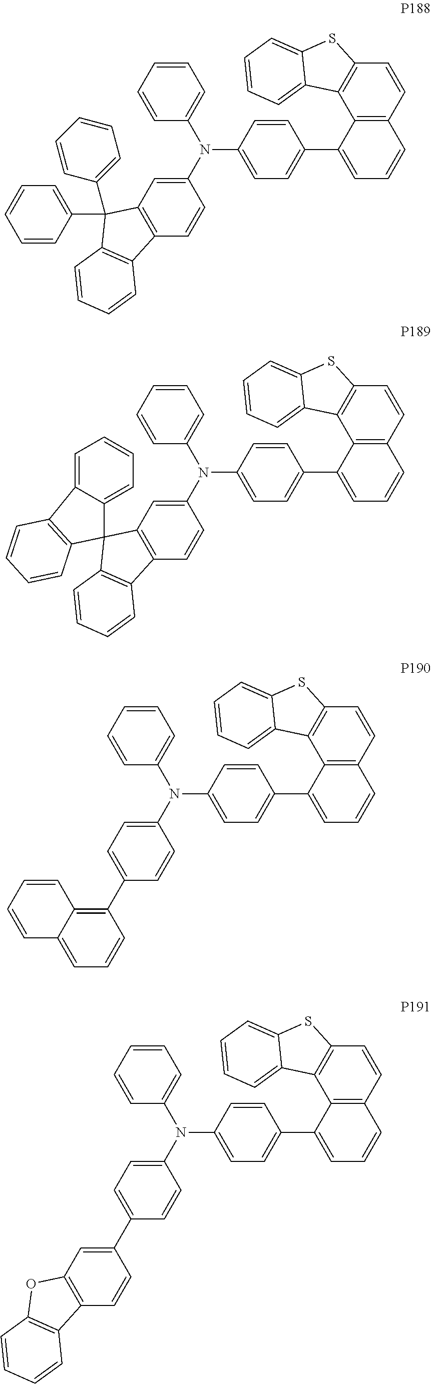 [SMILES: c1ccc(N(c2ccc(-c3cccc4ccc5sc6ccccc6c5c34)cc2)c2ccc3c(c2)C(c2ccccc2)(c2ccccc2)c2ccccc2-3)cc1 ; c1ccc(N(c2ccc(-c3cccc4ccc5sc6ccccc6c5c34)cc2)c2ccc3c(c2)C2(c4ccccc4-c4ccccc42)c2ccccc2-3)cc1 ; c1ccc(N(c2ccc(-c3cccc4ccccc34)cc2)c2ccc(-c3cccc4ccc5sc6ccccc6c5c34)cc2)cc1 ; c1ccc(N(c2ccc(-c3ccc4c(c3)oc3ccccc34)cc2)c2ccc(-c3cccc4ccc5sc6ccccc6c5c34)cc2)cc1]

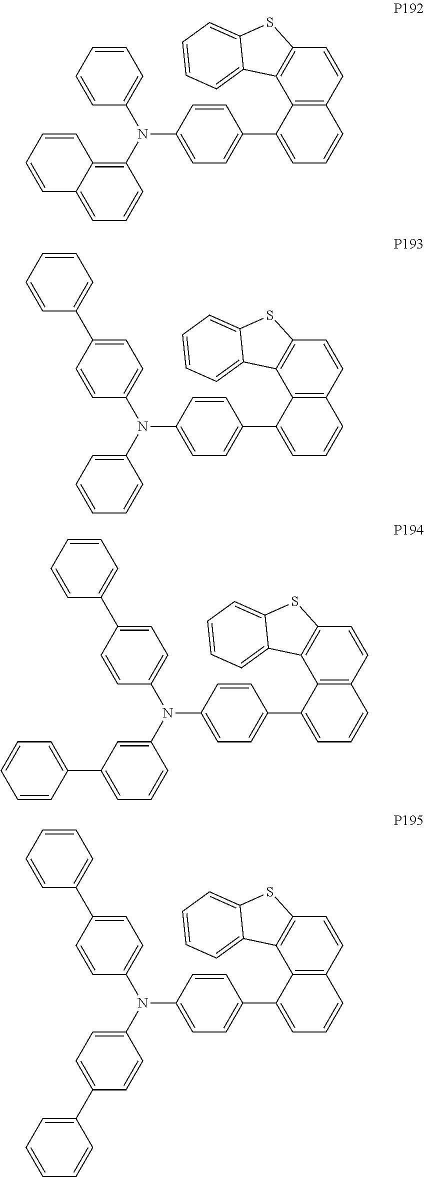

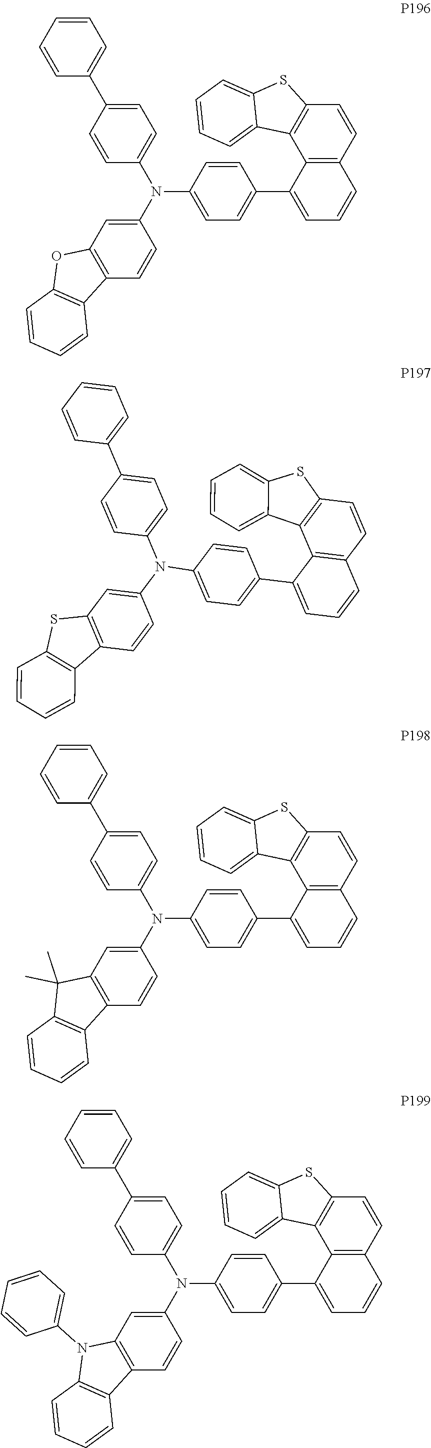

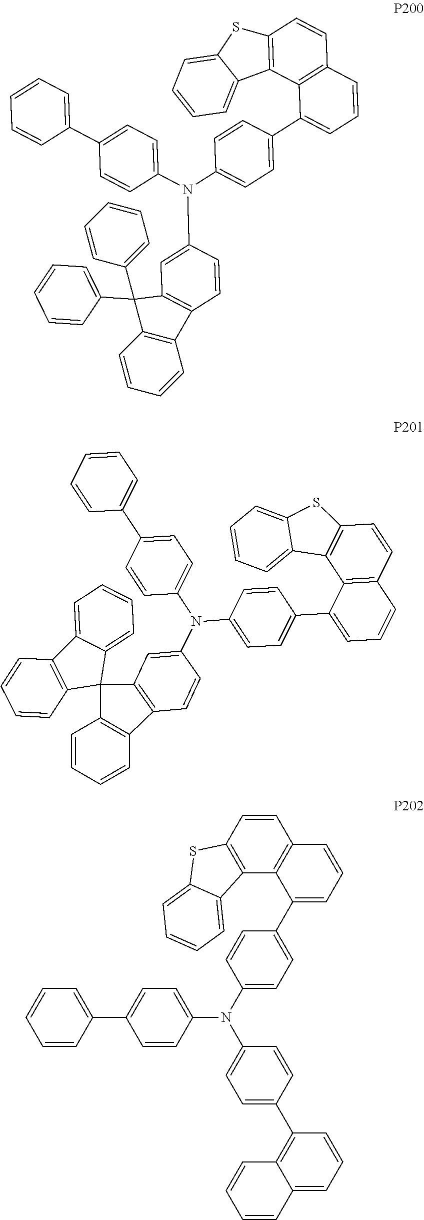 [SMILES: c1ccc(-c2ccc(N(c3ccc(-c4cccc5ccc6sc7ccccc7c6c45)cc3)c3ccc4c(c3)C(c3ccccc3)(c3ccccc3)c3ccccc3-4)cc2)cc1 ; c1ccc(-c2ccc(N(c3ccc(-c4cccc5ccc6sc7ccccc7c6c45)cc3)c3ccc4c(c3)C3(c5ccccc5-c5ccccc53)c3ccccc3-4)cc2)cc1 ; c1ccc(-c2ccc(N(c3ccc(-c4cccc5ccccc45)cc3)c3ccc(-c4cccc5ccc6sc7ccccc7c6c45)cc3)cc2)cc1]

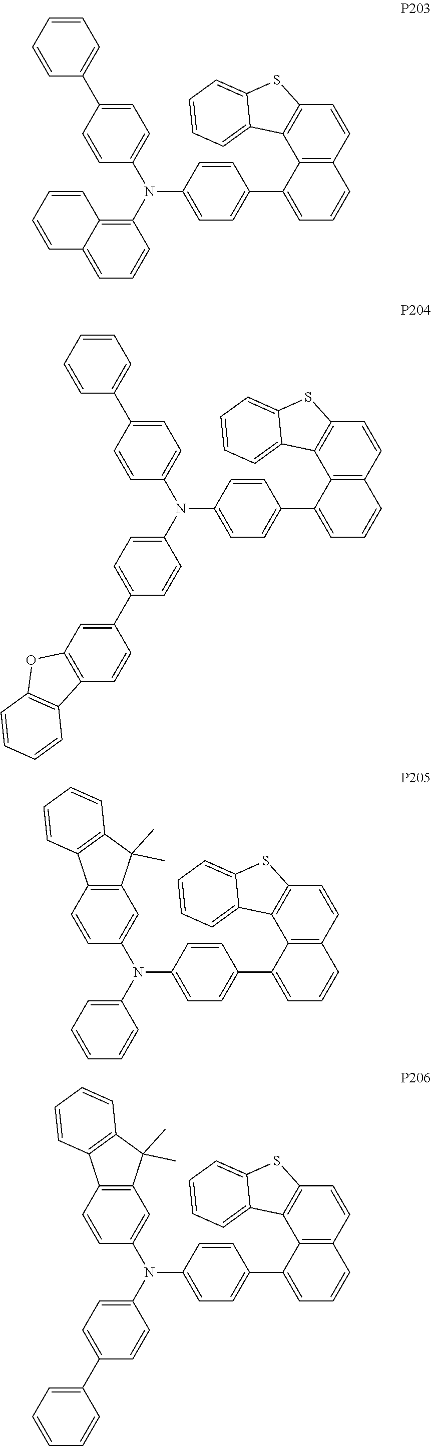

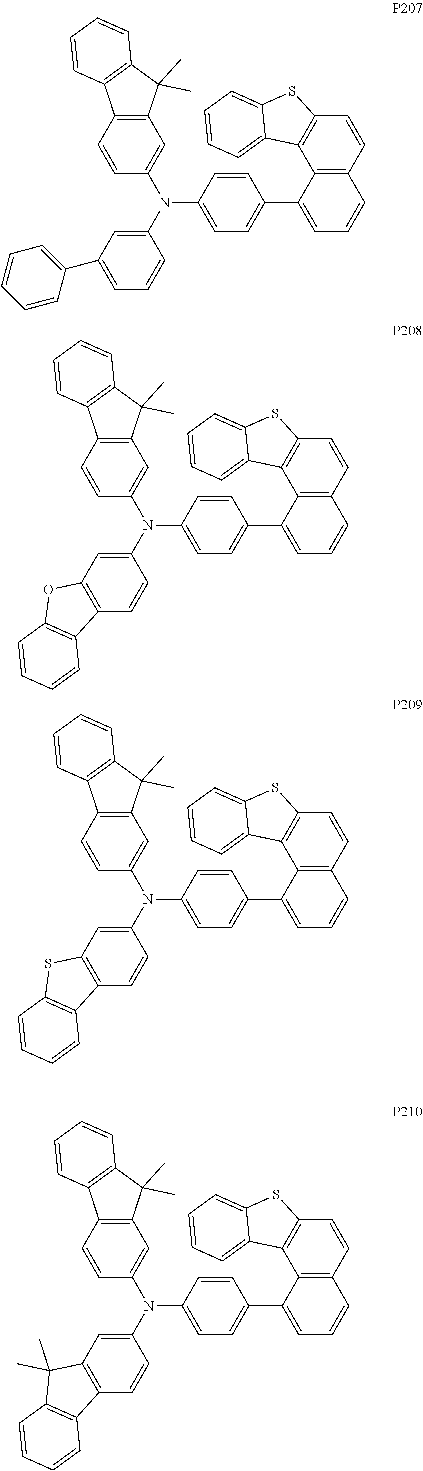

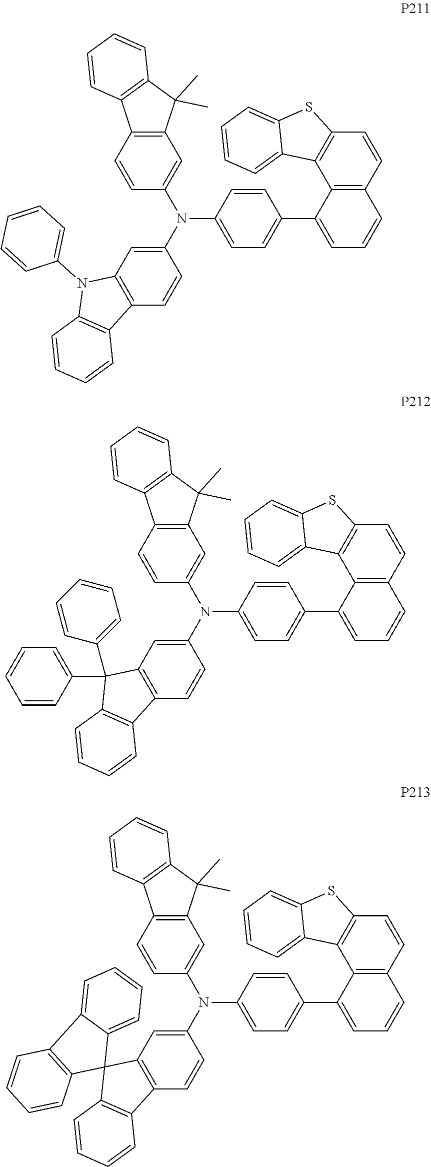 [SMILES: CC1(C)c2ccccc2-c2ccc(N(c3ccc(-c4cccc5ccc6sc7ccccc7c6c45)cc3)c3ccc4c5ccccc5n(-c5ccccc5)c4c3)cc21 ; CC1(C)c2ccccc2-c2ccc(N(c3ccc(-c4cccc5ccc6sc7ccccc7c6c45)cc3)c3ccc4c(c3)C(c3ccccc3)(c3ccccc3)c3ccccc3-4)cc21 ; CC1(C)c2ccccc2-c2ccc(N(c3ccc(-c4cccc5ccc6sc7ccccc7c6c45)cc3)c3ccc4c(c3)C3(c5ccccc5-c5ccccc53)c3ccccc3-4)cc21]

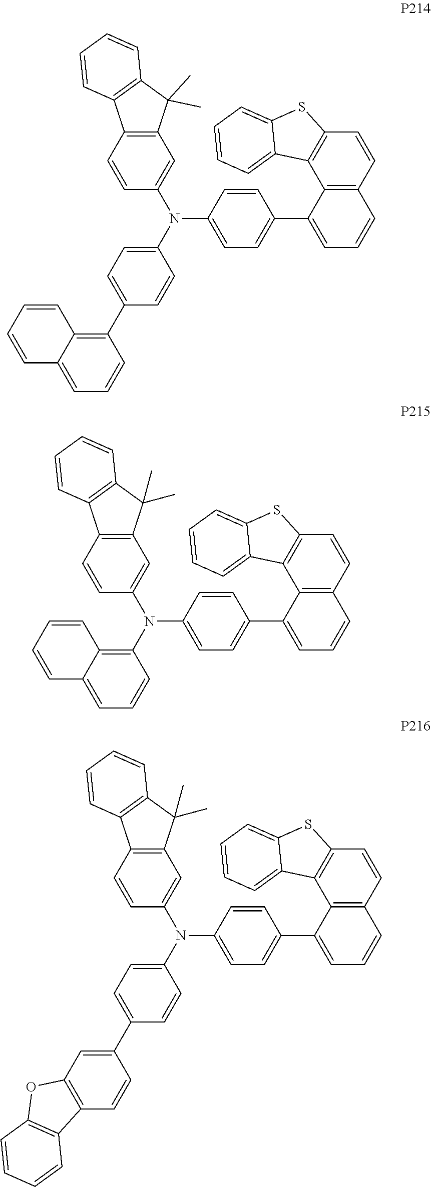

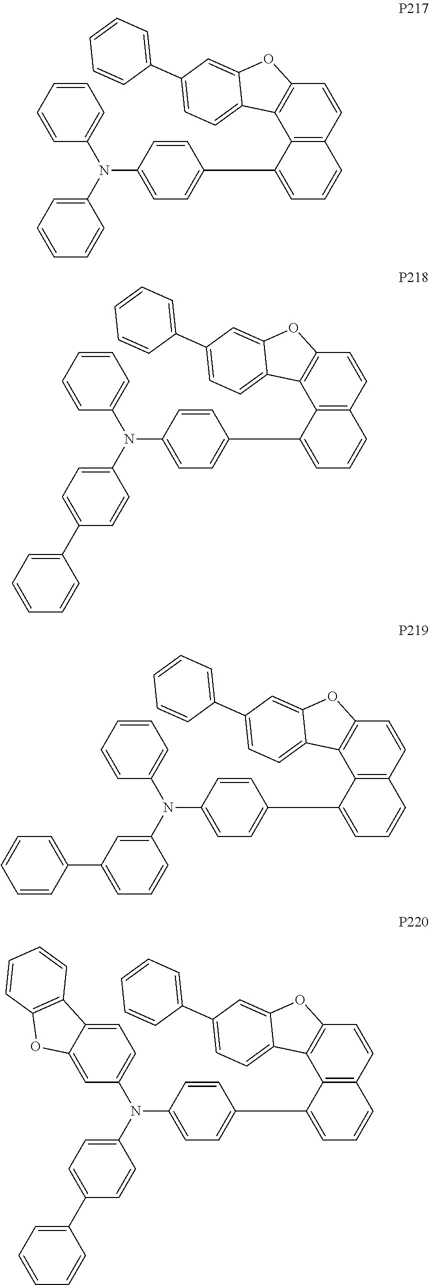 [SMILES: c1ccc(-c2ccc3c(c2)oc2ccc4cccc(-c5ccc(N(c6ccccc6)c6ccccc6)cc5)c4c23)cc1 ; c1ccc(-c2ccc(N(c3ccccc3)c3ccc(-c4cccc5ccc6oc7cc(-c8ccccc8)ccc7c6c45)cc3)cc2)cc1 ; c1ccc(-c2cccc(N(c3ccccc3)c3ccc(-c4cccc5ccc6oc7cc(-c8ccccc8)ccc7c6c45)cc3)c2)cc1 ; c1ccc(-c2ccc(N(c3ccc(-c4cccc5ccc6oc7cc(-c8ccccc8)ccc7c6c45)cc3)c3ccc4c(c3)oc3ccccc34)cc2)cc1]

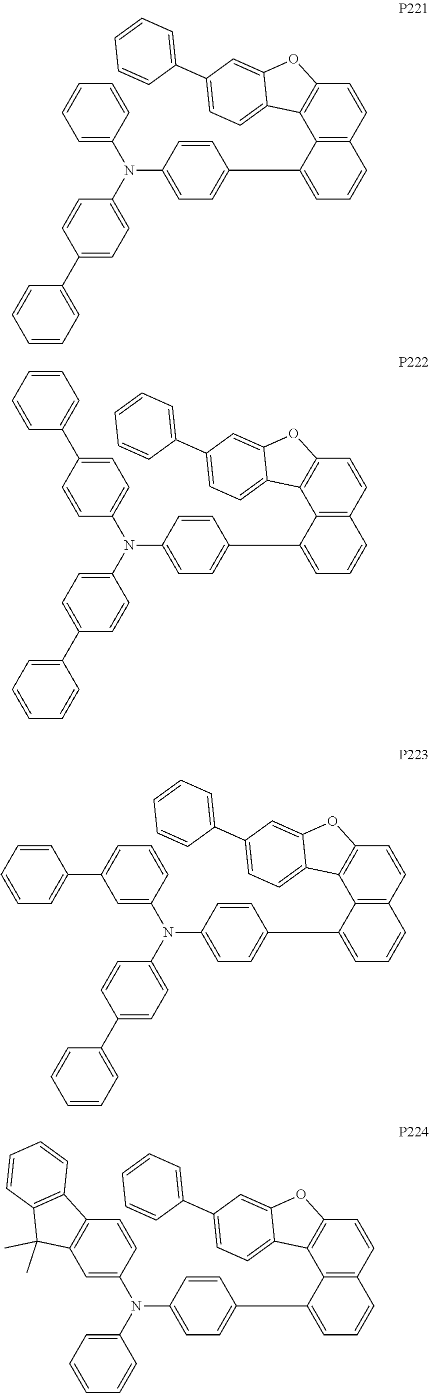

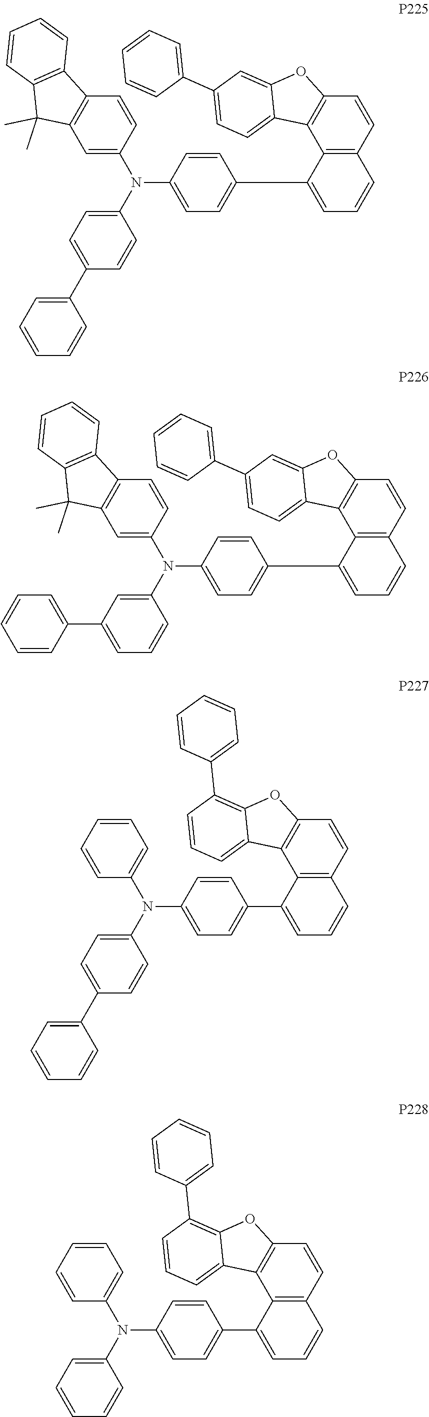

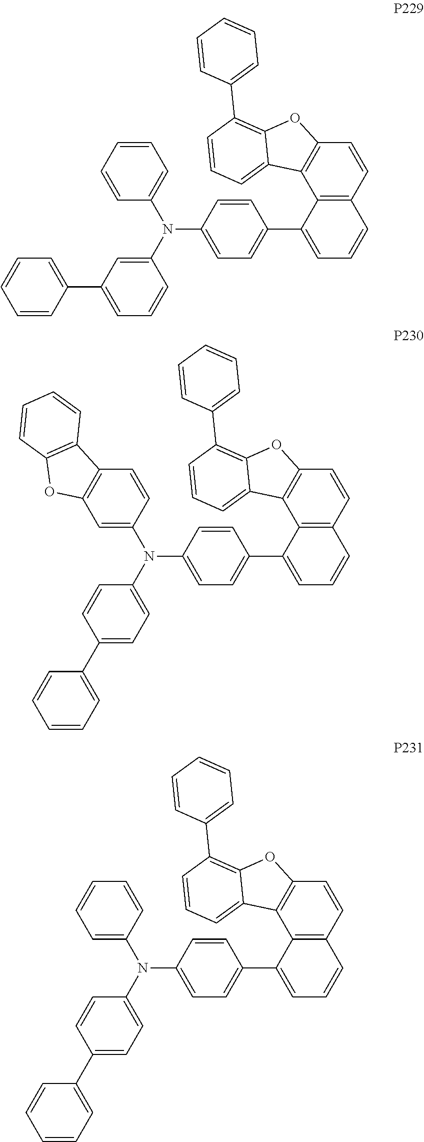 [SMILES: c1ccc(-c2cccc(N(c3ccccc3)c3ccc(-c4cccc5ccc6oc7c(-c8ccccc8)cccc7c6c45)cc3)c2)cc1 ; c1ccc(-c2ccc(N(c3ccc(-c4cccc5ccc6oc7c(-c8ccccc8)cccc7c6c45)cc3)c3ccc4c(c3)oc3ccccc34)cc2)cc1 ; c1ccc(-c2ccc(N(c3ccccc3)c3ccc(-c4cccc5ccc6oc7c(-c8ccccc8)cccc7c6c45)cc3)cc2)cc1]

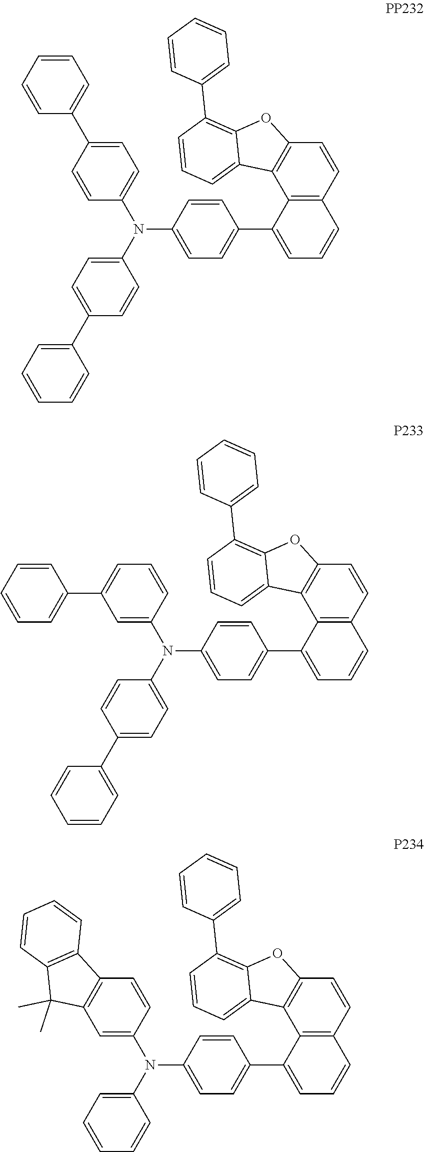 [SMILES: c1ccc(-c2ccc(N(c3ccc(-c4ccccc4)cc3)c3ccc(-c4cccc5ccc6oc7c(-c8ccccc8)cccc7c6c45)cc3)cc2)cc1 ; c1ccc(-c2ccc(N(c3ccc(-c4cccc5ccc6oc7c(-c8ccccc8)cccc7c6c45)cc3)c3cccc(-c4ccccc4)c3)cc2)cc1 ; CC1(C)c2ccccc2-c2ccc(N(c3ccccc3)c3ccc(-c4cccc5ccc6oc7c(-c8ccccc8)cccc7c6c45)cc3)cc21]

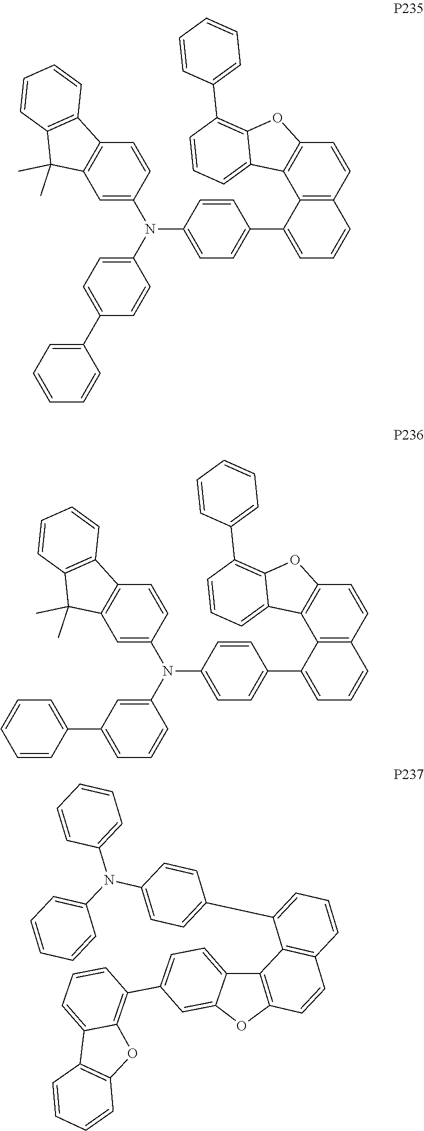

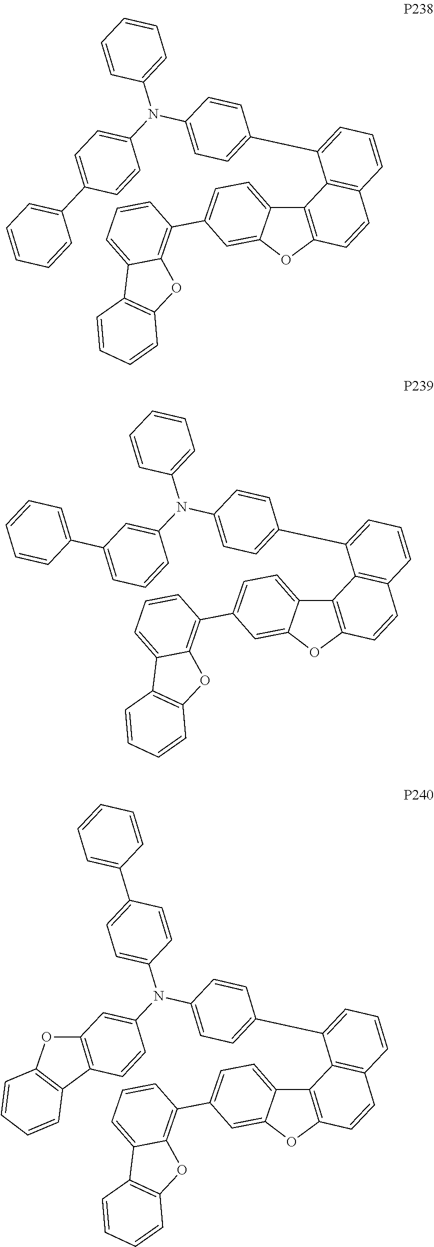 [SMILES: c1ccc(-c2ccc(N(c3ccccc3)c3ccc(-c4cccc5ccc6oc7cc(-c8cccc9c8oc8ccccc89)ccc7c6c45)cc3)cc2)cc1 ; c1ccc(-c2cccc(N(c3ccccc3)c3ccc(-c4cccc5ccc6oc7cc(-c8cccc9c8oc8ccccc89)ccc7c6c45)cc3)c2)cc1 ; c1ccc(-c2ccc(N(c3ccc(-c4cccc5ccc6oc7cc(-c8cccc9c8oc8ccccc89)ccc7c6c45)cc3)c3ccc4c(c3)oc3ccccc34)cc2)cc1]

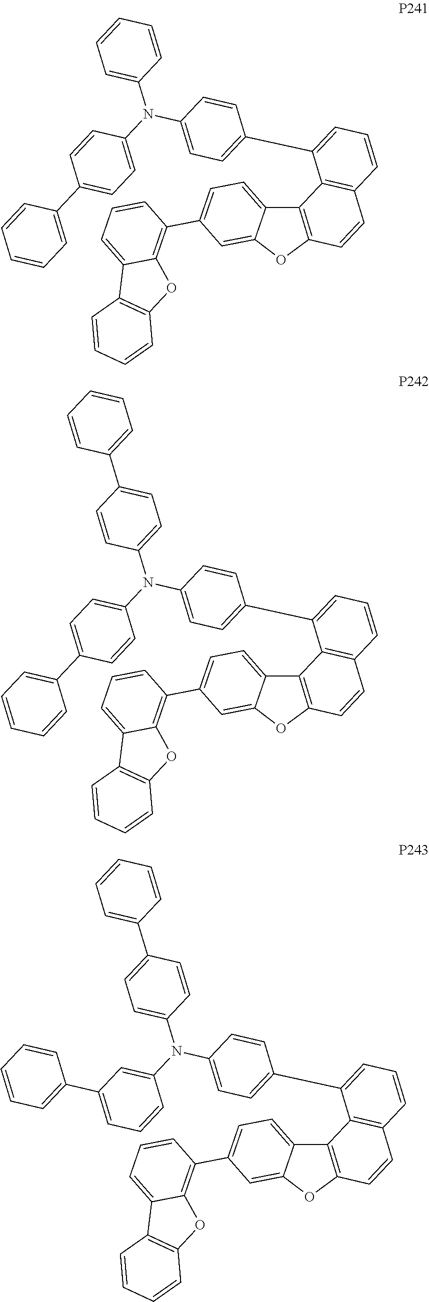

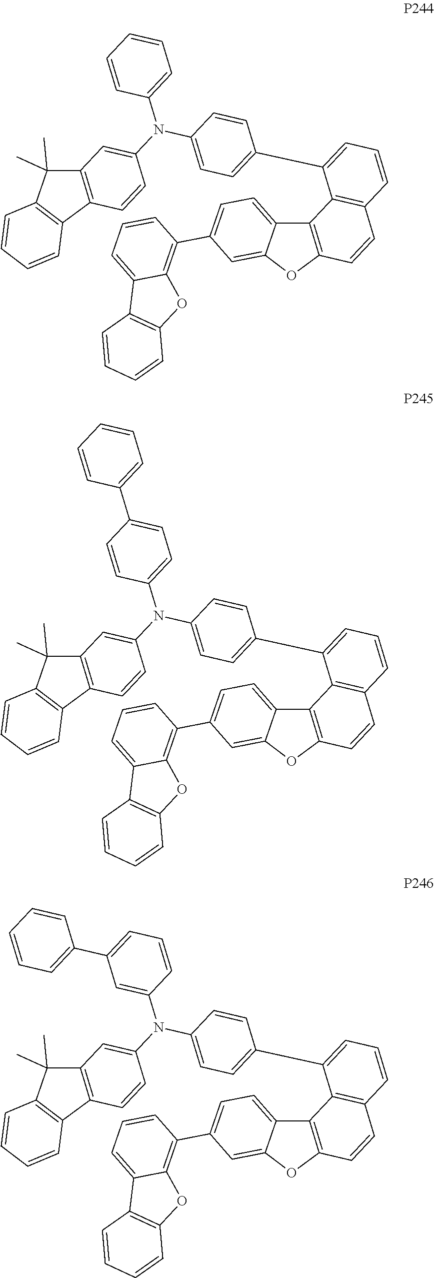 [SMILES: CC1(C)c2ccccc2-c2ccc(N(c3ccccc3)c3ccc(-c4cccc5ccc6oc7cc(-c8cccc9c8oc8ccccc89)ccc7c6c45)cc3)cc21 ; CC1(C)c2ccccc2-c2ccc(N(c3ccc(-c4ccccc4)cc3)c3ccc(-c4cccc5ccc6oc7cc(-c8cccc9c8oc8ccccc89)ccc7c6c45)cc3)cc21 ; CC1(C)c2ccccc2-c2ccc(N(c3ccc(-c4cccc5ccc6oc7cc(-c8cccc9c8oc8ccccc89)ccc7c6c45)cc3)c3cccc(-c4ccccc4)c3)cc21]

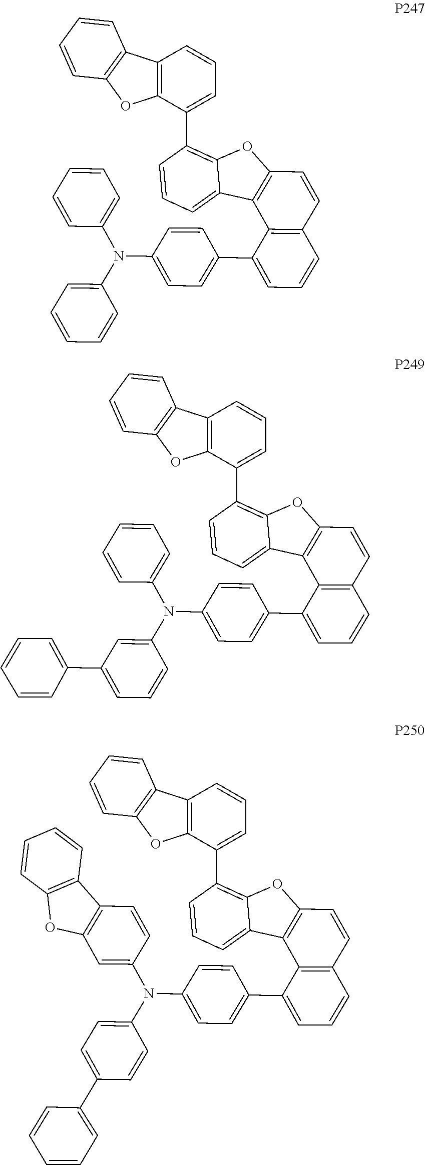

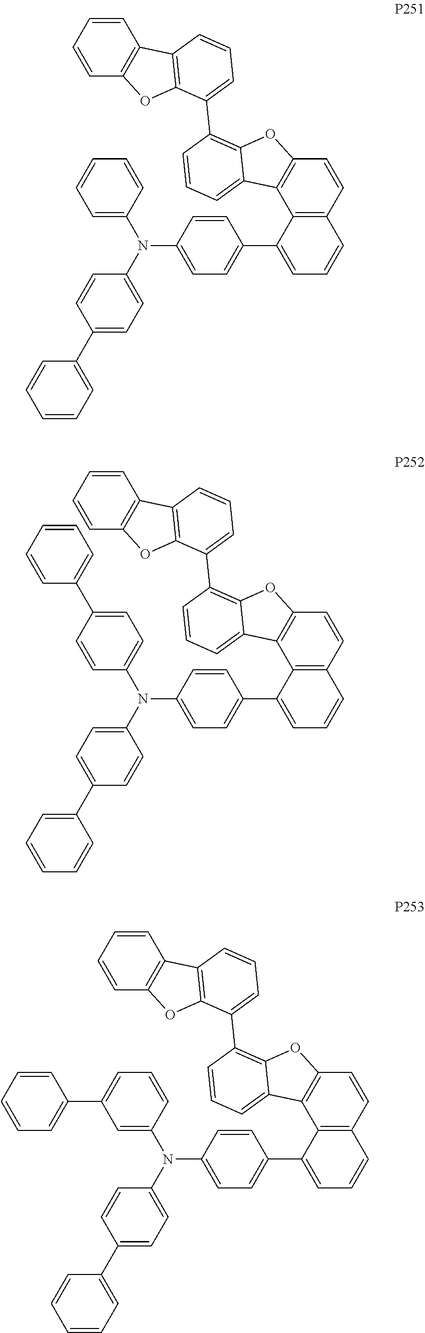 [SMILES: c1ccc(-c2ccc(N(c3ccccc3)c3ccc(-c4cccc5ccc6oc7c(-c8cccc9c8oc8ccccc89)cccc7c6c45)cc3)cc2)cc1 ; c1ccc(-c2ccc(N(c3ccc(-c4ccccc4)cc3)c3ccc(-c4cccc5ccc6oc7c(-c8cccc9c8oc8ccccc89)cccc7c6c45)cc3)cc2)cc1 ; c1ccc(-c2ccc(N(c3ccc(-c4cccc5ccc6oc7c(-c8cccc9c8oc8ccccc89)cccc7c6c45)cc3)c3cccc(-c4ccccc4)c3)cc2)cc1]

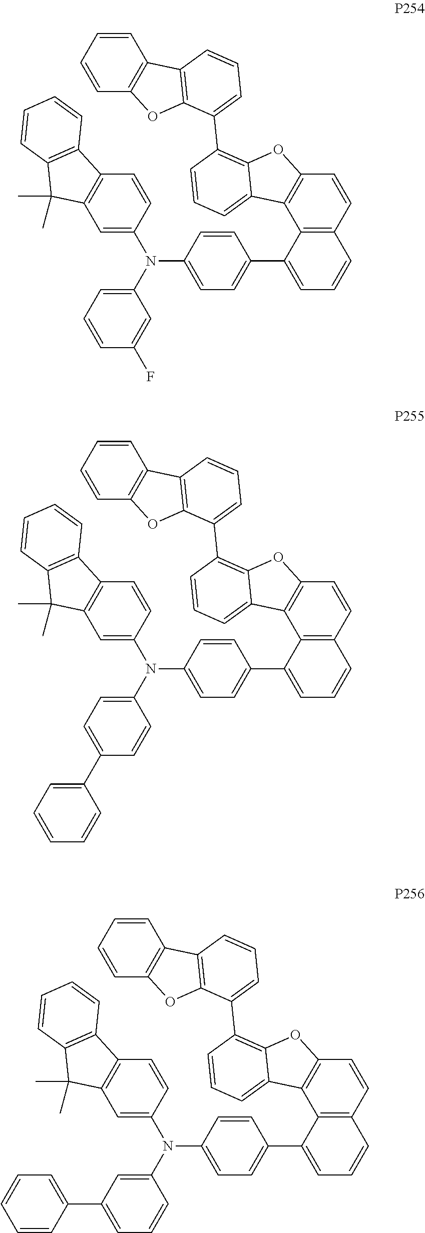 [SMILES: CC1(C)c2ccccc2-c2ccc(N(c3ccc(-c4cccc5ccc6oc7c(-c8cccc9c8oc8ccccc89)cccc7c6c45)cc3)c3cccc(F)c3)cc21 ; CC1(C)c2ccccc2-c2ccc(N(c3ccc(-c4ccccc4)cc3)c3ccc(-c4cccc5ccc6oc7c(-c8cccc9c8oc8ccccc89)cccc7c6c45)cc3)cc21 ; CC1(C)c2ccccc2-c2ccc(N(c3ccc(-c4cccc5ccc6oc7c(-c8cccc9c8oc8ccccc89)cccc7c6c45)cc3)c3cccc(-c4ccccc4)c3)cc21]

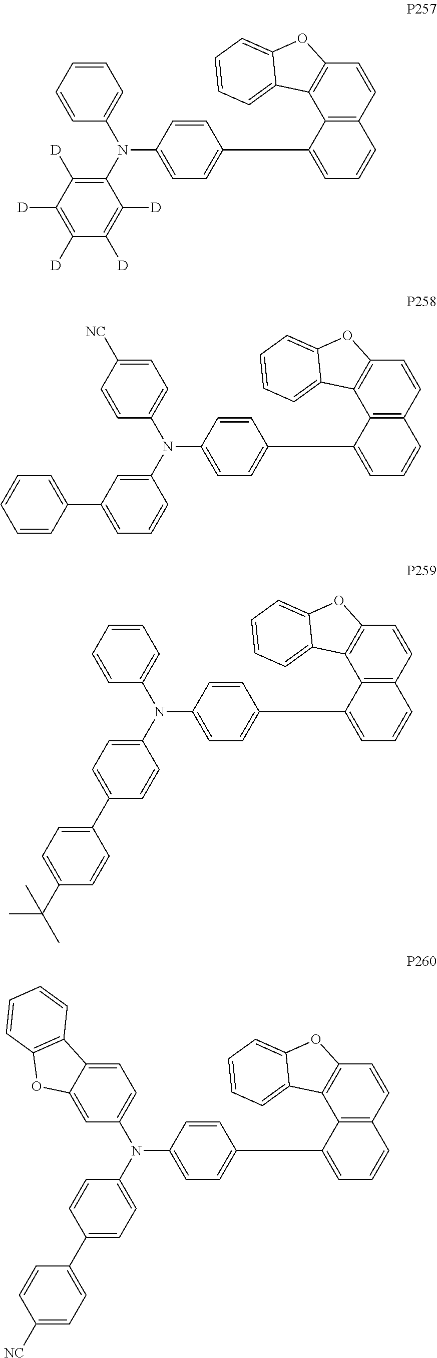 [SMILES: [2H]c1c([2H])c([2H])c(N(c2ccccc2)c2ccc(-c3cccc4ccc5oc6ccccc6c5c34)cc2)c([2H])c1[2H] ; N#Cc1ccc(N(c2ccc(-c3cccc4ccc5oc6ccccc6c5c34)cc2)c2cccc(-c3ccccc3)c2)cc1 ; CC(C)(C)c1ccc(-c2ccc(N(c3ccccc3)c3ccc(-c4cccc5ccc6oc7ccccc7c6c45)cc3)cc2)cc1 ; N#Cc1ccc(-c2ccc(N(c3ccc(-c4cccc5ccc6oc7ccccc7c6c45)cc3)c3ccc4c(c3)oc3ccccc34)cc2)cc1]

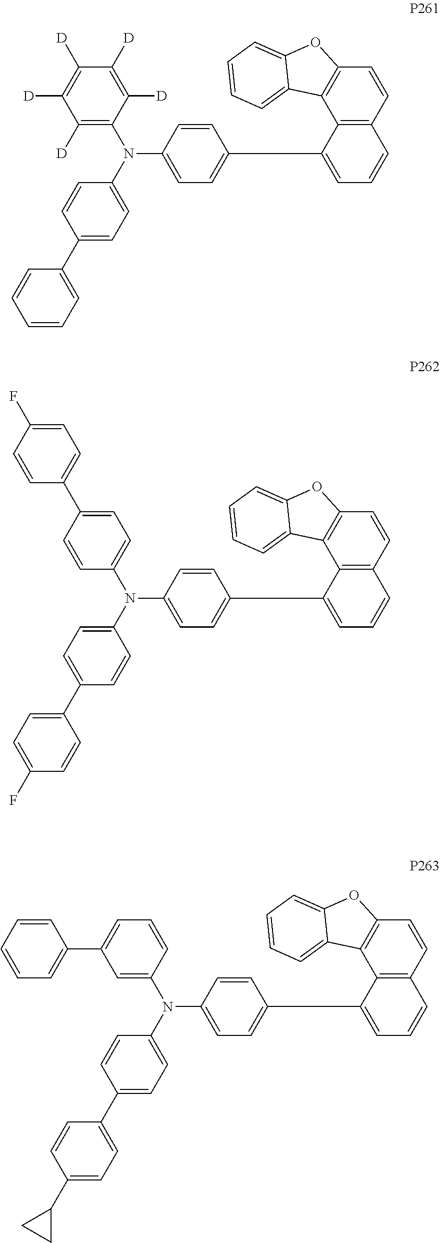 [SMILES: [2H]c1c([2H])c([2H])c(N(c2ccc(-c3ccccc3)cc2)c2ccc(-c3cccc4ccc5oc6ccccc6c5c34)cc2)c([2H])c1[2H] ; Fc1ccc(-c2ccc(N(c3ccc(-c4ccc(F)cc4)cc3)c3ccc(-c4cccc5ccc6oc7ccccc7c6c45)cc3)cc2)cc1 ; c1ccc(-c2cccc(N(c3ccc(-c4ccc(C5CC5)cc4)cc3)c3ccc(-c4cccc5ccc6oc7ccccc7c6c45)cc3)c2)cc1]

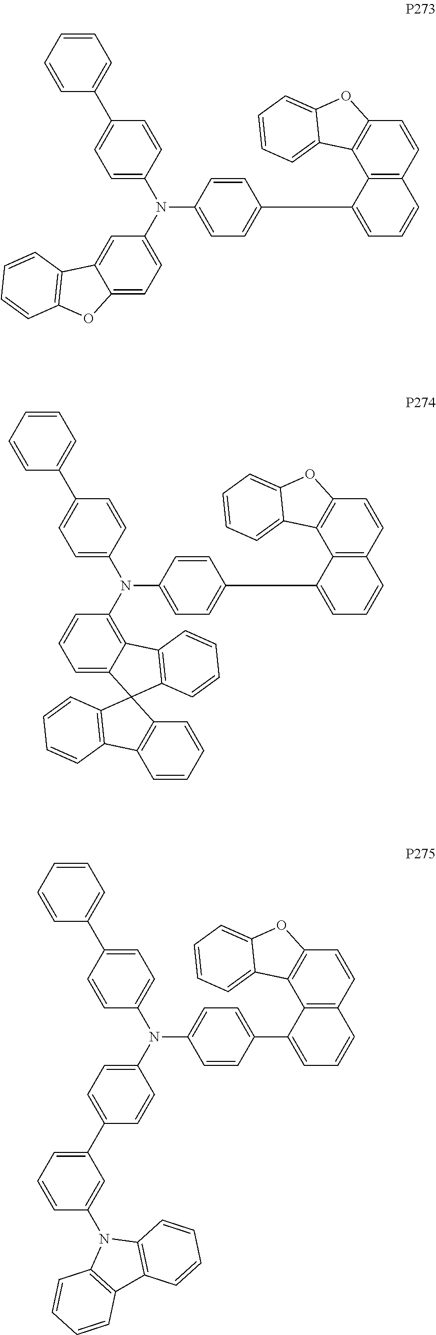 [SMILES: c1ccc(-c2ccc(N(c3ccc(-c4cccc5ccc6oc7ccccc7c6c45)cc3)c3ccc4oc5ccccc5c4c3)cc2)cc1 ; c1ccc(-c2ccc(N(c3ccc(-c4cccc5ccc6oc7ccccc7c6c45)cc3)c3cccc4c3-c3ccccc3C43c4ccccc4-c4ccccc43)cc2)cc1 ; c1ccc(-c2ccc(N(c3ccc(-c4cccc(-n5c6ccccc6c6ccccc65)c4)cc3)c3ccc(-c4cccc5ccc6oc7ccccc7c6c45)cc3)cc2)cc1]

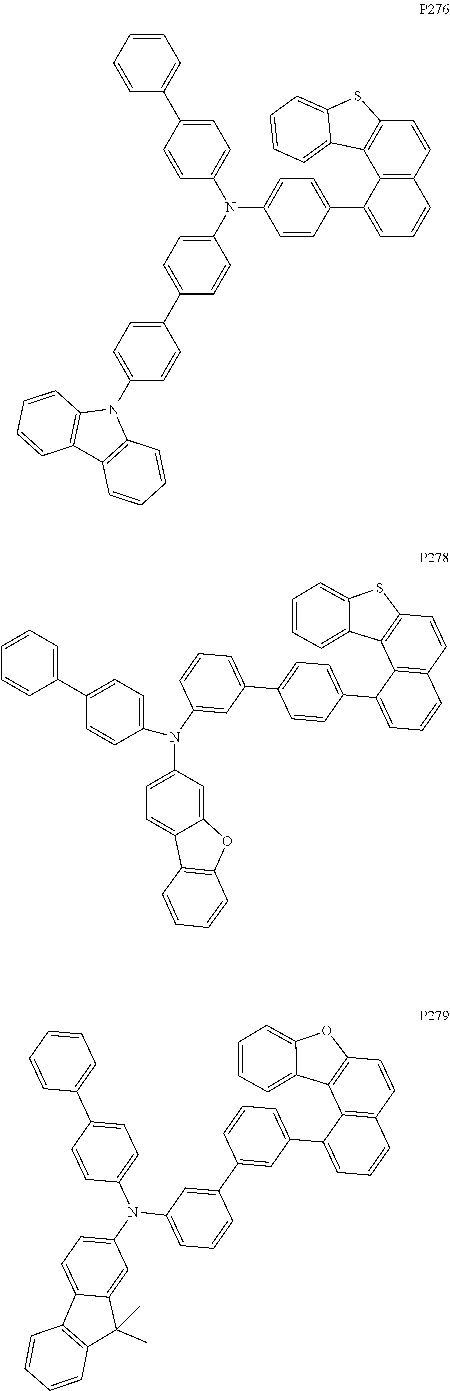 [SMILES: c1ccc(-c2ccc(N(c3ccc(-c4ccc(-n5c6ccccc6c6ccccc65)cc4)cc3)c3ccc(-c4cccc5ccc6sc7ccccc7c6c45)cc3)cc2)cc1 ; c1ccc(-c2ccc(N(c3cccc(-c4ccc(-c5cccc6ccc7sc8ccccc8c7c56)cc4)c3)c3ccc4c(c3)oc3ccccc34)cc2)cc1 ; CC1(C)c2ccccc2-c2ccc(N(c3ccc(-c4ccccc4)cc3)c3cccc(-c4cccc(-c5cccc6ccc7oc8ccccc8c7c56)c4)c3)cc21]

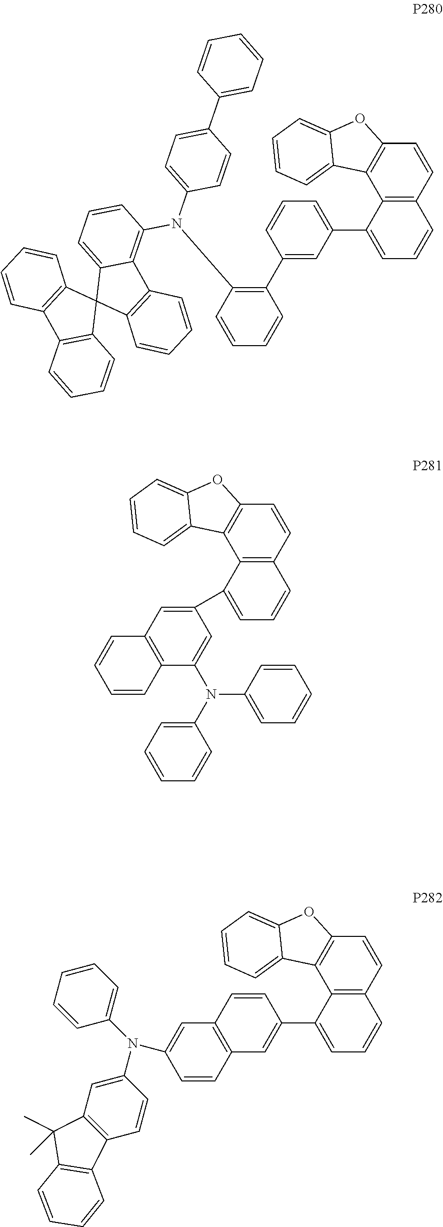 [SMILES: c1ccc(-c2ccc(N(c3ccccc3-c3cccc(-c4cccc5ccc6oc7ccccc7c6c45)c3)c3cccc4c3-c3ccccc3C43c4ccccc4-c4ccccc43)cc2)cc1 ; c1ccc(N(c2ccccc2)c2cc(-c3cccc4ccc5oc6ccccc6c5c34)cc3ccccc23)cc1 ; CC1(C)c2ccccc2-c2ccc(N(c3ccccc3)c3ccc4cc(-c5cccc6ccc7oc8ccccc8c7c56)ccc4c3)cc21]

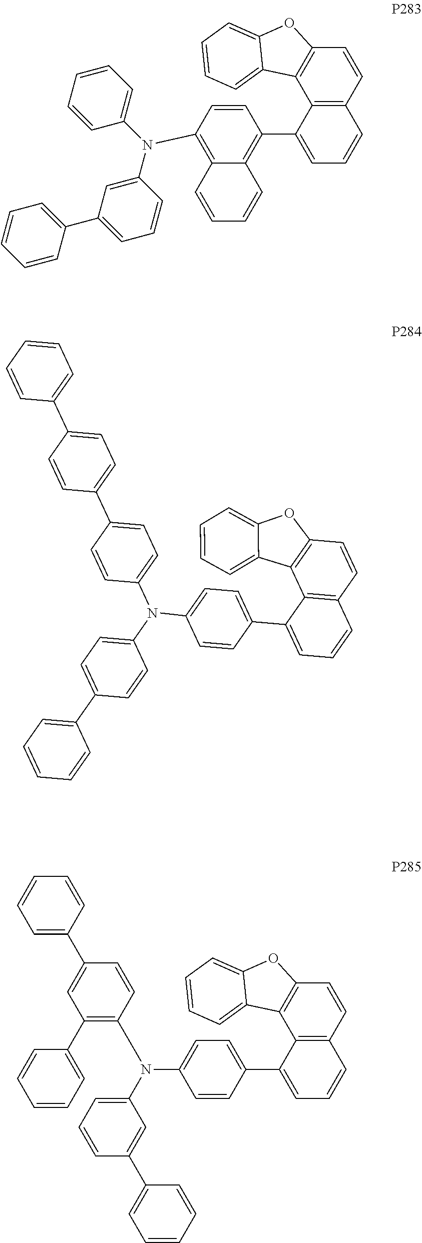 [SMILES: c1ccc(-c2cccc(N(c3ccccc3)c3ccc(-c4cccc5ccc6oc7ccccc7c6c45)c4ccccc34)c2)cc1 ; c1ccc(-c2ccc(-c3ccc(N(c4ccc(-c5ccccc5)cc4)c4ccc(-c5cccc6ccc7oc8ccccc8c7c56)cc4)cc3)cc2)cc1 ; c1ccc(-c2cccc(N(c3ccc(-c4cccc5ccc6oc7ccccc7c6c45)cc3)c3ccc(-c4ccccc4)cc3-c3ccccc3)c2)cc1]

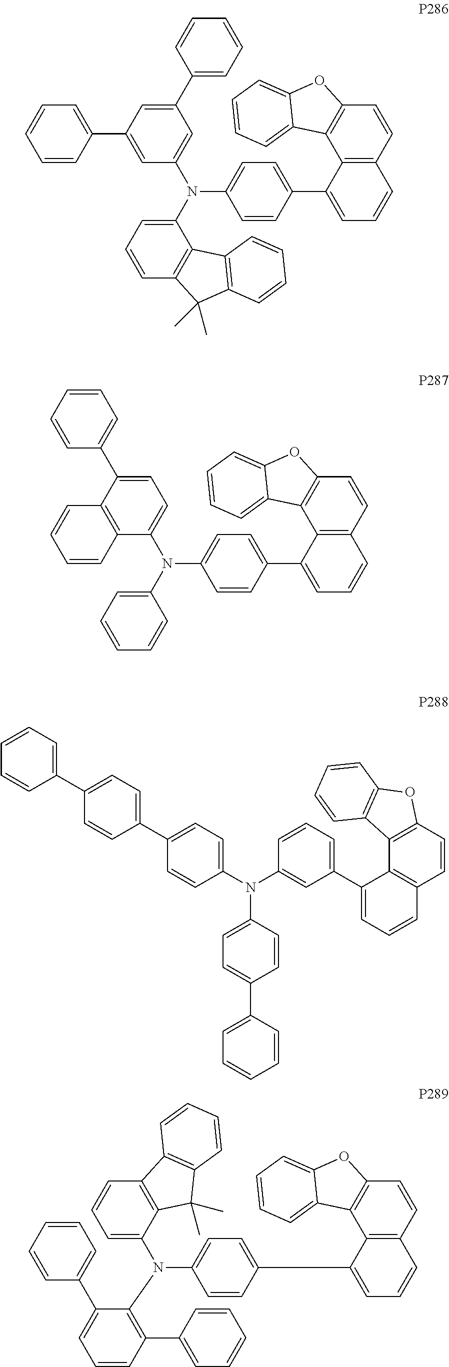 [SMILES: CC1(C)c2ccccc2-c2c(N(c3ccc(-c4cccc5ccc6oc7ccccc7c6c45)cc3)c3cc(-c4ccccc4)cc(-c4ccccc4)c3)cccc21 ; c1ccc(-c2ccc(N(c3ccccc3)c3ccc(-c4cccc5ccc6oc7ccccc7c6c45)cc3)c3ccccc23)cc1 ; c1ccc(-c2ccc(-c3ccc(N(c4ccc(-c5ccccc5)cc4)c4cccc(-c5cccc6ccc7oc8ccccc8c7c56)c4)cc3)cc2)cc1 ; CC1(C)c2ccccc2-c2cccc(N(c3ccc(-c4cccc5ccc6oc7ccccc7c6c45)cc3)c3c(-c4ccccc4)cccc3-c3ccccc3)c21]

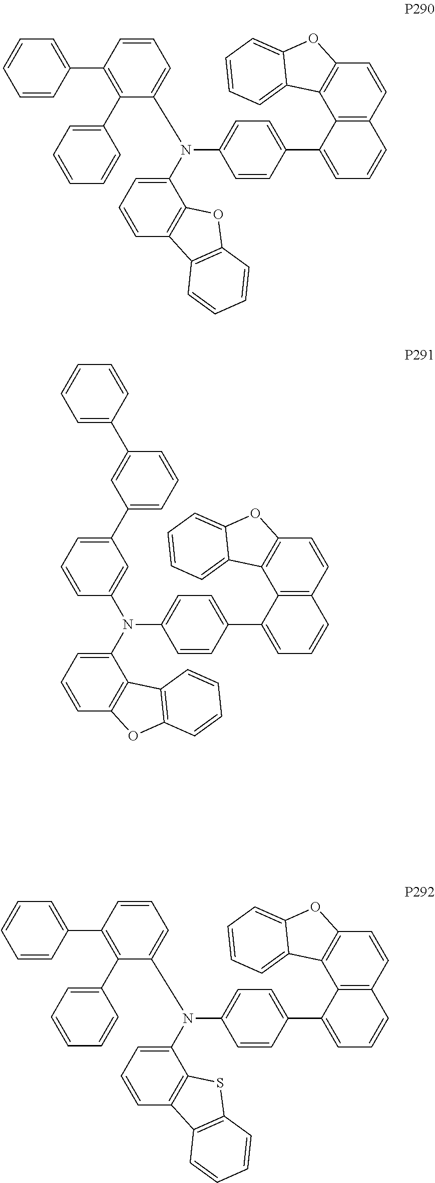 [SMILES: c1ccc(-c2cccc(N(c3ccc(-c4cccc5ccc6oc7ccccc7c6c45)cc3)c3cccc4c3oc3ccccc34)c2-c2ccccc2)cc1 ; c1ccc(-c2cccc(-c3cccc(N(c4ccc(-c5cccc6ccc7oc8ccccc8c7c56)cc4)c4cccc5oc6ccccc6c45)c3)c2)cc1 ; c1ccc(-c2cccc(N(c3ccc(-c4cccc5ccc6oc7ccccc7c6c45)cc3)c3cccc4c3sc3ccccc34)c2-c2ccccc2)cc1]

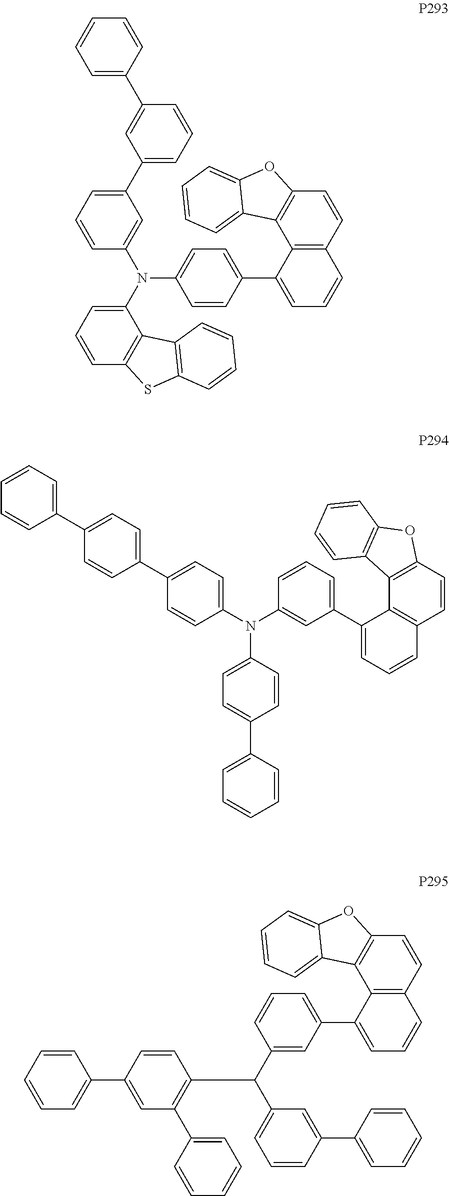 [SMILES: c1ccc(-c2cccc(-c3cccc(N(c4ccc(-c5cccc6ccc7oc8ccccc8c7c56)cc4)c4cccc5sc6ccccc6c45)c3)c2)cc1 ; c1ccc(-c2ccc(-c3ccc(N(c4ccc(-c5ccccc5)cc4)c4cccc(-c5cccc6ccc7oc8ccccc8c7c56)c4)cc3)cc2)cc1 ; c1ccc(-c2cccc(C(c3cccc(-c4cccc5ccc6oc7ccccc7c6c45)c3)c3ccc(-c4ccccc4)cc3-c3ccccc3)c2)cc1]

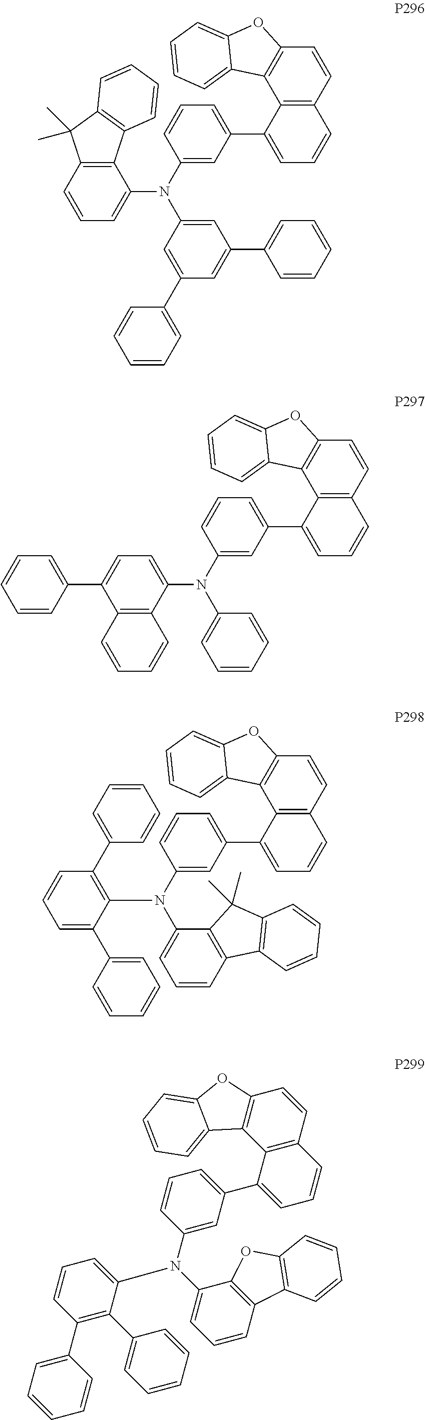 [SMILES: CC1(C)c2ccccc2-c2c(N(c3cc(-c4ccccc4)cc(-c4ccccc4)c3)c3cccc(-c4cccc5ccc6oc7ccccc7c6c45)c3)cccc21 ; c1ccc(-c2ccc(N(c3ccccc3)c3cccc(-c4cccc5ccc6oc7ccccc7c6c45)c3)c3ccccc23)cc1 ; CC1(C)c2ccccc2-c2cccc(N(c3cccc(-c4cccc5ccc6oc7ccccc7c6c45)c3)c3c(-c4ccccc4)cccc3-c3ccccc3)c21 ; c1ccc(-c2cccc(N(c3cccc(-c4cccc5ccc6oc7ccccc7c6c45)c3)c3cccc4c3oc3ccccc34)c2-c2ccccc2)cc1]

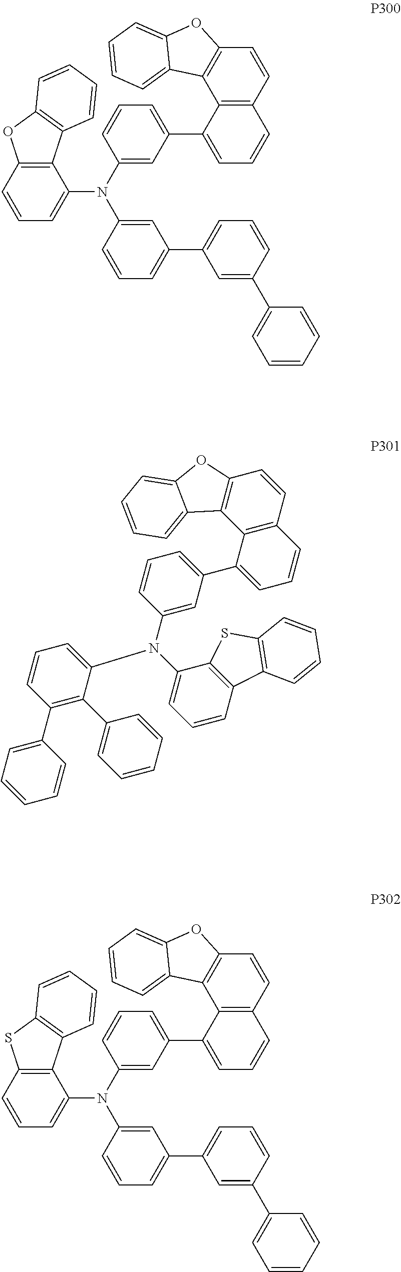 [SMILES: c1ccc(-c2cccc(-c3cccc(N(c4cccc(-c5cccc6ccc7oc8ccccc8c7c56)c4)c4cccc5oc6ccccc6c45)c3)c2)cc1 ; c1ccc(-c2cccc(N(c3cccc(-c4cccc5ccc6oc7ccccc7c6c45)c3)c3cccc4c3sc3ccccc34)c2-c2ccccc2)cc1 ; c1ccc(-c2cccc(-c3cccc(N(c4cccc(-c5cccc6ccc7oc8ccccc8c7c56)c4)c4cccc5sc6ccccc6c45)c3)c2)cc1]

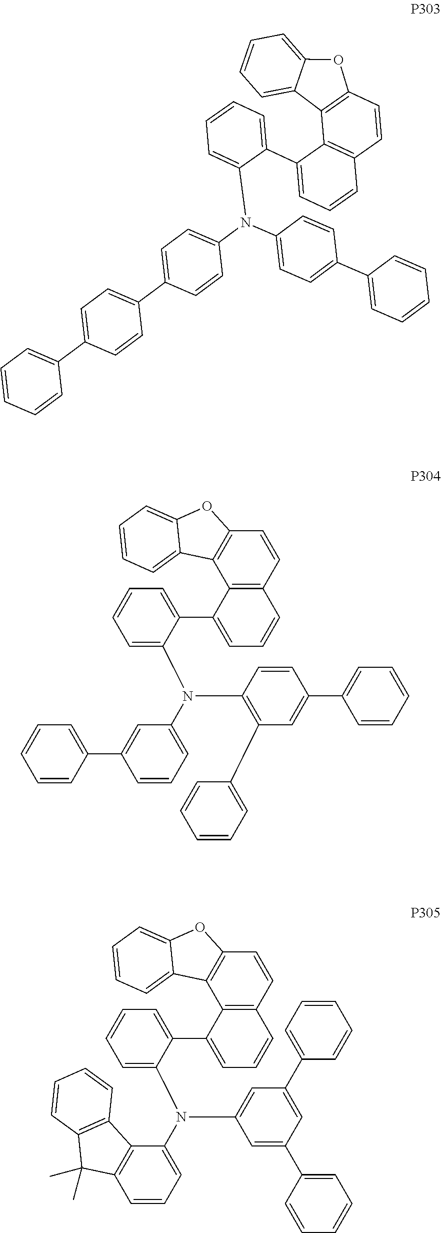 [SMILES: c1ccc(-c2ccc(-c3ccc(N(c4ccc(-c5ccccc5)cc4)c4ccccc4-c4cccc5ccc6oc7ccccc7c6c45)cc3)cc2)cc1 ; c1ccc(-c2cccc(N(c3ccc(-c4ccccc4)cc3-c3ccccc3)c3ccccc3-c3cccc4ccc5oc6ccccc6c5c34)c2)cc1 ; CC1(C)c2ccccc2-c2c(N(c3cc(-c4ccccc4)cc(-c4ccccc4)c3)c3ccccc3-c3cccc4ccc5oc6ccccc6c5c34)cccc21]

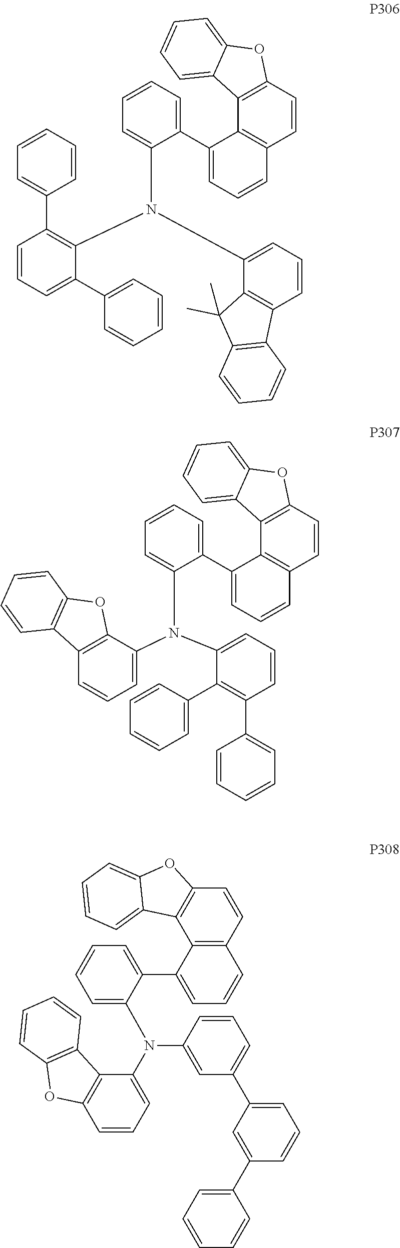

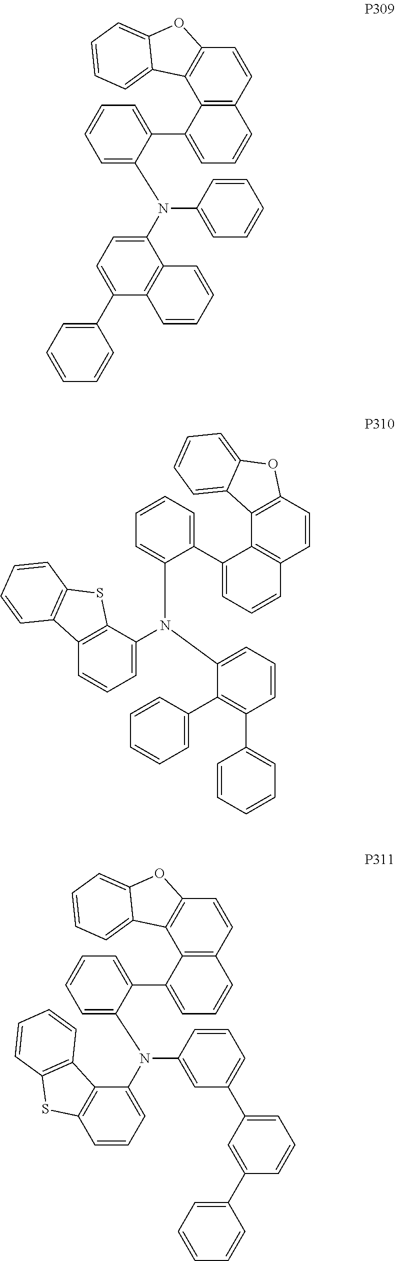 [SMILES: c1ccc(-c2ccc(N(c3ccccc3)c3ccccc3-c3cccc4ccc5oc6ccccc6c5c34)c3ccccc23)cc1 ; c1ccc(-c2cccc(N(c3ccccc3-c3cccc4ccc5oc6ccccc6c5c34)c3cccc4c3sc3ccccc34)c2-c2ccccc2)cc1 ; c1ccc(-c2cccc(-c3cccc(N(c4ccccc4-c4cccc5ccc6oc7ccccc7c6c45)c4cccc5sc6ccccc6c45)c3)c2)cc1]

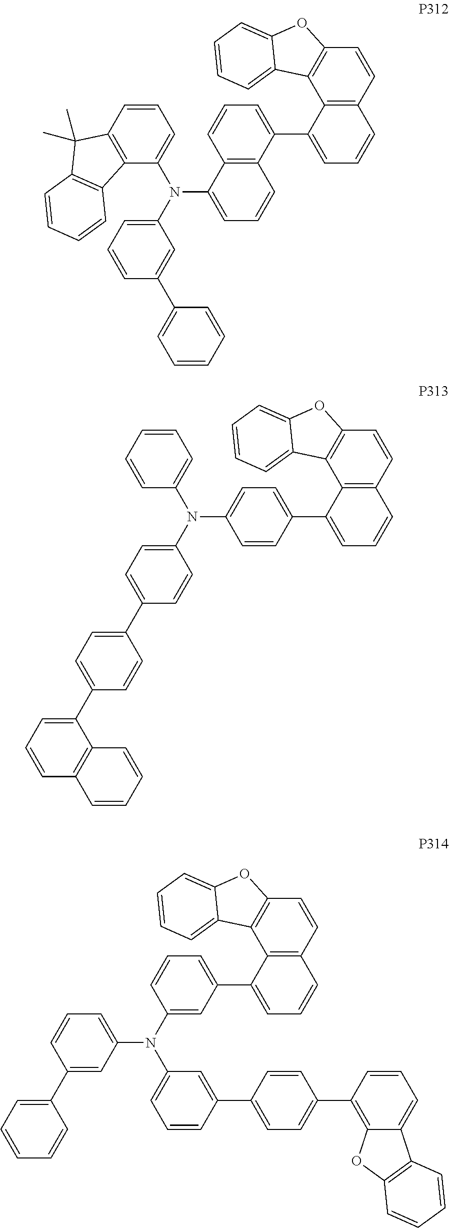 [SMILES: CC1(C)c2ccccc2-c2c(N(c3cccc(-c4ccccc4)c3)c3cccc4c(-c5cccc6ccc7oc8ccccc8c7c56)cccc34)cccc21 ; c1ccc(N(c2ccc(-c3ccc(-c4cccc5ccccc45)cc3)cc2)c2ccc(-c3cccc4ccc5oc6ccccc6c5c34)cc2)cc1 ; c1ccc(-c2cccc(N(c3cccc(-c4ccc(-c5cccc6c5oc5ccccc56)cc4)c3)c3cccc(-c4cccc5ccc6oc7ccccc7c6c45)c3)c2)cc1]

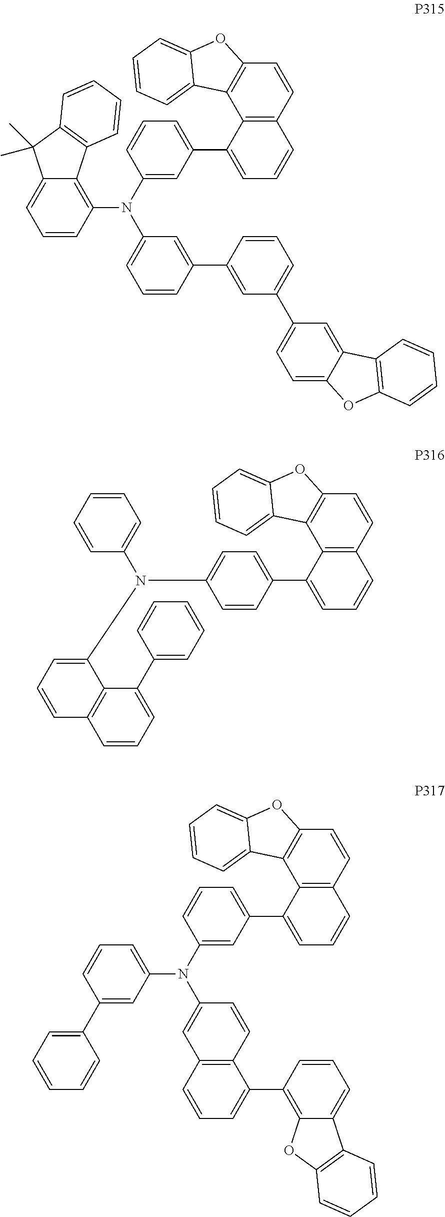 [SMILES: CC1(C)c2ccccc2-c2c(N(c3cccc(-c4cccc(-c5ccc6oc7ccccc7c6c5)c4)c3)c3cccc(-c4cccc5ccc6oc7ccccc7c6c45)c3)cccc21 ; c1ccc(-c2cccc3cccc(N(c4ccccc4)c4ccc(-c5cccc6ccc7oc8ccccc8c7c56)cc4)c23)cc1 ; c1ccc(-c2cccc(N(c3cccc(-c4cccc5ccc6oc7ccccc7c6c45)c3)c3ccc4c(-c5cccc6c5oc5ccccc56)cccc4c3)c2)cc1]

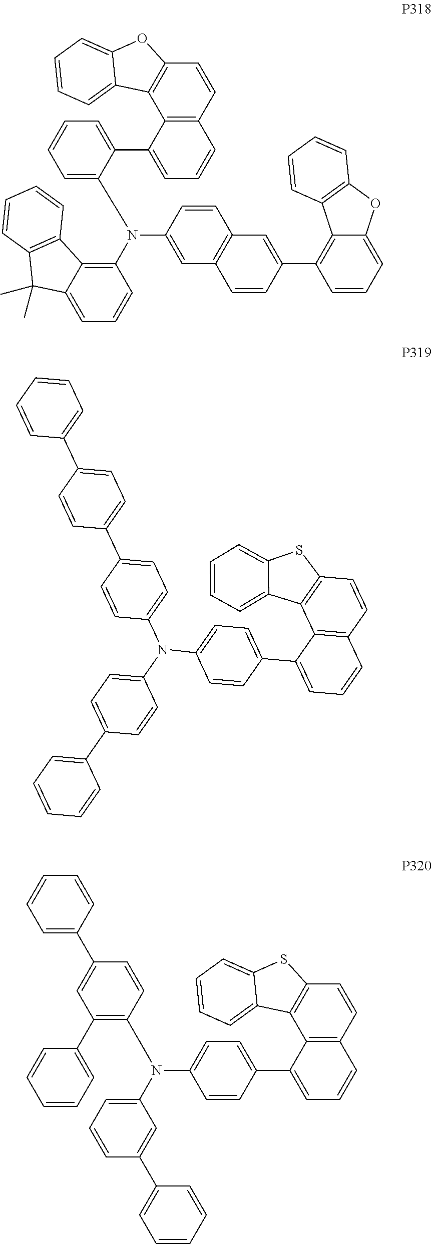 [SMILES: CC1(C)c2ccccc2-c2c(N(c3ccc4cc(-c5cccc6oc7ccccc7c56)ccc4c3)c3ccccc3-c3cccc4ccc5oc6ccccc6c5c34)cccc21 ; c1ccc(-c2ccc(-c3ccc(N(c4ccc(-c5ccccc5)cc4)c4ccc(-c5cccc6ccc7sc8ccccc8c7c56)cc4)cc3)cc2)cc1 ; c1ccc(-c2cccc(N(c3ccc(-c4cccc5ccc6sc7ccccc7c6c45)cc3)c3ccc(-c4ccccc4)cc3-c3ccccc3)c2)cc1]

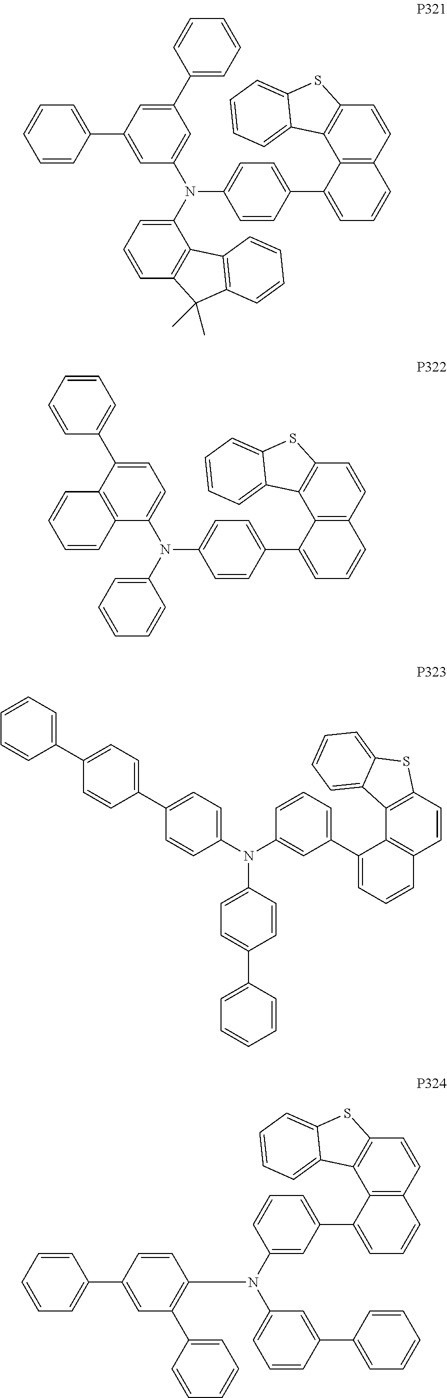 [SMILES: CC1(C)c2ccccc2-c2c(N(c3ccc(-c4cccc5ccc6sc7ccccc7c6c45)cc3)c3cc(-c4ccccc4)cc(-c4ccccc4)c3)cccc21 ; c1ccc(-c2ccc(N(c3ccccc3)c3ccc(-c4cccc5ccc6sc7ccccc7c6c45)cc3)c3ccccc23)cc1 ; c1ccc(-c2ccc(-c3ccc(N(c4ccc(-c5ccccc5)cc4)c4cccc(-c5cccc6ccc7sc8ccccc8c7c56)c4)cc3)cc2)cc1 ; c1ccc(-c2cccc(N(c3cccc(-c4cccc5ccc6sc7ccccc7c6c45)c3)c3ccc(-c4ccccc4)cc3-c3ccccc3)c2)cc1]

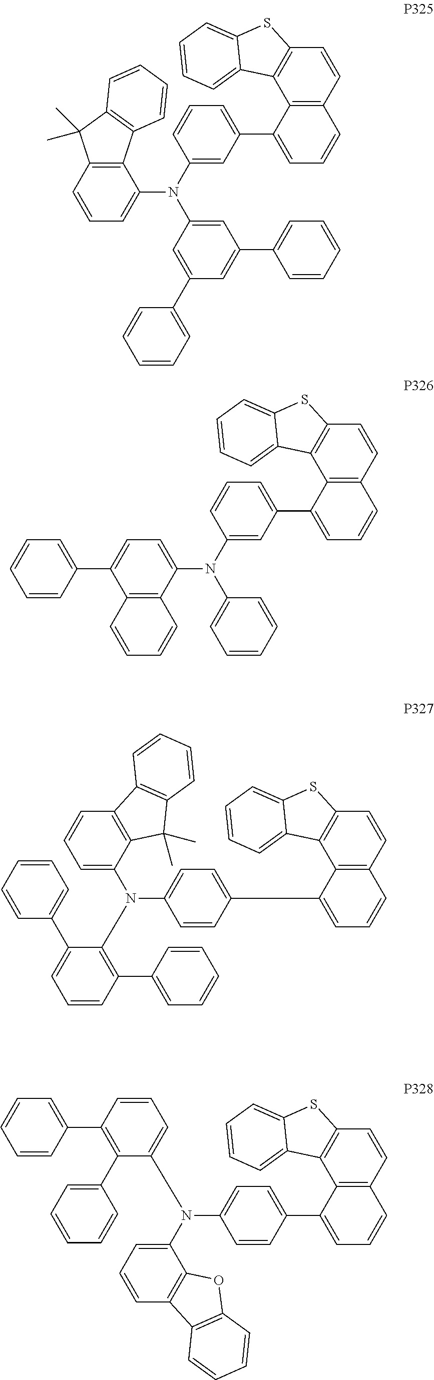 [SMILES: CC1(C)c2ccccc2-c2c(N(c3cc(-c4ccccc4)cc(-c4ccccc4)c3)c3cccc(-c4cccc5ccc6sc7ccccc7c6c45)c3)cccc21 ; c1ccc(-c2ccc(N(c3ccccc3)c3cccc(-c4cccc5ccc6sc7ccccc7c6c45)c3)c3ccccc23)cc1 ; CC1(C)c2ccccc2-c2cccc(N(c3ccc(-c4cccc5ccc6sc7ccccc7c6c45)cc3)c3c(-c4ccccc4)cccc3-c3ccccc3)c21 ; c1ccc(-c2cccc(N(c3ccc(-c4cccc5ccc6sc7ccccc7c6c45)cc3)c3cccc4c3oc3ccccc34)c2-c2ccccc2)cc1]

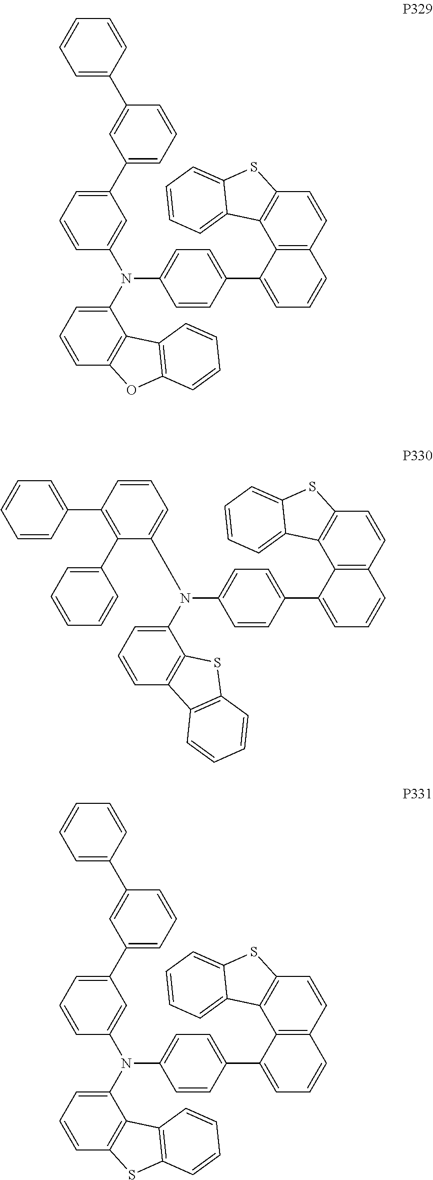 [SMILES: c1ccc(-c2cccc(-c3cccc(N(c4ccc(-c5cccc6ccc7sc8ccccc8c7c56)cc4)c4cccc5oc6ccccc6c45)c3)c2)cc1 ; c1ccc(-c2cccc(N(c3ccc(-c4cccc5ccc6sc7ccccc7c6c45)cc3)c3cccc4c3sc3ccccc34)c2-c2ccccc2)cc1 ; c1ccc(-c2cccc(-c3cccc(N(c4ccc(-c5cccc6ccc7sc8ccccc8c7c56)cc4)c4cccc5sc6ccccc6c45)c3)c2)cc1]

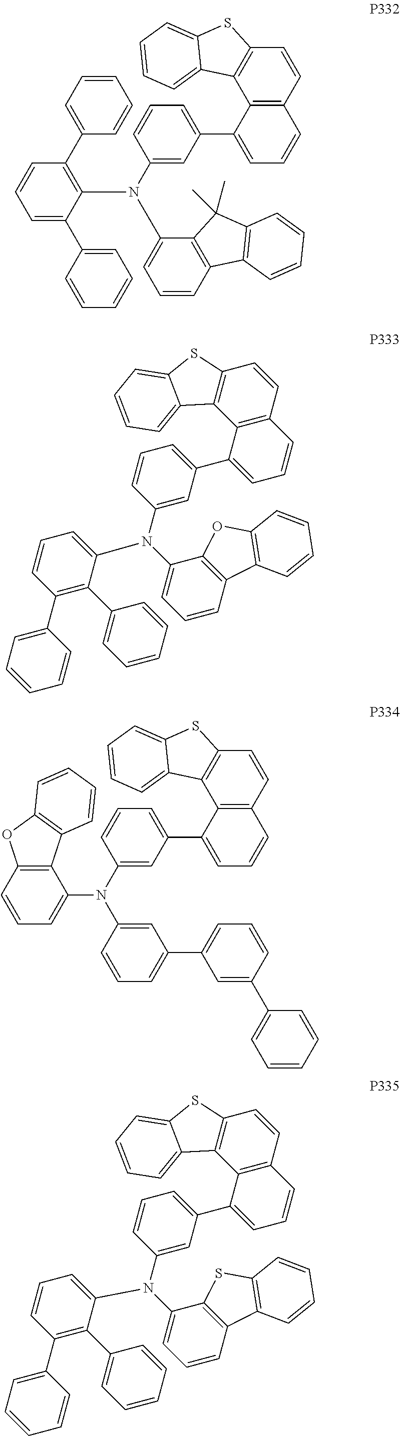

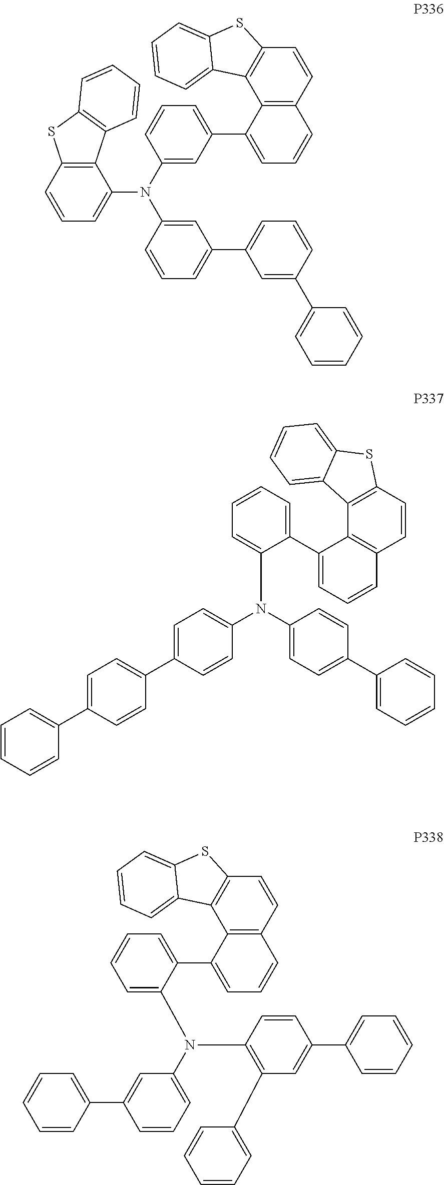 [SMILES: c1ccc(-c2cccc(-c3cccc(N(c4cccc(-c5cccc6ccc7sc8ccccc8c7c56)c4)c4cccc5sc6ccccc6c45)c3)c2)cc1 ; c1ccc(-c2ccc(-c3ccc(N(c4ccc(-c5ccccc5)cc4)c4ccccc4-c4cccc5ccc6sc7ccccc7c6c45)cc3)cc2)cc1 ; c1ccc(-c2cccc(N(c3ccc(-c4ccccc4)cc3-c3ccccc3)c3ccccc3-c3cccc4ccc5sc6ccccc6c5c34)c2)cc1]

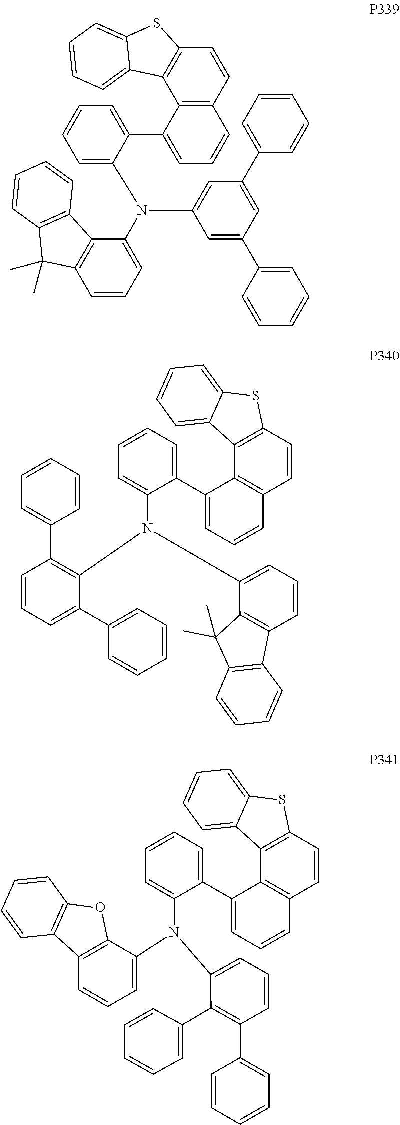

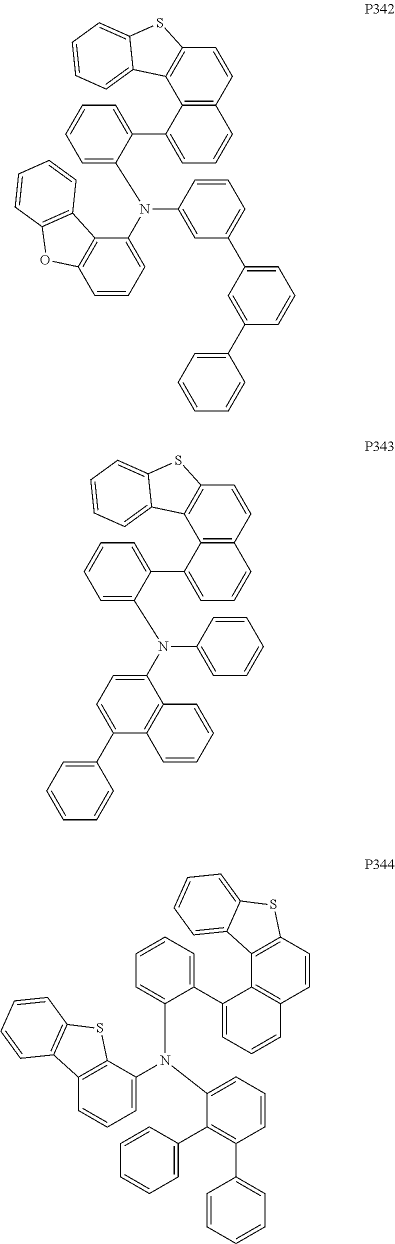 [SMILES: c1ccc(-c2cccc(-c3cccc(N(c4ccccc4-c4cccc5ccc6sc7ccccc7c6c45)c4cccc5oc6ccccc6c45)c3)c2)cc1 ; c1ccc(-c2ccc(N(c3ccccc3)c3ccccc3-c3cccc4ccc5sc6ccccc6c5c34)c3ccccc23)cc1 ; c1ccc(-c2cccc(N(c3ccccc3-c3cccc4ccc5sc6ccccc6c5c34)c3cccc4c3sc3ccccc34)c2-c2ccccc2)cc1]

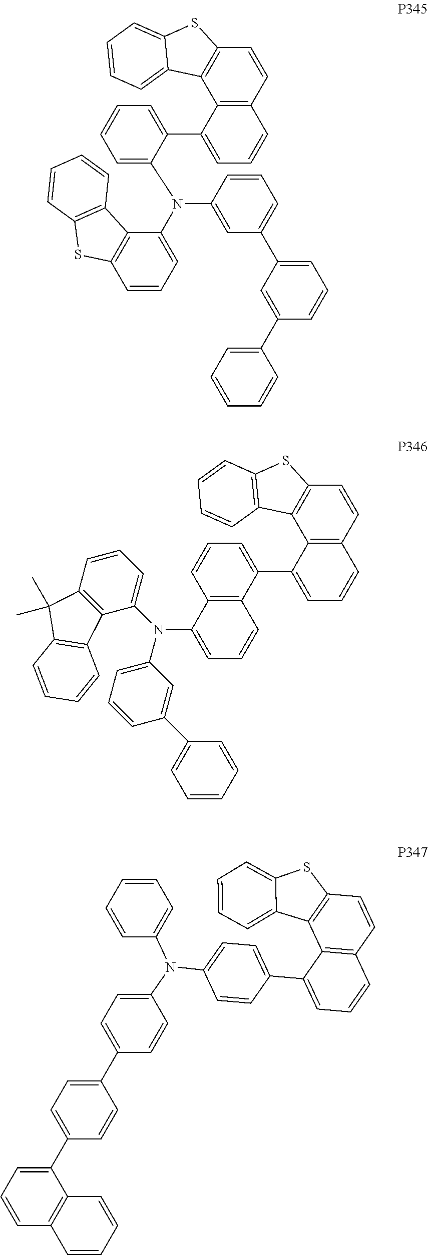 [SMILES: c1ccc(-c2cccc(-c3cccc(N(c4ccccc4-c4cccc5ccc6sc7ccccc7c6c45)c4cccc5sc6ccccc6c45)c3)c2)cc1 ; CC1(C)c2ccccc2-c2c(N(c3cccc(-c4ccccc4)c3)c3cccc4c(-c5cccc6ccc7sc8ccccc8c7c56)cccc34)cccc21 ; c1ccc(N(c2ccc(-c3ccc(-c4cccc5ccccc45)cc3)cc2)c2ccc(-c3cccc4ccc5sc6ccccc6c5c34)cc2)cc1]

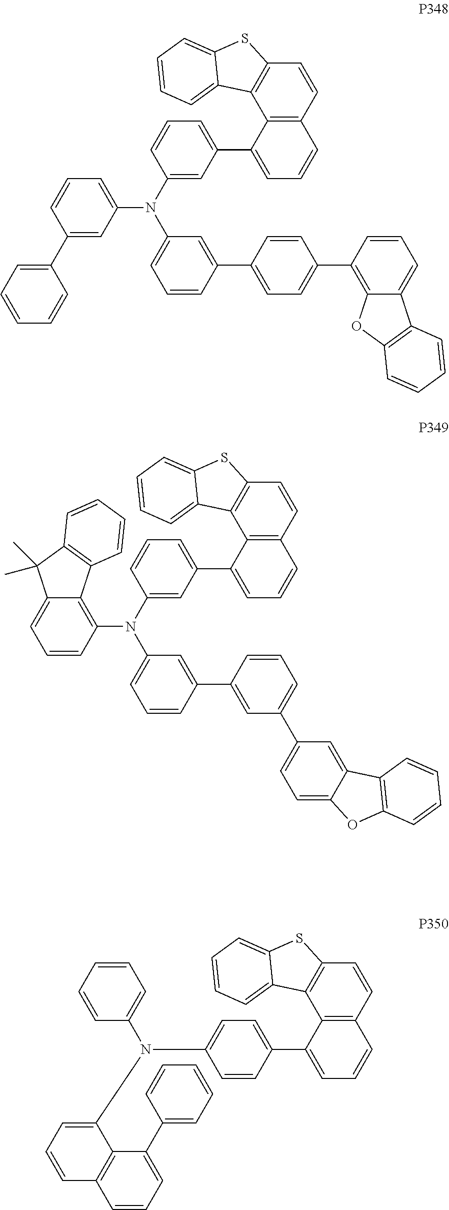 [SMILES: c1ccc(-c2cccc(N(c3cccc(-c4ccc(-c5cccc6c5oc5ccccc56)cc4)c3)c3cccc(-c4cccc5ccc6sc7ccccc7c6c45)c3)c2)cc1 ; CC1(C)c2ccccc2-c2c(N(c3cccc(-c4cccc(-c5ccc6oc7ccccc7c6c5)c4)c3)c3cccc(-c4cccc5ccc6sc7ccccc7c6c45)c3)cccc21 ; c1ccc(-c2cccc3cccc(N(c4ccccc4)c4ccc(-c5cccc6ccc7sc8ccccc8c7c56)cc4)c23)cc1]

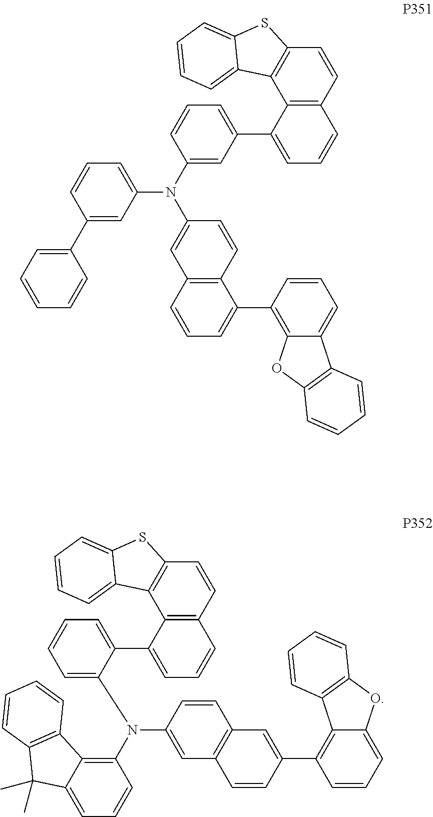 [SMILES: c1ccc(-c2cccc(N(c3cccc(-c4cccc5ccc6sc7ccccc7c6c45)c3)c3ccc4c(-c5cccc6c5oc5ccccc56)cccc4c3)c2)cc1 ; CC1(C)c2ccccc2-c2c(N(c3ccc4cc(-c5cccc6oc7ccccc7c56)ccc4c3)c3ccccc3-c3cccc4ccc5sc6ccccc6c5c34)cccc21]

26. An electronic element, comprising: an anode and a cathode that are arranged oppositely, and a functional layer arranged between the anode and the cathode, wherein the functional layer comprises the nitrogen-containing compound according to claim 18.

27. The electronic element according to claim 26, wherein the electronic element comprises a hole transport layer (HTL), and the HTL comprises the nitrogen-containing compound.

28. An electronic device comprising the electronic element according to claim 26.

29. An electronic device comprising the electronic element according to claim 27.
